SEQUENCE LISTING (371 sequences)

SEQ ID NO: 1 - DNA, 101 bp, Cannabis sativa
  source 1..101: mol_type = genomic DNA
aaataaataaaactaaagaaagctggcacgaggctttttcttgtgtactataaattagttaaatttgatatataaattttctctctctcttttcaagttga

SEQ ID NO: 2 - DNA, 101 bp, Cannabis sativa
  source 1..101: mol_type = genomic DNA
tggctttaatatcgtacattacaatattacattgtccaaactgcccccgagaccttttagcacattgccctgaatttggtaatcgaccaccattactgagc

SEQ ID NO: 3 - DNA, 101 bp, Cannabis sativa
  source 1..101: mol_type = genomic DNA
ggatcgaaatgatgagttagttggacgatccaaaagcgaaggagcacgttcgagttggttcactcgtcctgctacgtactcacctccgacagctcaaagta

SEQ ID NO: 4 - DNA, 101 bp, Cannabis sativa
  source 1..101: mol_type = genomic DNA
tcagccagcagctcagtcacggctccacccgagaagaaccaaacaacaatgcagcagcttctaacatggctggtaacacctcttttgttcaaaaattatct

SEQ ID NO: 5 - DNA, 101 bp, Cannabis sativa
  source 1..101: mol_type = genomic DNA
ttatgttcagaagttgaattgaggtgatttaagttgaattatgttaagtttgtggattttggtgatttttgggcaaggtttgagtttaagaactcaagctt

SEQ ID NO: 6 - DNA, 101 bp, Cannabis sativa
  source 1..101: mol_type = genomic DNA
acttgttgtatgaaaaatgaatgagctttggaaagaggaatcagttgtaagaagatgtgactgaggttgtagaaagaatatttcaaaaagacaattacatt

SEQ ID NO: 7 - DNA, 101 bp, Cannabis sativa
  source 1..101: mol_type = genomic DNA
catcgctcggcgcaaggtacgtaaaatcttgagcccattcgagtaataatcactttgagcatttatacttcaaattaattcagcattgtcttgcccaatcg

SEQ ID NO: 8 - DNA, 101 bp, Cannabis sativa
  source 1..101: mol_type = genomic DNA
tccccgaatcatgcgccattaaattatcacatagagatcttaccatgtctgtcaagtggttaatttcttctggaagtgatagtatatttttcacttcttca

SEQ ID NO: 9 - DNA, 101 bp, Cannabis sativa
  source 1..101: mol_type = genomic DNA
tgatattattttactatctttttgggataacatgtgagcttcaattatttgtaacaagaagagttaatgaggtcgaattattttaaaaaataaatttattt

SEQ ID NO: 10 - DNA, 101 bp, Cannabis sativa
  source 1..101: mol_type = genomic DNA
agagaattaaatctctttgttggagtcatgctctgtagttctccgttatgtatggctcacaaagtggcaagaatcacgcgcttgagcagagcgtggaagga

SEQ ID NO: 11 - DNA, 101 bp, Cannabis sativa
  source 1..101: mol_type = genomic DNA
aacccgagcatgcgcagtctcagtcttagtgttaagctgtaaaaccagttgttattatcttgttcttcatcttatctgagcaacaaattcttgctccattg

SEQ ID NO: 12 - DNA, 101 bp, Cannabis sativa
  source 1..101: mol_type = genomic DNA
ctctccactcctctctctcttaaattttctttcctttaatttttccaccataccaaacatagggtaagtatatttttttttttgtcacaaagcattctttt

SEQ ID NO: 13 - DNA, 101 bp, Cannabis sativa
  source 1..101: mol_type = genomic DNA
aagaaaaagatgagtacccttttagaggctgatgataaagcctcgaacttcgtagagagatgaagaattagtacatgaaaaattaaagctttgatgatttt

SEQ ID NO: 14 - DNA, 101 bp, Cannabis sativa
  source 1..101: mol_type = genomic DNA
tttcttgctactcactacaagaatgttaagagatttgaagaagcagaggtgtattgtacccgtcttctggattacaatggccctgtaagttctaatattgt

SEQ ID NO: 15 - DNA, 101 bp, Cannabis sativa
  source 1..101: mol_type = genomic DNA
tcggtagtgacaatactcagggatccagttgaccgtgtctttagtacttatgaattttcaatagaagtagcagctaggttcttggtgcatcctaatttaac

SEQ ID NO: 16 - DNA, 101 bp, Cannabis sativa
  source 1..101: mol_type = genomic DNA
tgagaagggcattagttcaaaatttgtgtatatagtcttaaccttgaaaatttggattggattggaccccaacccaacacccttacttgcaagccagttat

SEQ ID NO: 17 - DNA, 101 bp, Cannabis sativa
  source 1..101: mol_type = genomic DNA
agcaactgcagaaccactcccaccccaaaacaagtcaccaaacaaggagatggcccttaaatattcttagaggaaaaatgataagtgaataattaattata

SEQ ID NO: 18 - DNA, 101 bp, Cannabis sativa
  source 1..101: mol_type = genomic DNA
attataaacaataggaaaaaccaacaccagagataacacaaaaacatgtacacaactttataatgccgctctaccatgaatatgtccagtcttctcgcaaa

SEQ ID NO: 19 - DNA, 101 bp, Cannabis sativa
  source 1..101: mol_type = genomic DNA
attttccaggtgtttctttggacttcaaatctgatggcagcaatgaaattgctacagcaactatttcaatctcatcccattttgacagaggtgttaatgag

SEQ ID NO: 20 - DNA, 101 bp, Cannabis sativa
  source 1..101: mol_type = genomic DNA
aaaatgcatttctgagttattatgagaaactgttgggaactagtatgcattatagaaggaaggttctgcgatctgtagtcaatcttggccccaagatcaca

SEQ ID NO: 21 - DNA, 101 bp, Cannabis sativa
  source 1..101: mol_type = genomic DNA
agttctcatctaatgattgttgtgtgaaggacaaccaaacaaagaagaaagtgcttcaagggaagcttaaggaaggtctctatcaattcaactctcactgt

SEQ ID NO: 22 - DNA, 101 bp, Cannabis sativa
  source 1..101: mol_type = genomic DNA
tgatatggaatttttctcaggttggggttatttgtggataatctcaaacggagatgttcatttgactttgctgatgtgaattttctttcttttgggatctt

SEQ ID NO: 23 - DNA, 101 bp, Cannabis sativa
  source 1..101: mol_type = genomic DNA
tctaggtgatgcacttgagggctctcgaagaaggattcccaatgattgttaatgaagacccaatcaattcgttctttgatagtattggcagcagctcgatt

SEQ ID NO: 24 - DNA, 101 bp, Cannabis sativa
  source 1..101: mol_type = genomic DNA
aggaagaatgtcgcagaaaaagtagatttcgtaggacaatatagggtacagtaataaaatttatagggttattcttgttaaaattattgggaaatttacat

SEQ ID NO: 25 - DNA, 101 bp, Cannabis sativa
  source 1..101: mol_type = genomic DNA
ttatatggagaccattcaagaccttcttgatccatcaaatgataacatcccctttgtggaagatccaagaactggtgatgtttcagtacctggtgcgactc

SEQ ID NO: 26 - DNA, 101 bp, Cannabis sativa
  source 1..101: mol_type = genomic DNA
aaaaccctaccgtacaatgtgagtgtcttcgctgttaattctcttctcgttccgtacggattcgatctaatggcttccgagactcgacctccattaggttt

SEQ ID NO: 27 - DNA, 101 bp, Cannabis sativa
  source 1..101: mol_type = genomic DNA
aatttaagttctgggttttcatggatttgcctccttattgagaccaaccagtctttttcctgtttggctgcctggaaaatctcattggcgtaatgggtatt

SEQ ID NO: 28 - DNA, 101 bp, Cannabis sativa
  source 1..101: mol_type = genomic DNA
tgattatcattgttaattattgttttacctatatctatatatgtaattgttaactatatccattttaatagagacccaataactaaccaaatcttctaatt

SEQ ID NO: 29 - DNA, 101 bp, Cannabis sativa
  source 1..101: mol_type = genomic DNA
gtgtgttgttaggccttggaccaaataaagggcaataaaggtaaattgtctattgaagatctaaacatctctatgagacgaaagaattatacttaaataaa

SEQ ID NO: 30 - DNA, 101 bp, Cannabis sativa
  source 1..101: mol_type = genomic DNA
ttgtttgagctactcctttcagtctcaaatcaggattgcttttgtctagatattcgggtcgcagcaagctcgaattaaagcattgtgaaaacatcaggctt

SEQ ID NO: 31 - DNA, 101 bp, Cannabis sativa
  source 1..101: mol_type = genomic DNA
ctctggatgattaccgtcatgtgggttgttatcagccatttaatatgggtttgtatttgttttataaggaaggattttccaagtttttatcaccaagactc

SEQ ID NO: 32 - DNA, 101 bp, Cannabis sativa
  source 1..101: mol_type = genomic DNA
cgaggtagacaaacagctcttcaaggctagccaacgaacctgtgtttaggagtgaaaaatctatcccaaatgccatcataacgccatctaacagcacttcc

SEQ ID NO: 33 - DNA, 101 bp, Cannabis sativa
  source 1..101: mol_type = genomic DNA
tttctttgggctatctccactgcaaactttactaaagggctcgtaatgtcatctccttcaaatttcaagaatgttgcattcatcccattttcattactctc

SEQ ID NO: 34 - DNA, 101 bp, Cannabis sativa
  source 1..101: mol_type = genomic DNA
tagtcttcattgggtctctggtctttcttcactagtatatctagagatgagagcaaaccttagtgcaacatctgatcattggctactctctttaaacaaaa

SEQ ID NO: 35 - DNA, 101 bp, Cannabis sativa
  source 1..101: mol_type = genomic DNA
ttgctcttgctaattttgtagtactaaggattaatgaaagtggagaaaattgatgaactcccaagcaaaacattattataattttggcttattaattacaa

SEQ ID NO: 36 - DNA, 101 bp, Cannabis sativa
  source 1..101: mol_type = genomic DNA
tttgcagtatagatatttaaatgattagttatattaataatttaatatggccacttgatttattttagattcatacataggtaagcatccattaaattttc

SEQ ID NO: 37 - DNA, 101 bp, Cannabis sativa
  source 1..101: mol_type = genomic DNA
atttatacataataacagtcttttacaaacagcaagaacacgaaacgcaataggccgccctaccctaaagacgggcataacacaaaacaatccttcttaat

SEQ ID NO: 38 - DNA, 101 bp, Cannabis sativa
  source 1..101: mol_type = genomic DNA
tatgaagcagaagaacaacaaccaaaacaataatattcagcccttcatacctggttcagttattattttgttttgttattattttcagttatgcttggtat

SEQ ID NO: 39 - DNA, 101 bp, Cannabis sativa
  source 1..101: mol_type = genomic DNA
tcctctccttcctcaagatctctatgacaccatgtcactattatgattcttcctcctggggctgcaactctaaccaattcactcacaaactgcacactcac

SEQ ID NO: 40 - DNA, 101 bp, Cannabis sativa
  source 1..101: mol_type = genomic DNA
ttgggagttgaagaattgattcccatactcagttatcttcatcctggagagctctcctatgctaaatattatgctggttacttaatgaaggaggtaattaa

SEQ ID NO: 41 - DNA, 101 bp, Cannabis sativa
  source 1..101: mol_type = genomic DNA
tcattgtagagcttatattaatagtggctgagtcaagttccactcttcaatatttgactcaaaatattgacaatattattcaattaattatctgcattaaa

SEQ ID NO: 42 - DNA, 101 bp, Cannabis sativa
  source 1..101: mol_type = genomic DNA
tgaattgatttgtgtaattttagtttttgttgccttacttttacttgattatggaatcaggtcatttttggatgataataatcataggggctccccaaatg

SEQ ID NO: 43 - DNA, 101 bp, Cannabis sativa
  source 1..101: mol_type = genomic DNA
tctctgtcttgggcagaggatcattttcaacatattatgctattttttctgcttatcttctaaaaacggttggatcttgggtgtcctcctcagcctctgca

SEQ ID NO: 44 - DNA, 101 bp, Cannabis sativa
  source 1..101: mol_type = genomic DNA
tgagtcaactgaagtctacgagctctgactcttgcttggactctaaccaaggcttgcatacatctcattgtcatttgtgcttgttttctcacattgtggcc

SEQ ID NO: 45 - DNA, 101 bp, Cannabis sativa
  source 1..101: mol_type = genomic DNA
ctctttgtcgatctctataaaaattaaataaaaatcaagaagaaaaaactctcacgtacttcatcaattaattaattcttctccatattatatatcctaga

SEQ ID NO: 46 - DNA, 101 bp, Cannabis sativa
  source 1..101: mol_type = genomic DNA
atctctttcacctttgttattttctctcactacattcctaaacatttcccatatgcaccaagggccacaacaacaacaacaatcccttcatttagtttgac

SEQ ID NO: 47 - DNA, 101 bp, Cannabis sativa
  source 1..101: mol_type = genomic DNA
ggaaaaaaaatattcatgtgttttcagatgcataaatttagaaagaagtaataaaaactcctcttttttgtaactctgaatttgaacgatttgtgtctaaa

SEQ ID NO: 48 - DNA, 101 bp, Cannabis sativa
  source 1..101: mol_type = genomic DNA
actaaactattgtggctacaaagacaaataattaatccttgtagaataaacaaagcttcaagataattacaagctgattaagaaataaaaaaaataggact

SEQ ID NO: 49 - DNA, 101 bp, Cannabis sativa
  source 1..101: mol_type = genomic DNA
gaactggccaaagttttccacaaaatggcttggctagtcacaccaaggcatataccaaatgaagagatgggaaaccttagaagaaatggccatctcttgtc

SEQ ID NO: 50 - DNA, 101 bp, Cannabis sativa
  source 1..101: mol_type = genomic DNA
tacagtactaacatataagataattgcgtcattacattcgtaattttgtgagttagaattttagttttcaaggaattatttcgtccgtggcttgttttaat

SEQ ID NO: 51 - DNA, 101 bp, Cannabis sativa
  source 1..101: mol_type = genomic DNA
gatgacgattctttcccttaggaaaattgctcctttgttgacctttcccttaaaggagatcagagtgaactttacgtggatcttgagcctttttattcctt

SEQ ID NO: 52 - DNA, 101 bp, Cannabis sativa
  source 1..101: mol_type = genomic DNA
ttccatccccattctctatcgatatccattcaaaatgagtttcttctcccaaatcaagctttgtcacatgaaggatgcattacttccgcacttcgcagcca

SEQ ID NO: 53 - DNA, 101 bp, Cannabis sativa
  source 1..101: mol_type = genomic DNA
ttggtggcccggcccagagatgcatggaggaaaatgtttagtatgaatgagattgatgtggcaacaaatgggttctcaagagcaaatgtcattggtagtgg

SEQ ID NO: 54 - DNA, 101 bp, Cannabis sativa
  source 1..101: mol_type = genomic DNA
atattttccatatcttatttggcaacagttatcatatcagcacgcatgctgcatttcgtatttggtaccataggacatacatagtcacacttaagttttga

SEQ ID NO: 55 - DNA, 101 bp, Cannabis sativa
  source 1..101: mol_type = genomic DNA
caaacaggtttggatcggagatagtggtatactgttagaatattttacgttgagattacccagcagcagcgttgagcgaattggaaagatgctatcgtgca

SEQ ID NO: 56 - DNA, 101 bp, Cannabis sativa
  source 1..101: mol_type = genomic DNA
attttcattatgtttctttgacagctagatgaggacaaagggaaagtttctgctccaacttgtacaaaatctgtctctgaacgtatagaagaggaggtgtg

SEQ ID NO: 57 - DNA, 101 bp, Cannabis sativa
  source 1..101: mol_type = genomic DNA
ccttgtattttagccaaagcccaatgtattttagatgagtgggttatattcactaacttggtcttgaaggattgtaatttcaaattgggtcgtaagaggca

SEQ ID NO: 58 - DNA, 101 bp, Cannabis sativa
  source 1..101: mol_type = genomic DNA
agtagtgaagttcaaaccagtttcaaaagttccaaacacaaagtttgaatgagatcactatttggtaatataataaggaactttctcaagtaagtccacat

SEQ ID NO: 59 - DNA, 101 bp, Cannabis sativa
  source 1..101: mol_type = genomic DNA
ccattgccaaaaaaaaaagataacttgtgggtgaagtaggtacataatgtgtacgtgaaggaggaagattggtggaattagtattggcacaaactggttgc

SEQ ID NO: 60 - DNA, 101 bp, Cannabis sativa
  source 1..101: mol_type = genomic DNA
atgaattgatccaagccaaagaatacttctttcttttgttgcagaaattccactaaataacacaccgcaggatctgaaccacgcgaaacgcaggctgttga

SEQ ID NO: 61 - DNA, 101 bp, Cannabis sativa
  source 1..101: mol_type = genomic DNA
tctacaacacaccctgtgtatacagaccacaaaggcaagtagaaaaatttccaaaccagcatacttcaatgactaatccttttctgtttccaacagtgcaa

SEQ ID NO: 62 - DNA, 101 bp, Cannabis sativa
  source 1..101: mol_type = genomic DNA
atgtagaatgttcctcagcgacaatggttagggctcacttcatagatttaaattttgtgctcagattacctccattttttatcttgtgtatgcgaatgaaa

SEQ ID NO: 63 - DNA, 101 bp, Cannabis sativa
  source 1..101: mol_type = genomic DNA
tttagggttacaccatcgttatcttggaagatgaaaaggttgaagatgtgaaatattattcaattcaacacgtgctaactcaacaaaagtttaatcatttt

SEQ ID NO: 64 - DNA, 101 bp, Cannabis sativa
  source 1..101: mol_type = genomic DNA
aaaatatgaatcgagtcagcacgacatgcacaaacttaattagaacactagctggtagtgaggtaaacatattatatattatttcatttttctctctctct

SEQ ID NO: 65 - DNA, 101 bp, Cannabis sativa
  source 1..101: mol_type = genomic DNA
ggatgtccagaggatctaacctaacaatatgaatgcgttcctagacagagttgacgactttatcaagctagaggaagttttccagaaagcccagataggga

SEQ ID NO: 66 - DNA, 101 bp, Cannabis sativa
  source 1..101: mol_type = genomic DNA
acttgtcaatgggtgaacgaagtcaccttacacatcgtgccaattgttaagggttccaagcaatcaagcaagattaagtaaggagaaaatgaccagccagg

SEQ ID NO: 67 - DNA, 101 bp, Cannabis sativa
  source 1..101: mol_type = genomic DNA
acgaaactaaatatgtgtggcaggggcaccacaacattctcataaaaactcccacagagcccaaataagacacttgacagagctaatgaccaaaatttatg

SEQ ID NO: 68 - DNA, 101 bp, Cannabis sativa
  source 1..101: mol_type = genomic DNA
aattttctggatcattagtttctccttctctttcagcatccaccctaaaaactacttcttcctctttaggatcctttgggtcatctggttgttgtcaatgt

SEQ ID NO: 69 - DNA, 101 bp, Cannabis sativa
  source 1..101: mol_type = genomic DNA
ggttgcattgcactggttggcatggttaactaactagtgatgagaagtaaagttcatctctataatcgagaggtaatgtgtgtttattgtatattttttta

SEQ ID NO: 70 - DNA, 101 bp, Cannabis sativa
  source 1..101: mol_type = genomic DNA
aggtgcaagtggttacgatgttgctcatagtactagtttcgctgtgaggttacttgaattctaattgggaacatgccttcagtttcatatgtgagtgagaa

SEQ ID NO: 71 - DNA, 101 bp, Cannabis sativa
  source 1..101: mol_type = genomic DNA
atatttatggcaaaaataaggaatggactggcctagctatggctcaacaacaactaccagactcactacaatcactgaaagtatatgaaaagatatatgta

SEQ ID NO: 72 - DNA, 101 bp, Cannabis sativa
  source 1..101: mol_type = genomic DNA
ctccaggacccagagttgtccaagataaaaggttgcccaagtgatacccaaatgggtactcaaacggcctatcacggacgctaaccacccaaaaggatcgc

SEQ ID NO: 73 - DNA, 101 bp, Cannabis sativa
  source 1..101: mol_type = genomic DNA
tgaattcgctcaacagctcaggcagaaatgtccaaagccaaacaatgacataaccgcaggacagtttttggactcaacttcgtccagcttcgacaacgatt

SEQ ID NO: 74 - DNA, 101 bp, Cannabis sativa
  source 1..101: mol_type = genomic DNA
tctatattctcagtctctagtttttaatgataaactgaagactctattttcgtattcatcaacccttgtcatatgttctcaactgttcctgttactgcagg

SEQ ID NO: 75 - DNA, 101 bp, Cannabis sativa
  source 1..101: mol_type = genomic DNA
gtcatatatagctttccataatttcctaattttagcccgtatgacatataggcatcatgaaagatgtcgaaccatttctatggtttaagcgtactatatat

SEQ ID NO: 76 - DNA, 101 bp, Cannabis sativa
  source 1..101: mol_type = genomic DNA
tggttaactttattgaagcttgaccgttggtgtgggtctcattttgttgcggattaagtatgtgaagcttagttggcttttggtctaccaatatatatata

SEQ ID NO: 77 - DNA, 101 bp, Cannabis sativa
  source 1..101: mol_type = genomic DNA
aatcaagtggctcctaaaagtaacagggacttcaagaatatcctgtataacagactcatcaaaatgatttctcagattgcctatatcccaatacccagagt

SEQ ID NO: 78 - DNA, 101 bp, Cannabis sativa
  source 1..101: mol_type = genomic DNA
ttcaaaatatgggtgagtgtaatgtgtttgggttaggtggaattattcattcacgtgtttcttatgatggaaaggaattgtaatatatatatatatattat

SEQ ID NO: 79 - DNA, 101 bp, Cannabis sativa
  source 1..101: mol_type = genomic DNA
tatcaaacttaaatttaaagcttgcagtgagaagcccactataatcatgaggttagaaatgaacccaaaattaaggaacaagataagatcccttaattttt

SEQ ID NO: 80 - DNA, 101 bp, Cannabis sativa
  source 1..101: mol_type = genomic DNA
ttccaaggaaaacaaacaagcagtacgttaaagtctaaagggccgcaccaacgtattccacctttttacagataattgttattaatttctcaccattacca

SEQ ID NO: 81 - DNA, 101 bp, Cannabis sativa
  source 1..101: mol_type = genomic DNA
atcccaagtgatagcggaatcacatttaatgtcaaaccattaatgaaggccgtagaaattgctgagaaggcaagggatgccatacttagccggaaattcca

SEQ ID NO: 82 - DNA, 101 bp, Cannabis sativa
  source 1..101: mol_type = genomic DNA
agtctctgtatatagaatatacaaatttatattctttccaacggattagatgactgttgaacaatgagttttgaactcgtaaccatcctcatctacaatac

SEQ ID NO: 83 - DNA, 101 bp, Cannabis sativa
  source 1..101: mol_type = genomic DNA
caggtaacccaaacaattcgaaaccccaaccaaatcaaatctaaacctcaccaaaccttccatacctgcttaaaatacgccaataaacataaccgaagcaa

SEQ ID NO: 84 - DNA, 101 bp, Cannabis sativa
  source 1..101: mol_type = genomic DNA
ttttgatttgccgttgaacaatgtgattttcaatttctttatagttttattattgtttggtgctagtttattaatctttatttttttcaaatatttttgtt

SEQ ID NO: 85 - DNA, 101 bp, Cannabis sativa
  source 1..101: mol_type = genomic DNA
gtgatccacggctgaaggactagggaatgccattggcaaaaatcaaggcaatcgcccgaacccatccaaagtcgcaccactcatccatattttcaagagat

SEQ ID NO: 86 - DNA, 101 bp, Cannabis sativa
  source 1..101: mol_type = genomic DNA
caaaaaatcccgatggctgtaaaggaaataaggtgaattagatcaattaaattttgtggctagccgtataaaaccatccccttcaccaccactcagacata

SEQ ID NO: 87 - DNA, 101 bp, Cannabis sativa
  source 1..101: mol_type = genomic DNA
tactctatcttttgcgtgcagctctaatctcaggccctgattacatgtataatataaaccattgtacttccatgagattatatttccatttgcatttgtta

SEQ ID NO: 88 - DNA, 101 bp, Cannabis sativa
  source 1..101: mol_type = genomic DNA
agccagtcatggagtcaccagtcttaacggagcctcaaataacaaagggtccagagtaggagtcatactgatctccccacatggacattagctctagagtt

SEQ ID NO: 89 - DNA, 101 bp, Cannabis sativa
  source 1..101: mol_type = genomic DNA
tagtatcctgcatgaacagaagcagttttccttctggtccatctcctaaatagagttgttgaatttttaagcaggaggcaagatttttgccttccctttcc

SEQ ID NO: 90 - DNA, 101 bp, Cannabis sativa
  source 1..101: mol_type = genomic DNA
catgttacgaactttccttaagagatcttgtagagcagccggtagtggccgaagctcggcgaaacgaagacaaggatgcaaatataagtaggaacgatagt

SEQ ID NO: 91 - DNA, 101 bp, Cannabis sativa
  source 1..101: mol_type = genomic DNA
cctgcaccgtgaaacatggcttgccctttctatacgaatgctctacaaatggattcaattgccttcagaactttctttggcaaaatcactatttgactcca

SEQ ID NO: 92 - DNA, 101 bp, Cannabis sativa
  source 1..101: mol_type = genomic DNA
tatggatacgacatagtccgtttactaacgtgactccagtgtaactaattgggcgacaacactagacaaaaaagaattaaatctgaccttcgttgtgattc

SEQ ID NO: 93 - DNA, 101 bp, Cannabis sativa
  source 1..101: mol_type = genomic DNA
aaaactcttccgctgcatacctgatttagtagcataaaactaacagattttgctttaggtgtagttccaactcgtgaagttgtagagaaaacttgaattct

SEQ ID NO: 94 - DNA, 101 bp, Cannabis sativa
  source 1..101: mol_type = genomic DNA
aaaagtcagcatttaaaaccctaacgagaaaacataagaaattatgccttctgaataatgatacttcagttggtggctaatgaggtagtgtaataaagagc

SEQ ID NO: 95 - DNA, 101 bp, Cannabis sativa
  source 1..101: mol_type = genomic DNA
gtgtagcctttgagcaaacttaacaagtctttgatttttctcaagtaggccaccattctatcagctcgagctgtgtattcactcatgatttgattcacaac

SEQ ID NO: 96 - DNA, 101 bp, Cannabis sativa
  source 1..101: mol_type = genomic DNA
ttgatcaggtaagatatccacactctcgatggaagcaccagaccgttaacctccaaaagtaatcaaccgacaacaaagttgtatggatctgatgcttgcca

SEQ ID NO: 97 - DNA, 101 bp, Cannabis sativa
  source 1..101: mol_type = genomic DNA
ttcacaattaagccattgcttattgaaaattcataaataaggacctttttcctaaaggggataacttatgaggggtagctcctcaacttgagattcgaccc

SEQ ID NO: 98 - DNA, 101 bp, Cannabis sativa
  source 1..101: mol_type = genomic DNA
agttaaaataatatttgctactactagaggccaaactagatatcctctttaatttattataacttattacaaaaagaagataggcaaacaggggtgtccta

SEQ ID NO: 99 - DNA, 101 bp, Cannabis sativa
  source 1..101: mol_type = genomic DNA
aacaccggaagaagaaatggcatatgatgatcacattcatcacatgtgaaaattttgatcattaaagcgtgatgagcgctgattccctcgtcattaagccg

SEQ ID NO: 100 - DNA, 101 bp, Cannabis sativa
  source 1..101: mol_type = genomic DNA
ccagactgtatttcatggagtcgcggctcgattaagtgaagaagaagctagcaagctcgagaaagaagaaggtgtgctggccatattcccggaaaagaagt

SEQ ID NO: 101 - DNA, 101 bp, Cannabis sativa
  source 1..101: mol_type = genomic DNA
cttctcctttatttttcttttccgacctttgactttcctgtaggaacctctacagtttgaatactaacgaaaactttgatttctcaggtacagtaattaat

SEQ ID NO: 102 - DNA, 101 bp, Cannabis sativa
  source 1..101: mol_type = genomic DNA
gaagaactggcaactcatctcctgtttaatggaatcgctattgtcgaccttggtctttgtggtttttgggttcttgatttctgttgtcacccttcgtgtcg

SEQ ID NO: 103 - DNA, 101 bp, Cannabis sativa
  source 1..101: mol_type = genomic DNA
aagctgcctccaaaagtgaaaaagttgatgtgatgtgttggtataaggtgccttcttactatgattcaaattttgactaaaagggtgactgccaagtggtt

SEQ ID NO: 104 - DNA, 101 bp, Cannabis sativa
  source 1..101: mol_type = genomic DNA
gtagtctttgcagtgaacattgattggacctttatgttcatacatataagctcgggccacaatgttgttgatattgtcggagcagagatatactagctgat

SEQ ID NO: 105 - DNA, 101 bp, Cannabis sativa
  source 1..101: mol_type = genomic DNA
atccttcagatgacacttataaagattaaattcaaagaaaacttgattgtccatgacaaactttgacacactaagcaatttcttagttatctcaggaagat

SEQ ID NO: 106 - DNA, 101 bp, Cannabis sativa
  source 1..101: mol_type = genomic DNA
gctaatgcaaaaaaacttgctttcaacacaacaatggcaacaatttagtacctgaagatacctaaccactttttaatttcaactaggcatcttttactata

SEQ ID NO: 107 - DNA, 101 bp, Cannabis sativa
  source 1..101: mol_type = genomic DNA
tggatagcttctgccaaaactccaccagtaagtcaaacatggagtttcctctactatctctaataagcttgtaacaaggcattaacttccacactacttta

SEQ ID NO: 108 - DNA, 101 bp, Cannabis sativa
  source 1..101: mol_type = genomic DNA
agtttgaaattatatgtcatggtctactttttgtgaaactatgtgatttgcgcaggtcacgcattcaccagaaggaatggaaacacttcgttactttctct

SEQ ID NO: 109 - DNA, 101 bp, Cannabis sativa
  source 1..101: mol_type = genomic DNA
gtataaacatggggatccaatccataaacctactactttgacacaagacgtgtgagaaatgacaatatactcatggtaatgcagggcccttaagcctatga

SEQ ID NO: 110 - DNA, 101 bp, Cannabis sativa
  source 1..101: mol_type = genomic DNA
cgttatcaatgatatcttctttgtggatgcatcttggaaagagggtgtcgcgggcttagcagcaatgtggaggtctcaagatagggtctgctggtattcaa

SEQ ID NO: 111 - DNA, 101 bp, Cannabis sativa
  source 1..101: mol_type = genomic DNA
tctacgggaatggcaatgttgtggttcagcatcaacatcttgttcccaaccattatttctttgtttcggacagtggctttcactccatggccagtaatgga

SEQ ID NO: 112 - DNA, 101 bp, Cannabis sativa
  source 1..101: mol_type = genomic DNA
tcccaaaagagatggaaaccccatcacgtgtcctaaaattacaataccattaaccatttccactcattaatctatttcattttgagcctttaatcgttatt

SEQ ID NO: 113 - DNA, 101 bp, Cannabis sativa
  source 1..101: mol_type = genomic DNA
cggcgtaatatagccggagaccgcgaaaatttgcatttcagcaatacgaatggcaataacgttagggtcagaaattattcagtgcagcggggtcgatggaa

SEQ ID NO: 114 - DNA, 101 bp, Cannabis sativa
  source 1..101: mol_type = genomic DNA
cagcagacagatgatgacaagtggggattttggtttgtatatacatcaataagtttttgtataagagatttgtatttggactctaaccaagttgtcaaaga

SEQ ID NO: 115 - DNA, 101 bp, Cannabis sativa
  source 1..101: mol_type = genomic DNA
agcctaagtatctgtatggtggcttagtagttgttacttttgagttagacatatgccaaagttgtatttctttttccttttccatggatatagatgtcatt

SEQ ID NO: 116 - DNA, 101 bp, Cannabis sativa
  source 1..101: mol_type = genomic DNA
atataggtatgtatcaagacaaacttttatcttgagaatttttgtgcttgcttttctcacctgaagtatagagaaatgttttggggaagtactttttattt

SEQ ID NO: 117 - DNA, 101 bp, Cannabis sativa
  source 1..101: mol_type = genomic DNA
ggaaataagtggctctacaaaatgaaatttaatgcaaatggtacattttaaaggtacaaggctcgtcttgtagctaagggcttcgccaaatacttggggtt

SEQ ID NO: 118 - DNA, 101 bp, Cannabis sativa
  source 1..101: mol_type = genomic DNA
gcgatcgatgtttacatagccggtgaatactgtcattgggcaaaaggaaagttttgctgtggaggattagtgtgtcaaggtcctgcaatatattgttacta

SEQ ID NO: 119 - DNA, 101 bp, Cannabis sativa
  source 1..101: mol_type = genomic DNA
tagatccaagtggatcaagatcaaggacccgcgaacataacagatcaactaaaactcatgcccatgacaggcgaacatgagaggaaggtggattgcgacgt

SEQ ID NO: 120 - DNA, 101 bp, Cannabis sativa
  source 1..101: mol_type = genomic DNA
attccccgcaaatagacccacagtgatatttatttagttatgtttcccagagttatatatctaagtgctcaaatatctgaaacaacaagagcaaaaagagc

SEQ ID NO: 121 - DNA, 101 bp, Cannabis sativa
  source 1..101: mol_type = genomic DNA
atttggggcacaaaaacccaacaactacacagttgaagagagattaaattcattgattggtactacctatatcaacaaggtgcatcttgttttctagtagc

SEQ ID NO: 122 - DNA, 101 bp, Cannabis sativa
  source 1..101: mol_type = genomic DNA
gagtaatgcatataaaaaatgaggttgcataatgttttatgaccacaaaggaagtaattgtggagaaagtagttgcttaccaacagacttacaattccaat

SEQ ID NO: 123 - DNA, 101 bp, Cannabis sativa
  source 1..101: mol_type = genomic DNA
cttctcggcaataagaagctttttgttaacttcatccaattccattcggacggctccaagttgaacctgttgggttttatgccctaaataatactctttac

SEQ ID NO: 124 - DNA, 101 bp, Cannabis sativa
  source 1..101: mol_type = genomic DNA
tttgatctgattttaccctgaataagcattcatgaagcttaatatttcattccctgctattgcatccacaacctggtcgattcttggaagcggaaaacagt

SEQ ID NO: 125 - DNA, 101 bp, Cannabis sativa
  source 1..101: mol_type = genomic DNA
ttgtatatgcttactttaggcaaattagacttagagttagattagtagcgatggtccaagctagaatcattactcattactcattactcattgtatacagt

SEQ ID NO: 126 - DNA, 101 bp, Cannabis sativa
  source 1..101: mol_type = genomic DNA
gagttttgttttttggcagcgatgttaatagaggttgcatgcttccatgattggaaattttcttgaccagtcaagagaaatgaagcgatgaggttatttgg

SEQ ID NO: 127 - DNA, 101 bp, Cannabis sativa
  source 1..101: mol_type = genomic DNA
gttaatgtcacccaaatgtgatggtaatgggtcactagagtttagttctgttattgtcttttattgtgaagttgggtgttcggtgagctagatagcatagc

SEQ ID NO: 128 - DNA, 101 bp, Cannabis sativa
  source 1..101: mol_type = genomic DNA
ctgtttcaatcatttttcttttactgctgctctctgctctttattagtttttctttacagattttgttttctgatttcatttgctaagtatttcccaaagc

SEQ ID NO: 129 - DNA, 101 bp, Cannabis sativa
  source 1..101: mol_type = genomic DNA
atctagttgatcattctcattttttatgtgtacagactgcagtgatgcccctagcaggggaggaacttcatttggtagcaatgtattccaggaaaaatgat

SEQ ID NO: 130 - DNA, 101 bp, Cannabis sativa
  source 1..101: mol_type = genomic DNA
tagtaatatgtctcttatgattgattattcctatacacaccattgctactgtactagtgttgttcttgcttgattgatttgattaatcaatgctagatggt

SEQ ID NO: 131 - DNA, 101 bp, Cannabis sativa
  source 1..101: mol_type = genomic DNA
aattctagctacagttctcaagatgtttacaaagaattcagcaaagtgcttttcaattgagattatgattttaggtcccttccttctgtgaattcattcac

SEQ ID NO: 132 - DNA, 101 bp, Cannabis sativa
  source 1..101: mol_type = genomic DNA
agcttaagcaagaaatttaacttttcggttcagagttatcgccttattgcggtggcattcatgatagctttcggtttagctcatgcgcggttatgaattcg

SEQ ID NO: 133 - DNA, 101 bp, Cannabis sativa
  source 1..101: mol_type = genomic DNA
ttattacaggtttggaactggtttcaaaataggcgatatgctataagggcgaaattaagtaggaaccctggaaagttaagtgtctcatctatgcctcggga

SEQ ID NO: 134 - DNA, 101 bp, Cannabis sativa
  source 1..101: mol_type = genomic DNA
ctgattgagagagatgtatggaaggatagatggatttgattgctgactttcctctattaatcattttatttctttctcacttttctctctttatatatata

SEQ ID NO: 135 - DNA, 101 bp, Cannabis sativa
  source 1..101: mol_type = genomic DNA
tttgatcaggccccaaaccatgtctttccatctcattccaagtttgttcaactctttcttgacaatcccaatccataaacaacttcaacaacaaattatat

SEQ ID NO: 136 - DNA, 101 bp, Cannabis sativa
  source 1..101: mol_type = genomic DNA
ctaggctaggtgggccctaggcacaggcgtagcccgcctatgcccagggccgggcctaggtgcagagaattctagataccaaaaataaaaagcagttacat

SEQ ID NO: 137 - DNA, 101 bp, Cannabis sativa
  source 1..101: mol_type = genomic DNA
gggtttcaacggaaacatttgaatgggccttaaagggcccagtaatgatgaaaaagttgggggtgttgtggtccactagtgggtttgggccgttgggactt

SEQ ID NO: 138 - DNA, 101 bp, Cannabis sativa
  source 1..101: mol_type = genomic DNA
aaagttgagccaaaacaagccagaataacaaacaaattaaacacaacttgaaaggctaggccactaaattttaacgctctctgaagaaaatgtaagtttgg

SEQ ID NO: 139 - DNA, 101 bp, Cannabis sativa
  source 1..101: mol_type = genomic DNA
aaagaaatatgtgcatgtgattgtccactaaattctgctcaacttttactaaattgttttgatcttattgaaagttacaattttcaaatatatattatata

SEQ ID NO: 140 - DNA, 101 bp, Cannabis sativa
  source 1..101: mol_type = genomic DNA
caacatattacataaaacaattgatcttaaatctcccatatccataggcttcttcttcttaggaattaagattaaaatagttttgttaagatctataggaa

SEQ ID NO: 141 - DNA, 101 bp, Cannabis sativa
  source 1..101: mol_type = genomic DNA
actagtattctaatggagatcccattaagaagtacattgaaatacataaaacattattgtctttttggcatcaaaaagcaaaggtagcttggctaaaagaa

SEQ ID NO: 142 - DNA, 101 bp, Cannabis sativa
  source 1..101: mol_type = genomic DNA
tactacggtttagggccatctctaataggaatagctccatatattgctgtaaacttttgtgttttcgacttgtgagttcacttatactttcatctgttata

SEQ ID NO: 143 - DNA, 101 bp, Cannabis sativa
  source 1..101: mol_type = genomic DNA
aaatctcaagcgtgctcgcatactagtaaaatcacatgtattacactctattgttccaggtagcattctaaagttctgttcttaaaatattttttggttac

SEQ ID NO: 144 - DNA, 101 bp, Cannabis sativa
  source 1..101: mol_type = genomic DNA
aagaaccagtgaagctagagagaccactgacggtacaggtcttagtgatgctagagggaccaattatgtaaattaaccatgaagccactcctcctgatcat

SEQ ID NO: 145 - DNA, 101 bp, Cannabis sativa
  source 1..101: mol_type = genomic DNA
gaagatgaaggatatgaacttgctgatgagctaggcttgctggaagacataattatcttttgttgggttttctttcttttctgactacctttctctatctg

SEQ ID NO: 146 - DNA, 101 bp, Cannabis sativa
  source 1..101: mol_type = genomic DNA
aaaaataaccaagctttctccattggaggatggcttttcaaaacttgattaactgtgtatgaatcccatcttatagggaggccctgaagctgatctcgagc

SEQ ID NO: 147 - DNA, 101 bp, Cannabis sativa
  source 1..101: mol_type = genomic DNA
cccatctactaacaacaatttttggcgagttatcaaagaacaaagaagaactaacaatgaaacaaaactatgtcacacacacaaaactaaaacactcaaaa

SEQ ID NO: 148 - DNA, 101 bp, Cannabis sativa
  source 1..101: mol_type = genomic DNA
ggccagaacctagctatataatatctaataggcatagtgatgagtcatgagccatgtatctacaaatattcttggtgcattccccctacctatcattcatt

SEQ ID NO: 149 - DNA, 101 bp, Cannabis sativa
  source 1..101: mol_type = genomic DNA
catcttggcaatgttgcagggttcctacttactccaataatgctgtcatcgatggggatgtcaggtccattcattctcttctcgtcgtttgggttagtctg

SEQ ID NO: 150 - DNA, 101 bp, Cannabis sativa
  source 1..101: mol_type = genomic DNA
tctctttgtagacctacttacattctatggcttttctaccaggaggtaagggaaccttcacccaagttttcttttgagtaagagctttgatttcaatttgc

SEQ ID NO: 151 - DNA, 101 bp, Cannabis sativa
  source 1..101: mol_type = genomic DNA
aaagtgggttgtggagaagagaaaactcagttaatatcatcatcttacaaaatattttttgtacacttttatatttttttggggggtgggaagtgtgagat

SEQ ID NO: 152 - DNA, 101 bp, Cannabis sativa
  source 1..101: mol_type = genomic DNA
tgttggtcagatgaaggtttcggcatctgatttctcttgtacgagtgaactggacaatgctgagtgcgtgttgagagagtctgttctctttggggttgaac

SEQ ID NO: 153 - DNA, 101 bp, Cannabis sativa
  source 1..101: mol_type = genomic DNA
gaggctttaagttggataaaaacctctaatctttcgaacattgttattgacactcattgtcttattgctatacaagctttgcatagctcggaggatatgcc

SEQ ID NO: 154 - DNA, 101 bp, Cannabis sativa
  source 1..101: mol_type = genomic DNA
tttcaactctttctctaaaatttcaagatactcacttacttcttctaatgtctttttccgctcctccttaacccagaaagctttccatgcagttggcacga

SEQ ID NO: 155 - DNA, 101 bp, Cannabis sativa
  source 1..101: mol_type = genomic DNA
gaaggacttgttaaatacaactcaagatgtctaagtagagaagtgccacatgactactgacataaacatagcgtcaacacacacccaaaaggtgccttaaa

SEQ ID NO: 156 - DNA, 101 bp, Cannabis sativa
  source 1..101: mol_type = genomic DNA
atataattttctgattacagtgtgtactcaactcatgaaatattatgattaagctaagaaagaacaaactcacacactaccaccacaagagagagagagag

SEQ ID NO: 157 - DNA, 101 bp, Cannabis sativa
  source 1..101: mol_type = genomic DNA
actgataaacacaagtcaaccaatcaaacccaattggggctttgtaagtcatcccttcccactatttaaatagcctcattattagtttattagaaaatttt

SEQ ID NO: 158 - DNA, 101 bp, Cannabis sativa
  source 1..101: mol_type = genomic DNA
aaattcctttaaggagatgagcatcttgtctagctcgaagaagagaagacgaaccctcagcacaaagaatgaaaagaaaggggataaatgatccccttgtc

SEQ ID NO: 159 - DNA, 101 bp, Cannabis sativa
  source 1..101: mol_type = genomic DNA
atcggaagaatgaccagttggaacgcgaggctgcgactacaagagttggacgcaatgattcacctcctaaccaaccgaacacatccgctaggcgacctcga

SEQ ID NO: 160 - DNA, 101 bp, Cannabis sativa
  source 1..101: mol_type = genomic DNA
catcaatatggttctggctataagtattcaacatgacatcatctttatttttccataataaagtgatacctccactatgtccttgagagtccacagtgaac

SEQ ID NO: 161 - DNA, 101 bp, Cannabis sativa
  source 1..101: mol_type = genomic DNA
ccactctttttagtgaataattacattaatttagttttccaaaacaatttaaagataaaatgggaaactacttactttcccataatacaataatgacatgt

SEQ ID NO: 162 - DNA, 101 bp, Cannabis sativa
  source 1..101: mol_type = genomic DNA
gtccctgaaaaaaaaattatttttaggacagtcccaaattcttatatttggagtagtgagttttcgggagttaaaacagagattgattacagctttggtat

SEQ ID NO: 163 - DNA, 101 bp, Cannabis sativa
  source 1..101: mol_type = genomic DNA
ttagagaaaggatagtgactttttcaaaaatttgcgtgatgctgatgttggtaatggattgcagtttgctaccaaaattttacaactaatgattgcttttc

SEQ ID NO: 164 - DNA, 101 bp, Cannabis sativa
  source 1..101: mol_type = genomic DNA
ttgatctttttttttctccgaatctctgggcattttgctttcaatctgtactcatctaaagctttccacccgattagtagccagttctttggatttctact

SEQ ID NO: 165 - DNA, 101 bp, Cannabis sativa
  source 1..101: mol_type = genomic DNA
gtttaatataattataaaaaaagccctttaattattttttaatataaaaagtattttacttattttctatgcaggtgattggtgaactgtccattaaattt

SEQ ID NO: 166 - DNA, 101 bp, Cannabis sativa
  source 1..101: mol_type = genomic DNA
acaagcctttacccagtcggtgaaaggagcagtgaatccaagtctcaataggaccaatataatgaaatcccaatccatcttatcatacgccttttcgatgt

SEQ ID NO: 167 - DNA, 101 bp, Cannabis sativa
  source 1..101: mol_type = genomic DNA
tcttaccttttatattcaccattggatttcagttagaattagtcaacaatggttgaagaaatctttggttttcttctcgtttgcttcagccgaagctaaga

SEQ ID NO: 168 - DNA, 101 bp, Cannabis sativa
  source 1..101: mol_type = genomic DNA
cctccaaatactggcatatccaaaaattaaaaatttcttatactatgcaccaggtggtggtcacaccatgttcaagataaaacgacacaaggttaaaaaaa

SEQ ID NO: 169 - DNA, 101 bp, Cannabis sativa
  source 1..101: mol_type = genomic DNA
tattttcatccaccggatctcgattctcttttcgtaattccttctagaggccaagcctgtgcttagttaatagtagttagcctccttttttcagtagaatt

SEQ ID NO: 170 - DNA, 101 bp, Cannabis sativa
  source 1..101: mol_type = genomic DNA
atgccttgatgccagtgggaaaaatccaactacttgtcaccctaaaaggagaaaatagtgccaacgccttcaagcattgtacatttgcagtagtgaattgc

SEQ ID NO: 171 - DNA, 101 bp, Cannabis sativa
  source 1..101: mol_type = genomic DNA
caaattttagattccatctattttcgttattatttttctgttatatgttgcgtgtgttcattaaagtaacaaatatcatattttcatttgtccatccatat

SEQ ID NO: 172 - DNA, 101 bp, Cannabis sativa
  source 1..101: mol_type = genomic DNA
aataattgctccgcggaccctcaaagtgttggattttattttaccacgatctagatttactaacatgtatgtttgattaacaccctaaatatgaattcact

SEQ ID NO: 173 - DNA, 101 bp, Cannabis sativa
  source 1..101: mol_type = genomic DNA
gcattacaaaaataaatatataaataaatacagcagtatcaatgactaaatatttatcttcataaaccgaaaacatactttcagctcagtctcggggtggc

SEQ ID NO: 174 - DNA, 101 bp, Cannabis sativa
  source 1..101: mol_type = genomic DNA
ctcaaagatcacctaaaaactataactaatcctactgcatttcaagtgcagaaatattgtatttcagcaggatattgcttgcttagtcccagtcaaaatcg

SEQ ID NO: 175 - DNA, 101 bp, Cannabis sativa
  source 1..101: mol_type = genomic DNA
attatctataacttcagtatttaatagactttatatattaaaactataatcatgaacataccaagtaacataaaattcacttctatatcattcaatgatct

SEQ ID NO: 176 - DNA, 101 bp, Cannabis sativa
  source 1..101: mol_type = genomic DNA
caagtaggttatcatggacatgctgatttggtttcatcgatgcatttgtaaagtttctagtagcatcaaaacaagcttgatctgatgtctttccaatcaat

SEQ ID NO: 177 - DNA, 101 bp, Cannabis sativa
  source 1..101: mol_type = genomic DNA
caacagcctccctcagtttctccttgtgctcatcaaggttagtatgaagcccacagatgaaggaaacttatgcgatgttctctgtgtttctagcaccaagc

SEQ ID NO: 178 - DNA, 101 bp, Cannabis sativa
  source 1..101: mol_type = genomic DNA
cttacattgcagagaagagacatacaaaaataaataagaaaaatgaaaactggtcccaagtgaaaatcaaatcaattggaacatgacaattgtattcgtca

SEQ ID NO: 179 - DNA, 101 bp, Cannabis sativa
  source 1..101: mol_type = genomic DNA
tagaacccttaggattacaaggtataaatcaataatggcaaaactcaagtgttgagatgatgctagtgtaggcgtgtgagagagatgaagagaagagaagc

SEQ ID NO: 180 - DNA, 101 bp, Cannabis sativa
  source 1..101: mol_type = genomic DNA
atctgacagtgcttttcaaatgcaaaactttggtgaatggatcaactaggttgtcttctgtgtccactttctcgaccagtacatcccctcttgccacatac

SEQ ID NO: 181 - DNA, 101 bp, Cannabis sativa
  source 1..101: mol_type = genomic DNA
acatcacaaatgctgattttagggcattccatgtaggagcctgaaaattgcaggtgaaacccaatcagtcaaaaatagatgttaggaaaactttgtgagaa

SEQ ID NO: 182 - DNA, 101 bp, Cannabis sativa
  source 1..101: mol_type = genomic DNA
cgtattgccacgtcaaggtgcaaaaaataggataatagtaagctttgtatctttactggaccaagaagttaggccttcaattttcgacccaagtgtgatat

SEQ ID NO: 183 - DNA, 101 bp, Cannabis sativa
  source 1..101: mol_type = genomic DNA
tgatttgaggcgatcaagaattttatcaatgcacagattaagcctcatcagaacgtgggtgatcacctgctctagatagctagttttttccaagaagctaa

SEQ ID NO: 184 - DNA, 101 bp, Cannabis sativa
  source 1..101: mol_type = genomic DNA
atttaccattacatccttcaatgtattttatccttgaaatacatagctacctataaatgatattttagtaaactaatataattactgaaatgagtactcaa

SEQ ID NO: 185 - DNA, 101 bp, Cannabis sativa
  source 1..101: mol_type = genomic DNA
gttagaactatagttggtgtatatatgattgcatcttgatattgtgaatatatgtgtttggtaattcagaaagtatgaaccaatgcttcaaacactagtat

SEQ ID NO: 186 - DNA, 101 bp, Cannabis sativa
  source 1..101: mol_type = genomic DNA
atggaagaagacattatattctgctaccatcattgtaagattttccataattttatatgtatttatttcattattttaagaattttatgtttggggtggta

SEQ ID NO: 187 - DNA, 101 bp, Cannabis sativa
  source 1..101: mol_type = genomic DNA
atagacaaattaaaggccagagaaagctgatggtgaggtgacatctaattcggaaaatgtttacagcttgttgccttaagttgtgccaccgaaagcgacga

SEQ ID NO: 188 - DNA, 101 bp, Cannabis sativa
  source 1..101: mol_type = genomic DNA
agtcagtctgaagttggcatacacttactcccgagctaaaactactgcttccaagaacatggctctatccgacaccttaaaggcagaggcgggcttggcca

SEQ ID NO: 189 - DNA, 101 bp, Cannabis sativa
  source 1..101: mol_type = genomic DNA
ttaaaaaaattaaaaaattgagaaatcaaagaaaatgaggaaaaactcattcaaattgccacctcaattcgttaatgtttttctttatatatattaggaaa

SEQ ID NO: 190 - DNA, 101 bp, Cannabis sativa
  source 1..101: mol_type = genomic DNA
ccaagatggtcctttctggggggattttgctggacaagttttggacttaagccttgatcctatgactgattagttcacgagcttcctagggtcctcttcgg

SEQ ID NO: 191 - DNA, 101 bp, Cannabis sativa
  source 1..101: mol_type = genomic DNA
aaccctagccaggcatacaagatcacctagccagcctattatatccccttggcctactagacatgttggtacaactacttgatggtgtgtaactactttcg

SEQ ID NO: 192 - DNA, 101 bp, Cannabis sativa
  source 1..101: mol_type = genomic DNA
atattgctctacccatatactgcattatgcattttttatcattgctacgacgagtgtcttctcttccttgcattgattgagtatgagatactgtacgcctc

SEQ ID NO: 193 - DNA, 101 bp, Cannabis sativa
  source 1..101: mol_type = genomic DNA
tcgaccctttttacttatattattttaaagaattaataagatcataaggatttgggcaagtgagttttgttatcctgatttttggcactgacgtggcatgt

SEQ ID NO: 194 - DNA, 101 bp, Cannabis sativa
  source 1..101: mol_type = genomic DNA
tctctctcagttttatatatgattaaaaagattaaccactccacttgccctctaatataaagactcactctatttatattaaaaaataaaccgcccgccaa

SEQ ID NO: 195 - DNA, 101 bp, Cannabis sativa
  source 1..101: mol_type = genomic DNA
tccctatcccaaaaacagacttggacctatatagtgattaggatacgaacttggactcggactaagacccagactgggatcgaaatgctggaccaagatct

SEQ ID NO: 196 - DNA, 101 bp, Cannabis sativa
  source 1..101: mol_type = genomic DNA
actgaaatcgggacccgaacccaaactcagactagatgtcgaactttgactctaacaaaaacccaaacttgatctcagaataactagaaccgaactaggac

SEQ ID NO: 197 - DNA, 101 bp, Cannabis sativa
  source 1..101: mol_type = genomic DNA
caaactcaaacacatacacctagaatcagacccaaagttagatccgtacctggacccaaactcgaacccagaccaggatcagaaccaaggacttagaccca

SEQ ID NO: 198 - DNA, 101 bp, Cannabis sativa
  source 1..101: mol_type = genomic DNA
gacctagactcaagatgcgaacccgaacttagacctagactaggatctaccagccggactaggatgggggacctagaccagggacctaggccttggaacca

SEQ ID NO: 199 - DNA, 101 bp, Cannabis sativa
  source 1..101: mol_type = genomic DNA
aggccgagacccgtgatagtgatccaaataaagatacgtgactcgggtaagggggctggaacagaccagtagtcggtgtctagatctgggactcagactag

SEQ ID NO: 200 - DNA, 101 bp, Cannabis sativa
  source 1..101: mol_type = genomic DNA
tggtcttcagacagccctcctcagaccgagaaaactcggacaaaacctgtctgaaaccacacgcgtccgaggaaaaagaaggttccgcgcgcgtccgggtg

SEQ ID NO: 201 - DNA, 101 bp, Cannabis sativa
  source 1..101: mol_type = genomic DNA
ggtcacggtcctagtcttgttctagggcctagtttggcaattcttgttcatcttctaatttttcgtcctaggtctaggtttgtgttcgggtttggttcata

SEQ ID NO: 202 - DNA, 101 bp, Cannabis sativa
  source 1..101: mol_type = genomic DNA
tttctacagacacaactcacaaaaagagatagatagagaaacacaatacatatatcaacttggaaatagggatcatgtgagcgggttgttcccaatttaaa

SEQ ID NO: 203 - DNA, 101 bp, Cannabis sativa
  source 1..101: mol_type = genomic DNA
tcacatcgagtcgcgaatagacgcgcctgacacgtgtcacccattcaaacacaaggagagcttggatgaagtctacacgcaactttgagagtctcgtacat

SEQ ID NO: 204 - DNA, 101 bp, Cannabis sativa
  source 1..101: mol_type = genomic DNA
ttagaattgcgttaattaaataagataagactcgttgaaaattttgttttagttgatgtcactttgggtcgacattttcttattctacacacaagcttcac

SEQ ID NO: 205 - DNA, 101 bp, Cannabis sativa
  source 1..101: mol_type = genomic DNA
acattggcaattgtaacacgatcaactatttgtaaggttatattcatgtatcttagacaaatctaaaacatgtttgattatgtaatcatacacatcataca

SEQ ID NO: 206 - DNA, 101 bp, Cannabis sativa
  source 1..101: mol_type = genomic DNA
gaatttgccaatataagaaattatttacagaaaaaccatcccagaagagactacagatagaaagtgtgtaaactatttacattgcctcaatgatgaaacac

SEQ ID NO: 207 - DNA, 101 bp, Cannabis sativa
  source 1..101: mol_type = genomic DNA
atttgggaaaagagattgaaccgctccaacagcgagaatcaaagctcatcgtccaccacaagccggtcagattacgggctgagctgtaacgagaattcagc

SEQ ID NO: 208 - DNA, 101 bp, Cannabis sativa
  source 1..101: mol_type = genomic DNA
attggaaacaatcttctattcatttaaaatttcagattttactcttataaggtttcaatttttggttttgtcaatattattcttgttccaaaatgaaggct

SEQ ID NO: 209 - DNA, 101 bp, Cannabis sativa
  source 1..101: mol_type = genomic DNA
ctattcaaatggaaactttccacaatggtcttaatgtttctactcacaaattattgaatgctttacctaagggggtattctttctaagtcctacaatgagc

SEQ ID NO: 210 - DNA, 101 bp, Cannabis sativa
  source 1..101: mol_type = genomic DNA
cgatcatggggtctaggtttagtcccatatcttcccagtgtactcccctcaaaagggtccgccatgggcctcccttaaccatttcttcaaagcctcagcat

SEQ ID NO: 211 - DNA, 101 bp, Cannabis sativa
  source 1..101: mol_type = genomic DNA
atttctaaaagcacactaaaaactaatttaggcagattaaatgaaggccagggacaggtaacattataaactctgagctcacctgatgtatgtgatccaat

SEQ ID NO: 212 - DNA, 101 bp, Cannabis sativa
  source 1..101: mol_type = genomic DNA
ttgatcaggcatgagattgagggttttgcgatgtattgtatatgttcaggccaagctagaccacaagggttgggtctactgttaaatgccaaaatattaca

SEQ ID NO: 213 - DNA, 101 bp, Cannabis sativa
  source 1..101: mol_type = genomic DNA
gatttatataaaatgtaaaagacttgctcctaagccaggaagtctttattaattggatatacaaatgtgttggttcgaaagtcaaaagaataaccacactt

SEQ ID NO: 214 - DNA, 101 bp, Cannabis sativa
  source 1..101: mol_type = genomic DNA
ttgttgcttggatcaaagtttgatttagtgatctttatgcttgtgttgagatagctattttaattggtgtaatttaatttcagctgctgaaaaggattgga

SEQ ID NO: 215 - DNA, 101 bp, Cannabis sativa
  source 1..101: mol_type = genomic DNA
gatgtgattggtgctctgtcattgataatatactctctcactcttgttccacttctcaagtatgttatcgttgtgtgcaaggcaaatgacaatggtcaagg

SEQ ID NO: 216 - DNA, 101 bp, Cannabis sativa
  source 1..101: mol_type = genomic DNA
ccagttccaagcatttttctcaagaagtctcttggaattttatccattttcatctataacgaagatacaagtgctctcgggaagaaaacgagaccatttga

SEQ ID NO: 217 - DNA, 101 bp, Cannabis sativa
  source 1..101: mol_type = genomic DNA
gagtggtgtgatgatgaaaagcggttttggtgtagaaggagaagaaaatcttgggtttattatggtgaaatttaaaacagagaagtaggaagagtttgtgg

SEQ ID NO: 218 - DNA, 101 bp, Cannabis sativa
  source 1..101: mol_type = genomic DNA
agtgaactgtgttaatatcttctgcccatagacttgttgctaaagaggtgaaaccttttgtttttccataaactttctattttaattttataaagattgtt

SEQ ID NO: 219 - DNA, 101 bp, Cannabis sativa
  source 1..101: mol_type = genomic DNA
ttaagtttgtttggtattcatttcttgtaattttgctacatctggttgtagtggtctccattagtggaagcaatatcttgagtaacgtctccaacagccct

SEQ ID NO: 220 - DNA, 101 bp, Cannabis sativa
  source 1..101: mol_type = genomic DNA
agatttagacggtctgtaacatctccaactgcccaaatggaagggactgaagttcgagagtattcatcaacctaaagaaacaagtgaaggagaaaagcatt

SEQ ID NO: 221 - DNA, 101 bp, Cannabis sativa
  source 1..101: mol_type = genomic DNA
tgtgaaaattaaaggaaaaaaaatcattttctggttgtctacctcttaaattgaccccctctccctctctcaatcacttttgtaattttttagagaatctt

SEQ ID NO: 222 - DNA, 101 bp, Cannabis sativa
  source 1..101: mol_type = genomic DNA
aggagcgtctcttttgtagaaaatttcacgatcctaaggaagtagggacttaacttgattattagatttaattttatttatgttggataacattagacata

SEQ ID NO: 223 - DNA, 101 bp, Cannabis sativa
  source 1..101: mol_type = genomic DNA
ctactaatgaacctgttgttgcatctgcttctttagaaatggctgaattcgacttgcatttgggaagaatggagacaatgtttgttacaactctgcactct

SEQ ID NO: 224 - DNA, 101 bp, Cannabis sativa
  source 1..101: mol_type = genomic DNA
gaggattcatgcaacaagcatgcgagtgattagaaaagtctaaatctaggaagttttgtgacagggacgatggtgaagaagttaataaatccccctgataa

SEQ ID NO: 225 - DNA, 101 bp, Cannabis sativa
  source 1..101: mol_type = genomic DNA
ataagaatcatggcaatatctatgcttccgctgatagtagcataacaaaacgcagtgtttcctttgcaatcctgcaaagctagatcgttttcttccatcaa

SEQ ID NO: 226 - DNA, 101 bp, Cannabis sativa
  source 1..101: mol_type = genomic DNA
ttaaccgtttcccaactagtttatcgtttcttggaatttcgaggaaattaagaatcgtatttttgtcataacttttaactcgggtgtttgttttggacgtt

SEQ ID NO: 227 - DNA, 101 bp, Cannabis sativa
  source 1..101: mol_type = genomic DNA
ttaattcaacatactgttagtaaatctagatctttgtaaaatattcccaacagacacttgatgtacaacttgcatgcaagacaacaattcaagctgggaat

SEQ ID NO: 228 - DNA, 101 bp, Cannabis sativa
  source 1..101: mol_type = genomic DNA
gtggattgctggctgtgaggtagttaatgatgatagagtagtttgtggttgttgttgggtgaaataggaattgaatgggcgtgggggtggaggttgaaagc

SEQ ID NO: 229 - DNA, 101 bp, Cannabis sativa
  source 1..101: mol_type = genomic DNA
tttgacggggcagttctgatccaaacaaatggatttcttataagatctcggattgggaacccgatctggtacaaaattatccaaatctaatcagacccgtc

SEQ ID NO: 230 - DNA, 101 bp, Cannabis sativa
  source 1..101: mol_type = genomic DNA
ttttgacattacttagtcgttcaccagttctcggatatgttgttgtttcgcaaaattgagagactcgaaaatactcaatttgtgactagttcactttgccc

SEQ ID NO: 231 - DNA, 101 bp, Cannabis sativa
  source 1..101: mol_type = genomic DNA
tctacctctaccttggtttccacgaaaccatttactttgccctgaaccaccagatgtcgaggtaaccttcctttttagatcaatggtggagtcaccaccat

SEQ ID NO: 232 - DNA, 101 bp, Cannabis sativa
  source 1..101: mol_type = genomic DNA
taaagccactgaataatgatcactctcttctaatccattattctctccttatttgtctaacatgggtcctcaaagtggttttgctagggagcaacaggtaa

SEQ ID NO: 233 - DNA, 101 bp, Cannabis sativa
  source 1..101: mol_type = genomic DNA
tacagtaattataataacttttcctagctaattagctacaccaaccaaaacgccaagtttcagttgatagttggaccacctaaagaaaaaccaaaaaacta

SEQ ID NO: 234 - DNA, 101 bp, Cannabis sativa
  source 1..101: mol_type = genomic DNA
attcattatttaataattcatggcatatgattatgggtacagttgatagtggcattagcagccgcgggagacggtgcagctaccttcatggcagagttagg

SEQ ID NO: 235 - DNA, 101 bp, Cannabis sativa
  source 1..101: mol_type = genomic DNA
ctaattttttttgcacattttttgtaactaattaagaaaatgtatacattcatgtaaaatgccctaatttttaagcttactttagcatttttttgcctttg

SEQ ID NO: 236 - DNA, 101 bp, Cannabis sativa
  source 1..101: mol_type = genomic DNA
tacgtcatcgtatcgtgatgggtcgaatttctctggctctggaaaatattttgggcttttgtttgttgtactcactgtccagtatacctgcaaatttatat

SEQ ID NO: 237 - DNA, 101 bp, Cannabis sativa
  source 1..101: mol_type = genomic DNA
caattccttccccggccgattcatatccaccttgttggggccaagctccttgtggcaaacccaaaaactgaggtgtatgtgttgtcgccgttctaacggaa

SEQ ID NO: 238 - DNA, 101 bp, Cannabis sativa
  source 1..101: mol_type = genomic DNA
ggaaaaagaaacctactgttgacaaagagaacaagaaagatcagctgaatgagcaaagtgaaacagggggaattgttgggatgacagaggataaagaagct

SEQ ID NO: 239 - DNA, 101 bp, Cannabis sativa
  source 1..101: mol_type = genomic DNA
ttacaaaattcaaatggaaatataatagcatgtctttcaaaactaatattaggtatgtattttatttctatacatagatcactagatacaatatatatact

SEQ ID NO: 240 - DNA, 101 bp, Cannabis sativa
  source 1..101: mol_type = genomic DNA
aacctaaaaagacatctttccactccccagaatcaaccatcttttagatgccataattggcatgaaataatgagtttcatggacacgtatttaggttacaa

SEQ ID NO: 241 - DNA, 101 bp, Cannabis sativa
  source 1..101: mol_type = genomic DNA
cattatccttgagattatatctttcaagcatctttttctatgcttcgataaattcatcttcctcctcatgttcctatatacaatcaccaaagtctgttgag

SEQ ID NO: 242 - DNA, 101 bp, Cannabis sativa
  source 1..101: mol_type = genomic DNA
attgcaagtctaaaggagtggttggcagatccagccattggaaacaacccgatcctcagactgattgcaggaactatattctttcatgaacaagactataa

SEQ ID NO: 243 - DNA, 101 bp, Cannabis sativa
  source 1..101: mol_type = genomic DNA
tattagtttagcccaaggttcagctaaaggatgtgcaaacatatctgaatcagccacaagcacctataagaaataacaacagttgccaccaaccaatcaga

SEQ ID NO: 244 - DNA, 101 bp, Cannabis sativa
  source 1..101: mol_type = genomic DNA
tagtcaccagggcggcggtcaagaaaatcgactttgatattcaccccagttgccttcttacatgcttccacaaattccttcactgatctacctgaaaaaag

SEQ ID NO: 245 - DNA, 101 bp, Cannabis sativa
  source 1..101: mol_type = genomic DNA
ccaagtggggactgggctctgcttactgaggaacagtcgttgtgctgtaatgttatctgatctaagtactaggggtgttcaacaaaatttacaaaccgccc

SEQ ID NO: 246 - DNA, 101 bp, Cannabis sativa
  source 1..101: mol_type = genomic DNA
ggttagattggtgcattgtcaattcatattggaaagacctcttcccaagtgcatcccatttttacctccctttccatggatctaatcatagacctcttaag

SEQ ID NO: 247 - DNA, 101 bp, Cannabis sativa
  source 1..101: mol_type = genomic DNA
ctgactaccaagtagaatctactctccaagttcctatttattcaactagagaggatagtctcatttggggtcaccataattcaggtttgtttactgtcaac

SEQ ID NO: 248 - DNA, 101 bp, Cannabis sativa
  source 1..101: mol_type = genomic DNA
aacttacattagtacggtgctggagcatttttctaatggctaattctataggaagatttttggctcctagatatagattttttttatctaaggaacagttt

SEQ ID NO: 249 - DNA, 101 bp, Cannabis sativa
  source 1..101: mol_type = genomic DNA
aattaactttcctttgaacctcttgtgaatctatttccatttcaaaaatttgaagtttactgtctcacaccattgtagagctttcaataaagctataggtt

SEQ ID NO: 250 - DNA, 101 bp, Cannabis sativa
  source 1..101: mol_type = genomic DNA
acatttaagcccactttaaagtatattgaagaattagagatgatgttgtatatttaggggtattaatagaaaaaatcattaggtttaaagcttcagaaatc

SEQ ID NO: 251 - DNA, 101 bp, Cannabis sativa
  source 1..101: mol_type = genomic DNA
taaaataatttccaacacattaaaatacttattctgctcctgcattctaatagcagcacacaagcccttagaaacaagaagaaaggtgtaagaagagagag

SEQ ID NO: 252 - DNA, 101 bp, Cannabis sativa
  source 1..101: mol_type = genomic DNA
gttccaacgaattttcttactgctgcaaattcaaagtctgtcgctacaaatgcagtatctgtagaagcaaagtctctagtaattacaaaaagtgtaagatc

SEQ ID NO: 253 - DNA, 101 bp, Cannabis sativa
  source 1..101: mol_type = genomic DNA
aaagtcacttgaagtataacaaagttcaccgctattacaggccaccacccaaccgaggaagtttcaattggagctgagttgtatattcattgataagcaag

SEQ ID NO: 254 - DNA, 101 bp, Cannabis sativa
  source 1..101: mol_type = genomic DNA
tctagatggctcgctagtactataactttcttctcgaatcacacgttcaggaggaaaacaactgaattctcttagagcgacagacctaactcggcatcctc

SEQ ID NO: 255 - DNA, 101 bp, Cannabis sativa
  source 1..101: mol_type = genomic DNA
cgagctccacaagtagttgagttttatcattcactaatgaagagagattctaggaaggattcttcaaatggagggatatgtgatgttccagatgttgccaa

SEQ ID NO: 256 - DNA, 101 bp, Cannabis sativa
  source 1..101: mol_type = genomic DNA
ttttttttttagttttagctttctttggcacttctagttgagctaataatagtttgacttattgaagatagtactttggtgaatggttcaattgaagcaag

SEQ ID NO: 257 - DNA, 101 bp, Cannabis sativa
  source 1..101: mol_type = genomic DNA
aaagtagaacccgtttcgaaagcaccatgtagaatggctccatcagaactcaaggagctagaaatacaacttcaaggaatgcttgatttaggctttatccg

SEQ ID NO: 258 - DNA, 101 bp, Cannabis sativa
  source 1..101: mol_type = genomic DNA
aaagctctaaaactaacacgaattgaaaacatgaaaagtgcacgaaactggtttttgatctgattaaccggttttaccaggcctgtaaaactggttaaccg

SEQ ID NO: 259 - DNA, 101 bp, Cannabis sativa
  source 1..101: mol_type = genomic DNA
aatcttctaatttactaaaaaaaaatatagtggatattgttacagtagattagagaatatattgtgggtatatattgttagacatcatcaaggaggtattg

SEQ ID NO: 260 - DNA, 101 bp, Cannabis sativa
  source 1..101: mol_type = genomic DNA
aagtattgtgttggctgctggagctgttccatgggtttctcttcatataatattctttttgtgttttatgtgtgactataaaaactcatgctgttttgtgg

SEQ ID NO: 261 - DNA, 101 bp, Cannabis sativa
  source 1..101: mol_type = genomic DNA
ttagtccctaattaatacaagaatcttgtatgtattttgcaagttattttcccacttgcatggaaattggaatagtcattttggaggggagcacaagagaa

SEQ ID NO: 262 - DNA, 101 bp, Cannabis sativa
  source 1..101: mol_type = genomic DNA
ataaatgaatgggttgctttcacatggggaaggaaggattctctttttgtaaaagagaggaagtggtcagagaatcccagttaagttttgttgttttgtga

SEQ ID NO: 263 - DNA, 101 bp, Cannabis sativa
  source 1..101: mol_type = genomic DNA
actttattctaaaatatatcccataacccctgttacagttccattccttattataattagaaaaatgattcactaagcaacaacaaattatgtaacaaaaa

SEQ ID NO: 264 - DNA, 101 bp, Cannabis sativa
  source 1..101: mol_type = genomic DNA
gattatggttttattacacacgtacagatggaattgatagttcatatggattgtggtggttgcgaaagcaagataaagagagctcttctaaacttagaagg

SEQ ID NO: 265 - DNA, 101 bp, Cannabis sativa
  source 1..101: mol_type = genomic DNA
tctatggaattcatggcaggattatcttcaacgtgaaatccaggagcttttgtttctgcctggctctttatagatacttcttcggaaaggcttttccatgc

SEQ ID NO: 266 - DNA, 101 bp, Cannabis sativa
  source 1..101: mol_type = genomic DNA
cgaggaatgaatgacacaacacaattggaaaacaaaggtatcaaagcacggcattcatggaacacagggaaagtactccaagcaaatggtagaacacccga

SEQ ID NO: 267 - DNA, 101 bp, Cannabis sativa
  source 1..101: mol_type = genomic DNA
gtcgatgatatgttggtaaagtcggtataatgtgaagaccatagtcgccatacaaaggacctcgcagaatgtttcgaaattctaaaaaagtacaatatgaa

SEQ ID NO: 268 - DNA, 101 bp, Cannabis sativa
  source 1..101: mol_type = genomic DNA
acatgtttcaagtgctgcaattcttgtacaagtaatttattacctccacattgaacttgacccatcactatgttttcggacactccacatccaaacatgtt

SEQ ID NO: 269 - DNA, 101 bp, Cannabis sativa
  source 1..101: mol_type = genomic DNA
tatgacgacatcagtaaagatgcaaggaaattcttgactcccgatgtcccggaagatgagatttattaaagcttttggtgttgatacaagggtacattatt

SEQ ID NO: 270 - DNA, 101 bp, Cannabis sativa
  source 1..101: mol_type = genomic DNA
atcaatgatcttaagaactcgcctggaccctctaagaaggagcttcgaatcgcaacaaagcaagttgtggggctattacaggacaattaccctgagttggt

SEQ ID NO: 271 - DNA, 101 bp, Cannabis sativa
  source 1..101: mol_type = genomic DNA
aacttaaattagtgaggcaaggaggaatcatcccagtgaagctattattggccaaatctagtacactgattgtttctagcttgcaaatcaaagaaggtagc

SEQ ID NO: 272 - DNA, 101 bp, Cannabis sativa
  source 1..101: mol_type = genomic DNA
ttgcagaggaggtaatgaaaaagttggattgagaacaaaataggagaaggtgaaaacataataggctaaatgatataacttcatattaatcttttaaaatt

SEQ ID NO: 273 - DNA, 101 bp, Cannabis sativa
  source 1..101: mol_type = genomic DNA
actattaaaatatggatacatatattccaatcttccaattttctttcaaaattacctctcttttcaatacccccattctctctagctccctttttctatcc

SEQ ID NO: 274 - DNA, 101 bp, Cannabis sativa
  source 1..101: mol_type = genomic DNA
gccaagaggtcatctagtcagatcatgatcaaagaaaaactcactaatgccgcttatttgaaaattaaaaacatgcgcctattcttctatcaggctaatat

SEQ ID NO: 275 - DNA, 101 bp, Cannabis sativa
  source 1..101: mol_type = genomic DNA
aaagtccaaagaggagaaaaaggcagacacctttttcctttcttttaattgtttctttcctttgtcttttattccctcatcattgtacctcccccacacac

SEQ ID NO: 276 - DNA, 101 bp, Cannabis sativa
  source 1..101: mol_type = genomic DNA
ccccaagggtgtgggatatgtgcaaggagtctaatcaaactcacgagagtgctataatatagcactgaagctcgcaaaaaagaagaaatcaataaatgtta

SEQ ID NO: 277 - DNA, 101 bp, Cannabis sativa
  source 1..101: mol_type = genomic DNA
agcatttcggattttggattttgtttaattaattaatattatttttctctttctggtcagattctccatcactgatgacctaccccctcttatcagcttta

SEQ ID NO: 278 - DNA, 101 bp, Cannabis sativa
  source 1..101: mol_type = genomic DNA
tttgagttccctcttggaaacttgaacttgttccaattctatcatttgtgactgtctttacagtcaactggaactaggcacaaagtgctaacaagcttctc

SEQ ID NO: 279 - DNA, 101 bp, Cannabis sativa
  source 1..101: mol_type = genomic DNA
aaaacctaacattgagaagagcttgatcagacacaccacaagccttctaagtacaacactctgagaagaactagtctctccttctaaagatatatacctaa

SEQ ID NO: 280 - DNA, 101 bp, Cannabis sativa
  source 1..101: mol_type = genomic DNA
actactttgcggcttttctcacttggttttgatgtcttgatgataaaaaagtatatttgcaaagaatctcattccccttacttgtggccgacctgtgccac

SEQ ID NO: 281 - DNA, 101 bp, Cannabis sativa
  source 1..101: mol_type = genomic DNA
tgggcgggtttgagaaagtcccacccaagtactcagtacacactctcccagtttcccctctggaagtaacccaacaatcattcctatctagaatactacca

SEQ ID NO: 282 - DNA, 101 bp, Cannabis sativa
  source 1..101: mol_type = genomic DNA
cttgtaggtatgcatcttgaactttcgagtgatgaggaggtcatggatttcttatgagaaggcagaccctctcctccacttcaattctaagtcagttagct

SEQ ID NO: 283 - DNA, 101 bp, Cannabis sativa
  source 1..101: mol_type = genomic DNA
gcacaagattatcttctacatgtgtctttttcttgctatatggatttttaactattaacatatattttgtattccttttaataaattttttctttttacca

SEQ ID NO: 284 - DNA, 101 bp, Cannabis sativa
  source 1..101: mol_type = genomic DNA
atatttttcattctttcattcatttttctctcacaactttgttcatcaacacctttaaacgagttcctaaaatgcttccaaaaaaactctaataataactc

SEQ ID NO: 285 - DNA, 101 bp, Cannabis sativa
  source 1..101: mol_type = genomic DNA
ttaatgtaacaaattattacatgaatctttcaagacaattgtacaagaagctggctccctatgtttcaaccaatcctagagaaactttcttgaattatcga

SEQ ID NO: 286 - DNA, 101 bp, Cannabis sativa
  source 1..101: mol_type = genomic DNA
aaggaagataagatgttagttccctattaatattcccagtggagcagactttttttggtgatctaagttgagtgttcagttatttgaacatcagtatctta

SEQ ID NO: 287 - DNA, 101 bp, Cannabis sativa
  source 1..101: mol_type = genomic DNA
atttggggatgaaggagggtacgagtattgacaccactactagtaccagtaccaccaaaacatgtggatttactggatttactattgtctgcatttgagcg

SEQ ID NO: 288 - DNA, 101 bp, Cannabis sativa
  source 1..101: mol_type = genomic DNA
tcaagcggccataatatggactccatatggtggaaagatgagcgtgattccggaatctgattcccctttccctcataggaaaggaatcattttcatgagtc

SEQ ID NO: 289 - DNA, 101 bp, Cannabis sativa
  source 1..101: mol_type = genomic DNA
tatcaatgcccccacatgattcaaaattgacataatgtatgttctcttatttattttccatgtctgaatgtaagagataaaataagtttttattttaacat

SEQ ID NO: 290 - DNA, 101 bp, Cannabis sativa
  source 1..101: mol_type = genomic DNA
agtagtgagggtatgcaatagggcctaagggctccccacaattgtaattgctgtactcatacatataatacatacatttttagtgtttggctactattttg

SEQ ID NO: 291 - DNA, 101 bp, Cannabis sativa
  source 1..101: mol_type = genomic DNA
gtattgactaattgtaaaataaattgttatttttagtgtcatcatcatatcgtgtttgtgtggtgtggagtttttaaatgtattttagatgggcaacaaaa

SEQ ID NO: 292 - DNA, 101 bp, Cannabis sativa
  source 1..101: mol_type = genomic DNA
tcaaatataaaattttataaaaaaagttactatacgatgcaacatgataaatacaaaactatacaaaaacacgaaagaagcacaaacatacctatgaataa

SEQ ID NO: 293 - DNA, 101 bp, Cannabis sativa
  source 1..101: mol_type = genomic DNA
taagaaaatggaggttggttatttaaccttatatcactatataagatttatatatttcttttctataaagtcttcacaccaattgatacgttggagcatat

SEQ ID NO: 294 - DNA, 101 bp, Cannabis sativa
  source 1..101: mol_type = genomic DNA
tacacaactgatcagcacgagacttttcttccagtgctttctttcggttcatttctgaaagctttttatgttgttctgaattcaacttctccctgtcagcc

SEQ ID NO: 295 - DNA, 101 bp, Cannabis sativa
  source 1..101: mol_type = genomic DNA
attgattttttccttgttgcgatgaagccaccttagatttttgtggctcagcatggcttgactagtgaaactaaagaacaaataaatgtaactcataacat

SEQ ID NO: 296 - DNA, 101 bp, Cannabis sativa
  source 1..101: mol_type = genomic DNA
ttaaggtttgttttaaattaaatttcattattgaatttattcttgtcatttgcatctccttcctttggagcatatttcaacctttcatgttaccatgtaca

SEQ ID NO: 297 - DNA, 101 bp, Cannabis sativa
  source 1..101: mol_type = genomic DNA
gttaatggcttcccgtgatgtaagtagtagtgagtaacatcatacataacatatcctaataaaatccctccgaacaatgctggagcaactgatggtgatga

SEQ ID NO: 298 - DNA, 101 bp, Cannabis sativa
  source 1..101: mol_type = genomic DNA
aaatcgtcagaaattgtaactcagtcctaattaattacggatatagataacagatgtaatactcatcacaattgccgtacagtgtttgagtgtcttttacg

SEQ ID NO: 299 - DNA, 101 bp, Cannabis sativa
  source 1..101: mol_type = genomic DNA
gcaaacctgatccatggccaaccaaaatttgttcttggcccaggtactctgaattgttgatcagattttaagcatcaggtgtaaggtgatttgttgcacct

SEQ ID NO: 300 - DNA, 101 bp, Cannabis sativa
  source 1..101: mol_type = genomic DNA
atataggggcatcacccttcaagtattttcactgcaaattctacttatcacttagctaaatctcgtttcttttcacatgttccttaatcttcaaacgcttc

SEQ ID NO: 301 - DNA, 101 bp, Cannabis sativa
  source 1..101: mol_type = genomic DNA
gctgccattgattctcttaatgcttattgtggtctaagtatggttatcaggggactctttagtcgcttggaaagtagaagaaaattgcattccagaatggg

SEQ ID NO: 302 - DNA, 101 bp, Cannabis sativa
  source 1..101: mol_type = genomic DNA
aaagactctctcttccttctcacctatttgaaccctagaaaccagcaacagcaaagaagatttctccaccaatttcacaatttcttagctgaattgacaag

SEQ ID NO: 303 - DNA, 101 bp, Cannabis sativa
  source 1..101: mol_type = genomic DNA
gatgttgcatccatgcttgactattcccatcgtcaaacatgcctcgtaataagccactaagagctcgccattgctagaccttcacaacccctattattgaa

SEQ ID NO: 304 - DNA, 101 bp, Cannabis sativa
  source 1..101: mol_type = genomic DNA
ctttaaggtaggctatgttctgtttgagtggggatcctatcctttgtttcaagattgtgttaattgtttcaaggagtttgtgttgggttttatgccctaaa

SEQ ID NO: 305 - DNA, 101 bp, Cannabis sativa
  source 1..101: mol_type = genomic DNA
agttagttagttagtattttagctcgattttctcacttctttggaatccttgtttaggtgtcgatctggagagagattcaaatgaaaagattaagattgag

SEQ ID NO: 306 - DNA, 101 bp, Cannabis sativa
  source 1..101: mol_type = genomic DNA
agtcctcattggtttttgatgcaaatctgctatgagagctgagttgcagcctccatatcatcaattccccttgatgttgtagacttttctatggagctgcc

SEQ ID NO: 307 - DNA, 101 bp, Cannabis sativa
  source 1..101: mol_type = genomic DNA
tgggttaatgcttatttatttgcagtccgtttggttgccgagaaagtgctataaagataaagattctgctttctttttttgcttagcaagaagaataccct

SEQ ID NO: 308 - DNA, 101 bp, Cannabis sativa
  source 1..101: mol_type = genomic DNA
aaattctggtaaaaaaaattccatcaagcattgtttactacttttgctaaattttttgcttctattaatgatttctatgatttcttttttattggggttcc

SEQ ID NO: 309 - DNA, 101 bp, Cannabis sativa
  source 1..101: mol_type = genomic DNA
tatttttgtaaagattggaccaagaatcagtatttttgtaaatttcctttgatatattgttttaaacctaccactaatactattaatgcatataagacttc

SEQ ID NO: 310 - DNA, 101 bp, Cannabis sativa
  source 1..101: mol_type = genomic DNA
aatttttttgaacaaccctaatttgtgtgtgttgtgttttggttcttagggtttctaaacttttcttgagaagtcttttaagccgtgggttcaaacgacac

SEQ ID NO: 311 - DNA, 101 bp, Cannabis sativa
  source 1..101: mol_type = genomic DNA
tgaagcagtgaagtctttttgaaaagcacgtgatagtttccttttctccacgaggagattactacttcattttggcctttgcttaatgaggttggaaactt

SEQ ID NO: 312 - DNA, 101 bp, Cannabis sativa
  source 1..101: mol_type = genomic DNA
cttaatattctagcaagtatcatgaagatacatataggtaactggttatatgtaagatagtaacaagttacattgatggtaatcgggttaccttatataat

SEQ ID NO: 313 - DNA, 101 bp, Cannabis sativa
  source 1..101: mol_type = genomic DNA
aaagcatttggtagctggggattttgtatgtggtccaccaacacattacaatcagttcctatttggtagctgggcatagtgttgttgctcagttttgatat

SEQ ID NO: 314 - DNA, 101 bp, Cannabis sativa
  source 1..101: mol_type = genomic DNA
ttctacctagccccttgttcgaagtagtagccatgtctgagatagtgaacgaattttggcaaaagtatcttaaccaacttcatcttcatcctctcaggacc

SEQ ID NO: 315 - DNA, 101 bp, Cannabis sativa
  source 1..101: mol_type = genomic DNA
ccctaccttatttccttctgggtatgataatgatgatgttgtcgatgatgttgatgttgctgccaatgaagaagaagaagaaaatgatgatgatgatgatg

SEQ ID NO: 316 - DNA, 101 bp, Cannabis sativa
  source 1..101: mol_type = genomic DNA
atgatttttacgtggtttgagtgaatgaactctagtccacgaattaatagtattaagctagaaaagttttgaaatatttcatacaagtatttacgccatag

SEQ ID NO: 317 - DNA, 101 bp, Cannabis sativa
  source 1..101: mol_type = genomic DNA
accaatggagggctctaactcttggcaatctctactaatctgcaattatgaaaacataagaatgagataatgtatttgtgagtgtgtaacagtgaaccaga

SEQ ID NO: 318 - DNA, 101 bp, Cannabis sativa
  source 1..101: mol_type = genomic DNA
atgcttgctcgatgggtgcaataattttcatcataaccccatgcaagggtcggagaaccatcaaactcacatcgaactaacatcgggcctgtgcttttttt

SEQ ID NO: 319 - DNA, 101 bp, Cannabis sativa
  source 1..101: mol_type = genomic DNA
acttcacataacctctaaatagataagttttgtgctagcccaaagcctactcctccaagcaccaccaacaaagtcagaagtcaaaaccttgttaaactcta

SEQ ID NO: 320 - DNA, 101 bp, Cannabis sativa
  source 1..101: mol_type = genomic DNA
caagaacacattgtttagattcaagtttatttattttcaatattttctttatagcgaatagagtaaactttaacatcaccaatcaaagatttggtagcacg

SEQ ID NO: 321 - DNA, 101 bp, Cannabis sativa
  source 1..101: mol_type = genomic DNA
aggaaaacgggtactcctgatagcagggattccaagttcattgttcttcctcaggtggagataaaagctagtgatgatgtggtgagtactacggttatcta

SEQ ID NO: 322 - DNA, 101 bp, Cannabis sativa
  source 1..101: mol_type = genomic DNA
ctctctctcgcttcagctctctttctcgataacttcatcaagatgaagatcatcgccgcttacttgttagccgttttgggtggaaacgctgctccttccgc

SEQ ID NO: 323 - DNA, 101 bp, Cannabis sativa
  source 1..101: mol_type = genomic DNA
ccattttttggctagtttgcagtatgttattggctcatttgtgcatggattcaacttcaaagagtgttattataaattttcacagataagttttaatcttg

SEQ ID NO: 324 - DNA, 101 bp, Cannabis sativa
  source 1..101: mol_type = genomic DNA
aaaaaccaaagtggaactgtaattgcatacccataccaaccatcgctggtgccccaataagcagaagtaccaatagaagaccccaaaatcactacaaggaa

SEQ ID NO: 325 - DNA, 101 bp, Cannabis sativa
  source 1..101: mol_type = genomic DNA
gtgagagttgcagcaagtgttccaataggaattgaacgctgagtgtctttcagtgaggctgatcgattagaacctgccataattcctgttacagcagggaa

SEQ ID NO: 326 - DNA, 101 bp, Cannabis sativa
  source 1..101: mol_type = genomic DNA
agaattctccgaaggagaacccctatcctgggatgtatcttcgaattccacaaccattcccataaaattttctttggctgaaaacgaagctcttgatctaa

SEQ ID NO: 327 - DNA, 101 bp, Cannabis sativa
  source 1..101: mol_type = genomic DNA
gcagccttacagaaacaattggaaattgaaaagactcaatatcaaaattctgtgcaggcaaccaaagccatgactttaaacaatctgaaaacaagtctccc

SEQ ID NO: 328 - DNA, 101 bp, Cannabis sativa
  source 1..101: mol_type = genomic DNA
tcgccaagtttattttgttcactgaaatttttggttgcatgtttcaaatcattgtacttaaaatttactggaccttgtaattctgtacctcccaatatgtt

SEQ ID NO: 329 - DNA, 101 bp, Cannabis sativa
  source 1..101: mol_type = genomic DNA
gggatgctaaggtcagctttggacttgcctcttattttcttactccatcactataagatctataagttaaggagtccatattttgatactctccattatat

SEQ ID NO: 330 - DNA, 101 bp, Cannabis sativa
  source 1..101: mol_type = genomic DNA
gggggatgaaataataaattttttgatgatacagaggacaaataaagtttcggggtagcaacttggatttaacgagctaaaggatttaagggtgtatcatt

SEQ ID NO: 331 - DNA, 101 bp, Cannabis sativa
  source 1..101: mol_type = genomic DNA
gagttcactacagtcctcacaacaacaagtcacatgcatggattggatactgtataatatacaataatataaagacgaccagaccagactttgcatactga

SEQ ID NO: 332 - DNA, 101 bp, Cannabis sativa
  source 1..101: mol_type = genomic DNA
actagacatgattagccaaaaagaatttgccagtgttgcaaacacctaaaccgaatagaggagtcagcaattaaatcttgtgaaatttgtttctacttatg

SEQ ID NO: 333 - DNA, 101 bp, Cannabis sativa
  source 1..101: mol_type = genomic DNA
attagagactactttgctcggaataacttcgatatttcaccttatgaaatcattatcaatcttgcttgttaaggcctcgtcttgctcattagatgagacac

SEQ ID NO: 334 - DNA, 101 bp, Cannabis sativa
  source 1..101: mol_type = genomic DNA
gtcctccaaatgacagcattgggctttaatggcatgtttagtatgtactcgtatgcttccttaacaagaccagctctgcccaacaaatcaatcatgcaacc

SEQ ID NO: 335 - DNA, 101 bp, Cannabis sativa
  source 1..101: mol_type = genomic DNA
acatatgcagttcctatacagtagtatacagtattatcatctgagaaagaacagctaagtacggagcagccgtactcaaatgtatctaggggataagttga

SEQ ID NO: 336 - DNA, 101 bp, Cannabis sativa
  source 1..101: mol_type = genomic DNA
ctaagatagcaatataatactagttcaatgtcaaacttaaaccttgtaacaagctcagatgcttcagcatcccattggagaacacagaatttgtatctttc

SEQ ID NO: 337 - DNA, 101 bp, Cannabis sativa
  source 1..101: mol_type = genomic DNA
gtaaccaagaatgacagaccttccaaacaaaatgtttaattttaggaggccccattaccttccacaaagaagtccaccatgaattcatccgtcgaggatca

SEQ ID NO: 338 - DNA, 101 bp, Cannabis sativa
  source 1..101: mol_type = genomic DNA
tcaacaagctttttcacttctttcattacttgttccgaagcactagacttattgtacctagtggattcactcttagaagagtaaatgctcaaaaacccatg

SEQ ID NO: 339 - DNA, 101 bp, Cannabis sativa
  source 1..101: mol_type = genomic DNA
gagcaagggaaaagaagaacatggtgtgttttaagttataccccagggaattcacagttaggtagctcggaaatcaaggaagaggcgcagcttctgcggaa

SEQ ID NO: 340 - DNA, 101 bp, Cannabis sativa
  source 1..101: mol_type = genomic DNA
cggaaaggagaccattttctaggcaagggcaggcccaacccaacccccaacgttgacgacaaatagcaatttcgacctgacgactgagtagtacgaactta

SEQ ID NO: 341 - DNA, 101 bp, Cannabis sativa
  source 1..101: mol_type = genomic DNA
actaggagtggtgtgtacacaaattccttcactccaaagtaaccaaacattgtcacttggtataatatcatagctgcaaggattcgaaataccatgtgtgg

SEQ ID NO: 342 - DNA, 101 bp, Cannabis sativa
  source 1..101: mol_type = genomic DNA
cagacaaaagaatgtgctgacttcaatgttgtaacacaattgaaagtattagtttagtgatctaattcactaacaattatactttagaaaagattaaatat

SEQ ID NO: 343 - DNA, 101 bp, Cannabis sativa
  source 1..101: mol_type = genomic DNA
cggttgaagaaattaacaagaaattactgaaattgaggtaagtttttccttactttctatcggttctctttcaactgaagagaaaagatagataggataat

SEQ ID NO: 344 - DNA, 101 bp, Cannabis sativa
  source 1..101: mol_type = genomic DNA
aaccttacccattccaattccattcaagttttcattcccagaaaggtaatgaacttctaagctttcaaaattctcatgttttatccttttgtctagaaaga

SEQ ID NO: 345 - DNA, 101 bp, Cannabis sativa
  source 1..101: mol_type = genomic DNA
gatgctgttatgtaggttgctgcatcaacaaacagggaagtggatgcacatattcctaactaccctagcttaccgccacagcttatttgtcaacttcacaa

SEQ ID NO: 346 - DNA, 101 bp, Cannabis sativa
  source 1..101: mol_type = genomic DNA
aaaaaatataataaatatattattacaatattaaatgttgtagatatatgtttttaaataaaaaatgttacctagggatgcacacggggcgggaaatgggc

SEQ ID NO: 347 - DNA, 101 bp, Cannabis sativa
  source 1..101: mol_type = genomic DNA
caactgtaataaaagaaagcagttccagcgatcaagaagaattgcacaaacaataaaatcacgagcaactaagaaggcacgatactcctacgctgggcagc

SEQ ID NO: 348 - DNA, 101 bp, Cannabis sativa
  source 1..101: mol_type = genomic DNA
ggttgtttaggttgaggttgatcaaatatttgattagttaacatggaagccacttgtgccatggaaggtctatcagaagcaaagtcttgaacacaaagaag

SEQ ID NO: 349 - DNA, 101 bp, Cannabis sativa
  source 1..101: mol_type = genomic DNA
gaaagtaacgtaatatcagccttcaaatattgtacatttgtggtagtggactgtcccactgcatacaatgttatcttgggacgacctgctctagtagactt

SEQ ID NO: 350 - DNA, 101 bp, Cannabis sativa
  source 1..101: mol_type = genomic DNA
acacaagcacgatagaagaaaatcttaaactatctcaagccgatcactgcaaacacaaaataccatgtcacaaaacaagacgcttatattatattttccac

SEQ ID NO: 351 - DNA, 101 bp, Cannabis sativa
  source 1..101: mol_type = genomic DNA
tcggtccactggttttacaacaaacaaaccaatcaaacatcaagacaaaatgtacaaaagaaacatttcaacacaaatgaaaagcattcaaatacctgtta

SEQ ID NO: 352 - DNA, 101 bp, Cannabis sativa
  source 1..101: mol_type = genomic DNA
ccatttatgccactaatatctcttattttaatactatgaaatgggaactcatgtggcttcgatacaaattattcatagttgctgtaatatgcaaatgcagt

SEQ ID NO: 353 - DNA, 101 bp, Cannabis sativa
  source 1..101: mol_type = genomic DNA
ccttttctttcttctctagctatgtaggttgttgcacacaccgaaaggagcatgagattttaaagtaataaaagggtttgaatattttaatatcagaccct

SEQ ID NO: 354 - DNA, 101 bp, Cannabis sativa
  source 1..101: mol_type = genomic DNA
ttatttattttgaggatttgtgattgatattaattcaatcaacaaatcaactaggaaatgcattatgtccattgtccatgtcgaacttgatccttccatgt

SEQ ID NO: 355 - DNA, 101 bp, Cannabis sativa
  source 1..101: mol_type = genomic DNA
tcaagtttaattcaaaggttatggaaaaatggagaaatgtaaacgagtttcgagatcaatgcaatacctctagatacatgtccacagcacgataaacctca

SEQ ID NO: 356 - DNA, 101 bp, Cannabis sativa
  source 1..101: mol_type = genomic DNA
agagctcgtcttttggttgatgacagtgagggagaaaatgaaatggaggacgatgaacagactctttcgaaaaccgaatctacatttgaggatctgtgtgg

SEQ ID NO: 357 - DNA, 101 bp, Cannabis sativa
  source 1..101: mol_type = genomic DNA
aatgagtcgagacagttcagaatctatgaaaatggtgagttttggaatggtgatgacaaaccttttactcctaagtatttgcaaaccactactttatccag

SEQ ID NO: 358 - DNA, 101 bp, Cannabis sativa
  source 1..101: mol_type = genomic DNA
tttacccaaaaaaaaacatgataagaaaagagaggaagaattggttgagtgtcaatctaaacacatgacatcatcaatgcatgcaatgtgtgtgttacatg

SEQ ID NO: 359 - DNA, 101 bp, Cannabis sativa
  source 1..101: mol_type = genomic DNA
tgtaaggcttgtggtggcattttctttgggttacagatggacaaaaaggtcgctgagagaaccttaagggagcacaaaggtgatgctgttgctgctattag

SEQ ID NO: 360 - DNA, 101 bp, Cannabis sativa
  source 1..101: mol_type = genomic DNA
taccacacatggtcgaatctgggctaagacggattagacctgcgcctaagatccaccaaactatggggctcaaaaaatatattttttaattaaaattatac

SEQ ID NO: 361 - DNA, 101 bp, Cannabis sativa
  source 1..101: mol_type = genomic DNA
gacgctggcagggaatttcggaagcacgatacatctgtcaagaaggggcagagatcttgattggaaagggtgatattgatttgtgggataggccttggaaa

SEQ ID NO: 362 - DNA, 101 bp, Cannabis sativa
  source 1..101: mol_type = genomic DNA
agtgctgccagcaaggaagtcgtgagagcgagccctgagctgccctcaattcaagatgatgcttcggaggttttcttggacccgtgcgatgtactggaacc

SEQ ID NO: 363 - DNA, 101 bp, Cannabis sativa
  source 1..101: mol_type = genomic DNA
accataaaataactggaaaatgcggtattaggattttgcttccatctaaaaggtggaacagaatagagagctccaaccaaaatactaacacaatacagaga

SEQ ID NO: 364 - DNA, 101 bp, Cannabis sativa
  source 1..101: mol_type = genomic DNA
aacactaaagcgatgccttcatcaatggctgctcttccaacttgaggcgacccttcaaaggaactttgaccgtccggttctttgaaataggctacaacatt

SEQ ID NO: 365 - DNA, 101 bp, Cannabis sativa
  source 1..101: mol_type = genomic DNA
aattcaactttaatggtttttgttatttatggttctagaattaccaaatcttgtttgaatttgtgtccccaaaacagaaattatatatatgtgctacatag

SEQ ID NO: 366 - DNA, 101 bp, Cannabis sativa
  source 1..101: mol_type = genomic DNA
tgttggggttacaacttctctaacatttgtagcgactttaattttgttgggcccaaaatgtagccaagttgtgtaatgatctcttagcctattttgttggg

SEQ ID NO: 367 - DNA, 101 bp, Cannabis sativa
  source 1..101: mol_type = genomic DNA
aaaaagtactcaaccatgacacaagtttccactaaacttcatggtattgccaaatacttttcgatttgcttttcatttttaatttgccaacttgaattttg

SEQ ID NO: 368 - DNA, 101 bp, Cannabis sativa
  source 1..101: mol_type = genomic DNA
aatagggttctatggggtcttggatttgcgagcttcttgagccatgatttcaaaatccaagtcacctagtgaaaaagtaacagtgattgtaggtaaaaaca

SEQ ID NO: 369 - DNA, 101 bp, Cannabis sativa
  source 1..101: mol_type = genomic DNA
atttcagggaagtggaatctccaccaactgcttgtaagtcatttatctagttccttagcaaagttttcttagctatccttgcaaaatattttggacttgag

SEQ ID NO: 370 - DNA, 101 bp, Cannabis sativa
  source 1..101: mol_type = genomic DNA
agagtgagacagaaacgagtggctgcagttatgagttccataggccaaagcatattgatggcattcactgtcaccatgaacaaatacgcttcttcaaatat

SEQ ID NO: 371 - DNA, 101 bp, Cannabis sativa
  source 1..101: mol_type = genomic DNA
ggaaaaaaaaattgttttctaaattagattgcaagaacaaaaattatacaatcaaaatcattcaaactaccaatcaagtcatacgaataatcattcaaatt

(19) United States
(12) Patent Application Publication
(10) Pub. No.: US 2025/0127104 A1
(43) Pub. Date: Apr. 24, 2025

(54) CANNABINOID MARKERS

(71) Applicant: Phylos Bioscience, Inc., Portland, OR (US)

(72) Inventors: Erica Bakker, Portland, OR (US); Alisha Holloway, Portland, OR (US)

(73) Assignee: Phylos Bioscience, Inc., Portland, OR (US)

(21) Appl. No.: 18/837,849

(22) PCT Filed: Sep. 28, 2022
(86) PCT No.: PCT/US2022/077126

§ 371 Date: Aug. 12, 2024

Related U.S. Application Data
(60) Provisional application No. 63/250,067, filed on Sep. 29, 2021.

(51) Int. Cl. A01H 1/04; C12Q 1/6895

(52) U.S. Cl. CPC A01H 1/04; C12Q 1/6895; C12Q 2600/13; C12Q 2600/156

(57) ABSTRACT

Provided herein is the identification of genetic markers associated with modified cannabinoids relating to levels or ratios of one or more of tetrahydrocannabinol (THC), cannabidiol (CBD), cannabigerol (CBG), tetrahydrocannabivarin (THCV), cannabidivarin (CBDV), cannabichromene (CBC), or cannabigerovarin (CBGV). The markers are useful for breeding plants having modified cannabinoids by obtaining nucleic acids, detecting one or more markers that indicate modified cannabinoids, and establishing plant lines having such characteristics.

### CROSS REFERENCE TO RELATED APPLICATIONS

This application claims priority benefit to U.S. provisional application No. 63/250,067, filed, Sep. 29, 2021, the entire contents of which are hereby incorporated by reference.

### SEQUENCE LISTING REFERENCE

Pursuant to 37 CFR §§ 1.821-1.825, a Sequence Listing in the form of an ASCII-compliant text file (entitled “2007-WO1_ST26_Sequence_Listing.xml” created on Sep. 26, 2022 and 349 kilobytes in size), which will serve as both the paper copy required by 37 CFR § 1.821(c) and the computer readable form (CRF) required by 37 CFR § 1.821(e), is submitted concurrently with the instant application. The entire contents of the Sequence Listing are incorporated herein by reference.

### BACKGROUND OF THE INVENTION

Cannabinoids are substances found in cannabis plants, which bind to endogenous endocannabinoid receptors. The most commonly known cannabinoids are tetrahydrocannabinol (THC) and cannabidiol (CBD). Yet cannabis plants contain over a hundred known other “minor” cannabinoids, which may include cannabigerol (CBG), tetrahydrocannabivarin (THCV), cannabidivarin (CBDV), cannabichromene (CBC), or cannabigerovarin (CBGV).

Research and development as well as the sale of minor cannabinoid products has been limited due to low commonly occurring levels of minor cannabinoids in cannabis flower. As a consequence, the ability to produce cannabis with differential levels of cannabinoids will be useful in research, medical, and recreational settings.

The invention described herein solves the laborious and time-consuming issues of traditional breeding methods by providing cannabis breeders with a specific and efficient method for creating cannabis plants having modified cannabinoid levels.

### SUMMARY OF THE INVENTION

The present teachings relate to methods of selecting plants with modified cannabinoids. In an embodiment, a method for selecting one or more plants having one or more modified cannabinoids is provided. The method comprises i) obtaining nucleic acids from a sample plant or its germplasm; (ii) detecting one or more markers that indicate the modified cannabinoids, and (iii) indicating the modified cannabinoids. In an embodiment, the method comprises selecting the one or more plants indicating the modified cannabinoids. In an embodiment, the modified cannabinoids correlate to elevated levels of one or more of total tetrahydrocannabinol (THC), total cannabidiol (CBD), total cannabigerol (CBG), total tetrahydrocannabivarin (THCV), total cannabidivarin (CBDV), total cannabichromene (CBC), or total cannabigerovarin (CBGV) or their acidic cannabinoid forms thereof.

In an embodiment, the modified cannabinoids correlate to elevated levels of the combination of total THC, total CBD, total CBG, total THCV, total CBDV, total CBC, and total CBGV or their acidic cannabinoid forms thereof. In an embodiment, the one or more markers comprises a polymorphism relative to a reference genome at nucleotide position: (a) 1,605,949 on chromosome 1; (b) 511,858 on chromosome 2; (c) 68,168,149 on chromosome 3; (d) 822,718 on chromosome 2; or (e) 829175 on chromosome 2; or (f) 889775 on chromosome 2; or (g) 1981515 on chromosome 7; or (h) 8445770 on chromosome 6; or (i) 15287401 on chromosome 6; or (j) 26717252 on chromosome 6; or (k) 29145396 on chromosome 6; or (l) 43665911 on chromosome 6; or (m) 45567480 on chromosome 6; or (n) 57867261 on chromosome 6; or (o) 67382064 on chromosome 6; or (p) 60624 on chromosome 7; or (q) 3441223 on chromosome 7; or (r) 5752776 on chromosome 7; or (s) 6225622 on chromosome 7; or (t) 6340996 on chromosome 7; or (u) 7643910 on chromosome 7; or (v) 23406044 on chromosome 7; or (w) 7624628 on chromosome 9; wherein the reference genome is the Abacus Cannabis reference genome (version CsaAba2). In an embodiment, the nucleotide position comprises: (a) a T/T or T/A genotype at position 1,605,949 on chromosome 1; (b) a G/A or A/A genotype at position 511,858 on chromosome 2; (c) an A/A genotype at position 68,168,149 on chromosome 3; (d) a T/T or T/C genotype at position 822718 on chromosome 2; (e) a C/C or C/T genotype at position 829175 on chromosome 2; (f) a G/G or G/A genotype at position 889775 on chromosome 2; (g) a T/T or A/T genotype at position 1981515 on chromosome 7; (h) a C/C or A/C genotype at position 8445770 on chromosome 6; (i) a G/G or C/G genotype at position 15287401 on chromosome 6; (j) a G/G or G/A genotype at position 26717252 on chromosome 6; (k) a C/C or C/T genotype at position 29145396 on chromosome 6; (l) a T/T or T/G genotype at position 43665911 on chromosome 6; (m) a G/G or A/G genotype at position 45567480 on chromosome 6; (n) a C/C or T/C genotype at position 57867261 on chromosome 6; (o) a T/T or T/A genotype at position 67382064 on chromosome 6; (p) a C/C or T/C genotype at position 60624 on chromosome 7; (q) a A/A or A/G genotype at position 3441223 on chromosome 7; (r) a G/G or T/G genotype at position 5752776 on chromosome 7; (s) a T/T or A/T genotype at position 6225622 on chromosome 7; (t) a T/T or A/T genotype at position 6340996 on chromosome 7; (u) a T/T or G/T genotype at position 7643910 on chromosome 7; (v) a C/C or C/T genotype at position 23406044 on chromosome 7; (w) a A/A or A/G genotype at position 7624628 on chromosome 9; wherein the reference genome is the Abacus Cannabis reference genome (version CsaAba2). In an embodiment, the cannabinoid comprises any one of (a) type I cannabinoid; (b) type II cannabinoid; or (c) type III cannabinoid.

In an embodiment, the one or more markers comprises a polymorphism at position 51 of any one or more of SEQ ID NO:1; SEQ ID NO:11; SEQ ID NO:22; SEQ ID NO: 12; SEQ ID NO: 13; SEQ ID NO: 14; SEQ ID NO: 135; SEQ ID NO: 119; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 138; SEQ ID NO: 140; SEQ ID NO: 145; SEQ ID NO: 146; SEQ ID NO: 150; SEQ ID NO: 188; SEQ ID NO: 360. In an embodiment, the nucleotide position comprises: (a) a T/T or T/A genotype at position 51 of SEQ ID NO:1; (b) a G/A or A/A genotype at position 51 of SEQ ID NO:11; (c) an A/A genotype at position 51 of SEQ ID NO:22; (d) a T/T or T/C genotype at position 51 of SEQ ID NO: 12; (e) a C/C or C/T genotype at position 51 of SEQ ID NO: 13; (f) a G/G or G/A genotype at position 51 of SEQ ID NO: 14; (g) a T/T or A/T genotype at position 51 of SEQ ID NO: 135; (h) a C/C or A/C genotype at position 51 of SEQ ID NO: 119; (i) a G/G or C/G genotype at position 51 of SEQ ID NO: 121; (j) a G/G or G/A genotype at position 51 of SEQ ID NO: 122; (k) a C/C or C/T genotype at position 51 of SEQ ID NO: 123; (l) a T/T or T/G genotype at position 51 of SEQ ID NO: 124; (m) a G/G or A/G genotype at position 51 of SEQ ID NO: 125; (n) a C/C or T/C genotype at position 51 of SEQ ID NO: 126; (o) a T/T or T/A genotype at position 51 of SEQ ID NO: 127; (p) a C/C or T/C genotype at position 51 of SEQ ID NO: 128; (q) a A/A or A/G genotype at position 51 of SEQ ID NO: 138; (r) a G/G or T/G genotype at position 51 of SEQ ID NO: 140; (s) a T/T or A/T genotype at position 51 of SEQ ID NO: 145; (t) a T/T or A/T genotype at position 51 of SEQ ID NO: 146; (u) a T/T or G/T genotype at position 51 of SEQ ID NO: 150; (v) a C/C or C/T genotype at position 51 of SEQ ID NO: 188; (w) a A/A or A/G genotype at position 51 of SEQ ID NO: 360; wherein the reference genome is the Abacus Cannabis reference genome (version CsaAba2). In an embodiment, the one or more markers comprises a polymorphism relative to a reference genome within any one or more haplotypes wherein the haplotypes comprise the region: (a) between positions 1,593,859 and 1,611,839 on chromosome 1; (b) between positions 506060 and 518277 on chromosome 2; (c) between positions 68162527 and 68173892 on chromosome 3; (d) between positions 812504 and 850793 on chromosome 2; (e) between positions 812504 and 850793 on chromosome 2; (f) between positions 887683 and 891812 on chromosome 2; (g) between positions 1959582 and 2009523 on chromosome 7; (h) between positions 8437332 and 8460311 on chromosome 6; (i) between positions 15261229 and 15310117 on chromosome 6; (j) between positions 26684684 and 26723996 on chromosome 6; (k) between positions 29097877 and 29158571 on chromosome 6; (l) between positions 43662000 and 44089274 on chromosome 6; (m) between positions 45546384 and 45612738 on chromosome 6; (n) between positions 57860326 and 57877559 on chromosome 6; (o) between positions 67379156 and 67421842 on chromosome 6; (p) between positions 15258 and 115293 on chromosome 7; (q) between positions 3412885 and 3477646 on chromosome 7; (r) between positions 5746452 and 5845317 on chromosome 7; (s) between positions 6221113 and 6256430 on chromosome 7; (t) between positions 6337560 and 6410830 on chromosome 7; (u) between positions 7639988 and 7658574 on chromosome 7; (v) between positions 23372911 and 23432690 on chromosome 7; (w) between positions 7596844 and 7632863 on chromosome 9; wherein the reference genome is the Abacus Cannabis reference genome (version CsaAba2).

In an embodiment, the modified cannabinoids correlate to elevated levels of the combination of total THC and total THCV. In an embodiment, the one or more markers comprises a polymorphism relative to a reference genome at nucleotide position: (a) 1605949 on chromosome 1; or (b) 2187135 on chromosome 1; or (c) 68168149 on chromosome 3; or (d) 1981515 on chromosome 7; or (e) 8445770 on chromosome 6; or (f) 15287401 on chromosome 6; or (g) 26717252 on chromosome 6; or (h) 29145396 on chromosome 6; or (i) 43665911 on chromosome 6; or (j) 57867261 on chromosome 6; or (k) 67382064 on chromosome 6; or (l) 60624 on chromosome 7; or (m) 3441223 on chromosome 7; or (n) 5752776 on chromosome 7; or (o) 6225622 on chromosome 7; or (p) 6340996 on chromosome 7; or (q) 7643910 on chromosome 7; or (r) 23406044 on chromosome 7; or (s) 7624628 on chromosome 9; or (t) 38978759 on chromosome 1; or (u) 67769631 on chromosome 1; or (v) 16010588 on chromosome 3; or (w) 39837146 on chromosome 3; or (x) 2127802 on chromosome 4; or (y) 6705244 on chromosome 7; or (z) 11063067 on chromosome 7; or (aa) 11067412 on chromosome 7; or (ab) 17000256 on chromosome 7; or (ac) 17008461 on chromosome 7; or (ad) 28081703 on chromosome 7; or (ae) 28685688 on chromosome 7; or (af) 30520237 on chromosome 7; or (ag) 32259550 on chromosome 7; or (ah) 36589991 on chromosome 7; or (ai) 37130207 on chromosome 7; or (aj) 47324655 on chromosome 7; or (ak) 52923743 on chromosome 7; or (al) 54375898 on chromosome 7; or (am) 56032988 on chromosome 7; or (an) 56301604 on chromosome 7; or (ao) 56967275 on chromosome 7; or (ap) 58538433 on chromosome 7; or (aq) 46475498 on chromosome 9 wherein the reference genome is the Abacus Cannabis reference genome (version CsaAba2). In an embodiment, the nucleotide position comprises: (a) a T/T or T/A genotype at position 1605949 on chromosome 1; (b) a A/A genotype at position 2187135 on chromosome 1; (c) a A/A genotype at position 68168149 on chromosome 3; (d) a T/T or A/T genotype at position 1981515 on chromosome 7; (e) a C/C or A/C genotype at position 8445770 on chromosome 6; (f) a G/G or C/G genotype at position 15287401 on chromosome 6; (g) a G/G or G/A genotype at position 26717252 on chromosome 6; (h) a C/C or C/T genotype at position 29145396 on chromosome 6; (i) a T/T or T/G genotype at position 43665911 on chromosome 6; (j) a C/C or T/C genotype at position 57867261 on chromosome 6; (k) a T/T or T/A genotype at position 67382064 on chromosome 6; (l) a C/C or T/C genotype at position 60624 on chromosome 7; (m) a A/A or A/G genotype at position 3441223 on chromosome 7; (n) a G/G or T/G genotype at position 5752776 on chromosome 7; (o) a T/T or A/T genotype at position 6225622 on chromosome 7; (p) a T/T or A/T genotype at position 6340996 on chromosome 7; (q) a T/T or G/T genotype at position 7643910 on chromosome 7; (r) a C/C or C/T genotype at position 23406044 on chromosome 7; (s) a A/A or A/G genotype at position 7624628 on chromosome 9; (t) a A/A or G/A genotype at position 38978759 on chromosome 1; (u) a A/A or G/A genotype at position 67769631 on chromosome 1; (v) a A/A or T/A genotype at position 16010588 on chromosome 3; (w) a A/A or G/A genotype at position 39837146 on chromosome 3; (x) a G/G or A/G genotype at position 2127802 on chromosome 4; (y) a C/C or C/T genotype at position 6705244 on chromosome 7; (z) a T/T or C/T genotype at position 11063067 on chromosome 7; (aa) a A/A or G/A genotype at position 11067412 on chromosome 7; (ab) a T/T or G/T genotype at position 17000256 on chromosome 7; (ac) a C/C or T/C genotype at position 17008461 on chromosome 7; (ad) a C/C or G/C genotype at position 28081703 on chromosome 7; (ae) a T/T or C/T genotype at position 28685688 on chromosome 7; (af) a A/A or C/A genotype at position 30520237 on chromosome 7; (ag) a C/C or T/C genotype at position 32259550 on chromosome 7; (ah) a T/T genotype at position 36589991 on chromosome 7; (ai) a T/T or A/T genotype at position 37130207 on chromosome 7; (aj) a C/C or C/T genotype at position 47324655 on chromosome 7; (ak) a A/A or C/A genotype at position 52923743 on chromosome 7; (al) a T/T or T/C genotype at position 54375898 on chromosome 7; (am) a A/A or G/A genotype at position 56032988 on chromosome 7; (an) a A/A or G/A genotype at position 56301604 on chromosome 7; (ao) a T/T or A/T genotype at position 56967275 on chromosome 7; (ap) a T/T or C/T genotype at position 58538433 on chromosome 7; (aq) a A/A or G/A genotype at position 46475498 on chromosome 9 wherein the reference genome is the Abacus Cannabis reference genome (version CsaAba2).

In an embodiment, the cannabinoid comprises a type I or type IV cannabinoid. In an embodiment, the one or more markers comprises a polymorphism at position 51 of any one or more of SEQ ID NO:1; SEQ ID NO:2; SEQ ID NO: 4; SEQ ID NO: 8; SEQ ID NO: 20; SEQ ID NO: 21; SEQ ID NO: 22; SEQ ID NO: 23; SEQ ID NO: 119; SEQ ID NO: 121; SEQ ID NO: 122; SEQ ID NO: 123; SEQ ID NO: 124; SEQ ID NO: 126; SEQ ID NO: 127; SEQ ID NO: 128; SEQ ID NO: 135; SEQ ID NO: 138; SEQ ID NO: 140; SEQ ID NO: 145; SEQ ID NO: 146; SEQ ID NO: 147; SEQ ID NO: 150; SEQ ID NO: 164; SEQ ID NO: 165; SEQ ID NO: 179; SEQ ID NO: 180; SEQ ID NO: 188; SEQ ID NO: 191; SEQ ID NO: 192; SEQ ID NO: 200; SEQ ID NO: 202; SEQ ID NO: 212; SEQ ID NO: 213; SEQ ID NO: 231; SEQ ID NO: 288; SEQ ID NO: 293; SEQ ID NO: 310; SEQ ID NO: 314; SEQ ID NO: 328; SEQ ID NO: 348; SEQ ID NO: 360; SEQ ID NO: 361. In an embodiment, the nucleotide position comprises: (a) a T/T or T/A or genotype at position 51 of SEQ ID NO: 1; (b) a A/A or genotype at position 51 of SEQ ID NO: 2; (c) a A/A or genotype at position 51 of SEQ ID NO: 22; (d) a T/T or A/T genotype at position 51 of SEQ ID NO: 135; (e) a C/C or A/C genotype at position 51 of SEQ ID NO: 119; (f) a G/G or C/G genotype at position 51 of SEQ ID NO: 121; (g) a G/G or G/A genotype at position 51 of SEQ ID NO: 122; (h) a C/C or C/T genotype at position 51 of SEQ ID NO: 123; (i) a T/T or T/G genotype at position 51 of SEQ ID NO: 124; (j) a C/C or T/C genotype at position 51 of SEQ ID NO: 126; (k) a T/T or T/A genotype at position 51 of SEQ ID NO: 127; (l) a C/C or T/C genotype at position 51 of SEQ ID NO: 128; (m) a A/A or A/G genotype at position 51 of SEQ ID NO: 138; (n) a G/G or T/G genotype at position 51 of SEQ ID NO: 140; (o) a T/T or A/T genotype at position 51 of SEQ ID NO: 145; (p) a T/T or A/T genotype at position 51 of SEQ ID NO: 146; (q) a T/T or G/T genotype at position 51 of SEQ ID NO: 150; (r) a C/C or C/T genotype at position 51 of SEQ ID NO: 188; (s) a A/A or A/G genotype at position 51 of SEQ ID NO: 360; (t) a A/A or G/A genotype at position 51 of SEQ ID NO: 4; (u) a A/A or G/A genotype at position 51 of SEQ ID NO: 8; (v) a A/A or T/A genotype at position 51 of SEQ ID NO: 20; (w) a A/A or G/A genotype at position 51 of SEQ ID NO: 21; (x) a G/G or A/G genotype at position 51 of SEQ ID NO: 23; (y) a C/C or C/T genotype at position 51 of SEQ ID NO: 147; (z) a T/T or C/T genotype at position 51 of SEQ ID NO: 164; (aa) a A/A or G/A genotype at position 51 of SEQ ID NO: 165; (ab) a T/T or G/T genotype at position 51 of SEQ ID NO: 179; (ac) a C/C or T/C genotype at position 51 of SEQ ID NO: 180; (ad) a C/C or G/C genotype at position 51 of SEQ ID NO: 191; (ae) a T/T or C/T genotype at position 51 of SEQ ID NO: 192; (af) a A/A or C/A genotype at position 51 of SEQ ID NO: 200; (ag) a C/C or T/C genotype at position 51 of SEQ ID NO: 202; (ah) a T/T or genotype at position 51 of SEQ ID NO: 212; (ai) a T/T or A/T genotype at position 51 of SEQ ID NO: 213; (aj) a C/C or C/T genotype at position 51 of SEQ ID NO: 231; (ak) a A/A or C/A genotype at position 51 of SEQ ID NO: 288; (al) a T/T or T/C genotype at position 51 of SEQ ID NO: 293; (am) a A/A or G/A genotype at position 51 of SEQ ID NO: 310; (an) a A/A or G/A genotype at position 51 of SEQ ID NO: 314; (ao) a T/T or A/T genotype at position 51 of SEQ ID NO: 328; (ap) a T/T or C/T genotype at position 51 of SEQ ID NO: 348; (aq) a A/A or G/A genotype at position 51 of SEQ ID NO: 361 wherein the reference genome is the Abacus Cannabis reference genome (version CsaAba2). In an embodiment, the one or more markers comprises a polymorphism relative to a reference genome within any one or more haplotypes wherein the haplotypes comprise the region: (a) between positions 1593859 and 1611839 on chromosome 1; (b) between positions 2183741 and 2194270 on chromosome 1; (c) between positions 68162527 and 68173892 on chromosome 3; (d) between positions 1959582 and 2009523 on chromosome 7; (e) between positions 8437332 and 8460311 on chromosome 6; (f) between positions 15261229 and 15310117 on chromosome 6; (g) between positions 26684684 and 26723996 on chromosome 6; (h) between positions 29097877 and 29158571 on chromosome 6; (i) between positions 43662000 and 44089274 on chromosome 6; (j) between positions 57860326 and 57877559 on chromosome 6; (k) between positions 67379156 and 67421842 on chromosome 6; (l) between positions 15258 and 115293 on chromosome 7; (m) between positions 3412885 and 3477646 on chromosome 7; (n) between positions 5746452 and 5845317 on chromosome 7; (o) between positions 6221113 and 6256430 on chromosome 7; (p) between positions 6337560 and 6410830 on chromosome 7; (q) between positions 7639988 and 7658574 on chromosome 7; (r) between positions 23372911 and 23432690 on chromosome 7; (s) between positions 7596844 and 7632863 on chromosome 9; (t) between positions 38944628 and 39073783 on chromosome 1; (u) between positions 67761686 and 67892254 on chromosome 1; (v) between positions 15652280 and 16182525 on chromosome 3; (w) between positions 39699406 and 40350847 on chromosome 3; (x) between positions 2082401 and 2155110 on chromosome 4; (y) between positions 6622471 and 6808016 on chromosome 7; (z) between positions 10965365 and 11193689 on chromosome 7; (aa) between positions 10965365 and 11193689 on chromosome 7; (ab) between positions 16992324 and 17419297 on chromosome 7; (ac) between positions 16992324 and 17419297 on chromosome 7; (ad) between positions 27759260 and 28263307 on chromosome 7; (ae) between positions 28594408 and 29061134 on chromosome 7; (af) between positions 29891019 and 30608774 on chromosome 7; (ag) between positions 30608774 and 32279982 on chromosome 7; (ah) between positions 36579046 and 36880336 on chromosome 7; (ai) between positions 37049888 and 37211605 on chromosome 7; (aj) between positions 46967630 and 48198578 on chromosome 7; (ak) between positions 52544592 and 53396185 on chromosome 7; (al) between positions 53505022 and 54400345 on chromosome 7; (am) between positions 56018989 and 56076209 on chromosome 7; (an) between positions 56171548 and 56426824 on chromosome 7; (ao) between positions 56910768 and 57069404 on chromosome 7; (ap) between positions 58428139 and 58607780 on chromosome 7; (aq) between positions 46392138 and 46584908 on chromosome 9 wherein the reference genome is the Abacus Cannabis reference genome (version CsaAba2).

In an embodiment, the modified cannabinoids correlate to the ratio of total CBC to the total THC, total CBD, total CBG, total THCV, total CBDV, total CBC, and total CBGV or their acidic cannabinoid forms thereof. In an embodiment, the one or more markers comprises a polymorphism relative to a reference genome at nucleotide position: (a) 52923743 on chromosome 7; or (b) 55970630 on chromosome 7; or (c) 55984088 on chromosome 7; or (d) 56018989 on chromosome 7; or (e) 56032988 on chromosome 7; or (f) 56076209 on chromosome 7; or (g) 56171548 on chromosome 7; or (h) 56301604 on chromosome 7; or (i) 56426824 on chromosome 7; or (j) 56430375 on chromosome 7; or (k) 56440283 on chromosome 7; or (l) 56488125 on chromosome 7; or (m) 56492081 on chromosome 7; or (n) 56538007 on chromosome 7; or (o) 56700085 on chromosome 7; or (p) 56782341 on chromosome 7; or (q) 56872960 on chromosome 7; or (r) 56910768 on chromosome 7; or (s) 56967275 on chromosome 7; or (t) 57069404 on chromosome 7; or (u) 57080583 on chromosome 7; or (v) 57089709 on chromosome 7; or (w) 57104188 on chromosome 7; or (x) 57120122 on chromosome 7; or (y) 57152672 on chromosome 7; or (z) 57228643 on chromosome 7; or (aa) 57233796 on chromosome 7; or (ab) 58275951 on chromosome 7; or (ac) 58538433 on chromosome 7 wherein the reference genome is the Abacus Cannabis reference genome (version CsaAba2). In an embodiment, the nucleotide position comprises: (a) a C/C or C/A genotype at position 52923743 on chromosome 7; (b) a A/A or A/T genotype at position 55970630 on chromosome 7; (c) a A/A or A/T genotype at position 55984088 on chromosome 7; (d) a G/G or G/A genotype at position 56018989 on chromosome 7; (e) a G/G or G/A genotype at position 56032988 on chromosome 7; (f) a C/C or C/T genotype at position 56076209 on chromosome 7; (g) a A/A or A/G genotype at position 56171548 on chromosome 7; (h) a G/G or G/A genotype at position 56301604 on chromosome 7; (i) a T/T or T/G genotype at position 56426824 on chromosome 7; (j) a G/G or A/G genotype at position 56430375 on chromosome 7; (k) a C/C or C/T genotype at position 56440283 on chromosome 7; (l) a G/G or A/G genotype at position 56488125 on chromosome 7; (m) a G/G or T/G genotype at position 56492081 on chromosome 7; (n) a C/C or C/A genotype at position 56538007 on chromosome 7; (o) a C/C or T/C genotype at position 56700085 on chromosome 7; (p) a G/G or G/C genotype at position 56782341 on chromosome 7; (q) a C/C or C/A genotype at position 56872960 on chromosome 7; (r) a T/T or T/C genotype at position 56910768 on chromosome 7; (s) a A/A or A/T genotype at position 56967275 on chromosome 7; (t) a C/C or C/T genotype at position 57069404 on chromosome 7; (u) a C/C or C/T genotype at position 57080583 on chromosome 7; (v) a A/A or T/A genotype at position 57089709 on chromosome 7; (w) a C/C or C/T genotype at position 57104188 on chromosome 7; (x) a T/T or C/T genotype at position 57120122 on chromosome 7; (y) a A/A or G/A genotype at position 57152672 on chromosome 7; (z) a G/G or A/G genotype at position 57228643 on chromosome 7; (aa) a A/A or A/G genotype at position 57233796 on chromosome 7; (ab) a A/A or T/A genotype at position 58275951 on chromosome 7; (ac) a C/C or C/T genotype at position 58538433 on chromosome 7 wherein the reference genome is the Abacus Cannabis reference genome (version CsaAba2).

In an embodiment, the cannabinoid comprises type I or type IV cannabinoid. In an embodiment, the one or more markers comprises a polymorphism at position 26 of any one or more of SEQ ID NO: 321; SEQ ID NO: 322; SEQ ID NO: 323; SEQ ID NO: 324; SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 334; SEQ ID NO: 335; SEQ ID NO: 336; SEQ ID NO: 343; SEQ ID NO: 348. In an embodiment, the nucleotide position comprises: (a) a C/C or C/A genotype at position 51 of SEQ ID NO: 288; (b) a A/A or A/T genotype at position 51 of SEQ ID NO: 307; (c) a A/A or A/T genotype at position 51 of SEQ ID NO: 308; (d) a G/G or G/A genotype at position 51 of SEQ ID NO: 309; (e) a G/G or G/A genotype at position 51 of SEQ ID NO: 310; (f) a C/C or C/T genotype at position 51 of SEQ ID NO: 311; (g) a A/A or A/G genotype at position 51 of SEQ ID NO: 313; (h) a G/G or G/A genotype at position 51 of SEQ ID NO: 314; (i) a T/T or T/G genotype at position 51 of SEQ ID NO: 316; (j) a G/G or A/G genotype at position 51 of SEQ ID NO: 317; (k) a C/C or C/T genotype at position 51 of SEQ ID NO: 318; (l) a G/G or A/G genotype at position 51 of SEQ ID NO: 320; (m) a G/G or T/G genotype at position 51 of SEQ ID NO: 321; (n) a C/C or C/A genotype at position 51 of SEQ ID NO: 322; (o) a C/C or T/C genotype at position 51 of SEQ ID NO: 323; (p) a G/G or G/C genotype at position 51 of SEQ ID NO: 324; (q) a C/C or C/A genotype at position 51 of SEQ ID NO: 326; (r) a T/T or T/C genotype at position 51 of SEQ ID NO: 327; (s) a A/A or A/T genotype at position 51 of SEQ ID NO: 328; (t) a C/C or C/T genotype at position 51 of SEQ ID NO: 329; (u) a C/C or C/T genotype at position 51 of SEQ ID NO: 330; (v) a A/A or T/A genotype at position 51 of SEQ ID NO: 331; (w) a C/C or C/T genotype at position 51 of SEQ ID NO: 332; (x) a T/T or C/T genotype at position 51 of SEQ ID NO: 333; (y) a A/A or G/A genotype at position 51 of SEQ ID NO: 334; (z) a G/G or A/G genotype at position 51 of SEQ ID NO: 335; (aa) a A/A or A/G genotype at position 51 of SEQ ID NO: 336; (ab) a A/A or T/A genotype at position 51 of SEQ ID NO: 343; (ac) a C/C or C/T genotype at position 51 of SEQ ID NO: 348 wherein the reference genome is the Abacus Cannabis reference genome (version CsaAba2). In an embodiment, the one or more markers comprises a polymorphism relative to a reference genome within any one or more haplotypes wherein the haplotypes comprise the region: (a) between positions 52544592 and 53396185 on chromosome 7; (b) between positions 55939712 and 56081903 on chromosome 7; (c) between positions 55939712 and 56081903 on chromosome 7; (d) between positions 55939712 and 56081903 on chromosome 7; (e) between positions 55939712 and 56081903 on chromosome 7; (f) between positions 55939712 and 56081903 on chromosome 7; (g) between positions 56166983 and 56434732 on chromosome 7; (h) between positions 56166983 and 56434732 on chromosome 7; (i) between positions 56166983 and 56434732 on chromosome 7; (j) between positions 56166983 and 56434732 on chromosome 7; (k) between positions 56434732 and 56593122 on chromosome 7; (l) between positions 56434732 and 56593122 on chromosome 7; (m) between positions 56434732 and 56593122 on chromosome 7; (n) between positions 56434732 and 56593122 on chromosome 7; (o) between positions 56620519 and 56809638 on chromosome 7; (p) between positions 56620519 and 56809638 on chromosome 7; (q) between positions 56809638 and 57276534 on chromosome 7; (r) between positions 56809638 and 57276534 on chromosome 7; (s) between positions 56809638 and 57276534 on chromosome 7; (t) between positions 56809638 and 57276534 on chromosome 7; (u) between positions 56809638 and 57276534 on chromosome 7; (v) between positions 56809638 and 57276534 on chromosome 7; (w) between positions 56809638 and 57276534 on chromosome 7; (x) between positions 56809638 and 57276534 on chromosome 7; (y) between positions 56809638 and 57276534 on chromosome 7; (z) between positions 56809638 and 57276534 on chromosome 7; (aa) between positions 56809638 and 57276534 on chromosome 7; (ab) between positions 58244327 and 58353998 on chromosome 7; (ac) between positions 58428139 and 58607780 on chromosome 7 wherein the reference genome is the Abacus Cannabis reference genome (version CsaAba2).

In an embodiment, the modified cannabinoids correlate to elevated levels of the combination of total CBG and total CBGV. In an embodiment, the one or more markers comprises a polymorphism relative to a reference genome at nucleotide position: (a) 32398208 on chromosome 1; or (b) 1961209 on chromosome 2; or (c) 166141 on chromosome 3; or (d) 174381 on chromosome 3; or (e) 9387181 on chromosome 4; or (f) 1173474 on chromosome 5; or (g) 1181979 on chromosome 5; or (h) 2038965 on chromosome 5; or (i) 2208629 on chromosome 5; or (j) 4023145 on chromosome 6; or (k) 858349 on chromosome 7; or (l) 948328 on chromosome 7; or (m) 954115 on chromosome 7; or (n) 1155154 on chromosome 7; or (o) 1928237 on chromosome 7; or (p) 2243260 on chromosome 7; or (q) 2612238 on chromosome 7; or (r) 5348707 on chromosome 7; or (s) 5851251 on chromosome 7; or (t) 5900788 on chromosome 7; or (u) 9079344 on chromosome 7; or (v) 9272731 on chromosome 7; or (w) 9421290 on chromosome 7; or (x) 9595805 on chromosome 7; or (y) 9598562 on chromosome 7; or (z) 9619898 on chromosome 7; or (aa) 9668980 on chromosome 7; or (ab) 9716257 on chromosome 7; or (ac) 9868420 on chromosome 7; or (ad) 9922946 on chromosome 7; or (ae) 12851206 on chromosome 7; or (af) 13928304 on chromosome 7; or (ag) 13933289 on chromosome 7; or (ah) 14271073 on chromosome 7; or (ai) 14579677 on chromosome 7; or (aj) 14940971 on chromosome 7; or (ak) 15036406 on chromosome 7; or (al) 15091266 on chromosome 7; or (am) 15105660 on chromosome 7; or (an) 15187940 on chromosome 7; or (ao) 16603812 on chromosome 7; or (ap) 17355416 on chromosome 7; or (aq) 17450215 on chromosome 7; or (ar) 18095653 on chromosome 7; or (as) 21035025 on chromosome 7; or (at) 22964819 on chromosome 7; or (au) 24653415 on chromosome 7; or (av) 36579046 on chromosome 7; or (aw) 42289736 on chromosome 7; or (ax) 44227026 on chromosome 7; or (ay) 48089881 on chromosome 7; or (az) 49618132 on chromosome 7; or (ba) 50213053 on chromosome 7; or (bb) 50221231 on chromosome 7; or (bc) 50854826 on chromosome 7; or (bd) 51054719 on chromosome 7; or (be) 51173524 on chromosome 7; or (bf) 52285032 on chromosome 7; or (bg) 52296271 on chromosome 7; or (bh) 52554676 on chromosome 7; or (bi) 52561249 on chromosome 7; or (bj) 53231544 on chromosome 7; or (bk) 55716705 on chromosome 7; or (bl) 56018989 on chromosome 7; or (bm) 56076209 on chromosome 7; or (bn) 56309021 on chromosome 7; or (bo) 56802628 on chromosome 7; or (bp) 58933090 on chromosome 7; or (bq) 8502077 on chromosome 8; or (br) 8679838 on chromosome 8; or (bs) 9091526 on chromosome 8; or (bt) 9196738 on chromosome 8; or (bu) 9309715 on chromosome 8; or (bv) 21389309 on chromosome 8; or (bw) 4684198 on chromosome 9; or (bx) 55358400 on chromosome X; or (by) 55956503 on chromosome X; or (bz) 56164045 on chromosome X; or (ca) 56268500 on chromosome X; or (cb) 56281906 on chromosome X; or (cc) 56610941 on chromosome X; or (cd) 78814483 on chromosome X; or (ce) 79583866 on chromosome X; or (cf) 32398208 on chromosome 1; or (cg) 166081 on chromosome 5; or (ch) 796653 on chromosome 5; or (ci) 1181979 on chromosome 5; or (cj) 2038965 on chromosome 5; or (ck) 2208629 on chromosome 5; or (cl) 4023145 on chromosome 6; or (cm) 1755387 on chromosome 7; or (cn) 9421290 on chromosome 7; or (co) 9598562 on chromosome 7; or (cp) 9619898 on chromosome 7; or (cq) 9668980 on chromosome 7; or (cr) 15105660 on chromosome 7; or (cs) 44227026 on chromosome 7; or (ct) 50745259 on chromosome 7; or (cu) 50854826 on chromosome 7; or (cv) 51054719 on chromosome 7; or (cw) 51173524 on chromosome 7; or (cx) 52285032 on chromosome 7; or (cy) 52296271 on chromosome 7; or (cz) 52322834 on chromosome 7; or (da) 52332628 on chromosome 7; or (db) 52439705 on chromosome 7; or (dc) 52554676 on chromosome 7; or (dd) 52561249 on chromosome 7; or (de) 53231544 on chromosome 7; or (df) 56018989 on chromosome 7; or (dg) 56076209 on chromosome 7; or (dh) 8679838 on chromosome 8; or (di) 9309715 on chromosome 8; or (dj) 2690026 on chromosome 9; or (dk) 78814483 on chromosome X; or (dl) 122735 on chromosome 2; or (dm) 188542 on chromosome 2; or (dn) 1785269 on chromosome 2; or (do) 48670446 on chromosome 1; or (dp) 64341256 on chromosome 1; or (dq) 67666507 on chromosome 1; or (dr) 2132683 on chromosome 5; or (ds) 2177531 on chromosome 5; or (dt) 2366529 on chromosome 5; or (du) 2899343 on chromosome 5; or (dv) 3074649 on chromosome 5; or (dw) 3086874 on chromosome 5; or (dx) 3485895 on chromosome 5; or (dy) 3535297 on chromosome 5; or (dz) 3599637 on chromosome 5; or (ea) 3961139 on chromosome 5; or (eb) 4384123 on chromosome 5; or (ec) 19988534 on chromosome 5; or (ed) 20017410 on chromosome 5; or (ee) 20148519 on chromosome 5; or (ef) 36148442 on chromosome 5; or (eg) 53489757 on chromosome 5; or (eh) 81104593 on chromosome X; or (ei) 38978759 on chromosome 1; or (ej) 67769631 on chromosome 1; or (ek) 16010588 on chromosome 3; or (el) 39837146 on chromosome 3; or (em) 2127802 on chromosome 4; or (en) 12788565 on chromosome 6; or (eo) 6705244 on chromosome 7; or (ep) 11063067 on chromosome 7; or (eq) 11067412 on chromosome 7; or (er) 12636172 on chromosome 7; or (es) 17000256 on chromosome 7; or (et) 17008461 on chromosome 7; or (eu) 28081703 on chromosome 7; or (ev) 28685688 on chromosome 7; or (ew) 30520237 on chromosome 7; or (ex) 32259550 on chromosome 7; or (ey) 36589991 on chromosome 7; or (ez) 37130207 on chromosome 7; or (fa) 52923743 on chromosome 7; or (fb) 56032988 on chromosome 7; or (fc) 56301604 on chromosome 7; or (fd) 56967275 on chromosome 7; or (fe) 58538433 on chromosome 7; or (ff) 46475498 on chromosome 9 wherein the reference genome is the Abacus Cannabis reference genome (version CsaAba2). In an embodiment, the nucleotide position comprises: (a) a C/C or C/T genotype at position 32398208 on chromosome 1; (b) a A/A or T/A genotype at position 1961209 on chromosome 2; (c) a G/G or T/G genotype at position 166141 on chromosome 3; (d) a T/T or C/T genotype at position 174381 on chromosome 3; (e) a T/T or C/T genotype at position 9387181 on chromosome 4; (f) a T/T or T/C genotype at position 1173474 on chromosome 5; (g) a C/C or T/C genotype at position 1181979 on chromosome 5; (h) a C/C or T/C genotype at position 2038965 on chromosome 5; (i) a G/G or G/C genotype at position 2208629 on chromosome 5; (j) a G/G or G/A genotype at position 4023145 on chromosome 6; (k) a C/C or C/G genotype at position 858349 on chromosome 7; (l) a G/G or G/A genotype at position 948328 on chromosome 7; (m) a T/T or T/C genotype at position 954115 on chromosome 7; (n) a G/G or G/A genotype at position 1155154 on chromosome 7; (o) A/A or C/A genotype at position 1928237 on chromosome 7; (p) a T/T or C/T genotyp at position 2243260 on chromosome 7; (q) a G/G or A/G genotype at position 261223 on chromosome 7; (r) a A/A or A/G genotype at position 5348707 on chromosome 7; (a A/A or A/G genotype at position 5851251 on chromosome 7; (t) a A/A or A/C genotype at position 5900788 on chromosome 7; (u) a C/C or C/G genotype at position 9079344 on chromosome 7; (v) a T/T or T/C genotype at position 9272731 on chromosome 7; (w) a T/T or T/C genotype at position 9421290 on chromosome 7; (x) a A/A or A/G genotype at position 9595805 on chromosome 7; (y) a A/A or A/T genotype at position 9598562 on chromosome 7; (z) a G/G or G/A genotype at position 9619898 on chromosome 7; (aa) a T/T or T/C genotype at position 9668980 on chromosome 7; (ab) a A/A or A/G genotype at position 9716257 on chromosome 7; (ac) a G/G or G/T genotype at position 9868420 on chromosome 7; (ad) a G/G or G/A genotype at position 9922946 on chromosome 7; (ae) a G/G or G/A genotype at position 12851206 on chromosome 7; (af) a T/T or C/T genotype at position 13928304 on chromosome 7; (ag) a A/A or G/A genotype at position 13933289 on chromosome 7; (ah) a C/C or C/A genotype at position 14271073 on chromosome 7; (ai) a T/T or C/T genotype at position 14579677 on chromosome 7; (aj) a T/T or T/C genotype at position 14940971 on chromosome 7; (ak) a G/G or G/A genotype at position 15036406 on chromosome 7; (al) a T/T or C/T genotype at position 15091266 on chromosome 7; (am) a C/C or A/C genotype at position 15105660 on chromosome 7; (an) a T/T or C/T genotype at position 15187940 on chromosome 7; (ao) a C/C or T/C genotype at position 16603812 on chromosome 7; (ap) a G/G or C/G genotype at position 17355416 on chromosome 7; (aq) a T/T or C/T genotype at position 17450215 on chromosome 7; (ar) a A/A or G/A genotype at position 18095653 on chromosome 7; (as) a T/T or T/C genotype at position 21035025 on chromosome 7; (at) a C/C or C/T genotype at position 22964819 on chromosome 7; (au) a T/T or T/A genotype at position 24653415 on chromosome 7; (av) a A/A or G/A genotype at position 36579046 on chromosome 7; (aw) a A/A or G/A genotype at position 42289736 on chromosome 7; (ax) a A/A or A/G genotype at position 44227026 on chromosome 7; (ay) a G/G or A/G genotype at position 48089881 on chromosome 7; (az) a T/T or T/G genotype at position 49618132 on chromosome 7; (ba) a T/T or T/C genotype at position 50213053 on chromosome 7; (bb) a C/C or C/T genotype at position 50221231 on chromosome 7; (bc) a A/A or A/C genotype at position 50854826 on chromosome 7; (bd) a C/C or C/T genotype at position 51054719 on chromosome 7; (be) a G/G or G/A genotype at position 51173524 on chromosome 7; (bf) a G/G or G/A genotype at position 52285032 on chromosome 7; (bg) a T/T or T/C genotype at position 52296271 on chromosome 7; (bh) a T/T or T/A genotype at position 52554676 on chromosome 7; (bi) a A/A or A/G genotype at position 52561249 on chromosome 7; (bj) a A/A or C/A genotype at position 53231544 on chromosome 7; (bk) a A/A or A/G genotype at position 55716705 on chromosome 7; (bl) a G/G or G/A genotype at position 56018989 on chromosome 7; (bm) a C/C or C/genotype at position 56076209 on chromosome 7; (bn) a T/T or T/A genotype at position 56309021 on chromosome 7; (bo) a T/T or C/T genotype at position 56802628 o chromosome 7; (bp) a T/T or C/T genotype at position 58933090 on chromosome 7; (bq a G/G or A/G genotype at position 8502077 on chromosome 8; (br) a T/T or C/9091526 on chromosome 8; (bt) a T/T or C/T genotype at position 9196738 o chromosome 8; (bu) a T/T or C/T genotype at position 9309715 on chromosome 8; (bv a C/C or T/C genotype at position 21389309 on chromosome 8; (bw) a C/C or C/genotype at position 4684198 on chromosome 9; (bx) a G/G or A/G genotype at position 55358400 on chromosome X; (by) a T/T or C/T genotype at position 55956503 o chromosome X; (bz) a C/C or T/C genotype at position 56164045 on chromosome X (ca) a C/C or G/C genotype at position 56268500 on chromosome X; (cb) a A/A or C/genotype at position 56281906 on chromosome X; (cc) a T/T or C/T genotype at position 56610941 on chromosome X; (cd) a C/C or T/C genotype at position 78814483 on chromosome X; (ce) a T/T or C/T genotype at position 79583866 on chromosome X; (cf) a C/C or C/T genotype at position 32398208 on chromosome 1; (cg) a A/A or T/A genotype at position 166081 on chromosome 5; (ch) a G/G or G/A genotype at position 796653 on chromosome 5; (ci) a C/C or T/C genotype at position 1181979 on chromosome 5; (cj) a C/C or T/C genotype at position 2038965 on chromosome 5; (ck) a G/G or G/C genotype at position 2208629 on chromosome 5; (cl) a G/G or G/A genotype at position 4023145 on chromosome 6; (cm) a G/G or G/A genotype at position 1755387 on chromosome 7; (cn) a T/T or T/C genotype at position 9421290 on chromosome 7; (co) a A/A or A/T genotype at position 9598562 on chromosome 7; (cp) a G/G or G/A genotype at position 9619898 on chromosome 7; (cq) a T/T or T/C genotype at position 9668980 on chromosome 7; (cr) a C/C or A/C genotype at position 15105660 on chromosome 7; (cs) a A/A or A/G genotype at position 44227026 on chromosome 7; (ct) a T/T genotype at position 50745259 on chromosome 7; (cu) a A/A or A/C genotype at position 50854826 on chromosome 7; (cv) a C/C or C/T genotype at position 51054719 on chromosome 7; (cw) a G/G or G/A genotype at position 51173524 on chromosome 7; (cx) a G/G or G/A genotype at position 52285032 on chromosome 7; (cy) a T/T or T/C genotype at position 52296271 on chromosome 7; (cz) a A/A or A/G genotype at position 52322834 on chromosome 7; (da) a G/G or G/A genotype at position 52332628 on chromosome 7; (db) a G/G or G/A genotype at position 52439705 on chromosome 7; (dc) a T/T or T/A genotype at position 52554676 on chromosome 7; (dd) a A/A or A/G genotype at position 52561249 on chromosome 7; (de) a A/A or C/A genotype at position 53231544 on chromosome 7; (df) a G/G or G/A genotype at position 56018989 on chromosome 7; (dg) a C/C or C/T genotype at position 56076209 on chromosome 7; (dh) a T/T or C/T genotype at position 8679838 on chromosome 8; (di) a T/T or C/T genotype at position 9309715 on chromosome 8; (dj) a G/G or G/T genotype at position 2690026 on chromosome 9; (dk) a C/C or T/C genotype at position 78814483 on chromosome X; (dl) a A/A or G/A genotype at position 122735 on chromosome 2; (dm) a C/C or T/C genotype at position 188542 on chromosome 2; (dn) a C/C or T/C genotype at position 1785269 on chromosome 2; (do) a C/C or T/C genotype at position 48670446 on chromosome 1; (dp) a C/C or G/C genotype at position 64341256 on chromosome 1; (dq) a T/T or C/T genotype at position 67666507 on chromosome 1; (dr) a G/G or A/G genotype at position 2132683 on chromosome 5; (ds) a C/C or A/C genotype at position 2177531 on chromosome 5; (dt) a T/T or C/T genotype at position 2366529 on chromosome 5; (du) a C/C or T/C genotype at position 2899343 on chromosome 5; (dv) a T/T or C/T genotype at position 3074649 on chromosome 5; (dw) a C/C or T/C genotype at position 3086874 on chromosome 5; (dx) a C/C or T/C genotype at position 3485895 on chromosome 5; (dy) a G/G or A/G genotype at position 3535297 on chromosome 5; (dz) a A/A or G/A genotype at position 3599637 on chromosome 5; (ea) a G/G or C/G genotype at position 3961139 on chromosome 5; (eb) a T/T or A/T genotype at position 4384123 on chromosome 5; (ec) a T/T or C/T genotype at position 19988534 on chromosome 5; (ed) a G/G or T/G genotype at position 20017410 on chromosome 5; (ee) a A/A or G/A genotype at position 20148519 on chromosome 5; (ef) a T/T or C/T genotype at position 36148442 on chromosome 5; (eg) a A/A or C/A genotype at position 53489757 on chromosome 5; (eh) a C/C genotype at position 81104593 on chromosome X; (ei) a G/G or G/A genotype at position 38978759 on chromosome 1; (ej) a G/G or G/A genotype at position 67769631 on chromosome 1; (ek) a T/T or T/A genotype at position 16010588 on chromosome 3; (el) a G/G or G/A genotype at position 39837146 on chromosome 3; (em) a A/A or A/G genotype at position 2127802 on chromosome 4; (en) a A/A or A/T genotype at position 12788565 on chromosome 6; (eo) a T/T or C/T genotype at position 6705244 on chromosome 7; (ep) a C/C or C/T genotype at position 11063067 on chromosome 7; (eq) a G/G or G/A genotype at position 11067412 on chromosome 7; (er) a A/A or G/A genotype at position 12636172 on chromosome 7; (es) a G/G or G/T genotype at position 17000256 on chromosome 7; (et) a T/T or T/C genotype at position 17008461 on chromosome 7; (eu) a C/C or G/C genotype at position 28081703 on chromosome 7; (ev) a T/T or C/T genotype at position 28685688 on chromosome 7; (ew) a C/C or C/A genotype at position 30520237 on chromosome 7; (ex) a T/T or T/C genotype at position 32259550 on chromosome 7; (ey) a C/C or C/T genotype at position 36589991 on chromosome 7; (ez) a A/A or A/T genotype at position 37130207 on chromosome 7; (fa) a C/C or C/A genotype at position 52923743 on chromosome 7; (fb) a G/G or G/A genotype at position 56032988 on chromosome 7; (fc) a G/G or G/A genotype at position 56301604 on chromosome 7; (fd) a A/A or A/T genotype at position 56967275 on chromosome 7; (fe) a C/C or C/T genotype at position 58538433 on chromosome 7; (ff) a G/G or G/A genotype at position 46475498 on chromosome 9 wherein the reference genome is the Abacus Cannabis reference genome (version CsaAba2).

In an embodiment, the cannabinoid comprises a type I, II, III, or IV cannabinoid. In an embodiment, the one or more markers comprises a polymorphism at position 51 of any one or more of SEQ ID NO: 3; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 15; SEQ ID NO: 16; SEQ ID NO: 17; SEQ ID NO: 18; SEQ ID NO: 20; SEQ ID NO: 21; SEQ ID NO: 23; SEQ ID NO: 25; SEQ ID NO: 26; SEQ ID NO: 27; SEQ ID NO: 28; SEQ ID NO: 29; SEQ ID NO: 29; SEQ ID NO: 31; SEQ ID NO: 31; SEQ ID NO: 32; SEQ ID NO: 33; SEQ ID NO: 34; SEQ ID NO: 34; SEQ ID NO: 36; SEQ ID NO: 37; SEQ ID NO: 38; SEQ ID NO: 39; SEQ ID NO: 41; SEQ ID NO: 42; SEQ ID NO: 43; SEQ ID NO: 45; SEQ ID NO: 47; SEQ ID NO: 77; SEQ ID NO: 78; SEQ ID NO: 79; SEQ ID NO: 95; SEQ ID NO: 104; SEQ ID NO: 118; SEQ ID NO: 118; SEQ ID NO: 120; SEQ ID NO: 129; SEQ ID NO: 130; SEQ ID NO: 131; SEQ ID NO: 132; SEQ ID NO: 133; SEQ ID NO: 134; SEQ ID NO: 136; SEQ ID NO: 137; SEQ ID NO: 139; SEQ ID NO: 141; SEQ ID NO: 142; SEQ ID NO: 147; SEQ ID NO: 153; SEQ ID NO: 154; SEQ ID NO: 155; SEQ ID NO: 155; SEQ ID NO: 156; SEQ ID NO: 157; SEQ ID NO: 157; SEQ ID NO: 158; SEQ ID NO: 158; SEQ ID NO: 160; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 186; SEQ ID NO: 187; SEQ ID NO: 189; SEQ ID NO: 191; SEQ ID NO: 192; SEQ ID NO: 200; SEQ ID NO: 202; SEQ ID NO: 211; SEQ ID NO: 212; SEQ ID NO: 213; SEQ ID NO: 223; SEQ ID NO: 226; SEQ ID NO: 226; SEQ ID NO: 232; SEQ ID NO: 252; SEQ ID NO: 260; SEQ ID NO: 261; SEQ ID NO: 268; SEQ ID NO: 273; SEQ ID NO: 273; SEQ ID NO: 274; SEQ ID NO: 274; SEQ ID NO: 275; SEQ ID NO: 275; SEQ ID NO: 276; SEQ ID NO: 276; SEQ ID NO: 277; SEQ ID NO: 277; SEQ ID NO: 278; SEQ ID NO: 279; SEQ ID NO: 281; SEQ ID NO: 286; SEQ ID NO: 286; SEQ ID NO: 287; SEQ ID NO: 287; SEQ ID NO: 288; SEQ ID NO: 290; SEQ ID NO: 290; SEQ ID NO: 304; SEQ ID NO: 309; SEQ ID NO: 309; SEQ ID NO: 310; SEQ ID NO: 311; SEQ ID NO: 311; SEQ ID NO: 314; SEQ ID NO: 315; SEQ ID NO: 325; SEQ ID NO: 328; SEQ ID NO: 348; SEQ ID NO: 349; SEQ ID NO: 352; SEQ ID NO: 353; SEQ ID NO: 353; SEQ ID NO: 354; SEQ ID NO: 355; SEQ ID NO: 356; SEQ ID NO: 356; SEQ ID NO: 357; SEQ ID NO: 358; SEQ ID NO: 359; SEQ ID NO: 361; SEQ ID NO: 363; SEQ ID NO: 364; SEQ ID NO: 365; SEQ ID NO: 366; SEQ ID NO: 367; SEQ ID NO: 368; SEQ ID NO: 369; SEQ ID NO: 369; SEQ ID NO: 370; SEQ ID NO: 371. In an embodiment, the nucleotide position comprises: (a) a C/C or C/T genotype at position 51 of SEQ ID NO: 3; (b) a A/A or T/A genotype at position 51 of SEQ ID NO: 16; (c) a G/G or T/G genotype at position 51 of SEQ ID NO: 17; (d) a T/T or C/T genotype at position 51 of SEQ ID NO: 18; (e) a T/T or C/T genotype at position 51 of SEQ ID NO: 25; (f) a T/T or T/C genotype at position 51 of SEQ ID NO: 28; (g) a C/C or T/C genotype at position 51 of SEQ ID NO: 29; (h) a C/C or T/C genotype at position 51 of SEQ ID NO: 31; (i) a G/G or G/C genotype at position 51 of SEQ ID NO: 34; (j) a G/G or G/A genotype at position 51 of SEQ ID NO: 118; (k) a C/C or C/G genotype at position 51 of SEQ ID NO: 129; (l) a G/G or G/A genotype at position 51 of SEQ ID NO: 130; (m) a T/T or T/C genotype at position 51 of SEQ ID NO: 131; (n) a G/G or G/A genotype at position 51 of SEQ ID NO: 132; (o) a A/A or C/A genotype at position 51 of SEQ I NO: 134; (p) a T/T or C/T genotype at position 51 of SEQ ID NO: 136; (q) a G/G or A/genotype at position 51 of SEQ ID NO: 137; (r) a A/A or A/G genotype at position 51 o SEQ ID NO: 139; (s) a A/A or A/G genotype at position 51 of SEQ ID NO: 141; (t) a A/or A/C genotype at position 51 of SEQ ID NO: 142; (u) a C/C or C/G genotype at position 51 of SEQ ID NO: 153; (v) a T/T or T/C genotype at position 51 of SEQ ID NO: 154; (w) T/T or T/C genotype at position 51 of SEQ ID NO: 155; (x) a A/A or A/G genotype a position 51 of SEQ ID NO: 156; (y) a A/A or A/T genotype at position 51 of SEQ ID NO 157; (z) a G/G or G/A genotype at position 51 of SEQ ID NO: 158; (aa) a T/T or T/genotype at position 51 of SEQ ID NO: 160; (ab) a A/A or A/G genotype at position 51 o SEQ ID NO: 161; (ac) a G/G or G/T genotype at position 51 of SEQ ID NO: 162; (ad) a G/G or G/A genotype at position 51 o SEQ ID NO: 163; (ae) a G/G or G/A genotype at position 51 of SEQ ID NO: 167; (af) a T/T or C/T genotype at position 51 of SEQ ID NO: 169; (ag) a A/A or G/A genotype at position 51 of SEQ ID NO: 170; (ah) a C/C or C/A genotype at position 51 of SEQ ID NO: 171; (ai) a T/T or C/T genotype at position 51 of SEQ ID NO: 172; (aj) a T/T or T/C genotype at position 51 of SEQ ID NO: 173; (ak) a G/G or G/A genotype at position 51 of SEQ ID NO: 174; (al) a T/T or C/T genotype at position 51 of SEQ ID NO: 175; (am) a C/C or A/C genotype at position 51 of SEQ ID NO: 176; (an) a T/T or C/T genotype at position 51 of SEQ ID NO: 177; (ao) a C/C or T/C genotype at position 51 of SEQ ID NO: 178; (ap) a G/G or C/G genotype at position 51 of SEQ ID NO: 181; (aq) a T/T or C/T genotype at position 51 of SEQ ID NO: 182; (ar) a A/A or G/A genotype at position 51 of SEQ ID NO: 183; (as) a T/T or T/C genotype at position 51 of SEQ ID NO: 186; (at) a C/C or C/T genotype at position 51 of SEQ ID NO: 187; (au) a T/T or T/genotype at position 51 of SEQ ID NO: 189; (av) a A/A or G/A genotype at position 51 of SEQ ID NO: 211; (aw) a A/A or G/A genotype at position 51 of SEQ ID NO: 223; (ax) a A/A or A/G genotype at position 51 of SEQ ID NO: 226; (ay) a G/G or A/G genotype at position 51 of SEQ ID NO: 232; (az) a T/T or T/G genotype at position 51 of SEQ ID NO: 252; (ba) a T/T or T/C genotype at position 51 of SEQ ID NO: 260; (bb) a C/C or C/T genotype at position 51 of SEQ ID NO: 261; (bc) a A/A or A/C genotype at position 51 of SEQ ID NO: 273; (bd) a C/C or C/T genotype at position 51 of SEQ ID NO: 274; (be) a G/G or G/A genotype at position 51 of SEQ ID NO: 275; (bf) a G/G or G/A genotype at position 51 of SEQ ID NO: 276; (bg) a T/T or T/C genotype at position 51 of SEQ ID NO: 277; (bh) a T/T or T/A genotype at position 51 of SEQ ID NO: 286; (bi) a A/A or A/G genotype at position 51 of SEQ ID NO: 287; (bj) a A/A or C/A genotype at position 51 of SEQ ID NO: 290; (bk) a A/A or A/G genotype at position 51 of SEQ ID NO: 304; (bl) a G/G or G/A genotype at position 51 of SEQ ID NO: 309; (bm) a C/C or C/T genotype at position 51 of SEQ ID NO: 311; (bn) a T/T or T/A genotype at position 51 of SEQ ID NO: 315; (bo) a T/T or C/T genotype at position 51 of SEQ ID NO: 325; (bp) a T/T or C/T genotype at position 51 of SEQ ID NO: 349; (bq) a G/G or A/G genotype at position 51 of SEQ ID NO: 352; (br) a T/T or C/T genotype at position 51 of SEQ ID NO: 353; (bs) a T/T or C/T genotype at position 51 of SEQ ID NO: 354; (bt) a T/T or C/T genotype at position 51 of SEQ ID NO: 355; (bu) a T/T or C/T genotype at position 51 of SEQ ID NO: 356; (bv) a C/C or T/C genotype at position 51 of SEQ ID NO: 357; (bw) a C/C or C/T genotype at position 51 of SEQ ID NO: 359; (bx) a G/G or A/G genotype at position 51 of SEQ ID NO: 363; (by) a T/T or C/T genotype at position 51 of SEQ ID NO: 364; (bz) a C/C or T/C genotype at position 51 of SEQ ID NO: 365; (ca) a C/C or G/C genotype at position 51 of SEQ ID NO: 366; (cb) a A/A or C/A genotype at position 51 of SEQ ID NO: 367; (cc) a T/T or C/T genotype at position 51 of SEQ ID NO: 368; (cd) a C/C or T/C genotype at position 51 of SEQ ID NO: 369; (ce) a T/T or C/T genotype at position 51 of SEQ ID NO: 370; (cf) a C/C or C/T genotype at position 51 of SEQ ID NO: 3; (cg) a A/A or T/A genotype at position 51 of SEQ ID NO: 26; (ch) a G/G or G/A genotype at position 51 of SEQ ID NO: 27; (ci) a C/C or T/C genotype at position 51 of SEQ ID NO: 29; (cj) a C/C or T/C genotype at position 51 of SEQ ID NO: 31; (ck) a G/G or G/C genotype at position 51 of SEQ ID NO: 34; (cl) a G/G or G/A genotype at position 51 of SEQ ID NO: 118; (cm) a G/G or G/A genotype at position 51 of SEQ ID NO: 133; (cn) a T/T or T/C genotype at position 51 of SEQ ID NO: 155; (co) a A/A or A/T genotype at position 51 of SEQ ID NO: 157; (cp) a G/G or G/A genotype at position 51 of SEQ ID NO: 158; (cq) a T/T or T/C genotype at position 51 of SEQ ID NO: 160; (cr) a C/C or A/C genotype at position 51 of SEQ ID NO: 176; (cs) a A/A or A/G genotype at position 51 of SEQ ID NO: 226; (ct) a T/T genotype at position 51 of SEQ ID NO: 268; (cu) a A/A or A/C genotype at position 51 of SEQ ID NO: 273; (cv) a C/C or C/T genotype at position 51 of SEQ ID NO: 274; (cw) a G/G or G/A genotype at position 51 of SEQ ID NO: 275; (cx) a G/G or G/A genotype at position 51 of SEQ ID NO: 276; (cy) a T/T or T/C genotype at position 51 of SEQ ID NO: 277; (cz) a A/A or A/G genotype at position 51 of SEQ ID NO: 278; (da) a G/G or G/A genotype at position 51 of SEQ ID NO: 279; (db) a G/G or G/A genotype at position 51 of SEQ ID NO: 281; (dc) a T/T or T/A genotype at position 51 of SEQ ID NO: 286; (dd) a A/A or A/G genotype at position 51 of SEQ ID NO: 287; (de) a A/A or C/A genotype at position 51 of SEQ ID NO: 290; (df) a G/G or G/A genotype at position 51 of SEQ ID NO: 309; (dg) a C/C or C/T genotype at position 51 of SEQ ID NO: 311; (dh) a T/T or C/T genotype at position 51 of SEQ ID NO: 353; (di) a T/T or C/T genotype at position 51 of SEQ ID NO: 356; (dj) a G/G or G/T genotype at position 51 of SEQ ID NO: 358; (dk) a C/C or T/C genotype at position 51 of SEQ ID NO: 369; (dl) a A/A or G/A genotype at position 51 of SEQ ID NO: 9; (dm) a C/C or T/C genotype at position 51 of SEQ ID NO: 10; (dn) a C/C or T/C genotype at position 51 of SEQ ID NO: 15; (do) a C/C or T/C genotype at position 51 of SEQ ID NO: 5; (dp) a C/C or G/C genotype at position 51 of SEQ ID NO: 6; (dq) a T/T or C/T genotype at position 51 of SEQ ID NO: 7; (dr) a G/G or A/G genotype at position 51 of SEQ ID NO: 32; (ds) a C/C or A/C genotype at position 51 of SEQ ID NO: 33; (dt) a T/T or C/T genotype at position 51 of SEQ ID NO: 36; (du) a C/C or T/C genotype at position 51 of SEQ ID NO: 37; (dv) a T/T or C/T genotype at position 51 of SEQ ID NO: 38; (dw) a C/C or T/C genotype at position 51 of SEQ ID NO: 39; (dx) a C/C or T/C genotype at position 51 of SEQ ID NO: 41; (dy) a G/G or A/G genotype at position 51 of SEQ ID NO: 42; (dz) a A/A or G/A genotype at position 51 of SEQ ID NO: 43; (ea) a G/G or C/G genotype at position 51 of SEQ ID NO: 45; (eb) a T/T or A/T genotype at position 51 of SEQ ID NO: 47; (ec) a T/T or C/T genotype at position 51 of SEQ ID NO: 77; (ed) a G/G or T/G genotype at position 51 of SEQ ID NO: 78; (ee) a A/A or G/A genotype at position 51 of SEQ ID NO: 79; (ef) a T/T or C/T genotype at position 51 of SEQ ID NO: 95; (eg) a A/A or C/A genotype at position 51 of SEQ ID NO: 104; (eh) a C/C genotype at position 51 of SEQ ID NO: 371; (ei) a G/G or G/A genotype at position 51 of SEQ ID NO: 4; (ej) a G/G or G/A genotype at position 51 of SEQ ID NO: 8; (ek) a T/T or T/A genotype at position 51 of SEQ ID NO: 20; (el) a G/G or G/A genotype at position 51 of SEQ ID NO: 21; (em) a A/A or A/G genotype at position 51 of SEQ ID NO: 23; (en) a A/A or A/T genotype at position 51 of SEQ ID NO: 120; (eo) a T/T or C/T genotype at position 51 of SEQ ID NO: 147; (ep) a C/C or C/T genotype at position 51 of SEQ ID NO: 164; (eq) a G/G or G/A genotype at position 51 of SEQ ID NO: 165; (er) a A/A or G/A genotype at position 51 of SEQ ID NO: 166; (es) a G/G or G/T genotype at position 51 of SEQ ID NO: 179; (et) a T/T or T/C genotype at position 51 of SEQ ID NO: 180; (eu) a C/C or G/C genotype at position 51 of SEQ ID NO: 191; (ev) a T/T or C/T genotype at position 51 of SEQ ID NO: 192; (ew) a C/C or C/A genotype at position 51 of SEQ ID NO: 200; (ex) a T/T or T/C genotype at position 51 of SEQ ID NO: 202; (ey) a C/C or C/T genotype at position 51 of SEQ ID NO: 212; (ez) a A/A or A/T genotype at position 51 of SEQ ID NO: 213; (fa) a C/C or C/A genotype at position 51 of SEQ ID NO: 288; (fb) a G/G or G/A genotype at position 51 of SEQ ID NO: 310; (fc) a G/G or G/A genotype at position 51 of SEQ ID NO: 314; (fd) a A/A or A/T genotype at position 51 of SEQ ID NO: 328; (fe) a C/C or C/T genotype at position 51 of SEQ ID NO: 348; (ff) a G/G or G/A genotype at position 51 of SEQ ID NO: 361 wherein the reference genome is the Abacus Cannabis reference genome (version CsaAba2). In an embodiment, the one or more markers comprises a polymorphism relative to a reference genome within any one or more haplotypes wherein the haplotypes comprise the region: (a) between positions 32414393 and 32414393 on chromosome 1; (b) between positions 1966129 and 1966129 on chromosome 2; (c) between positions 168861 and 168861 on chromosome 3; (d) between positions 175515 and 175515 on chromosome 3; (e) between positions 9395794 and 9395794 on chromosome 4; (f) between positions 1194306 and 1194306 on chromosome 5; (g) between positions 1194306 and 1194306 on chromosome 5; (h) between positions 2065182 and 2065182 on chromosome 5; (i) between positions 2288919 and 2288919 on chromosome 5; (j) between positions 4037935 and 4037935 on chromosome 6; (k) between positions 866330 and 866330 on chromosome 7; (l) between positions 975143 and 975143 on chromosome 7; (m) between positions 975143 and 975143 on chromosome 7; (n) between positions 1170814 and 1170814 on chromosome 7; (o) between positions 1932407 and 1932407 on chromosome 7; (p) between positions 2262669 and 2262669 on chromosome 7; (q) between positions 2670280 and 2670280 on chromosome 7; (r) between positions 5389490 and 5389490 on chromosome 7; (s) between positions 5871508 and 5871508 on chromosome 7; (t) between positions 5908268 and 5908268 on chromosome 7; (u) between positions 9082114 and 9082114 on chromosome 7; (v) between positions 9295164 and 9295164 on chromosome 7; (w) between positions 9432681 and 9432681 on chromosome 7; (x) between positions 9609554 and 9609554 on chromosome 7; (y) between positions 9609554 and 9609554 on chromosome 7; (z) between positions 9625781 and 9625781 on chromosome 7; (aa) between positions 9677657 and 9677657 on chromosome 7; (ab) between positions 9716591 and 9716591 on chromosome 7; (ac) between positions 9891604 and 9891604 on chromosome 7; (ad) between positions 9951332 and 9951332 on chromosome 7; (ae) between positions 12855919 and 12855919 on chromosome 7 (af) between positions 14002688 and 14002688 on chromosome 7; (ag) between positions 14002688 and 14002688 on chromosome 7; (ah) between positions 14288814 and 14288814 on chromosome 7; (ai) between positions 14592996 and 14592996 on chromosome 7; (aj) between positions 14945873 and 14945873 on chromosome 7; (ak) between positions 15048022 and 15048022 on chromosome 7; (al) between positions 15110059 and 15110059 on chromosome 7; (am) between positions 15110059 and 15110059 on chromosome 7; (an) between positions 15223104 and 15223104 on chromosome 7; (ao) between positions 16609331 and 16609331 on chromosome 7; (ap) between position 17363692 and 17363692 on chromosome 7; (aq) between positions 17459892 an 17459892 on chromosome 7; (ar) between positions 18109800 and 18109800 o chromosome 7; (as) between positions 21047228 and 21047228 on chromosome 7; (at) between positions 22984143 and 22984143 on chromosome 7; (au) between positions 24673734 and 24673734 on chromosome 7; (av) between positions 36584469 and 36584469 on chromosome 7; (aw) between positions 42379636 and 42379636 on chromosome 7; (ax) between positions 44231810 and 44231810 on chromosome 7; (ay) between positions 48095767 and 48095767 on chromosome 7; (az) between positions 49628859 and 49628859 on chromosome 7; (ba) between positions 50302149 and 50302149 on chromosome 7; (bb) between positions 50302149 and 50302149 on chromosome 7; (bc) between positions 50861059 and 50861059 on chromosome 7; (bd) between positions 51062076 and 51062076 on chromosome 7; (be) between positions 51226826 and 51226826 on chromosome 7; (bf) between positions 52291349 and 52291349 on chromosome 7; (bg) between positions 52322834 and 52322834 on chromosome 7; (bh) between positions 52760459 and 52760459 on chromosome 7; (bi) between positions 52760459 and 52760459 on chromosome 7; (bj) between positions 53283191 and 53283191 on chromosome 7; (bk) between positions 55731905 and 55731905 on chromosome 7; (bl) between positions 56032988 and 56032988 on chromosome 7; (bm) between positions 56081903 and 56081903 on chromosome 7; (bn) between positions 56329245 and 56329245 on chromosome 7; (bo) between positions 56806750 and 56806750 on chromosome 7; (bp) between positions 58943140 and 58943140 on chromosome 7; (bq) between positions 8507767 and 8507767 on chromosome 8; (br) between positions 8717782 and 8717782 on chromosome 8; (bs) between positions 9094186 and 9094186 on chromosome 8; (bt) between positions 9199573 and 9199573 on chromosome 8; (bu) between positions 9316329 and 9316329 on chromosome 8; (bv) between positions 21404217 and 21404217 on chromosome 8; (bw) between positions 4692766 and 4692766 on chromosome 9; (bx) between positions 55391085 and 55391085 on chromosome X; (by) between positions 55962857 and 55962857 on chromosome X; (bz) between positions 56169204 and 56169204 on chromosome X; (ca) between positions 56305767 and 56305767 on chromosome X; (cb) between positions 56305767 and 56305767 on chromosome X; (cc) between positions 56652118 and 56652118 on chromosome X; (cd) between positions 78823688 and 78823688 on chromosome X; (ce) between positions 79599466 and 79599466 on chromosome X; (cf) between positions 32379705 and 32422316 on chromosome 1; (cg) between positions 158841 and 176716 on chromosome 5; (ch) between positions 790537 and 801459 on chromosome 5; (ci) between positions 1173474 and 1210033 on chromosome 5; (cj) between positions 2011766 and 2065182 on chromosome 5; (ck) between positions 2177531 and 2288919 on chromosome 5; (cl) between positions 4002406 and 4037935 on chromosome 6; (cm) between positions 1739821 and 1760293 on chromosome 7; (cn) between positions 9404565 and 9432681 on chromosome 7; (co) between positions 9595805 and 9609554 on chromosome 7; (cp) between positions 9609554 and 9625781 on chromosome 7; (cq) between positions 9662890 and 9677657 on chromosome 7; (cr) between positions 15091266 and 15110059 on chromosome 7; (cs) between positions 44203159 and 44231810 on chromosome 7; (ct) between positions 50724154 and 50747700 on chromosome 7; (cu) between positions 50822516 and 50861059 on chromosome 7; (cv) between positions 51022401 and 52322834 on chromosome 7; (cw) between positions 51022401 and 52322834 on chromosome 7; (cx) between positions 51022401 and 52322834 on chromosome 7; (cy) between positions 51022401 and 52322834 on chromosome 7; (cz) between positions 52296271 and 52332628 on chromosome 7; (da) between positions 52322834 and 52441872 on chromosome 7; (db) between positions 52322834 and 52441872 on chromosome 7; (dc) between positions 52552192 and 52561249 on chromosome 7; (dd) between positions 52554676 and 52760459 on chromosome 7; (de) between positions 53210452 and 53283191 on chromosome 7; (df) between positions 56001500 and 56032988 on chromosome 7; (dg) between positions 56067360 and 56081903 on chromosome 7; (dh) between positions 8670961 and 8717782 on chromosome 8; (di) between positions 9305133 and 9316329 on chromosome 8; (dj) between positions 2680294 and 2697330 on chromosome 9; (dk) between positions 78812503 and 78823688 on chromosome X; (dl) between positions 119953 and 136319 on chromosome 2; (dm) between positions 181346 and 196868 on chromosome 2; (dn) between positions 1767164 and 1791640 on chromosome 2; (do) between positions 48665164 and 48727602 on chromosome 1; (dp) between positions 64338238 and 64349232 on chromosome 1; (dq) between positions 67660656 and 67691271 on chromosome 1; (dr) between positions 2120881 and 2167503 on chromosome 5; (ds) between positions 2167503 and 2208629 on chromosome 5; (dt) between positions 2364964 and 2534579 on chromosome 5; (du) between positions 2844078 and 2908474 on chromosome 5; (dv) between positions 3061958 and 3081773 on chromosome 5; (dw) between positions 3081773 and 3089662 on chromosome 5; (dx) between positions 3454995 and 3493107 on chromosome 5; (dy) between positions 3526980 and 3541316 on chromosome 5; (dz) between positions 3585965 and 3604863 on chromosome 5; (ea) between positions 3945751 and 3965771 on chromosome 5; (eb) between positions 4376633 and 4391586 on chromosome 5; (ec) between positions 19976402 and 20026964 on chromosome 5; (ed) between positions 19976402 and 20026964 on chromosome 5; (ee) between positions 20145940 and 20173122 on chromosome 5; (ef) between positions 36089359 and 36288826 on chromosome 5; (eg) between positions 53299267 and 53496045 on chromosome 5; (eh) between positions 81098479 and 81109058 on chromosome X; (ei) between positions 38944628 and 39073783 on chromosome 1; (ej) between positions 67761686 and 67892254 on chromosome 1; (ek) between positions 15652280 and 16182525 on chromosome 3; (el) between positions 39699406 and 40350847 on chromosome 3; (em) between positions 2082401 and 2155110 on chromosome 4; (en) between positions 12779880 and 12825612 on chromosome 6; (eo) between positions 6622471 and 6808016 on chromosome 7; (ep) between positions 10965365 and 11193689 on chromosome 7; (eq) between positions 10965365 and 11193689 on chromosome 7; (er) between positions 12588065 and 12851206 on chromosome 7; (es) between positions 16992324 and 17419297 on chromosome 7; (et) between positions 16992324 and 17419297 on chromosome 7; (eu) between positions 27759260 and 28263307 on chromosome 7; (ev) between positions 28594408 and 29061134 on chromosome 7; (ew) between positions 29891019 and 30608774 on chromosome 7; (ex) between positions 30608774 and 32279982 on chromosome 7; (ey) between positions 36579046 and 36880336 on chromosome 7; (ez) between positions 37049888 and 37211605 on chromosome 7; (fa) between positions 52544592 and 53396185 on chromosome 7; (fb) between positions 56018989 and 56076209 on chromosome 7; (fc) between positions 56171548 and 56426824 on chromosome 7; (fd) between positions 56910768 and 57069404 on chromosome 7; (fe) between positions 58428139 and 58607780 on chromosome 7; (ff) between positions 46392138 and 46584908 on chromosome 9 wherein the reference genome is the Abacus Cannabis reference genome (version CsaAba2).

In an embodiment, the modified cannabinoids correlate to an increased ratio of the combination of total THC, total CBD, total CBG, total THCV, total CBDV, total CBC, and total CBGV to the combination of total CBG and CBGV. In an embodiment, the one or more markers comprises a polymorphism relative to a reference genome at nucleotide position: (a) 32398208 on chromosome 1; or (b) 1276491 on chromosome 3; or (c) 4023145 on chromosome 6; or (d) 6147814 on chromosome 7; or (e) 7857512 on chromosome 7; or (f) 9595805 on chromosome 7; or (g) 9668980 on chromosome 7; or (h) 18616969 on chromosome 7; or (i) 20028244 on chromosome 7; or (j) 29686440 on chromosome 7; or (k) 34956997 on chromosome 7; or (l) 35637532 on chromosome 7; or (m) 35653590 on chromosome 7; or (n) 36003907 on chromosome 7; or (o) 37994590 on chromosome 7; or (p) 38736896 on chromosome 7; or (q) 41296609 on chromosome 7; or (r) 41986329 on chromosome 7; or (s) 42051322 on chromosome 7; or (t) 42152092 on chromosome 7; or (u) 42289736 on chromosome 7; or (v) 42415983 on chromosome 7; or (w) 42793823 on chromosome 7; or (x) 44942488 on chromosome 7; or (y) 46093742 on chromosome 7; or (z) 48399881 on chromosome 7; or (aa) 48949721 on chromosome 7; or (ab) 49294749 on chromosome 7; or (ac) 50582962 on chromosome 7; or (ad) 50745259 on chromosome 7; or (ae) 50820763 on chromosome 7; or (af) 50854826 on chromosome 7; or (ag) 51054719 on chromosome 7; or (ah) 51173524 on chromosome 7; or (ai) 52285032 on chromosome 7; or (aj) 52296271 on chromosome 7; or (ak) 52322834 on chromosome 7; or (al) 52332628 on chromosome 7; or (am) 52439705 on chromosome 7; or (an) 52554676 on chromosome 7; or (ao) 52561249 on chromosome 7; or (ap) 54400345 on chromosome 7; or (aq) 58233054 on chromosome 7; or (ar) 59740097 on chromosome 7 wherein the reference genome is the Abacus Cannabis reference genome (version CsaAba2). In an embodiment, the nucleotide position comprises: (a) a C/C or C/T genotype at position 32398208 on chromosome 1; (b) a G/G or G/T genotype at position 1276491 on chromosome 3; (c) a A/A or G/A genotype at position 4023145 on chromosome 6; (d) a T/T or C/T genotype at position 6147814 on chromosome 7; (e) a A/A or A/T genotype at position 7857512 on chromosome 7; (f) a G/G or A/G genotype at position 9595805 on chromosome 7; (g) a C/C or T/C genotype at position 9668980 on chromosome 7; (h) a G/G or C/G genotype at position 18616969 on chromosome 7; (i) a A/A or T/A genotype at position 20028244 on chromosome 7; (j) a C/C or T/C genotype at position 29686440 on chromosome 7; (k) a A/A or G/A genotype at position 34956997 on chromosome 7; (l) a G/G or G/T genotype at position 35637532 on chromosome 7; (m) a T/T or T/A genotype at position 35653590 on chromosome 7; (n) a G/G or A/G genotype at position 36003907 on chromosome 7; (o) a T/T or A/T genotype at position 37994590 on chromosome 7; (p) a A/A or C/A genotype at position 38736896 on chromosome 7; (q) a G/G or T/G genotype at position 41296609 on chromosome 7; (r) a G/G or A/G genotype at position 41986329 on chromosome 7; (s) a T/T or T/C genotype at position 42051322 on chromosome 7; (t) a C/C or T/C genotype at position 42152092 on chromosome 7; (u) a G/G or G/A genotype at position 42289736 on chromosome 7; (v) a A/A or A/G genotype at position 42415983 on chromosome 7; (w) a T/T or C/T genotype at position 42793823 on chromosome 7; (x) a A/A or G/A genotype at position 44942488 on chromosome 7; (y) a C/C or C/A genotype at position 46093742 on chromosome 7; (z) a T/T or C/T genotype at position 48399881 on chromosome 7; (aa) a G/G or A/G genotype at position 48949721 on chromosome 7; (ab) a G/G or G/C genotype at position 49294749 on chromosome 7; (ac) a C/C or T/C genotype at position 50582962 on chromosome 7; (ad) a T/T or T/C genotype at position 50745259 on chromosome 7; (ae) a C/C or G/C genotype at position 50820763 on chromosome 7; (af) a C/C or A/C genotype at position 50854826 on chromosome 7; (ag) a T/T or C/T genotype at position 51054719 on chromosome 7; (ah) a A/A or G/A genotype at position 51173524 on chromosome 7; (ai) a A/A or G/A genotype at position 52285032 on chromosome 7; (aj) a C/C or T/C genotype at position 52296271 on chromosome 7; (ak) a G/G or A/G genotype at position 52322834 on chromosome 7; (al) a A/A or G/A genotype at position 52332628 on chromosome 7; (am) a A/A or G/A genotype at position 52439705 on chromosome 7; (an) a A/A or T/A genotype at position 52554676 on chromosome 7; (ao) a G/G or A/G genotype at position 52561249 on chromosome 7; (ap) a G/G or A/G genotype at position 54400345 on chromosome 7; (aq) a T/T or C/T genotype at position 58233054 on chromosome 7; (ar) a G/G or A/G genotype at position 59740097 on chromosome 7 wherein the reference genome is the Abacus Cannabis reference genome (version CsaAba2).

In an embodiment, the cannabinoid comprises a type I, II, or III cannabinoid. In an embodiment, the one or more markers comprises a polymorphism at position 26 of any one or more of SEQ ID NO: 3; SEQ ID NO: 19; SEQ ID NO: 118; SEQ ID NO: 144; SEQ ID NO: 151; SEQ ID NO: 156; SEQ ID NO: 160; SEQ ID NO: 184; SEQ ID NO: 185; SEQ ID NO: 194; SEQ ID NO: 207; SEQ ID NO: 208; SEQ ID NO: 209; SEQ ID NO: 210; SEQ ID NO: 215; SEQ ID NO: 216; SEQ ID NO: 217; SEQ ID NO: 220; SEQ ID NO: 221; SEQ ID NO: 222; SEQ ID NO: 223; SEQ ID NO: 224; SEQ ID NO: 225; SEQ ID NO: 229; SEQ ID NO: 230; SEQ ID NO: 235; SEQ ID NO: 239; SEQ ID NO: 246; SEQ ID NO: 264; SEQ ID NO: 268; SEQ ID NO: 271; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 281; SEQ ID NO: 286; SEQ ID NO: 287; SEQ ID NO: 294; SEQ ID NO: 340; SEQ ID NO: 350. In an embodiment, the nucleotide position comprises: (a) a C/C or C/T genotype at position 51 of SEQ ID NO: 3; (b) a G/G or G/T genotype at position 51 of SEQ ID NO: 19; (c) a A/A or G/A genotype at position 51 of SEQ ID NO: 118; (d) a T/T or C/T genotype at position 51 of SEQ ID NO: 144; (e) a A/A or A/T genotype at position 51 of SEQ ID NO: 151; (f) a G/G or A/G genotype at position 51 of SEQ ID NO: 156; (g) a C/C or T/C genotype at position 51 of SEQ ID NO: 160; (h) a G/G or C/G genotype at position 51 of SEQ ID NO: 184; (i) a A/A or T/A genotype at position 51 of SEQ ID NO: 185; (j) a C/C or T/C genotype at position 51 of SEQ ID NO: 194; (k) a A/A or G/A genotype at position 51 of SEQ ID NO: 207; (l) a G/G or G/T genotype at position 51 of SEQ ID NO: 208; (m) a T/T or T/A genotype at position 51 of SEQ ID NO: 209; (n) a G/G or A/G genotype at position 51 of SEQ ID NO: 210; (o) a T/T or A/T genotype at position 51 of SEQ ID NO: 215; (p) a A/A or C/A genotype at position 51 of SEQ ID NO: 216; (q) a G/G or T/G genotype at position 51 of SEQ ID NO: 217; (r) a G/G or A/G genotype at position 51 of SEQ ID NO: 220; (s) a T/T or T/C genotype at position 51 of SEQ ID NO: 221; (t) a C/C or T/C genotype at position 51 of SEQ ID NO: 222; (u) a G/G or G/A genotype at position 51 of SEQ ID NO: 223; (v) a A/A or A/G genotype at position 51 of SEQ ID NO: 224; (w) a T/T or C/T genotype at position 51 of SEQ ID NO: 225; (x) a A/A or G/A genotype at position 51 of SEQ ID NO: 229; (y) a C/C or C/A genotype at position 51 of SEQ ID NO: 230; (z) a T/T or C/T genotype at position 51 of SEQ ID NO: 235; (aa) a G/G or A/G genotype at position 51 of SEQ ID NO: 239; (ab) a G/G or G/C genotype at position 51 of SEQ ID NO: 246; (ac) a C/C or T/C genotype at position 51 of SEQ ID NO: 264; (ad) a T/T or T/C genotype at position 51 of SEQ ID NO: 268; (ae) a C/C or G/C genotype at position 51 of SEQ ID NO: 271; (af) a C/C or A/C genotype at position 51 of SEQ ID NO: 273; (ag) a T/T or C/T genotype at position 51 of SEQ ID NO: 274; (ah) a A/A or G/A genotype at position 51 of SEQ ID NO: 275; (ai) a A/A or G/A genotype at position 51 of SEQ ID NO: 276; (aj) a C/C or T/C genotype at position 51 of SEQ ID NO: 277; (ak) a G/G or A/G genotype at position 51 of SEQ ID NO: 278; (al) a A/A or G/A genotype at position 51 of SEQ ID NO: 279; (am) a A/A or G/A genotype at position 51 of SEQ ID NO: 281; (an) a A/A or T/A genotype at position 51 of SEQ ID NO: 286; (ao) a G/G or A/G genotype at position 51 of SEQ ID NO: 287; (ap) a G/G or A/G genotype at position 51 of SEQ ID NO: 294; (aq) a T/T or C/T genotype at position 51 of SEQ ID NO: 340; (ar) a G/G or A/G genotype at position 51 of SEQ ID NO: 350 wherein the reference genome is the Abacus Cannabis reference genome (version CsaAba2). In an embodiment, the one or more markers comprises a polymorphism relative to a reference genome within any one or more haplotypes wherein the haplotypes comprise the region: (a) between positions 32382605 and 32414393 on chromosome 1; (b) between positions 1276073 and 1284641 on chromosome 3; (c) between positions 4002406 and 4037935 on chromosome 6; (d) between positions 6125186 and 6165666 on chromosome 7; (e) between positions 7822322 and 7864334 on chromosome 7; (f) between positions 9585902 and 9598562 on chromosome 7; (g) between positions 9662890 and 9677657 on chromosome 7; (h) between positions 18581032 and 18640383 on chromosome 7; (i) between positions 20022570 and 20050272 on chromosome 7; (j) between positions 29661512 and 29686508 on chromosome 7; (k) between positions 34941497 and 34997619 on chromosome 7; (l) between positions 35592132 and 35695775 on chromosome 7; (m) between positions 35592132 and 35695775 on chromosome 7; (n) between positions 35799742 and 36153842 on chromosome 7; (o) between positions 37898780 and 38009095 on chromosome 7; (p) between positions 38729407 and 38794976 on chromosome 7; (q) between positions 41248971 and 41343647 on chromosome 7; (r) between positions 41982953 and 41988552 on chromosome 7; (s) between positions 42043493 and 42096029 on chromosome 7; (t) between positions 42096029 and 42156112 on chromosome 7; (u) between positions 42175605 and 42379636 on chromosome 7; (v) between positions 42379636 and 42418406 on chromosome 7; (w) between positions 42758132 and 42819971 on chromosome 7; (x) between positions 44924842 and 44959410 on chromosome 7; (y) between positions 46090347 and 46097781 on chromosome 7; (z) between positions 48393114 and 48443534 on chromosome 7; (aa) between positions 48862965 and 49016897 on chromosome 7; (ab) between positions 49288761 and 49297082 on chromosome 7; (ac) between positions 50541967 and 50605020 on chromosome 7; (ad) between positions 50737827 and 50747700 on chromosome 7; (ae) between positions 50818092 and 50822516 on chromosome 7; (af) between positions 50832568 and 50861059 on chromosome 7; (ag) between positions 51022401 and 51062076 on chromosome 7; (ah) between positions 51141514 and 51226826 on chromosome 7; (ai) between positions 51909282 and 52291349 on chromosome 7; (aj) between positions 52291349 and 52412182 on chromosome 7; (ak) between positions 52291349 and 52412182 on chromosome 7; (al) between positions 52291349 and 52412182 on chromosome 7; (am) between positions 52435669 and 52441872 on chromosome 7; (an) between positions 52552192 and 52760459 on chromosome 7; (ao) between positions 52552192 and 52760459 on chromosome 7; (ap) between positions 54394199 and 54410973 on chromosome 7; (aq) between positions 58220261 and 58237329 on chromosome 7; (ar) between positions 59728563 and 59762135 on chromosome 7 wherein the reference genome is the Abacus Cannabis reference genome (version CsaAba2).

In an embodiment, the modified cannabinoids correlate to an increased ratio of the combination of total THC and total THCV to the combination of total CBG and total CBGV. In an embodiment, the one or more markers comprises a polymorphism relative to a reference genome at nucleotide position: (a) 32398208 on chromosome 1; or (b) 1276491 on chromosome 3; or (c) 1845149 on chromosome 5; or (d) 2038965 chromosome 5; or (e) 2364964 on chromosome 5; or (f) 4023145 on chromosome 6; or (g) 6147814 on chromosome 7; or (h) 7085058 on chromosome 7; or (i) 7857512 on chromosome 7; or (j) 8656466 on chromosome 7; or (k) 9595805 on chromosome 7; or (l) 9625781 on chromosome 7; or (m) 9668980 on chromosome 7; or (n) 9716257 on chromosome 7; or (o) 18616969 on chromosome 7; or (p) 20028244 on chromosome 7; or (q) 24653415 on chromosome 7; or (r) 25703806 on chromosome 7; or (s) 29420823 on chromosome 7; or (t) 29686440 on chromosome 7; or (u) 30033193 on chromosome 7; or (v) 30196905 on chromosome 7; or (w) 30306362 on chromosome 7; or (x) 30318939 on chromosome 7; or (y) 30411594 on chromosome 7; or (z) 30696835 on chromosome 7; or (aa) 33474698 on chromosome 7; or (ab) 33580732 on chromosome 7; or (ac) 33990610 on chromosome 7; or (ad) 34059134 on chromosome 7; or (ae) 34956997 on chromosome 7; or (af) 35637532 on chromosome 7; or (ag) 35653590 on chromosome 7; or (ah) 36003907 on chromosome 7; or (ai) 38736896 on chromosome 7; or (aj) 41296609 on chromosome 7; or (ak) 41852842 on chromosome 7; or (al) 41986329 on chromosome 7; or (am) 42051322 on chromosome 7; or (an) 42152092 on chromosome 7; or (ao) 42289736 on chromosome 7; or (ap) 42415983 on chromosome 7; or (aq) 42793823 on chromosome 7; or (ar) 44700019 on chromosome 7; or (as) 44942488 on chromosome 7; or (at) 46093742 on chromosome 7; or (au) 48399881 on chromosome 7; or (av) 48949721 on chromosome 7; or (aw) 49294749 on chromosome 7; or (ax) 50745259 on chromosome 7; or (ay) 50820763 on chromosome 7; or (az) 50854826 on chromosome 7; or (ba) 51054719 on chromosome 7; or (bb) 51173524 on chromosome 7; or (bc) 52285032 on chromosome 7; or (bd) 52296271 on chromosome 7; or (be) 52322834 on chromosome 7; or (bf) 52332628 on chromosome 7; or (bg) 52412182 on chromosome 7; or (bh) 52439705 on chromosome 7; or (bi) 52554676 on chromosome 7; or (bj) 52561249 on chromosome 7; or (bk) 52932343 on chromosome 7; or (bl) 54400345 on chromosome 7; or (bm) 56018989 on chromosome 7; or (bn) 56076209 on chromosome 7; or (bo) 56081903 on chromosome 7; or (bp) 56448133 on chromosome 7; or (bq) 57770425 on chromosome 7; or (br) 58178218 on chromosome 7; or (bs) 58190719 on chromosome 7; or (bt) 58233054 on chromosome 7; or (bu) 58244327 on chromosome 7; or (bv) 58271366 on chromosome 7; or (bw) 58317539 on chromosome 7; or (bx) 58450928 on chromosome 7; or (by) 58481147 on chromosome 7; or (bz) 59740097 on chromosome 7; or (ca) 60366254 on chromosome 7; or (cb) 32398208 on chromosome 1; or (cc) 4023145 on chromosome 6; or (cd) 5908268 on chromosome 7; or (ce) 7639988 on chromosome 7; or (cf) 13731414 on chromosome 7; or (cg) 18616969 on chromosome 7; or (ch) 30411594 on chromosome 7; or (ci) 37505904 on chromosome 7; or (cj) 41659351 on chromosome 7; or (ck) 44916971 on chromosome 7; or (cl) 49114154 on chromosome 7; or (cm) 50745259 on chromosome 7; or (cn) 50767962 on chromosome 7; or (co) 50820763 on chromosome 7; or (cp) 50854826 on chromosome 7; or (cq) 51054719 on chromosome 7; or (cr) 51173524 on chromosome 7; or (cs) 52285032 on chromosome 7; or (ct) 52296271 on chromosome 7; or (cu) 52322834 on chromosome 7; or (cv) 52332628 on chromosome 7; or (cw) 52554676 on chromosome 7; or (cx) 52561249 on chromosome 7; or (cy) 58317539 on chromosome 7; or (cz) 58528791 on chromosome 7; or (da) 2366529 on chromosome 5; or (db) 2899343 on chromosome 5; or (dc) 3074649 on chromosome 5; or (dd) 3086874 on chromosome 5; or (de) 3432405 on chromosome 5; or (df) 3485895 on chromosome 5; or (dg) 3535297 on chromosome 5; or (dh) 3599637 on chromosome 5; or (di) 3673686 on chromosome 5; or (dj) 3961139 or chromosome 5; or (dk) 4118245 on chromosome 5; or (dl) 4384123 on chromosome 5; 0 (dm) 6491377 on chromosome 5; or (dn) 6666604 on chromosome 5; or (do) 7129622 on chromosome 5; or (dp) 7245930 on chromosome 5; or (dq) 7904873 on chromosome 5; or (dr) 8078921 on chromosome 5; or (ds) 9111167 on chromosome 5; or (dt) 9113994 on chromosome 5; or (du) 9472805 on chromosome 5; or (dv) 9565471 on chromosome 5; or (dw) 9734723 on chromosome 5; or (dx) 9752109 on chromosome 5; or (dy) 10155920 on chromosome 5; or (dz) 10439554 on chromosome 5; or (ea) 10451559 on chromosome 5; or (eb) 10469451 on chromosome 5; or (ec) 10548740 on chromosome 5; or (ed) 11161216 on chromosome 5; or (ee) 11245818 on chromosome 5; or (ef) 11257676 on chromosome 5; or (eg) 11310169 on chromosome 5; or (eh) 12446524 on chromosome 5; or (ei) 12932181 on chromosome 5; or (ej) 13378138 on chromosome 5; or (ek) 13758916 on chromosome 5; or (el) 14847788 on chromosome 5; or (em) 15194741 on chromosome 5; or (en) 16033894 on chromosome 5; or (eo) 18706160 on chromosome 5; or (ep) 19988534 on chromosome 5; or (eq) 20017410 on chromosome 5; or (er) 20148519 on chromosome 5; or (es) 20537946 on chromosome 5; or (et) 20790022 on chromosome 5; or (eu) 21524826 on chromosome 5; or (ev) 21563573 on chromosome 5; or (ew) 23962791 on chromosome 5; or (ex) 23968827 on chromosome 5; or (ey) 23975759 on chromosome 5; or (ez) 24897787 on chromosome 5; or (fa) 25356228 on chromosome 5; or (fb) 25364836 on chromosome 5; or (fc) 25387741 on chromosome 5; or (fd) 25536887 on chromosome 5; or (fe) 28880563 on chromosome 5; or (ff) 32311818 on chromosome 5; or (ga) 32387532 on chromosome 5; or (gb) 36148442 on chromosome 5; or (gc) 40532113 on chromosome 5; or (gd) 42410724 on chromosome 5; or (ge) 42557566 on chromosome 5; or (gf) 43499675 on chromosome 5; or (gg) 44062719 on chromosome 5; or (gh) 45687721 on chromosome 5; or (gi) 48977178 on chromosome 5; or (gj) 52819178 on chromosome 5; or (gk) 53489757 on chromosome 5; or (gl) 53719271 on chromosome 5; or (gm) 54339213 on chromosome 5; or (gn) 56258287 on chromosome 5; or (go) 57537902 on chromosome 5; or (gp) 57827101 on chromosome 5; or (gq) 58026390 on chromosome 5; or (gr) 58596336 on chromosome 5; or (gs) 59820696 on chromosome 5; or (gt) 60280385 on chromosome 5; or (gu) 60295701 on chromosome 5; or (gv) 61249054 on chromosome 5; or (gw) 64186528 on chromosome 5; or (gx) 64884077 on chromosome 5; or (gy) 23406044 on chromosome 7; or (gz) 38978759 on chromosome 1; or (ha) 48365472 on chromosome 7; or (hb) 48366628 on chromosome 7; or (hc) 48587800 on chromosome 7; or (hd) 48688297 on chromosome 7; or (he) 48694159 on chromosome 7; or (hf) 49044262 on chromosome 7; or (hg) 49065141 on chromosome 7; or (hh) 49134977 on chromosome 7; or (hi) 49139036 on chromosome 7; or (hj) 49272668 on chromosome 7; or (hk) 49297082 on chromosome 7; or (hl) 49304484 on chromosome 7; or (hm) 49331724 on chromosome 7; or (hn) 49344499 on chromosome 7; or (ho) 49394880 on chromosome 7; or (hp) 49628859 on chromosome 7; or (hq) 49634834 on chromosome 7; or (hr) 49664112 on chromosome 7; or (hs) 49666319 on chromosome 7; or (ht) 49843878 on chromosome 7; or (hu) 49979803 on chromosome 7; or (hv) 50084463 on chromosome 7; or (hw) 50302149 on chromosome 7; or (hx) 50515764 on chromosome 7; or (hy) 50582962 on chromosome 7; or (hz) 50630217 on chromosome 7; or (ia) 50662578 on chromosome 7; or (ib) 50715700 on chromosome 7; or (ic) 50767962 on chromosome 7; or (id) 50818092 on chromosome 7; or (ie) 52439705 on chromosome 7; or (if) 52441872 on chromosome 7; or (ig) 52507913 on chromosome 7; or (ih) 52510830 on chromosome 7; or (ii) 52517301 on chromosome 7; or (ij) 52923743 on chromosome 7; or (ik) 53449873 on chromosome 7; or (il) 53505022 on chromosome 7; or (im) 54400345 on chromosome 7; or (in) 54464358 on chromosome 7; or (io) 54509399 on chromosome 7; or (ip) 54513057 on chromosome 7; or (iq) 54555051 on chromosome 7; or (ir) 54693540 on chromosome 7; or (is) 54722302 on chromosome 7; or (it) 54915189 on chromosome 7; or (iu) 55277957 on chromosome 7; or (iv) 55452244 on chromosome 7; or (iw) 55716705 on chromosome 7; or (ix) 55735556 on chromosome 7; or (iy) 55836464 on chromosome 7; or (iz) 55970630 on chromosome 7; or (ja) 55984088 on chromosome 7; or (jb) 56018989 on chromosome 7; or (jc) 56032988 on chromosome 7; or (jd) 56076209 on chromosome 7; or (je) 56081903 on chromosome 7; or (jf) 56171548 on chromosome 7; or (jg) 56301604 on chromosome 7; or (jh) 56426824 on chromosome 7; or (ji) 56430375 on chromosome 7; or (jj) 56440283 on chromosome 7; or (jk) 56488125 on chromosome 7; or (jl) 56492081 on chromosome 7; or (jm) 56538007 on chromosome 7; or (jn) 56700085 on chromosome 7; or (jo) 56782341 on chromosome 7; or (jp) 56872960 on chromosome 7; or (jq) 56910768 on chromosome 7; or (jr) 56967275 on chromosome 7; or (js) 57069404 on chromosome 7; or (jt) 57080583 on chromosome 7; or (ju) 57089709 on chromosome 7; or (jv) 57104188 on chromosome 7; or (jw) 57120122 on chromosome 7; or (jx) 57152672 on chromosome 7; or (jy) 57228643 on chromosome 7; or (jz) 57233796 on chromosome 7; or (ha) 58538433 on chromosome 7 wherein the reference genome is the Abacus Cannabis reference genome (version CsaAba2). In an embodiment, the nucleotide position comprises: (a) a T/T or C/T genotype at position 32398208 on chromosome 1; (b) a T/T or G/T genotype at position 1276491 on chromosome 3; (c) a T/T or T/C genotype at position 1845149 on chromosome 5; (d) a T/T genotype at position 2038965 on chromosome 5; (e) a C/C or T/C genotype at position 2364964 on chromosome 5; (f) a A/A or G/A genotype at position 4023145 on chromosome 6; (g) a T/T or C/T genotype at position 6147814 on chromosome 7; (h) a A/A or G/A genotype at position 7085058 on chromosome 7; (i) a A/A or A/T genotype at position 7857512 on chromosome 7; (j) a C/C or T/C genotype at position 8656466 on chromosome 7; (k) a G/G or A/G genotype at position 9595805 on chromosome 7; (l) a A/A or C/A genotype at position 9625781 on chromosome 7; (m) a C/C or T/C genotype at position 9668980 on chromosome 7; (n) a G/G or A/G genotype at position 9716257 on chromosome 7; (o) a G/G or C/G genotype at position 18616969 on chromosome 7; (p) a A/A or T/A genotype at position 20028244 on chromosome 7; (q) a A/A or T/A genotype at position 24653415 on chromosome 7; (r) a A/A or G/A genotype at position 25703806 on chromosome 7; (s) a G/G or T/G genotype at position 29420823 on chromosome 7; (t) a C/C or T/C genotype at position 29686440 on chromosome 7; (u) a C/C or T/C genotype at position 30033193 on chromosome 7; (v) a C/C or T/C genotype at position 30196905 on chromosome 7; (w) a C/C or T/C genotype at position 30306362 on chromosome 7; (x) a A/A or C/A genotype at position 30318939 on chromosome 7; (y) a C/C or G/C genotype at position 30411594 on chromosome 7; (z) a G/G or T/G genotype at position 30696835 on chromosome 7; (aa) a G/G or A/G genotype at position 33474698 on chromosome 7; (ab) a C/C or A/C genotype at position 33580732 on chromosome 7; (ac) a A/A or T/A genotype at position 33990610 on chromosome 7; (ad) a G/G or C/G genotype at position 34059134 on chromosome 7; (ae) a A/A or G/A genotype at position 34956997 on chromosome 7; (af) a G/G or G/T genotype at position 35637532 on chromosome 7; (ag) a T/T or T/A genotype at position 35653590 on chromosome 7; (ah) a A/A or A/G genotype at position 36003907 on chromosome 7; (ai) a A/A or C/A genotype at position 38736896 on chromosome 7; (aj) a G/G or T/G genotype at position 41296609 on chromosome 7; (ak) a A/A or G/A genotype at position 41852842 on chromosome 7; (al) a G/G or A/G genotype at position 41986329 on chromosome 7; (am) a T/T or T/C genotype at position 42051322 on chromosome 7; (an) a T/T or T/C genotype at position 42152092 on chromosome 7; (ao) a G/G or G/A genotype at position 42289736 on chromosome 7 (ap) a A/A or A/G genotype at position 42415983 on chromosome 7; (aq) a T/T or C/T genotype at position 42793823 on chromosome 7; (ar) a T/T or C/T genotype at position 44700019 on chromosome 7; (as) a A/A or G/A genotype at position 44942488 on chromosome 7; (at) a C/C or C/A genotype at position 46093742 on chromosome 7; (au) a T/T or C/T genotype at position 48399881 on chromosome 7; (av) a A/A or A/G genotype at position 48949721 on chromosome 7; (aw) a C/C or G/C genotype at position 49294749 on chromosome 7; (ax) a C/C or T/C genotype at position 50745259 on chromosome 7; (ay) a C/C or G/C genotype at position 50820763 on chromosome 7; (az) a C/C or A/C genotype at position 50854826 on chromosome 7; (ba) a T/T or C/T genotype at position 51054719 on chromosome 7; (bb) a A/A or G/A genotype at position 51173524 on chromosome 7; (bc) a A/A or G/A genotype at position 52285032 on chromosome 7; (bd) a C/C or T/C genotype at position 52296271 on chromosome 7; (be) a G/G or A/G genotype at position 52322834 on chromosome 7; (bf) a A/A or G/A genotype at position 52332628 on chromosome 7; (bg) a G/G or G/T genotype at position 52412182 on chromosome 7; (bh) a A/A or G/A genotype at position 52439705 on chromosome 7; (bi) a A/A or T/A genotype at position 52554676 on chromosome 7; (bj) a G/G or A/G genotype at position 52561249 on chromosome 7; (bk) a G/G or T/G genotype at position 52932343 on chromosome 7; (bl) a G/G or A/G genotype at position 54400345 on chromosome 7; (bm) a A/A or G/A genotype at position 56018989 on chromosome 7; (bn) a T/T or C/T genotype at position 56076209 on chromosome 7; (bo) a C/C or T/C genotype at position 56081903 on chromosome 7; (bp) a C/C or T/C genotype at position 56448133 on chromosome 7; (bq) a A/A or C/A genotype at position 57770425 on chromosome 7; (br) a A/A or A/G genotype at position 58178218 on chromosome 7; (bs) a T/T or T/C genotype at position 58190719 on chromosome 7; (bt) a T/T or C/T genotype at position 58233054 on chromosome 7; (bu) a T/T or T/G genotype at position 58244327 on chromosome 7; (bv) a A/A or A/G genotype at position 58271366 on chromosome 7; (bw) a G/G or G/A genotype at position 58317539 on chromosome 7; (bx) a C/C or T/C genotype at position 58450928 on chromosome 7; (by) a C/C or T/C genotype at position 58481147 on chromosome 7; (bz) a G/G or A/G genotype at position 59740097 on chromosome 7; (ca) a A/A genotype at position 60366254 on chromosome 7; (cb) a T/T or C/T genotype at position 32398208 on chromosome 1; (cc) a A/A or G/A genotype at position 4023145 on chromosome 6; (cd) a C/C or T/C genotype at position 5908268 on chromosome 7; (ce) a A/A or G/A genotype at position 7639988 on chromosome 7; (cf) a C/A genotype at position 13731414 on chromosome 7; (cg) a G/G or C/G genotype at position 18616969 on chromosome 7; (ch) a C/C or G/C genotype at position 30411594 on chromosome 7; (ci) a A/A or A/G genotype at position 37505904 on chromosome 7; (cj) a G/G or A/G genotype at position 41659351 on chromosome 7; (ck) a G/G or G/A genotype at position 44916971 on chromosome 7; (cl) a T/T or G/T genotype at position 49114154 on chromosome 7; (cm) a C/C or T/C genotype at position 50745259 on chromosome 7; (cn) a G/G or G/C genotype at position 50767962 on chromosome 7; (co) a C/C or G/C genotype at position 50820763 on chromosome 7; (cp) a C/C or A/C genotype at position 50854826 on chromosome 7; (cq) a T/T or C/T genotype at position 51054719 on chromosome 7; (cr) a A/A or G/A genotype at position 51173524 on chromosome 7; (cs) a A/A or G/A genotype at position 52285032 on chromosome 7; (ct) a C/C or T/C genotype at position 52296271 on chromosome 7; (cu) a G/G or A/G genotype at position 52322834 on chromosome 7; (cv) a A/A or G/A genotype at position 52332628 on chromosome 7; (cw) a A/A or T/A genotype at position 52554676 on chromosome 7; (cx) a G/G or A/G genotype at position 52561249 on chromosome 7; (cy) a G/G or G/A genotype at position 58317539 on chromosome 7; (cz) a T/T or C/T genotype at position 58528791 on chromosome 7; (da) a C/C or C/T genotype at position 2366529 on chromosome 5; (db) a T/T genotype at position 2899343 on chromosome 5; (dc) a C/C or C/T genotype at position 3074649 on chromosome 5; (dd) a T/T or T/C genotype at position 3086874 on chromosome 5; (de) a G/G or G/A genotype at position 3432405 on chromosome 5; (df) a T/T genotype at position 3485895 on chromosome 5; (dg) a A/A or A/G genotype at position 3535297 on chromosome 5; (dh) a G/G or G/A genotype at position 3599637 on chromosome 5; (di) a G/G or G/A genotype at position 3673686 on chromosome 5; (dj) a C/C or C/G genotype at position 3961139 on chromosome 5; (dk) a A/A or A/G genotype at position 4118245 on chromosome 5; (dl) a A/A genotype at position 4384123 on chromosome 5; (dm) a C/C or C/T genotype at position 6491377 on chromosome 5; (dn) a T/T or T/G genotype at position 6666604 on chromosome 5; (do) a A/A or A/C genotype at position 7129622 on chromosome 5; (dp) a T/T or T/C genotype at position 7245930 on chromosome 5; (dq) a A/A or A/G genotype at position 7904873 on chromosome 5; (dr) a G/G or G/A genotype at position 8078921 on chromosome 5; (ds) a G/G or G/A genotype at position 9111167 on chromosome 5; (dt) a T/T or T/C genotype at position 9113994 on chromosome 5; (du) a T/T or T/C genotype at position 9472805 on chromosome 5; (dv) a C/C or C/T genotype at position 9565471 on chromosome 5; (dw) a G/G or G/C genotype at position 9734723 on chromosome 5 (dx) a G/G or G/T genotype at position 9752109 on chromosome 5; (dy) a C/C or C/T genotype at position 10155920 on chromosome 5; (dz) a C/C or C/T genotype at position 10439554 on chromosome 5; (ea) a A/A or A/G genotype at position 10451559 on chromosome 5; (eb) a A/A or A/G genotype at position 10469451 on chromosome 5; (ec) a G/G or G/A genotype at position 10548740 on chromosome 5; (ed) a T/T or T/C genotype at position 11161216 on chromosome 5; (ee) a G/G or G/A genotype at position 11245818 on chromosome 5; (ef) a C/C or C/A genotype at position 11257676 on chromosome 5; (eg) a A/A or A/G genotype at position 11310169 on chromosome 5; (eh) a A/A or A/T genotype at position 12446524 on chromosome 5; (ei) a T/T or T/G genotype at position 12932181 on chromosome 5; (ej) a C/C or C/T genotype at position 13378138 on chromosome 5; (ek) a A/A or A/G genotype at position 13758916 on chromosome 5; (el) a T/T or T/A genotype at position 14847788 on chromosome 5; (em) a C/C or C/G genotype at position 15194741 on chromosome 5; (en) a G/G or G/A genotype at position 16033894 on chromosome 5; (eo) a G/G or G/A genotype at position 18706160 on chromosome 5; (ep) a C/C or C/T genotype at position 19988534 on chromosome 5; (eq) a T/T or T/G genotype at position 20017410 on chromosome 5; (er) a G/G or G/A genotype at position 20148519 on chromosome 5; (es) a A/A or A/T genotype at position 20537946 on chromosome 5; (et) a C/C or C/A genotype at position 20790022 on chromosome 5; (eu) a T/T or T/C genotype at position 21524826 on chromosome 5; (ev) a C/C or C/T genotype at position 21563573 on chromosome 5; (ew) a T/T or T/G genotype at position 23962791 on chromosome 5; (ex) a A/A or A/G genotype at position 23968827 on chromosome 5; (ey) a A/A or A/T genotype at position 23975759 on chromosome 5; (ez) a A/A or A/C genotype at position 24897787 on chromosome 5; (fa) a C/C or C/T genotype at position 25356228 on chromosome 5; (fb) a T/T or T/C genotype at position 25364836 on chromosome 5; (fc) a G/G or G/C genotype at position 25387741 on chromosome 5; (fd) a G/G or G/A genotype at position 25536887 on chromosome 5; (fe) a G/G or G/T genotype at position 28880563 on chromosome 5; (ff) a T/T or T/C genotype at position 32311818 on chromosome 5; (ga) a C/C or C/T genotype at position 32387532 on chromosome 5; (gb) a C/C or C/T genotype at position 36148442 on chromosome 5; (gc) a C/C or C/T genotype at position 40532113 on chromosome 5; (gd) a C/C or C/T genotype at position 42410724 on chromosome 5; (ge) a A/A or A/G genotype at position 42557566 on chromosome 5; (gf) a A/A or A/C genotype at position 43499675 on chromosome 5; (gg) a G/G or G/A genotype at position 44062719 on chromosome 5; (gh) a T/T or T/C genotype at position 45687721 on chromosome 5; (gi) a T/T or T/A genotype at position 48977178 on chromosome 5; (gj) a C/C or C/T genotype at position 52819178 on chromosome 5; (gk) a C/C or C/A genotype at position 53489757 on chromosome 5; (gl) a C/C or C/T genotype at position 53719271 on chromosome 5; (gm) a C/C or C/T genotype at position 54339213 on chromosome 5; (gn) a C/C or C/T genotype at position 56258287 on chromosome 5; (go) a C/C or C/A genotype at position 57537902 on chromosome 5; (gp) a T/T or T/A genotype at position 57827101 on chromosome 5; (gq) a C/C or C/T genotype at position 58026390 on chromosome 5; (gr) a C/C or C/T genotype at position 58596336 on chromosome 5; (gs) a T/T or T/A genotype at position 59820696 on chromosome 5; (gt) a T/T or T/C genotype at position 60280385 on chromosome 5; (gu) a A/A or A/G genotype at position 60295701 on chromosome 5; (gv) a A/A or A/G genotype at position 61249054 on chromosome 5; (gw) a C/C genotype at position 64186528 on chromosome 5; (gx) a A/A or A/G genotype at position 64884077 on chromosome 5; (gy) a C/C or C/T genotype at position 23406044 on chromosome 7; (gz) a A/A or G/A genotype at position 38978759 on chromosome 1; (ha) a T/T or C/T genotype at position 48365472 on chromosome 7; (hb) a A/A or G/A genotype at position 48366628 on chromosome 7; (hc) a T/T or T/C genotype at position 48587800 on chromosome 7; (hd) a T/T or T/A genotype at position 48688297 on chromosome 7; (he) a T/T or G/T genotype at position 48694159 on chromosome 7; (hf) a C/C or C/G genotype at position 49044262 on chromosome 7; (hg) a A/A or A/T genotype at position 49065141 on chromosome 7; (hh) a C/C or C/A genotype at position 49134977 on chromosome 7; (hi) a T/T or T/C genotype at position 49139036 on chromosome 7; (hj) a T/T or T/A genotype at position 49272668 on chromosome 7; (hk) a G/G or G/T genotype at position 49297082 on chromosome 7; (hl) a A/A or G/A genotype at position 49304484 on chromosome 7; (hm) a G/G or T/G genotype at position 49331724 on chromosome 7; (hn) a C/C or T/C genotype at position 49344499 on chromosome 7; (ho) a T/T or T/A genotype at position 49394880 on chromosome 7; (hp) a G/G or A/G genotype at position 49628859 on chromosome 7; (hq) a T/T or G/T genotype at position 49634834 on chromosome 7; (hr) a G/G or T/G genotype at position 49664112 on chromosome 7; (hs) a A/A or A/T genotype at position 49666319 on chromosome 7; (ht) a C/C or C/G genotype at position 49843878 on chromosome 7; (hu) a G/G or G/T genotype at position 49979803 on chromosome 7; (hv) a T/T or T/G genotype at position 50084463 on chromosome 7; (hw) a A/A or A/G genotype at position 50302149 on chromosome 7; (hx) a A/A or T/A genotype at position 50515764 on chromosome 7; (hy) a C/C or T/C genotype at position 50582962 on chromosome 7; (hz) a C/C or T/C genotype at position 50630217 on chromosome 7; (ia) a G/G or G/A genotype at position 50662578 on chromosome 7; (ib) a G/G or T/G genotype at position 50715700 on chromosome 7; (ic) a G/G or G/C genotype at position 50767962 on chromosome 7; (id) a C/C or C/A genotype at position 50818092 on chromosome 7; (ie) a A/A or G/A genotype at position 52439705 on chromosome 7; (if) a G/G or C/G genotype at position 52441872 on chromosome 7; (ig) a T/T or A/T genotype at position 52507913 on chromosome 7; (ih) a C/C or A/C genotype at position 52510830 on chromosome 7; (ii) a A/A or C/A genotype at position 52517301 on chromosome 7; (ij) a A/A or C/A genotype at position 52923743 on chromosome 7; (ik) a C/C or C/T genotype at position 53449873 on chromosome 7; (il) a G/G or A/G genotype at position 53505022 on chromosome 7; (im) a G/G or A/G genotype at position 54400345 on chromosome 7; (in) a G/G or G/A genotype at position 54464358 on chromosome 7; (io) a T/T or T/G genotype at position 54509399 on chromosome 7; (ip) a G/G or A/G genotype at position 54513057 on chromosome 7; (iq) a T/T or C/T genotype at position 54555051 on chromosome 7; (ir) a A/A or G/A genotype at position 54693540 on chromosome 7; (is) a A/A or C/A genotype at position 54722302 on chromosome 7; (it) a A/A or G/A genotype at position 54915189 on chromosome 7; (iu) a A/A or G/A genotype at position 55277957 on chromosome 7; (iv) a A/A or A/T genotype at position 55452244 on chromosome 7; (iw) a G/G or A/G genotype at position 55716705 on chromosome 7; (ix) a A/A or T/A genotype at position 55735556 on chromosome 7; (iy) a T/T or C/T genotype at position 55836464 on chromosome 7; (iz) a T/T or A/T genotype at position 55970630 on chromosome 7; (ja) a T/T or A/T genotype at position 55984088 on chromosome 7; (jb) a A/A or G/A genotype at position 56018989 on chromosome 7; (jc) a A/A or G/A genotype at position 56032988 on chromosome 7; (jd) a T/T or C/T genotype at position 56076209 on chromosome 7; (je) a C/C or T/C genotype at position 56081903 on chromosome 7; (jf) a G/G or A/G genotype at position 56171548 on chromosome 7; (jg) a A/A or G/A genotype at position 56301604 on chromosome 7; (jh) a G/G or T/G genotype at position 56426824 on chromosome 7; (ji) a A/A or A/G genotype at position 56430375 on chromosome 7; (jj) a T/T or C/T genotype at position 56440283 on chromosome 7; (jk) a A/A or A/G genotype at position 56488125 on chromosome 7; (jl) a T/T or T/G genotype at position 56492081 on chromosome 7; (jm) a A/A or C/A genotype at position 56538007 on chromosome 7; (jn) a T/T or T/C genotype at position 56700085 on chromosome 7; (jo) a C/C or G/C genotype at position 56782341 on chromosome 7; (jp) a A/A or C/A genotype at position 56872960 on chromosome 7; (jq) a C/C or T/C genotype at position 56910768 on chromosome 7; (jr) a T/T or A/T genotype at position 56967275 on chromosome 7; (js) a T/T or C/T genotype at position 57069404 on chromosome 7; (jt) a T/T or C/T genotype at position 57080583 on chromosome 7; (ju) a T/T or T/A genotype at position 57089709 on chromosome 7; (jv) a T/T or C/T genotype at position 57104188 on chromosome 7; (jw) a C/C or C/T genotype at position 57120122 on chromosome 7; (jx) a G/G or G/A genotype at position 57152672 on chromosome 7; (jy) a A/A or A/G genotype at position 57228643 on chromosome 7; (jz) a G/G or A/G genotype at position 57233796 on chromosome 7; (ha) a T/T or C/T genotype at position 58538433 on chromosome 7 wherein the reference genome is the Abacus Cannabis reference genome (version CsaAba2). In an embodiment, the cannabinoid comprises a type I and IV cannabinoid. In an embodiment, the one or more markers comprises a polymorphism at position 26 of any one or more of SEQ ID NO: 3; SEQ ID NO: 4; SEQ ID NO: 19; SEQ ID NO: 30; SEQ ID NO: 31; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 143; SEQ ID NO: 144; SEQ ID NO: 148; SEQ ID NO: 149; SEQ ID NO: 151; SEQ ID NO: 152; SEQ ID NO: 156; SEQ ID NO: 159; SEQ ID NO: 160; SEQ ID NO: 161; SEQ ID NO: 168; SEQ ID NO: 184; SEQ ID NO: 185; SEQ ID NO: 188; SEQ ID NO: 189; SEQ ID NO: 190; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 201; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 214; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 227; SEQ ID NO: 228; SEQ ID NO: 229; SEQ ID NO: 230; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 291; SEQ ID NO: 292; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO: 314; SEQ ID NO: 316; SEQ ID NO: 317; SEQ ID NO: 318; SEQ ID NO: 319; SEQ ID NO: 320; SEQ ID NO: 321; SEQ ID NO: 322; SEQ ID NO: 323; SEQ ID NO: 324; SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 334; SEQ ID NO: 335; SEQ ID NO: 336; SEQ ID NO: 337; SEQ ID NO: 338; SEQ ID NO: 339; SEQ ID NO: 340; SEQ ID NO: 341; SEQ ID NO: 342; SEQ ID NO: 344; SEQ ID NO: 345; SEQ ID NO: 346; SEQ ID NO: 347; SEQ ID NO: 348; SEQ ID NO: 350; SEQ ID NO: 351. In an embodiment, the nucleotide position comprises: (a) a T/T or C/T genotype at position 51 of SEQ ID NO: 3; (b) a T/T or G/T genotype at position 51 of SEQ ID NO: 19; (c) a T/T or T/C genotype at position 51 of SEQ ID NO: 30; (d) a T/T genotype at position 51 of SEQ ID NO: 31; (e) a C/C or T/C genotype at position 51 of SEQ ID NO: 35; (f) a A/A or G/A genotype at position 51 of SEQ ID NO: 118; (g) a T/T or C/T genotype at position 51 of SEQ ID NO: 144; (h) a A/A or G/A genotype at position 51 of SEQ ID NO: 148; (i) a A/A or A/T genotype at position 51 of SEQ ID NO: 151; (j) a C/C or T/C genotype at position 51 of SEQ ID NO: 152; (k) a G/G or A/G genotype at position 51 of SEQ ID NO: 156; (l) a A/A or C/A genotype at position 51 of SEQ ID NO: 159; (m) a C/C or T/C genotype at position 51 of SEQ ID NO: 160; (n) a G/G or A/G genotype at position 51 of SEQ ID NO: 161; (o) a G/G or C/G genotype at position 51 of SEQ ID NO: 184; (p) a A/A or T/A genotype at position 51 of SEQ ID NO: 185; (q) a A/A or T/A genotype at position 51 of SEQ ID NO: 189; (r) a A/A or G/A genotype at position 51 of SEQ ID NO: 190; (s) a G/G or T/G genotype at position 51 of SEQ ID NO: 193; (t) a C/C or T/C genotype at position 51 of SEQ ID NO: 194; (u) a C/C or T/C genotype at position 51 of SEQ ID NO: 195; (v) a C/C or T/C genotype at position 51 of SEQ ID NO: 196; (w) a C/C or T/C genotype at position 51 of SEQ ID NO: 197; (x) a A/A or C/A genotype at position 51 of SEQ ID NO: 198; (y) a C/C or G/C genotype at position 51 of SEQ ID NO: 199; (z) a G/G or T/G genotype at position 51 of SEQ ID NO: 201; (aa) a G/G or A/G genotype at position 51 of SEQ ID NO: 203; (ab) a C/C or A/C genotype at position 51 of SEQ ID NO: 204; (ac) a A/A or T/A genotype at position 51 of SEQ ID NO: 205; (ad) a G/G or C/G genotype at position 51 of SEQ ID NO: 206; (ae) a A/A or G/A genotype at position 51 of SEQ ID NO: 207; (af) a G/G or G/T genotype at position 51 of SEQ ID NO: 208; (ag) a T/T or T/A genotype at position 51 of SEQ ID NO: 209; (ah) a A/A or A/G genotype at position 51 of SEQ ID NO: 210; (ai) a A/A or C/A genotype at position 51 of SEQ ID NO: 216; (aj) a G/G or T/G genotype at position 51 of SEQ ID NO: 217; (ak) a A/A or G/A genotype at position 51 of SEQ ID NO: 219; (al) a G/G or A/G genotype at position 51 of SEQ ID NO: 220; (am) a T/T or T/C genotype at position 51 of SEQ ID NO: 221; (an) a T/T or T/C genotype at position 51 of SEQ ID NO: 222; (ao) a G/G or G/A genotype at position 51 of SEQ ID NO: 223; (ap) a A/A or A/G genotype at position 51 of SEQ ID NO: 224; (aq) a T/T or C/T genotype at position 51 of SEQ ID NO: 225; (ar) a T/T or C/T genotype at position 51 of SEQ ID NO: 227; (as) a A/A or G/A genotype at position 51 of SEQ ID NO: 229; (at) a C/C or C/A genotype at position 51 of SEQ ID NO: 230; (au) a T/T or C/T genotype at position 51 of SEQ ID NO: 235; (av) a A/A or A/G genotype at position 51 of SEQ ID NO: 239; (aw) a C/C or G/C genotype at position 51 of SEQ ID NO: 246; (ax) a C/C or T/C genotype at position 51 of SEQ ID NO: 268; (ay) a C/C or G/C genotype at position 51 of SEQ ID NO: 271; (az) a C/C or A/C genotype at position 51 of SEQ ID NO: 273; (ba) a T/T or C/T genotype at position 51 of SEQ ID NO: 274; (bb) a A/A or G/A genotype at position 51 of SEQ ID NO: 275; (bc) a A/A or G/A genotype at position 51 of SEQ ID NO: 276; (bd) a C/C or T/C genotype at position 51 of SEQ ID NO: 277; (be) a G/G or A/G genotype at position 51 of SEQ ID NO: 278; (bf) a A/A or G/A genotype at position 51 of SEQ ID NO: 279; (bg) a G/G or G/T genotype at position 51 of SEQ ID NO: 280; (bh) a A/A or G/A genotype at position 51 of SEQ ID NO: 281; (bi) a A/A or T/A genotype at position 51 of SEQ ID NO: 286; (bj) a G/G or A/G genotype at position 51 of SEQ ID NO: 287; (bk) a G/G or T/G genotype at position 51 of SEQ ID NO: 289; (bl) a G/G or A/G genotype at position 51 of SEQ ID NO: 294; (bm) a A/A or G/A genotype at position 51 of SEQ ID NO: 309; (bn) a T/T or C/T genotype at position 51 of SEQ ID NO: 311; (bo) a C/C or T/C genotype at position 51 of SEQ ID NO: 312; (bp) a C/C or T/C genotype at position 51 of SEQ ID NO: 319; (bq) a A/A or C/A genotype at position 51 of SEQ ID NO: 337; (br) a A/A or A/G genotype at position 51 of SEQ ID NO: 338; (bs) a T/T or T/C genotype at position 51 of SEQ ID NO: 339; (bt) a T/T or C/T genotype at position 51 of SEQ ID NO: 340; (bu) a T/T or T/G genotype at position 51 of SEQ ID NO: 341; (bv) a A/A or A/G genotype at position 51 of SEQ ID NO: 342; (bw) a G/G or G/A genotype at position 51 of SEQ ID NO: 344; (bx) a C/C or T/C genotype at position 51 of SEQ ID NO: 345; (by) a C/C or T/C genotype at position 51 of SEQ ID NO: 346; (bz) a G/G or A/G genotype at position 51 of SEQ ID NO: 350; (ca) a A/A genotype at position 51 of SEQ ID NO: 351; (cb) a T/T or C/T genotype at position 51 of SEQ ID NO: 3; (cc) a A/A or G/A genotype at position 51 of SEQ ID NO: 118; (cd) a C/C or T/C genotype at position 51 of SEQ ID NO: 143; (ce) a A/A or G/A genotype at position 51 of SEQ ID NO: 149; (cf) a C/A genotype at position 51 of SEQ ID NO: 168; (cg) a G/G or C/G genotype at position 51 of SEQ ID NO: 184; (ch) a C/C or G/C genotype at position 51 of SEQ ID NO: 199; (ci) a A/A or A/G genotype at position 51 of SEQ ID NO: 214; (cj) a G/G or A/G genotype at position 51 of SEQ ID NO: 218; (ck) a G/G or G/A genotype at position 51 of SEQ ID NO: 228; (cl) a T/T or G/T genotype at position 51 of SEQ ID NO: 242; (cm) a C/C or T/C genotype at position 51 of SEQ ID NO: 268; (cn) a G/G or G/C genotype at position 51 of SEQ ID NO: 269; (co) a C/C or G/C genotype at position 51 of SEQ ID NO: 271; (cp) a C/C or A/C genotype at position 51 of SEQ ID NO: 273; (cq) a T/T or C/T genotype at position 51 of SEQ ID NO: 274; (cr) a A/A or G/A genotype at position 51 of SEQ ID NO: 275; (cs) a A/A or G/A genotype at position 51 of SEQ ID NO: 276; (ct) a C/C or T/C genotype at position 51 of SEQ ID NO: 277; (cu) a G/G or A/G genotype at position 51 of SEQ ID NO: 278; (cv) a A/A or G/A genotype at position 51 of SEQ ID NO: 279; (cw) a A/A or T/A genotype at position 51 of SEQ ID NO: 286; (cx) a G/G or A/G genotype at position 51 of SEQ ID NO: 287; (cy) a G/G or G/A genotype at position 51 of SEQ ID NO: 344; (cz) a T/T or C/T genotype at position 51 of SEQ ID NO: 347; (da) a C/C or C/T genotype at position 51 of SEQ ID NO: 36; (db) a T/T genotype at position 51 of SEQ ID NO: 37; (dc) a C/C or C/T genotype at position 51 of SEQ ID NO: 38; (dd) a T/T or T/C genotype at position 51 of SEQ ID NO: 39; (de) a G/G or G/A genotype at position 51 of SEQ ID NO: 40; (df) a T/T genotype at position 51 of SEQ ID NO: 41; (dg) a A/A or A/G genotype at position 51 of SEQ ID NO: 42; (dh) a G/G or G/A genotype at position 51 of SEQ ID NO: 43; (di) a G/G or G/A genotype at position 51 of SEQ ID NO: 44; (dj) a C/C or C/G genotype at position 51 of SEQ ID NO: 45; (dk) a A/A or A/G genotype at position 51 of SEQ ID NO: 46; (dl) a A/A genotype at position 51 of SEQ ID NO: 47; (dm) a C/C or C/T genotype at position 51 of SEQ ID NO: 48; (dn) a T/T or T/G genotype at position 51 of SEQ ID NO: 49; (do) a A/A or A/C genotype at position 51 of SEQ ID NO: 50; (dp) a T/T or T/C genotype at position 51 of SEQ ID NO: 51; (dq) a A/A or A/G genotype at position 51 of SEQ ID NO: 52; (dr) a G/G or G/A genotype at position 51 of SEQ ID NO: 53; (ds) a G/G or G/A genotype at position 51 of SEQ ID NO: 54; (dt) a T/T or T/C genotype at position 51 of SEQ ID NO: 55; (du) a T/T or T/C genotype at position 51 of SEQ ID NO: 56; (dv) a C/C or C/T genotype at position 51 of SEQ ID NO: 57; (dw) a G/G or G/C genotype at position 51 of SEQ ID NO: 58; (dx) a G/G or G/T genotype at position 51 of SEQ ID NO: 59; (dy) a C/C or C/T genotype at position 51 of SEQ ID NO: 60; (dz) a C/C or C/T genotype at position 51 of SEQ ID NO: 61; (ea) a A/A or A/G genotype at position 51 of SEQ ID NO: 62; (eb) a A/A or A/G genotype at position 51 of SEQ ID NO: 63; (ec) a G/G or G/A genotype at position 51 of SEQ ID NO: 64; (ed) a T/T or T/C genotype at position 51 of SEQ ID NO: 65; (ee) a G/G or G/A genotype at position 51 of SEQ ID NO: 66; (ef) a C/C or C/A genotype at position 51 of SEQ ID NO: 67; (eg) a A/A or A/G genotype at position 51 of SEQ ID NO: 68; (eh) a A/A or A/T genotype at position 51 of SEQ ID NO: 69; (ei) a T/T or T/G genotype at position 51 of SEQ ID NO: 70; (ej) a C/C or C/T genotype at position 51 of SEQ ID NO: 71; (ek) a A/A or A/G genotype at position 51 of SEQ ID NO: 72; (el) a T/T or T/A genotype at position 51 of SEQ ID NO: 73; (em) a C/C or C/G genotype at position 51 of SEQ ID NO: 74; (en) a G/G or G/A genotype at position 51 of SEQ ID NO: 75; (eo) a G/G or G/A genotype at position 51 of SEQ ID NO: 76; (ep) a C/C or C/T genotype at position 51 of SEQ ID NO: 77; (eq) a T/T or T/G genotype at position 51 of SEQ ID NO: 78; (er) a G/G or G/A genotype at position 51 of SEQ ID NO: 79; (es) a A/A or A/T genotype at position 51 of SEQ ID NO: 80; (et) a C/C or C/A genotype at position 51 of SEQ ID NO: 81; (eu) a T/T or T/C genotype at position 51 of SEQ ID NO: 82; (ev) a C/C or C/T genotype at position 51 of SEQ ID NO: 83; (ew) a T/T or T/G genotype at position 51 of SEQ ID NO: 84; (ex) a A/A or A/G genotype at position 51 of SEQ ID NO: 85; (ey) a A/A or A/T genotype at position 51 of SEQ ID NO: 86; (ez) a A/A or A/C genotype at position 51 of SEQ ID NO: 87; (fa) a C/C or C/T genotype at position 51 of SEQ ID NO: 88; (fb) a T/T or T/C genotype at position 51 of SEQ ID NO: 89; (fc) a G/G or G/C genotype at position 51 of SEQ ID NO: 90; (fd) a G/G or G/A genotype at position 51 of SEQ ID NO: 91; (fe) a G/G or G/T genotype at position 51 of SEQ ID NO: 92; (ff) a T/T or T/C genotype at position 51 of SEQ ID NO: 93; (ga) a C/C or C/T genotype at position 51 of SEQ ID NO: 94; (gb) a C/C or C/T genotype at position 51 of SEQ ID NO: 95; (gc) a C/C or C/T genotype at position 51 of SEQ ID NO: 96; (gd) a C/C or C/T genotype at position 51 of SEQ ID NO: 97; (ge) a A/A or A/G genotype at position 51 of SEQ ID NO: 98; (gf) a A/A or A/C genotype at position 51 of SEQ ID NO: 99; (gg) a G/G or G/A genotype at position 51 of SEQ ID NO: 100; (gh) a T/T or T/C genotype at position 51 of SEQ ID NO: 101; (gi) a T/T or T/A genotype at position 51 of SEQ ID NO: 102; (gj) a C/C or C/T genotype at position 51 of SEQ ID NO: 103; (gk) a C/C or C/A genotype at position 51 of SEQ ID NO: 104; (gl) a C/C or C/T genotype at position 51 of SEQ ID NO: 105; (gm) a C/C or C/T genotype at position 51 of SEQ ID NO: 106; (gn) a C/C or C/T genotype at position 51 of SEQ ID NO: 107; (go) a C/C or C/A genotype at position 51 of SEQ ID NO: 108; (gp) a T/T or T/A genotype at position 51 of SEQ ID NO: 109; (gq) a C/C or C/T genotype at position 51 of SEQ ID NO: 110; (gr) a C/C or C/T genotype at position 51 of SEQ ID NO: 111; (gs) a T/T or T/A genotype at position 51 of SEQ ID NO: 112; (gt) a T/T or T/C genotype at position 51 of SEQ ID NO: 113; (gu) a A/A or A/G genotype at position 51 of SEQ ID NO: 114; (gv) a A/A or A/G genotype at position 51 of SEQ ID NO: 115; (gw) a C/C genotype at position 51 of SEQ ID NO: 116; (gx) a A/A or A/G genotype at position 51 of SEQ ID NO: 117; (gy) a C/C or C/T genotype at position 51 of SEQ ID NO: 188; (gz) a A/A or G/A genotype at position 51 of SEQ ID NO: 4; (ha) a T/T or C/T genotype at position 51 of SEQ ID NO: 233; (hb) a A/A or G/A genotype at position 51 of SEQ ID NO: 234; (hc) a T/T or T/C genotype at position 51 of SEQ ID NO: 236; (hd) a T/T or T/A genotype at position 51 of SEQ ID NO: 237; (he) a T/T or G/T genotype at position 51 of SEQ ID NO: 238; (hf) a C/C or C/G genotype at position 51 of SEQ ID NO: 240; (hg) a A/A or A/T genotype at position 51 of SEQ ID NO: 241; (hh) a C/C or C/A genotype at position 51 of SEQ ID NO: 243; (hi) a T/T or T/C genotype at position 51 of SEQ ID NO: 244; (hj) a T/T or T/A genotype at position 51 of SEQ ID NO: 245; (hk) a G/G or G/T genotype at position 51 of SEQ ID NO: 247; (hl) a A/A or G/A genotype at position 51 of SEQ ID NO: 248; (hm) a G/G or T/G genotype at position 51 of SEQ ID NO: 249; (hn) a C/C or T/C genotype at position 51 of SEQ ID NO: 250; (ho) a T/T or T/A genotype at position 51 of SEQ ID NO: 251; (hp) a G/G or A/G genotype at position 51 of SEQ ID NO: 253; (hq) a T/T or G/T genotype at position 51 of SEQ ID NO: 254; (hr) a G/G or T/G genotype at position 51 of SEQ ID NO: 255 (hs) a A/A or A/T genotype at position 51 of SEQ ID NO: 256; (ht) a C/C or C/G genotype at position 51 of SEQ ID NO: 257; (hu) a G/G or G/T genotype at position 51 of SEQ ID NO: 258; (hv) a T/T or T/G genotype at position 51 of SEQ ID NO: 259; (hw) a A/A or A/G genotype at position 51 of SEQ ID NO: 262; (hx) a A/A or T/A genotype at position 51 of SEQ ID NO: 263; (hy) a C/C or T/C genotype at position 51 of SEQ ID NO: 264; (hz) a C/C or T/C genotype at position 51 of SEQ ID NO: 265; (ia) a G/G or G/A genotype at position 51 of SEQ ID NO: 266; (ib) a G/G or T/G genotype at position 51 of SEQ ID NO: 267; (ic) a G/G or G/C genotype at position 51 of SEQ ID NO: 269; (id) a C/C or C/A genotype at position 51 of SEQ ID NO: 270; (ie) a A/A or G/A genotype at position 51 of SEQ ID NO: 281; (if) a G/G or C/G genotype at position 51 of SEQ ID NO: 282; (ig) a T/T or A/T genotype at position 51 of SEQ ID NO: 283; (ih) a C/C or A/C genotype at position 51 of SEQ ID NO: 284; (ii) a A/A or C/A genotype at position 51 of SEQ ID NO: 285; (ij) a A/A or C/A genotype at position 51 of SEQ ID NO: 288; (ik) a C/C or C/T genotype at position 51 of SEQ ID NO: 291; (il) a G/G or A/G genotype at position 51 of SEQ ID NO: 292; (im) a G/G or A/G genotype at position 51 of SEQ ID NO: 294; (in) a G/G or G/A genotype at position 51 of SEQ ID NO: 295; (io) a T/T or T/G genotype at position 51 of SEQ ID NO: 296; (ip) a G/G or A/G genotype at position 51 of SEQ ID NO: 297; (iq) a T/T or C/T genotype at position 51 of SEQ ID NO: 298; (ir) a A/A or G/A genotype at position 51 of SEQ ID NO: 299; (is) a A/A or C/A genotype at position 51 of SEQ ID NO: 300; (it) a A/A or G/A genotype at position 51 of SEQ ID NO: 301; (iu) a A/A or G/A genotype at position 51 of SEQ ID NO: 302; (iv) a A/A or A/T genotype at position 51 of SEQ ID NO: 303; (iw) a G/G or A/G genotype at position 51 of SEQ ID NO: 304; (ix) a A/A or T/A genotype at position 51 of SEQ ID NO: 305; (iy) a T/T or C/T genotype at position 51 of SEQ ID NO: 306; (iz) a T/T or A/T genotype at position 51 of SEQ ID NO: 307; (ja) a T/T or A/T genotype at position 51 of SEQ ID NO: 308; (jb) a A/A or G/A genotype at position 51 of SEQ ID NO: 309; (jc) a A/A or G/A genotype at position 51 of SEQ ID NO: 310; (jd) a T/T or C/T genotype at position 51 of SEQ ID NO: 311; (je) a C/C or T/C genotype at position 51 of SEQ ID NO: 312; (jf) a G/G or A/G genotype at position 51 of SEQ ID NO: 313; (jg) a A/A or G/A genotype at position 51 of SEQ ID NO: 314; (jh) a G/G or T/G genotype at position 51 of SEQ ID NO: 316; (ji) a A/A or A/G genotype at position 51 of SEQ ID NO: 317; (jj) a T/T or C/T genotype at position 51 of SEQ ID NO: 318; (jk) a A/A or A/G genotype at position 51 of SEQ ID NO: 320; (jl) a T/T or T/G genotype at position 51 of SEQ ID NO: 321; (jm) a A/A or C/A genotype at position 51 of SEQ ID NO: 322; (jn) a T/T or T/C genotype at position 51 of SEQ ID NO: 323; (jo) a C/C or G/C genotype at position 51 of SEQ ID NO: 324; (jp) a A/A or C/A genotype at position 51 of SEQ ID NO: 326; (jq) a C/C or T/C genotype at position 51 of SEQ ID NO: 327; (jr) a T/T or A/T genotype at position 51 of SEQ ID NO: 328; (js) a T/T or C/T genotype at position 51 of SEQ ID NO: 329; (jt) a T/T or C/T genotype at position 51 of SEQ ID NO: 330; (ju) a T/T or T/A genotype at position 51 of SEQ ID NO: 331; (jv) a T/T or C/T genotype at position 51 of SEQ ID NO: 332; (jw) a C/C or C/T genotype at position 51 of SEQ ID NO: 333; (jx) a G/G or G/A genotype at position 51 of SEQ ID NO: 334; (jy) a A/A or A/G genotype at position 51 of SEQ ID NO: 335; (jz) a G/G or A/G genotype at position 51 of SEQ ID NO: 336; (ha) a T/T or C/T genotype at position 51 of SEQ ID NO: 348 wherein the reference genome is the Abacus Cannabis reference genome (version CsaAba2). In an embodiment, the one or more markers comprises a polymorphism relative to a reference genome within any one or more haplotypes wherein the haplotypes comprise the region: (a) between positions 32379705 and 32422316 on chromosome 1; (b) between positions 1271584 and 1284641 on chromosome 3; (c) between positions 1840325 and 1848408 on chromosome 5; (d) between positions 2011766 and 2065182 on chromosome 5; (e) between positions 2360380 and 2366529 on chromosome 5; (f) between positions 4002406 and 4037935 on chromosome 6; (g) between positions 6125186 and 6165666 on chromosome 7; (h) between positions 7083154 and 7103045 on chromosome 7; (i) between positions 7822322 and 7864334 on chromosome 7; (j) between positions 8652992 and 8669729 on chromosome 7; (k) between positions 9585902 and 9598562 on chromosome 7; (l) between positions 9619898 and 9646812 on chromosome 7; (m) between positions 9662890 and 9677657 on chromosome 7; (n) between positions 9701317 and 9716591 on chromosome 7; (o) between positions 18581032 and 18640383 on chromosome 7; (p) between positions 20022570 and 20050272 on chromosome 7; (q) between positions 24593497 and 24673734 on chromosome 7; (r) between positions 25478783 and 25784004 on chromosome 7; (s) between positions 29416077 and 29482333 on chromosome 7; (t) between positions 29661512 and 29686508 on chromosome 7; (u) between positions 29985750 and 30047823 on chromosome 7; (v) between positions 30115726 and 30346994 on chromosome 7; (w) between positions 30115726 and 30346994 on chromosome 7; (x) between positions 30115726 and 30346994 on chromosome 7; (y) between positions 30346994 and 30489245 on chromosome 7; (z) between positions 30670182 and 30816363 on chromosome 7; (aa) between positions 33455822 and 33524411 on chromosome 7; (ab) between positions 33577092 and 33700189 on chromosome 7; (ac) between positions 33969097 and 34028788 on chromosome 7; (ad) between positions 34028788 and 34136591 on chromosome 7; (ae) between positions 34941497 and 34997619 on chromosome 7; (af) between positions 35592132 and 35695775 on chromosome 7; (ag) between positions 35592132 and 35695775 on chromosome 7; (ah) between positions 35799742 and 36153842 on chromosome 7; (ai) between positions 38729407 and 38794976 on chromosome 7; (aj) between positions 41248971 and 41343647 on chromosome 7; (ak) between positions 41820799 and 41931751 on chromosome 7; (al) between positions 41982953 and 41988552 on chromosome 7; (am) between positions 42043493 and 42096029 on chromosome 7; (an) between positions 42096029 and 42156112 on chromosome 7; (ao) between positions 42175605 and 42379636 on chromosome 7; (ap) between positions 42379636 and 42418406 on chromosome 7; (aq) between positions 42758132 and 42819971 on chromosome 7; (ar) between positions 44692277 and 44723890 on chromosome 7; (as) between positions 44924842 and 44959410 on chromosome 7; (at) between positions 46090347 and 46097781 on chromosome 7; (au) between positions 48393114 and 48443534 on chromosome 7; (av) between positions 48870733 and 49016897 on chromosome 7; (aw) between positions 49288761 and 49297082 on chromosome 7; (ax) between positions 50724154 and 50747700 on chromosome 7; (ay) between positions 50818092 and 50822516 on chromosome 7; (az) between positions 50822516 and 50861059 on chromosome 7; (ba) between positions 51022401 and 51062076 on chromosome 7; (bb) between positions 51141514 and 51226826 on chromosome 7; (bc) between positions 51909282 and 52435669 on chromosome 7; (bd) between positions 51909282 and 52435669 on chromosome 7; (be) between positions 51909282 and 52435669 on chromosome 7; (bf) between positions 51909282 and 52435669 on chromosome 7; (bg) between positions 51909282 and 52435669 on chromosome 7; (bh) between positions 52435669 and 52441872 on chromosome 7; (bi) between positions 52552192 and 52760459 on chromosome 7; (bj) between positions 52552192 and 52760459 on chromosome 7; (bk) between positions 52926238 and 53035545 on chromosome 7; (bl) between positions 54394199 and 54410973 on chromosome 7; (bm) between positions 56001500 and 56032988 on chromosome 7; (bn) between positions 56067360 and 56102656 on chromosome 7; (bo) between positions 56067360 and 56102656 on chromosome 7; (bp) between positions 56440283 and 56450990 on chromosome 7; (bq) between positions 57760733 and 57805989 on chromosome 7; (br) between positions 58146715 and 58181665 on chromosome 7; (bs) between positions 58186525 and 58197878 on chromosome 7; (bt) between positions 58220261 and 58237329 on chromosome 7; (bu) between positions 58237682 and 58252232 on chromosome 7; (bv) between positions 58266320 and 58275951 on chromosome 7; (bw) between positions 58305695 and 58324561 on chromosome 7; (bx) between positions 58450413 and 58456572 on chromosome 7; (by) between positions 58467957 and 58522091 on chromosome 7; (bz) between positions 59728563 and 59762135 on chromosome 7; (ca) between positions 60363767 and 60366472 on chromosome 7; (cb) between positions 32363260 and 32422316 on chromosome 1; (cc) between positions 4002406 and 4087346 on chromosome 6; (cd) between positions 5900788 and 5921575 on chromosome 7; (ce) between positions 7630020 and 7643910 on chromosome 7; (cf) between positions 13715325 and 13740309 on chromosome 7; (cg) between positions 18581032 and 18640383 on chromosome 7; (ch) between positions 30318939 and 30489245 on chromosome 7; (ci) between positions 37495060 and 37639835 on chromosome 7; (cj) between positions 41651577 and 41675164 on chromosome 7; (ck) between positions 44911280 and 44924842 on chromosome 7; (cl) between positions 49085607 and 49134977 on chromosome 7; (cm) between positions 50724154 and 50765250 on chromosome 7; (cn) between positions 50765250 and 50777808 on chromosome 7; (co) between positions 50818092 and 50822516 on chromosome 7; (cp) between positions 50822516 and 50959554 on chromosome 7; (cq) between positions 51022401 and 51062076 on chromosome 7; (cr) between positions 51141514 and 51226826 on chromosome 7; (cs) between positions 51262584 and 52291349 on chromosome 7; (ct) between positions 52291349 and 52412182 on chromosome 7; (cu) between positions 52291349 and 52412182 on chromosome 7; (cv) between positions 52291349 and 52412182 on chromosome 7; (cw) between positions 52552192 and 52760459 on chromosome 7; (cx) between positions 52552192 and 52760459 on chromosome 7; (cy) between positions 58305695 and 58328372 on chromosome 7; (cz) between positions 58524187 and 58538433 on chromosome 7; (da) between positions 2364964 and 2534579 on chromosome 5; (db) between positions 2844078 and 2908474 on chromosome 5; (dc) between positions 3061958 and 3081773 on chromosome 5; (dd) between positions 3081773 and 3089662 on chromosome 5; (de) between positions 3395357 and 3454995 on chromosome 5; (df) between positions 3454995 and 3493107 on chromosome 5; (dg) between positions 3526980 and 3541316 on chromosome 5; (dh) between positions 3585965 and 3604863 on chromosome 5; (di) between positions 3656543 and 3680073 on chromosome 5; (dj) between positions 3945751 and 3965771 on chromosome 5; (dk) between positions 4109676 and 4130254 on chromosome 5; (dl) between positions 4376633 and 4391586 on chromosome 5; (dm) between positions 6489377 and 6516042 on chromosome 5; (dn) between positions 6636230 and 6668100 on chromosome 5; (do) between positions 7067915 and 7147487 on chromosome 5; (dp) between positions 7226971 and 7257339 on chromosome 5; (dq) between positions 7897923 and 7933626 on chromosome 5; (dr) between positions 8074202 and 8089196 on chromosome 5; (ds) between positions 9091191 and 9164971 on chromosome 5; (dt) between positions 9091191 and 9164971 on chromosome 5; (du) between positions 9453509 and 9478633 on chromosome 5; (dv) between positions 9542524 and 9570961 on chromosome 5; (dw) between positions 9718369 and 9771730 on chromosome 5; (dx) between positions 9718369 and 9771730 on chromosome 5; (dy) between positions 10143433 and 10200275 on chromosome 5; (dz) between positions 10402308 and 10486467 on chromosome 5; (ea) between positions 10402308 and 10486467 on chromosome 5; (eb) between positions 10402308 and 10486467 on chromosome 5; (ec) between positions 10531323 and 10586049 on chromosome 5; (ed) between positions 11131783 and 11193898 on chromosome 5; (ee) between positions 11217176 and 11252809 on chromosome 5; (ef) between positions 11252809 and 11322876 on chromosome 5; (eg) between positions 11252809 and 11322876 on chromosome 5; (eh) between positions 12444383 and 12472642 on chromosome 5; (ei) between positions 12829839 and 12944302 on chromosome 5; (ej) between positions 13375598 and 13383733 on chromosome 5; (ek) between positions 13754147 and 13766692 on chromosome 5; (el) between positions 14759093 and 14860844 on chromosome 5; (em) between positions 15085497 and 15201574 on chromosome 5; (en) between positions 15975544 and 16037097 on chromosome 5; (eo) between positions 18699991 and 18716207 on chromosome 5; (ep) between positions 19976402 and 20026964 on chromosome 5; (eq) between positions 19976402 and 20026964 on chromosome 5; (er) between positions 20145940 and 20173122 on chromosome 5; (es) between positions 20483019 and 20546290 on chromosome 5; (et) between positions 20721287 and 20835904 on chromosome 5; (eu) between positions 21516323 and 21554970 on chromosome 5; (ev) between positions 21554970 and 21626778 on chromosome 5; (ew) between positions 23872960 and 24016565 on chromosome 5; (ex) between positions 23872960 and 24016565 on chromosome 5; (ey) between positions 23872960 and 24016565 on chromosome 5; (ez) between positions 24799598 and 24906306 on chromosome 5; (fa) between positions 25342928 and 25387614 on chromosome 5; (fb) between positions 25342928 and 25387614 on chromosome 5; (fc) between positions 25387614 and 25391623 on chromosome 5; (fd) between positions 25391623 and 25548921 on chromosome 5; (fe) between positions 28847049 and 28956941 on chromosome 5; (ff) between positions 32296352 and 32322620 on chromosome 5; (ga) between positions 32322620 and 32390660 on chromosome 5; (gb) between positions 36089359 and 36288826 on chromosome 5; (gc) between positions 40385082 and 40557005 on chromosome 5; (gd) between positions 42375856 and 42528615 on chromosome 5; (ge) between positions 42550360 and 42563140 on chromosome 5; (gf) between positions 42604248 and 43518393 on chromosome 5; (gg) between positions 44026725 and 44095222 on chromosome 5; (gh) between positions 45678680 and 45734373 on chromosome 5; (gi) between positions 48970067 and 48984121 on chromosome 5; (gj) between positions 52798015 and 52858896 on chromosome 5; (gk) between positions 53299267 and 53496045 on chromosome 5; (gl) between positions 53705956 and 53815660 on chromosome 5; (gm) between positions 54304731 and 54406395 on chromosome 5; (gn) between positions 56185902 and 56294577 on chromosome 5; (go) between positions 57493670 and 57540049 on chromosome 5; (gp) between positions 57816878 and 57845773 on chromosome 5; (gq) between positions 57996384 and 58043891 on chromosome 5; (gr) between positions 58582581 and 58598359 on chromosome 5; (gs) between positions 59813998 and 59876892 on chromosome 5; (gt) between positions 60254151 and 60285659 on chromosome 5; (gu) between positions 60285659 and 60313853 on chromosome 5; (gv) between positions 61225099 and 61253927 on chromosome 5; (gw) between positions 64168576 and 64227025 on chromosome 5; (gx) between positions 64881518 and 64888996 on chromosome 5; (gy) between positions 23372911 and 23432690 on chromosome 7; (gz) between positions 38944628 and 39073783 on chromosome 1; (ha) between positions 48198578 and 49711589 on chromosome 7; (hb) between positions 48198578 and 49711589 on chromosome 7; (hc) between positions 48198578 and 49711589 on chromosome 7; (hd) between positions 48198578 and 49711589 on chromosome 7; (he) between positions 48198578 and 49711589 on chromosome 7; (hf) between positions 48198578 and 49711589 on chromosome 7; (hg) between positions 48198578 and 49711589 on chromosome 7; (hh) between positions 48198578 and 49711589 on chromosome 7; (hi) between positions 48198578 and 49711589 on chromosome 7; (hj) between positions 48198578 and 49711589 on chromosome 7; (hk) between positions 48198578 and 49711589 on chromosome 7; (hl) between positions 48198578 and 49711589 on chromosome 7; (hm) between positions 48198578 and 49711589 on chromosome 7; (hn) between positions 48198578 and 49711589 on chromosome 7; (ho) between positions 48198578 and 49711589 on chromosome 7; (hp) between positions 48198578 and 49711589 on chromosome 7; (hq) between positions 48198578 and 49711589 on chromosome 7; (hr) between positions 48198578 and 49711589 on chromosome 7; (hs) between positions 48198578 and 49711589 on chromosome 7; (ht) between positions 49711589 and 50350437 on chromosome 7; (hu) between positions 49711589 and 50350437 on chromosome 7; (hv) between positions 49711589 and 50350437 on chromosome 7; (hw) between positions 49711589 and 50350437 on chromosome 7; (hx) between positions 50350437 and 50528030 on chromosome 7; (hy) between positions 50528030 and 50822516 on chromosome 7; (hz) between positions 50528030 and 50822516 on chromosome 7; (ia) between positions 50528030 and 50822516 on chromosome 7; (ib) between positions 50528030 and 50822516 on chromosome 7; (ic) between positions 50528030 and 50822516 on chromosome 7; (id) between positions 50528030 and 50822516 on chromosome 7; (ie) between positions 50822516 and 52523987 on chromosome 7; (if) between positions 50822516 and 52523987 on chromosome 7; (ig) between positions 50822516 and 52523987 on chromosome 7; (ih) between positions 50822516 and 52523987 on chromosome 7; (ii) between positions 50822516 and 52523987 on chromosome 7; (ij) between positions 52544592 and 53396185 on chromosome 7; (ik) between positions 53396185 and 54375898 on chromosome 7; (il) between positions 53396185 and 54375898 on chromosome 7; (im) between positions 54375898 and 54452078 on chromosome 7; (in) between positions 54452078 and 54646726 on chromosome 7; (io) between positions 54452078 and 54646726 on chromosome 7; (ip) between positions 54452078 and 54646726 on chromosome 7; (iq) between positions 54452078 and 54646726 on chromosome 7; (ir) between positions 54683300 and 54714058 on chromosome 7; (is) between positions 54714058 and 54860264 on chromosome 7; (it) between positions 54860264 and 55029197 on chromosome 7; (iu) between positions 55255666 and 55316629 on chromosome 7; (iv) between positions 55316629 and 55939712 on chromosome 7; (iw) between positions 55316629 and 55939712 on chromosome 7; (ix) between positions 55316629 and 55939712 on chromosome 7; (iy) between positions 55316629 and 55939712 on chromosome 7; (iz) between positions 55939712 and 56102656 on chromosome 7; (ja) between positions 55939712 and 56102656 on chromosome 7; (jb) between positions 55939712 and 56102656 on chromosome 7; (jc) between positions 55939712 and 56102656 on chromosome 7; (jd) between positions 55939712 and 56102656 on chromosome 7; (je) between positions 55939712 and 56102656 on chromosome 7; (jf) between positions 56166983 and 56434732 on chromosome 7; (jg) between positions 56166983 and 56434732 on chromosome 7; (jh) between positions 56166983 and 56434732 on chromosome 7; (ji) between positions 56166983 and 56434732 on chromosome 7; (jj) between positions 56434732 and 56593122 on chromosome 7; (jk) between positions 56434732 and 56593122 on chromosome 7; (jl) between positions 56434732 and 56593122 on chromosome 7; (jm) between positions 56434732 and 56593122 on chromosome 7; (jn) between positions 56620519 and 56809638 on chromosome 7; (jo) between positions 56620519 and 56809638 on chromosome 7; (jp) between positions 56809638 and 57276534 on chromosome 7; (jq) between positions 56809638 and 57276534 on chromosome 7; (jr) between positions 56809638 and 57276534 on chromosome 7; (js) between positions 56809638 and 57276534 on chromosome 7; (jt) between positions 56809638 and 57276534 on chromosome 7; (ju) between positions 56809638 and 57276534 on chromosome 7; (jv) between positions 56809638 and 57276534 on chromosome 7; (jw) between positions 56809638 and 57276534 on chromosome 7; (jx) between positions 56809638 and 57276534 on chromosome 7; (jy) between positions 56809638 and 57276534 on chromosome 7; (jz) between positions 56809638 and 57276534 on chromosome 7; (ha) between positions 58428139 and 58607780 on chromosome 7 wherein the reference genome is the Abacus Cannabis reference genome (version CsaAba2).

In an embodiment, the modified cannabinoids correlate to increased ratio of total CBC to the combination of total CBG and total CBGV. In an embodiment, the one or more markers comprises a polymorphism relative to a reference genome at nucleotide position: (a) 4023145 on chromosome 6; or (b) 7639988 on chromosome 7; or (c) 41659351 on chromosome 7; or (d) 50854826 on chromosome 7; or (e) 51054719 on chromosome 7; or (f) 51173524 on chromosome 7; or (g) 52296271 on chromosome 7; or (h) 52554676 on chromosome 7; or (i) 52561249 on chromosome 7; or (j) 58528791 on chromosome 7; or (k) 20017410 on chromosome 5; or (l) 36148442 on chromosome 5; or (m) 38978759 on chromosome 1; or (n) 67769631 on chromosome 1; or (o) 28081703 on chromosome 7; or (p) 28685688 on chromosome 7; or (q) 50822516 on chromosome 7; or (r) 52923743 on chromosome 7; or (s) 53505022 on chromosome 7; or (t) 54400345 on chromosome 7; or (u) 54464358 on chromosome 7; or (v) 54509399 on chromosome 7; or (w) 54513057 on chromosome 7; or (x) 54555051 on chromosome 7; or (y) 54693540 on chromosome 7; or (z) 54722302 on chromosome 7; or (aa) 54915189 on chromosome 7; or (ab) 55277957 on chromosome 7; or (ac) 55452244 on chromosome 7; or (ad) 56032988 on chromosome 7; or (ae) 56301604 on chromosome 7; or (af) 56967275 on chromosome 7; or (ag) 58538433 on chromosome 7 wherein the reference genome is the Abacus Cannabis reference genome (version CsaAba2). In an embodiment, the nucleotide position comprises: (a) a A/A or G/A genotype at position 4023145 on chromosome 6; (b) a A/A or G/A genotype at position 7639988 on chromosome 7; (c) a G/G or A/G genotype at position 41659351 on chromosome 7; (d) a C/C or A/C genotype at position 50854826 on chromosome 7; (e) a T/T or C/T genotype at position 51054719 on chromosome 7; (f) a A/A or G/A genotype at position 51173524 on chromosome 7; (g) a C/C or T/C genotype at position 52296271 on chromosome 7; (h) a A/A or T/A genotype at position 52554676 on chromosome 7; (i) a G/G or A/G genotype at position 52561249 on chromosome 7; (j) a T/T or C/T genotype at position 58528791 on chromosome 7; (k) a T/T or T/G genotype at position 20017410 on chromosome 5; (l) a C/C or C/T genotype at position 36148442 on chromosome 5; (m) a A/A or G/A genotype at position 38978759 on chromosome 1; (n) a A/A or G/A genotype at position 67769631 on chromosome 1; (o) a C/C or G/C genotype at position 28081703 on chromosome 7; (p) a T/T or C/T genotype at position 28685688 on chromosome 7; (q) a T/T or T/A genotype at position 50822516 on chromosome 7; (r) a A/A or C/A genotype at position 52923743 on chromosome 7; (s) a G/G or A/G genotype at position 53505022 on chromosome 7; (t) a G/G or A/G genotype at position 54400345 on chromosome 7; (u) a G/G or G/A genotype at position 54464358 on chromosome 7; (v) a T/T or T/G genotype at position 54509399 on chromosome 7; (w) a G/G or A/G genotype at position 54513057 on chromosome 7; (x) a T/T or C/T genotype at position 54555051 on chromosome 7; (y) a A/A or G/A genotype at position 54693540 on chromosome 7; (z) a A/A or C/A genotype at position 54722302 on chromosome 7; (aa) a A/A or G/A genotype at position 54915189 on chromosome 7; (ab) a A/A or G/A genotype at position 55277957 on chromosome 7; (ac) a A/A or A/T genotype at position 55452244 on chromosome 7; (ad) a A/A or G/A genotype at position 56032988 on chromosome 7; (ae) a A/A or G/A genotype at position 56301604 on chromosome 7; (af) a T/T or A/T genotype at position 56967275 on chromosome 7; (ag) a T/T or C/T genotype at position 58538433 on chromosome 7 wherein the reference genome is the Abacus Cannabis reference genome (version CsaAba2). In an embodiment, the cannabinoid comprises a type I or IV cannabinoid. In an embodiment, the one or more markers comprises a polymorphism at position 26 of any one or more of SEQ ID NO: 4; SEQ ID NO: 8; SEQ ID NO: 78; SEQ ID NO: 95; SEQ ID NO: 118; SEQ ID NO: 149; SEQ ID NO: 191; SEQ ID NO: 192; SEQ ID NO: 218; SEQ ID NO: 272; SEQ ID NO: 273; SEQ ID NO: 274; SEQ ID NO: 275; SEQ ID NO: 277; SEQ ID NO: 286; SEQ ID NO: 287; SEQ ID NO: 288; SEQ ID NO: 292; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 310; SEQ ID NO: 314; SEQ ID NO: 328; SEQ ID NO: 347; SEQ ID NO: 348. In an embodiment, the nucleotide position comprises: (a) a A/A or G/A genotype at position 51 of SEQ ID NO: 118; (b) a A/A or G/A genotype at position 51 of SEQ ID NO: 149; (c) a G/G or A/G genotype at position 51 of SEQ ID NO: 218; (d) a C/C or A/C genotype at position 51 of SEQ ID NO: 273; (e) a T/T or C/T genotype at position 51 of SEQ ID NO: 274; (f) a A/A or G/A genotype at position 51 of SEQ ID NO: 275; (g) a C/C or T/C genotype at position 51 of SEQ ID NO: 277; (h) a A/A or T/A genotype at position 51 of SEQ ID NO: 286; (i) a G/G or A/G genotype at position 51 of SEQ ID NO: 287; (j) a T/T or C/T genotype at position 51 of SEQ ID NO: 347; (k) a T/T or T/G genotype at position 51 of SEQ ID NO: 78; (l) a C/C or C/T genotype at position 51 of SEQ ID NO: 95; (m) a A/A or G/A genotype at position 51 of SEQ ID NO: 4; (n) a A/A or G/A genotype at position 51 of SEQ ID NO: 8; (o) a C/C or G/C genotype at position 51 of SEQ ID NO: 191; (p) a T/T or C/T genotype at position 51 of SEQ ID NO: 192; (q) a T/T or T/A genotype at position 51 of SEQ ID NO: 272; (r) a A/A or C/A genotype at position 51 of SEQ ID NO: 288; (s) a G/G or A/G genotype at position 51 of SEQ ID NO: 292; (t) a G/G or A/G genotype at position 51 of SEQ ID NO: 294; (u) a G/G or G/A genotype at position 51 of SEQ ID NO: 295; (v) a T/T or T/G genotype at position 51 of SEQ ID NO: 296; (w) a G/G or A/G genotype at position 51 of SEQ ID NO: 297; (x) a T/T or C/T genotype at position 51 of SEQ ID NO: 298; (y) a A/A or G/A genotype at position 51 of SEQ ID NO: 299; (z) a A/A or C/A genotype at position 51 of SEQ ID NO: 300; (aa) a A/A or G/A genotype at position 51 of SEQ ID NO: 301; (ab) a A/A or G/A genotype at position 51 of SEQ ID NO: 302; (ac) a A/A or A/T genotype at position 51 of SEQ ID NO: 303; (ad) a A/A or G/A genotype at position 51 of SEQ ID NO: 310; (ae) a A/A or G/A genotype at position 51 of SEQ ID NO: 314; (af) a T/T or A/T genotype at position 51 of SEQ ID NO: 328; (ag) a T/T or C/T genotype at position 51 of SEQ ID NO: 348 wherein the reference genome is the Abacus Cannabis reference genome (version CsaAba2). In an embodiment, the one or more markers comprises a polymorphism relative to a reference genome within any one or more haplotypes wherein the haplotypes comprise the region: (a) between positions 4002406 and 4087346 on chromosome 6; (b) between positions 7630020 and 7643910 on chromosome 7; (c) between positions 41651577 and 41675164 on chromosome 7; (d) between positions 50822516 and 50959554 on chromosome 7; (e) between positions 51022401 and 51062076 on chromosome 7; (f) between positions 51141514 and 51226826 on chromosome 7; (g) between positions 52291349 and 52322834 on chromosome 7; (h) between positions 52552192 and 52760459 on chromosome 7; (i) between positions 52552192 and 52760459 on chromosome 7; (j) between positions 58524187 and 58538433 on chromosome 7; (k) between positions 19988534 and 20026964 on chromosome 5; (l) between positions 36089359 and 36288826 on chromosome 5; (m) between positions 38944628 and 39073783 on chromosome 1; (n) between positions 67761686 and 67892254 on chromosome 1; (o) between positions 27759260 and 28263307 on chromosome 7; (p) between positions 28594408 and 29061134 on chromosome 7; (q) between positions 50818092 and 52439705 on chromosome 7; (r) between positions 52544592 and 53396185 on chromosome 7; (s) between positions 53449873 and 54375898 on chromosome 7; (t) between positions 54375898 and 54452078 on chromosome 7; (u) between positions 54452078 and 54646726 on chromosome 7; (v) between positions 54452078 and 54646726 on chromosome 7; (w) between positions 54452078 and 54646726 on chromosome 7; (x) between positions 54452078 and 54646726 on chromosome 7; (y) between positions 54683300 and 54714058 on chromosome 7; (z) between positions 54714058 and 54860264 on chromosome 7; (aa) between positions 54860264 and 55029197 on chromosome 7; (ab) between positions 55255666 and 55316629 on chromosome 7; (ac) between positions 55316629 and 55716705 on chromosome 7; (ad) between positions 56018989 and 56076209 on chromosome 7; (ae) between positions 56171548 and 56426824 on chromosome 7; (af) between positions 56910768 and 57069404 on chromosome 7; (ag) between positions 58428139 and 58607780 on chromosome 7 wherein the reference genome is the Abacus Cannabis reference genome (version CsaAba2).

In an embodiment, the selecting comprises marker assisted selection. In an embodiment, the detecting comprises an oligonucleotide probe. In an embodiment, the method comprises crossing the one or more plants comprising the indicated modified cannabinoids to produce one or more F1 or additional progeny plants, wherein at least one of the F1 or additional progeny plants comprises the indicated modified cannabinoids. In an embodiment, the crossing comprises selfing, sibling crossing, or backcrossing. In an embodiment, the at least one additional progeny plant comprising the indicated modified cannabinoids comprises an F2-F7 progeny plant. In an embodiment, the selfing, sibling crossing, or backcrossing comprises marker-assisted selection. In an embodiment, the selfing, sibling crossing, or backcrossing comprises marker-assisted selection for at least two generations. In an embodiment, the plant comprises a Cannabis plant. In an embodiment, the method comprises replacing a nucleic acid sequence of a parent plant with a nucleic acid sequence conferring modified cannabinoids. In an embodiment, a method is provided wherein the method comprises generating a processed cannabis product comprising the use of F1 hybrid plant, or plant part thereof, of claim 2. In an embodiment, a cannabis product is provided wherein the product is produced using the method of claim 62. In an embodiment, the product of claim 63 wherein the product is a fief, hashish, bubble hash, an edible product, solvent reduced oil, sludge, e-juice, or tincture.

### BRIEF DESCRIPTION OF THE DRAWINGS

The skilled artisan will understand that the drawings, described below, are for illustration purposes only. The drawings are not intended to limit the scope of the present teachings in any way.

FIG. 1 illustrates THC to CBG Ratio NAM results for type I accessions (n=682).

### DETAILED DESCRIPTION OF THE INVENTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The present teachings relate generally to producing or developing Cannabis varieties having modified cannabinoid levels or ratios by selecting plants having markers indicating such activity.

The terminology used in the disclosure herein is for the purpose of describing particular embodiments only and is not intended to be limiting of the disclosure. As used in the description of the embodiments of the disclosure and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, amount, dose, time, temperature, for example, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs.

### Definitions

The term “Abacus” as used herein refers to the Cannabis reference genome known as the Abacus reference genome (version CsaAba2).

The term “acidic cannabinoid” refers to a cannabinoid having one or more carboxylic acid functional groups. Examples of acidic cannabinoids include, but are not limited to, tetrahydrocannabinolic acid (THCA), cannabidiolic acid (CBDA), tetrahydrocannabivarinic acid (THCVA), and cannabichromenic acid (CBCA). Acidic cannabinoids are frequently the predominant cannabinoids found in raw (i.e., unprocessed) cannabis plant material.

The term “alternative nucleotide call” is a nucleotide polymorphism relative to a reference nucleotide for a SNP marker that is significantly associated with the causative SNP(s) that confer(s) a desired phenotype.

The term “backcrossing” or “to backcross” refers to the crossing of an F1 hybrid with one of the original parents. A backcross is used to maintain the identity of one parent (species) and to incorporate a particular trait from a second parent (species). The best strategy is to cross the F1 hybrid back to the parent possessing the most desirable traits. Two or more generations of backcrossing may be necessary, but this is practical only if the desired characteristic or trait is present in the F1.

The term “beneficial” as used herein refers to an allele conferring a modified cannabinoid phenotype.

The term “CBC” means cannabichromene.

The term “CBCA” means cannabichromenic acid.

The term “CBD” means cannabidiol.

The term “CBDA” means cannabidiolic acid.

The term “CBG” means cannabigerol.

The term “CBGA” means cannabigerolic acid.

The term “CBDV” means cannabidivarin.

The term “CBDVA” means cannabidivarinic acid.

The term “CBGV” means cannabigerivarin.

The term “CBGVA” means cannabigerivarinic acid.

The term “cannabinoid” refers to the class of compounds found in cannabis. Non-limiting examples include THC and CBD, but can also include any of the other hundred plus distinct cannabinoids isolated from cannabis.

The term “cannabinoid type I” or “a type I cannabinoid” refers to Total THC:Total CBD ratios, or plants having said ratios, of greater than 3.

The term “cannabinoid type II” or “a type II cannabinoid” refers to Total THC:Total CBD ratios, or plants having said ratios, of between 0.33 and 3.

The term “cannabinoid type III” or “a type III cannabinoid” refers to Total THC:Total CBD ratios, or plants having said ratios, of less than 0.33.

The term “cannabinoid type IV” or “a type IV cannabinoid” refers to Total THC less than or equal to 0.5%, Total CBD less than or equal to 0.5%, and CBG levels greater than or equal to 3%, or plants having said percentages.

The term “Cannabis” refers to plants of the genus Cannabis, including Cannabis sativa, Cannabis indica, and Cannabis ruderalis.

The term “cell” refers to a prokaryotic or eukaryotic cell, including plant cells, capable of replicating DNA, transcribing RNA, translating polypeptides, and secreting proteins.

The term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

The terms “construct,” “plasmid,” “vector,” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. The term “recombinant DNA construct” or “recombinant expression construct” is used interchangeably and refers to a discrete polynucleotide into which a nucleic acid sequence or fragment can be moved. Preferably, it is a plasmid vector or a fragment thereof comprising the promoters of the present invention. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

The term “copy number” refers to the number of physical copies of a genetic determinant, such as a gene, or region of the genome of an organism.

The term “cross”, “crossing”, “cross pollination” or “cross-breeding” refer to the process by which the pollen of one flower on one plant is applied (artificially or naturally) to the ovule (stigma) of a flower on another plant. Backcrossing is a process in which a breeder repeatedly crosses hybrid progeny, for example a first generation hybrid (F1), back to one of the parents of the hybrid progeny. Backcrossing can be used to introduce one or more single locus conversions from one genetic background into another.

The term “cultivar” means a group of similar plants that by structural features and performance (e.g., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain, plant and race are often used interchangeably by plant breeders, agronomists and farmers.

The term “detect” or “detecting” refers to any of a variety of methods for determining the presence of a nucleic acid.

The term “expression” or “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s). Elevated levels refers to higher than average levels of gene expression in comparison to a reference genome, e.g., the Abacus reference genome.

The term “expression cassette” refers to a discrete nucleic acid fragment into which a nucleic acid sequence or fragment can be moved.

The term “functional” as used herein refers to DNA or amino acid sequences which are of sufficient size and sequence to have the desired function (i.e. the ability to cause expression of a gene resulting in gene activity expected of the gene found in a reference genome, e.g., the Abacus reference genome.)

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

The term “genetic modification” or “genetic alteration” as used herein refers to a change from the wild-type or reference sequence of one or more nucleic acid molecules. Genetic modifications or alterations include without limitation, base pair substitutions, additions and deletions of at least one nucleotide from a nucleic acid molecule of known sequence.

The term “genome” as it applies to plant cells encompasses not only chromosomal DNA found within the nucleus, but organelle DNA found within subcellular components (e.g., mitochondrial, plastid) of the cell.

The term “genotype” refers to the genetic makeup of an individual cell, cell culture, tissue, organism (e.g., a plant), or group of organisms.

The term “germplasm” refers to genetic material of or from an individual (e.g., a plant), a group of individuals (e.g., a plant line, variety, or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants can be grown, as well as plant parts, such as leafs, stems, pollen, or cells that can be cultured into a whole plant.

The term “haplotype” refers to the genotype of a plant at a plurality of genetic loci, e.g., a combination of alleles or markers. Haplotype can refer to sequence polymorphisms at a particular locus, such as a single marker locus, or sequence polymorphisms at multiple loci along a chromosomal segment in a given genome. As used herein, a haplotype can be a nucleic acid region spanning two markers.

A plant is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “hybrid” refers to a variety or cultivar that is the result of a cross of plants of two different varieties. A hybrid, as described here, can refer to plants that are genetically different at any particular loci. A hybrid can further include a plant that is a variety that has been bred to have at least one different characteristic from the parent. “F1 hybrid” refers to the first generation hybrid, “F2 hybrid” the second generation hybrid, “F3 hybrid” the third generation, and so on. A hybrid refers to any progeny that is either produced, or developed using research and development to create a new line having at least one distinct characteristic.

The terms “hybridizing specifically to”, “specific hybridization”, and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. A “stringent hybridization” and “stringent hybridization wash conditions” in the context of nucleic acid hybridization (e.g., as in array, Southern or Northern hybridizations) are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, Ch. 2, “Overview of principles of hybridization and the strategy of nucleic acid probe assays,” Elsevier, N.Y. (“Tijssen”). Generally, highly stringent hybridization and wash conditions are selected to be about 5.degree. C. lower than the thermal melting point (T.sub.m) for the specific sequence at a defined ionic strength and pH. The T.sub.m is the temperature (under defined ionic strength and pH) at which 50% of the target sequence hybridizes to a perfectly matched probe. Very stringent conditions are selected to be equal to the T.sub.m for a particular probe. An example of stringent hybridization conditions for hybridization of complementary nucleic acids which have more than 100 complementary residues on an array or on a filter in a Southern or northern blot is 42.degree. C. using standard hybridization solutions (see, e.g., Sambrook and Russell (2001) Molecular Cloning: A Laboratory Manual (3rd ed.) Vol. 1-3, Cold Spring Harbor Laboratory, Cold Spring Harbor Press, NY, and detailed discussion, below).

As used herein, the term “inbreeding” refers to the production of offspring via the mating between relatives. The plants resulting from the inbreeding process are referred to herein as “inbred plants” or “inbreds.”

The terms “initiate transcription,” “initiate expression,” “drive transcription,” and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “introduced” refers to a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The term “isolated” as used herein means having been removed from its natural environment, or removed from other compounds present when the compound is first formed. The term “isolated” embraces materials isolated from natural sources as well as materials (e.g., nucleic acids and proteins) recovered after preparation by recombinant expression in a host cell, or chemically-synthesized compounds such as nucleic acid molecules, proteins, and peptides.

The term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The term “marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Other examples of such markers are restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, cleaved amplified polymorphic sequence (CAPS) markers or isozyme markers or combinations of the markers described herein which defines a specific genetic and chromosomal location.

The term “marker assisted selection” refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The term “nucleotide” refers to an organic molecule that serves as a monomeric unit of DNA and RNA. The nucleotide position is the position along a chromosome wherein any particular monomeric unit of DNA or RNA is positioned relative to the other monomeric units of DNA or RNA.

The term “probe” or “nucleic acid probe,” as used herein, is defined to be a collection of one or more nucleic acid fragments whose specific hybridization to a nucleic acid sample comprising a region of interest can be detected. The probe may be unlabeled or labeled as described below so that its binding to the target nucleic acid of interest can be detected. What “probe” refers to specifically is clear from the context in which the word is used. The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23:1087-1092; Kern (1997) Biotechniques 23:120-124; U.S. Pat. No. 5,143,854). One of skill will recognize that the precise sequence of the particular probes described herein can be modified to a certain degree to produce probes that are “substantially identical” to the disclosed probes, but retain the ability to specifically bind to (i.e., hybridize specifically to) the same targets or samples as the probe from which they were derived (see discussion above). Such modifications are specifically covered by reference to the individual probes described herein.

The term “offspring” refers to any plant resulting as progeny from a vegetative or sexual reproduction from one or more parent plants or descendants thereof. For instance an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The term “oligonucleotide probe” refers to any kind of nucleotide molecule synthesized to match (i.e., be complementary to) a nucleotide sequence of interest which can be used to detect, analyse, and/or visualize said nucleotide sequence on a molecular level. An oligonucleotide probe according to the present disclosure generally refers to a molecule comprising several nucleotides, in general at least 10, 15, and even at least 20 nucleotides, for example, and having at least one label. Optionally, the oligonucleotide probe may also comprise any suitable non-nucleotide units and/or linking reagent which may be suitable to incorporate the label. It should be understood that the oligonucleotide probe has a length suitable to provide the required specificity. In general, the probe may be a DNA oligonucleotide probe or a RNA oligonucleotide probe. Further, it should also be understood that a nucleotide includes all kind of structures composed of a nucleobase (i.e. a nitrogenous base), a five carbon sugar which may be either a ribose, a 2′-deoxyribose, or any derivative thereof, and a phosphate group. The nucleobase and the sugar constitute a unit referred to as a nucleoside.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “percent sequence identity” or “percent identity” or “identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

The term “plant” refers to a whole plant and any descendant, cell, tissue, or part of a plant. A class of plant that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root. A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein. In an embodiment described herein are plants in the genus of Cannabis and plants derived thereof, which can be produced asexual or sexual reproduction.

The term “plant part” or “plant tissue” refers to any part of a plant including but not limited to, an embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen. Plant part may also include certain extracts such as kief, oil, or hash which includes cannabis trichomes or glands.

The terms “polynucleotide,” “polynucleotide sequence,” “nucleotide,” “nucleotide sequence,” “nucleic acid sequence,” “nucleic acid fragment,” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA comprises one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. An “isolated polynucleotide” refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The terms “PCR” or “Polymerase Chain Reaction” refers to a technique for the synthesis of large quantities of specific DNA segments, consisting of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps comprises a cycle.

The term “polymorphism” refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirements of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “probe” or “nucleic acid probe,” as used herein, is defined to be a collection of one or more nucleic acid fragments whose specific hybridization to a nucleic acid sample comprising a region of interest can be detected. The probe may be unlabeled or labeled as described below so that its binding to the target nucleic acid of interest can be detected. What “probe” refers to specifically is clear from the context in which the word is used. The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23:1087-1092; Kern (1997) Biotechniques 23:120-124; U.S. Pat. No. 5,143,854). One of skill will recognize that the precise sequence of the particular probes described herein can be modified to a certain degree to produce probes that are “substantially identical” to the disclosed probes, but retain the ability to specifically bind to (i.e., hybridize specifically to) the same targets or samples as the probe from which they were derived (see discussion above). Such modifications are specifically covered by reference to the individual probes described herein.

The term “progeny” refers to any subsequent generation of a plant. Progeny is measured using the following nomenclature: F1 refers to the first generation progeny, F2 refers to the second generation progeny, F3 refers to the third generation progeny, and so on.

The term “promoter” refers to a nucleic acid fragment capable of controlling transcription of another nucleic acid fragment. A promoter is capable of controlling the expression of a coding sequence or functional RNA. Functional RNA includes, but is not limited to, transfer RNA (tRNA) and ribosomal RNA (rRNA). The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. New promoters of various types useful in plant cells are constantly being discovered; numerous examples may be found in the compilation by Okamuro and Goldberg (Biochemistry of Plants 15:1-82 (1989)). It is further recognized that since in most cases the exact boundaries of regulatory sequences have not been completely defined, DNA fragments of some variation may have identical promoter activity.

The term “protein” refers to amino acid polymers that contain at least five constituent amino acids that are covalently joined by peptide bonds. The constituent amino acids can be from the group of amino acids that are encoded by the genetic code, which include: alanine, valine, leucine, isoleucine, methionine, phenylalanine, tyrosine, tryptophan, serine, threonine, asparagine, glutamine, cysteine, glycine, proline, arginine, histidine, lysine, aspartic acid, and glutamic acid. As used herein, the term “protein” is synonymous with the related terms “peptide” and “polypeptide.”

The term “purified” as used herein relates to the isolation of a molecule or compound in a form that is substantially free of contaminants normally associated with the molecule or compound in a native or natural environment, or substantially enriched in concentration relative to other compounds present when the compound is first formed, and means having been increased in purity as a result of being separated from other components of the original composition. The term “purified nucleic acid” is used herein to describe a nucleic acid sequence which has been separated, produced apart from, or purified away from other biological compounds including, but not limited to polypeptides, lipids and carbohydrates, while effecting a chemical or functional change in the component (e.g., a nucleic acid may be purified from a chromosome by removing protein contaminants and breaking chemical bonds connecting the nucleic acid to the remaining DNA in the chromosome).

The term “quantitative trait loci” or “QTL” refers to the genetic elements controlling a quantitative trait.

The term “reference plant” or “reference genome” refers to a wild-type or reference sequence that SNPs or other markers in a test sample can be compared to in order to detect a modification of the sequence in the test sample.

The term “RNA transcript” refers to a product resulting from RNA polymerase-catalyzed transcription of a DNA sequence. When an RNA transcript is a perfect complementary copy of a DNA sequence, it is referred to as a primary transcript or it may be a RNA sequence derived from posttranscriptional processing of a primary transcript and is referred to as a mature RNA. “Messenger RNA” (“mRNA”) refers to RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA refers to RNA transcript that includes mRNA and so can be translated into protein within a cell or in vitro. “Antisense RNA” refers to a RNA transcript that is complementary to all or part of a target primary transcript or mRNA and that blocks expression or transcripts accumulation of a target gene (U.S. Pat. No. 5,107,065). The complementarity of an antisense RNA may be with any part of the specific gene transcript, i.e. at the 5′ non-coding sequence, 3′ non-coding sequence, introns, or the coding sequence. “Functional RNA” refers to antisense RNA, ribozyme RNA, or other RNA that may not be translated but yet has an effect on cellular processes.

The terms “similar,” “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. A “substantially homologous sequence” refers to variants of the disclosed sequences such as those that result from site-directed mutagenesis, as well as synthetically derived sequences. A substantially homologous sequence of the present invention also refers to those fragments of a particular promoter nucleotide sequence disclosed herein that operate to promote the constitutive expression of an operably linked heterologous nucleic acid fragment. These promoter fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequence disclosed herein. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989. Again, variants of these promoter fragments, such as those resulting from site-directed mutagenesis, are encompassed by the compositions of the present invention.

The term “single nucleotide polymorphism (SNP)” refers to a change in which a single base in the DNA differs from the usual base at that position. These single base changes are called SNPs or “snips.”

The term “target region” or “nucleic acid target” refers to a nucleotide sequence that resides at a specific chromosomal location. The “target region” or “nucleic acid target” is specifically recognized by a probe.

The term “total cannabinoids” refers to the combination of total THC, total CBD, total CBG, total THCV, total CBDV, total CBC, and total CBGV.

The term “total CBC” refers to the approximate calculation of (0.877*CBCA)+CBC.

The term “total CBD” refers to the approximate calculation of (0.877*CBDA)+CBD.

The term “total CBG” refers to the approximate calculation of (0.878*CBGA)+CBG.

The term “total CBDV” refers to the approximate calculation of (0.877*CBDVA)+CBDV.

The term “total CBGV” refers to the approximate calculation of (0.878*CBGVA)+CBGV.

The term “total THC” refers to the approximate calculation of (0.877*THCA)+THC.

The term “total THCV” refers to the approximate calculation of (0.877*THCVA)+THCV.

The term “transition” as used herein refers to the transition of a nucleotide at any specific genomic position with that of a different nucleotide.

The term “transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. The term “transgenic plant” refers to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct. A “transgene” is a gene that has been introduced into the genome by a transformation procedure.

The term “translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D., Molecular Biotechnology 3:225 (1995)).

The term “THC” means tetrahydrocannabinol.

The term “THCA” means tetrahydrocannabinolic acid.

The term “THCV” means tetrahydrocannabivarin.

The term “THCVA” mean tetrahydrocannabivarinic acid.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

### Cannabis

Cannabis has long been used for drug and industrial purposes, fiber (hemp), for seed and seed oils, for medicinal purposes, and for recreational purposes. Industrial hemp products are made from Cannabis plants selected to produce an abundance of fiber. Some Cannabis varieties have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Cannabis is an annual, dioecious, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

The life cycle of Cannabis varies with each variety but can be generally summarized into germination, vegetative growth, and reproductive stages. Because of heavy breeding and selection by humans, most Cannabis seeds have lost dormancy mechanisms and do not require any pre-treatments or winterization to induce germination (See Clarke, R C et al. “Cannabis: Evolution and Ethnobotany” University of California Press 2013). Seeds placed in viable growth conditions are expected to germinate in about 3 to 7 days. The first true leaves of a Cannabis plant contain a single leaflet, with subsequent leaves developing in opposite formation with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, Cannabis plants can grow up to 2.5 inches a day, and are capable of reaching heights of up to 20 feet. Indoor growth pruning techniques tend to limit Cannabis size through careful pruning of apical or side shoots.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al, “The draft genome and transcriptome of Cannabis sativa” Genome Biology 12: R102).

All known varieties of Cannabis are wind-pollinated and the fruit is an achene. Most varieties of Cannabis are short day plants, with the possible exception of C. sativa subsp. sativa var. spontanea (=C. ruderalis), which is commonly described as “auto-flowering” and may be day-neutral.

The genus Cannabis was formerly placed in the Nettle (Urticaceae) or Mulberry (Moraceae) family, and later, along with the Humulus genus (hops), in a separate family, the Hemp family (Cannabaceae sensu stricto). Recent phylogenetic studies based on cpDNA restriction site analysis and gene sequencing strongly suggest that the Cannabaceae sensu stricto arose from within the former Celtidaceae family, and that the two families should be merged to form a single monophyletic family, the Cannabaceae sensu lato.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the Cannabis plant (Rudolf Brenneisen, 2007, Chemistry and Analysis of Phytocannabinoids (cannabinoids produced produced by Cannabis) and other Cannabis Constituents, In Marijuana and the Cannabinoids, ElSohly, ed.; incorporated herein by reference) and at least 85 different cannabinoids have been isolated from the plant (El-Alfy, Abir T, et al., 2010, “Antidepressant-like effect of delta-9-tetrahydrocannabinol and other cannabinoids isolated from Cannabis sativa L”, Pharmacology Biochemistry and Behavior 95 (4): 434-42; incorporated herein by reference). The two cannabinoids usually produced in greatest abundance are cannabidiol (CBD) and/or A9-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

Cannabinoids are the most studied group of secondary metabolites in Cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

Detection of neutral and acidic forms of cannabinoids are dependent on the detection method utilized. Two popular detection methods are high-performance liquid chromatography (HPLC) and gas chromatography (GC). HPLC separates, identifies, and quantifies different components in a mixture, and passes a pressurized liquid solvent containing the sample mixture through a column filled with a solid adsorbent material. Each molecular component in a sample mixture interacts differentially with the adsorbent material, thus causing different flow rates for the different components and therefore leading to separation of the components. In contrast, GC separates components of a sample through vaporization. The vaporization required for such separation occurs at high temperature. Thus, the main difference between GC and HPLC is that GC involves thermal stress and mainly resolves analytes by boiling points while HPLC does not involve heat and mainly resolves analytes by polarity. The consequence of utilizing different methods for cannabinoid detection therefore is that HPLC is more likely to detect acidic cannabinoid precursors, whereas GC is more likely to detect decarboxylated neutral cannabinoids.

The cannabinoids in cannabis plants s include, but are not limited to, Δ9-Tetrahydrocannabinol (Δ9-THC), Δ8-Tetrahydrocannabinol (Δ8-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), Δ9-Tetrahydrocannabivarin (THCV), cannabichromevarin (CBCV), and cannabigerovarin (CBGV). See Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779), each of which is herein incorporated by reference in its entirety for all purposes. Non-THC cannabinoids can be collectively referred to as “CBs”, wherein CBs can be one of THCV, CBDV, CBGV, CBCV, CBD, CBC, CBE, CBG, CBN, CBND, and CBT cannabinoids.

### Cannabinoid Markers and Haplotypes

The present invention describes the discovery of novel markers indicating modified cannabinoids, the method comprising i) obtaining nucleic acids from a sample plant or its germplasm; (ii) detecting one or more markers that indicate modified cannabinoids, and (iii) indicating the modified cannabinoids. The modified cannabinoids may correlate to increased levels of one or more of total tetrahydrocannabinol (THC), total cannabidiol (CBD), total cannabigerol (CBG), total tetrahydrocannabivarin (THCV), total cannabidivarin (CBDV), total cannabichromene (CBC), or total cannabigerovarin (CBGV). The modified cannabinoids may correlate to increased levels of the combination of total tetrahydrocannabinol (THC), total cannabidiol (CBD), total cannabigerol (CBG), total tetrahydrocannabivarin (THCV), total cannabidivarin (CBDV), total cannabichromene (CBC), or total cannabigerovarin (CBGV). An embodiment further describes selecting the one or more plants indicating modified cannabinoids.

The markers of the present invention were discovered as described herein, which comprise polymorphisms relative to the Abacus Cannabis reference genome (version CsaAba2). In an embodiment, as described in Table 4, the markers identify polymorphisms that modify levels of total cannabinoids in cannabinoid type I, II, and III plants. Table 4 describes the markers and sequence identifiers, and the positioning on their respective chromosomes. Table 4 further describes the reference call of the nucleotide at the respective position within the reference genome, as well as the alternate call describing the polymorphism in plants having the modified cannabinoids. Table 4 further describes the beneficial genotype with respect to the described markers. In an embodiment the modification is elevated levels of the respective cannabinoids.

In an embodiment, as described in Table 5, 7, 9, and 10, the markers identify polymorphisms that modify levels of total THC and total THCV in cannabinoid type I plants. Tables 5, 7, 9, and 10 describe the markers and sequence identifiers, and the positioning on their respective chromosomes. Table 5 further describes the reference call of the nucleotide at the respective position within the reference genome, as well as the alternate call describing the polymorphism in plants having the modified cannabinoids. Table 5 further describes the beneficial genotype with respect to the described markers. In an embodiment the modification is elevated levels of the respective cannabinoids.

In an embodiment, as described in Tables 6 and 8, the markers identify polymorphisms that modify levels of total cannabinoids. Tables 6 and 8 further describe the reference call of the nucleotide at the respective position within the reference genome, as well as the alternate call describing the polymorphism in plants having the modified cannabinoids. Tables 6 and 8 further describe the beneficial genotype with respect to the described markers. In an embodiment the modification is elevated levels of the respective cannabinoids.

In an embodiment, as described in Table 11, the markers identify polymorphisms that modify ratios of CBC to total cannabinoids. Table 11 further describes the reference call of the nucleotide at the respective position within the reference genome, as well as the alternate call describing the polymorphism in plants having the modified cannabinoids. Table 11 further describes the beneficial genotype with respect to the described markers. In an embodiment the modification is elevated levels of the respective cannabinoids.

In an embodiment, as described in Tables 12, 13, 14, 15, and 16, the markers identify polymorphisms that modify levels of the combination of total CBG and total CBGV. Tables 12, 13, 14, 15, and 16 further describe the reference call of the nucleotide at the respective position within the reference genome, as well as the alternate call describing the polymorphism in plants having the modified cannabinoids. Tables 12, 13, 14, 15, and 16 further describe the beneficial genotype with respect to the described markers. In an embodiment the modification is elevated levels of the respective cannabinoids.

In an embodiment, as described in Table 17, the markers identify polymorphisms that modify ratios of total cannabinoids to total CBG. Table 17 further describes the reference call of the nucleotide at the respective position within the reference genome, as well as the alternate call describing the polymorphism in plants having the modified cannabinoids. Table 17 further describes the beneficial genotype with respect to the described markers. In an embodiment the modification is elevated levels of the respective cannabinoids.

In an embodiment, as described in Tables 18, 19, 20, and 21, the markers identify polymorphisms that modify ratios of THC to CBG. Tables 18, 19, 20, and 21 further describe the reference call of the nucleotide at the respective position within the reference genome, as well as the alternate call describing the polymorphism in plants having the modified cannabinoids. Tables 18, 19, 20, and 21 further describe the beneficial genotype with respect to the described markers. In an embodiment the modification is elevated levels of the respective cannabinoids.

In an embodiment, as described in Tables 22, 23, and 24, the markers identify polymorphisms that modify ratios of CBC to CBG. Table 22, 23, and 24 further describe the reference call of the nucleotide at the respective position within the reference genome, as well as the alternate call describing the polymorphism in plants having the modified cannabinoids. Tables 22, 23, and 24 further describe the beneficial genotype with respect to the described markers. In an embodiment the modification is elevated levels of the respective cannabinoids.

The markers may be used interchangeably to discover either or both the decarboxylated cannabinoid or its acidic precursor version. To illustrate, for non-limiting exemplary purposes, marker 90_707845 as described in Table 4 can be used to select plants having either modified Total THC, or THCA.

### Quantitative Trait Loci

The term chromosome interval designates a contiguous linear span of genomic DNA that resides on a single chromosome. A chromosome interval may comprise a quantitative trait locus (“QTL”) linked with a genetic trait and the QTL may comprise a single gene or multiple genes associated with the genetic trait. The boundaries of a chromosome interval comprising a QTL are drawn such that a marker that lies within the chromosome interval can be used as a marker for the genetic trait, as well as markers genetically linked thereto. Each interval comprising a QTL comprises at least one gene conferring a given trait, however knowledge of how many genes are in a particular interval is not necessary to make or practice the invention, as such an interval will segregate at meiosis as a linkage block. In accordance with the invention, a chromosomal interval comprising a QTL may therefore be readily introgressed and tracked in a given genetic background using the methods and compositions provided herein.

Identification of chromosomal intervals and QTL is therefore beneficial for detecting and tracking a genetic trait, such as modified cannabinoid activity, in plant populations. In some embodiments, this is accomplished by identification of markers linked to a particular QTL. The principles of QTL analysis and statistical methods for calculating linkage between markers and useful QTL include penalized regression analysis, ridge regression, single point marker analysis, complex pedigree analysis, Bayesian MCMC, identity-by-descent analysis, interval mapping, composite interval mapping (CIM), and Haseman-Elston regression. QTL analyses may be performed with the help of a computer and specialized software available from a variety of public and commercial sources known to those of skill in the art.

### Detection of Markers

The present invention describes the use of detecting cannabinoid markers. Marker detection is well known in the art. For example, amplification of a target polynucleotide (e.g., by PCR) using a particular amplification primer pair that permit the primer pair to hybridize to the target polynucleotide to which a primer having the corresponding sequence (or its complement) would bind and preferably to produce an identifiable amplification product (the amplicon) having a marker is well known in the art.

Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Methods of amplification are further described in U.S. Pat. Nos. 4,683,195, 4,683,202 and Chen et al. (1994) PNAS 91:5695-5699. These methods as well as other methods known in the art of DNA amplification may be used in the practice of the embodiments of the present invention. It will be appreciated that suitable primers to be used with the invention can be designed using any suitable method. It is not intended that the invention be limited to any particular primer or primer pair. It is not intended that the primers of the invention be limited to generating an amplicon of any particular size. For example, the primers used to amplify the marker loci and alleles herein are not limited to amplifying the entire region of the relevant locus. The primers can generate an amplicon of any suitable length that is longer or shorter than those disclosed herein. In some embodiments, marker amplification produces an amplicon at least 20 nucleotides in length, or alternatively, at least 50 nucleotides in length, or alternatively, at least 100 nucleotides in length, or alternatively, at least 200 nucleotides in length. It is understood that a number of parameters in a specific PCR protocol may need to be adjusted to specific laboratory conditions and may be slightly modified and yet allow for the collection of similar results. The primers of the invention may be radiolabeled, or labeled by any suitable means (e.g., using a non-radioactive fluorescent tag), to allow for rapid visualization of the different size amplicons following an amplification reaction without any additional labeling step or visualization step. The known nucleic acid sequences for the genes described herein are sufficient to enable one of skill in the art to routinely select primers for amplification of the gene of interest.

Other suitable amplification methods include, but are not limited to, ligase chain reaction (LCR) (see, Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Natl. Acad. Sci. USA 87:1874), dot PCR, and linker adapter PCR, etc.

An amplicon is an amplified nucleic acid, e.g., a nucleic acid that is produced by amplifying a template nucleic acid by any available amplification method (e.g., PCR, LCR, transcription, or the like). A genomic nucleic acid is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A template nucleic acid is a nucleic acid that serves as a template in an amplification reaction (e.g., a polymerase based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST. Details regarding the use of these and other amplification methods can be found in any of a variety of standard texts. Many available biology texts also have extended discussions regarding PCR and related amplification methods and one of skill will appreciate that essentially any RNA can be converted into a double stranded DNA suitable for restriction digestion, PCR expansion and sequencing using reverse transcriptase and a polymerase.

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan™ probes are oligonucleotides that have a label and a quencher, where the label is released during amplification by the exonuclease action of the polymerase used in amplification, providing a real time measure of amplification during synthesis. A variety of TaqMan™ reagents are commercially available, e.g., from Applied Biosystems as well as from a variety of specialty vendors such as Biosearch Technologies.

In general, synthetic methods for making oligonucleotides, including probes, primers, molecular beacons, PNAS, LNAs (locked nucleic acids), etc., are well known. For example, oligonucleotides can be synthesized chemically according to the solid phase phosphoramidite triester method described. Oligonucleotides, including modified oligonucleotides, can also be ordered from a variety of commercial sources.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes of the invention be limited to any particular size.

Amplification is not always a requirement for marker detection (e.g. Southern blotting and RFLP detection). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

### Cannabinoid Genes

In an embodiment, candidate genes based conferring modified cannabinoids based on the markers described herein may be provided.

Preferred substantially similar nucleic acid sequences encompassed by this invention are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 72% to 100%, such as 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In an embodiment, an isolated polynucleotide is provided comprising a nucleotide sequence having at least 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity compared to the claimed sequence, based on the Clustal V method of alignment with pairwise alignment default parameters (KTUPLE=2, GAP PENALTY=5, WINDOW=4 and DIAGONALS SAVED=4).

Local sequence alignment programs are similar in their calculation, but only compare aligned fragments of the sequences rather than utilizing an end-to-end analysis. Local sequence alignment programs such as BLAST can be used to compare specific regions of two sequences. A BLAST comparison of two sequences results in an E-value, or expectation value, that represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST match is considered herein as having an E-value for the top BLAST hit of less than 1E-30; a medium BLASTX E-value is 1E-30 to 1E-8; and a low BLASTX E-value is greater than 1E-8. The protein function assignment in the present invention is determined using combinations of E-values, percent identity, query coverage and hit coverage. Query coverage refers to the percent of the query sequence that is represented in the BLAST alignment. Hit coverage refers to the percent of the database entry that is represented in the BLAST alignment. In one embodiment of the invention, function of a query polypeptide is inferred from function of a protein homolog where either (1) hit_p<1e-30 or % identity>35% AND query_coverage>50% AND hit_coverage>50%, or (2) hit_p<1e-8 AND query_coverage>70% AND hit_coverage>70%. The following abbreviations are produced during a BLAST analysis of a sequence. SEQ_NUM provides the SEQ ID NO for the listed recombinant polynucleotide sequences. CONTIG_ID provides an arbitrary sequence name taken from the name of the clone from which the cDNA sequence was obtained. PROTEIN_NUM provides the SEQ ID NO for the recombinant polypeptide sequence NCBI_GI provides the GenBank ID number for the top BLAST hit for the sequence. The top BLAST hit is indicated by the National Center for Biotechnology Information GenBank Identifier number. NCBI_GI_DESCRIPTION refers to the description of the GenBank top BLAST hit for sequence. E_VALUE provides the expectation value for the top BLAST match. MATCH_LENGTH provides the length of the sequence which is aligned in the top BLAST match TOP_HIT_PCT_IDENT refers to the percentage of identically matched nucleotides (or residues) that exist along the length of that portion of the sequences which is aligned in the top BLAST match. CAT_TYPE indicates the classification scheme used to classify the sequence. GO_BP=Gene Ontology Consortium—biological process; GO_CC=Gene Ontology Consortium—cellular component; GO_MF=Gene Ontology Consortium molecular function; KEGG=KEGG functional hierarchy (KEGG=Kyoto Encyclopedia of Genes and Genomes); EC=Enzyme Classification from ENZYME data bank release 25.0; POI=Pathways of Interest. CAT_DESC provides the classification scheme subcategory to which the query sequence was assigned. PRODUCT_CAT_DESC provides the FunCAT annotation category to which the query sequence was assigned. PRODUCT_HIT_DESC provides the description of the BLAST hit which resulted in assignment of the sequence to the function category provided in the cat_desc column. HIT_E provides the E value for the BLAST hit in the hit_desc column. PCT_IDENT refers to the percentage of identically matched nucleotides (or residues) that exist along the length of that portion of the sequences which is aligned in the BLAST match provided in hit_desc. QRY_RANGE lists the range of the query sequence aligned with the hit. HIT_RANGE lists the range of the hit sequence aligned with the query. provides the percent of query sequence length that matches QRY_CVRG provides the percent of query sequence length that matches to the hit (NCBI) sequence in the BLAST match (% qry cvrg=(match length/query total length)×100). HIT_CVRG provides the percent of hit sequence length that matches to the query sequence in the match generated using BLAST (% hit cvrg=(match lengthy hit total length)×100).

Methods for aligning sequences for comparison are well-known in the art. Various programs and alignment algorithms are described. In an embodiment, the subject disclosure relates to calculating percent identity between two polynucleotides or amino acid sequences using an AlignX alignment program of the Vector NTI suite (Invitrogen, Carlsbad, Calif.). The AlignX alignment program is a global sequence alignment program for polynucleotides or proteins. In an embodiment, the subject disclosure relates to calculating percent identity between two polynucleotides or amino acid sequences using the MegAlign program of the LASERGENE bioinformatics computing suite (MegAlign™ (.COPYRGT.1993-2016). DNASTAR. Madison, Wis.). The MegAlign program is a global sequence alignment program for polynucleotides or proteins.

### Cannabis Breeding

Cannabis is an important and valuable crop. Thus, a continuing goal of Cannabis plant breeders is to develop stable, high yielding Cannabis cultivars that are agronomically sound. To accomplish this goal, the Cannabis breeder preferably selects and develops Cannabis plants with traits that result in superior cultivars. The plants described herein can be used to produce new plant varieties. In some embodiments, the plants are used to develop new, unique, and superior varieties or hybrids with desired phenotypes.

The development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

Details of existing Cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011), each of which is herein incorporated by reference in its entirety for all purposes.

Pedigree selection, where both single plant selection and mass selection practices are employed, may be used for the generating varieties as described herein. Pedigree selection, also known as the “Vilmorin system of selection,” is described in Fehr, Walter; Principles of Cultivar Development, Volume I, Macmillan Publishing Co., which is hereby incorporated by reference. Pedigree breeding is used commonly for the improvement of self-pollinating crops or inbred lines of cross-pollinating crops. Two parents which possess favorable, complementary traits are crossed to produce an F1. An F2 population is produced by selfing one or several F1's or by intercrossing two F1's (sib mating). Selection of the best individuals usually begins in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

A single-seed descent procedure refers to planting a segregating population, harvesting a sample of one seed per plant, and using the one-seed sample to plant the next generation. When the population has advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The complexity of inheritance also influences the choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety for all purposes. Cannabis genome has been sequenced (Bakel et al., The draft genome and transcriptome of Cannabis sativa, Genome Biology, 12 (10): R102, 2011). Molecular markers for Cannabis plants are described in Datwyler et al. (Genetic variation in hemp and marijuana (Cannabis sativa L.) according to amplified fragment length polymorphisms, J Forensic Sci. 2006 March; 51 (2): 371-5), Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10 (4), 2007), Datwyler et al., (Genetic Variation in Hemp and Marijuana (Cannabis sativa L.) According to Amplified Fragment Length Polymorphisms, J Forensic Sci, March 2006, 51 (2): 371-375), Gilmore et al. (Isolation of microsatellite markers in Cannabis sativa L. (marijuana), Molecular Ecology Notes, 3(1):105-107, March 2003), Pacifico et al., (Genetics and marker-assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726), each of which is herein incorporated by reference in its entirety for all purposes.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

### Marker Assisted Selection Breeding

In an embodiment, marker assisted selection (MAS) is used to produce plants with desired traits. MAS is a powerful shortcut to selecting for desired phenotypes and for introgressing desired traits into cultivars (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

Introgression refers to the transmission of a desired allele of a genetic locus from one genetic background to another, which is significantly assisted through MAS. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like.

The introgression of one or more desired loci from a donor line into another is achieved via repeated backcrossing to a recurrent parent accompanied by selection to retain one or more loci from the donor parent. Markers associated with modified cannabinoids may be assayed in progeny and those progeny with one or more desired markers are selected for advancement. In another aspect, one or more markers can be assayed in the progeny to select for plants with the genotype of the agronomically elite parent. This invention anticipates that trait introgressed modified cannabinoids will require more than one generation, wherein progeny are crossed to the recurrent (agronomically elite) parent or selfed. Selections are made based on the presence of one or more modified cannabinoid markers and can also be made based on the recurrent parent genotype, wherein screening is performed on a genetic marker and/or phenotype basis. In another embodiment, markers of this invention can be used in conjunction with other markers, ideally at least one on each chromosome of the Cannabis genome, to track the modified cannabinoid phenotypes.

Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. The present invention provides the means to identify plants that exhibit modified cannabinoid by identifying plants having modified cannabinoid-specific markers.

In general, MAS uses polymorphic markers that have been identified as having a significant likelihood of co-segregation with a desired trait. Such markers are presumed to map near a gene or genes that give the plant its desired phenotype, and are considered indicators for the desired trait, and are termed QTL markers. Plants are tested for the presence or absence of a desired allele in the QTL marker.

Identification of plants or germplasm that include a marker locus or marker loci linked to a desired trait or traits provides a basis for performing MAS. Plants that comprise favorable markers or favorable alleles are selected for, while plants that comprise markers or alleles that are negatively correlated with the desired trait can be selected against. Desired markers and/or alleles can be introgressed into plants having a desired (e.g., elite or exotic) genetic background to produce an introgressed plant or germplasm having the desired trait. In some aspects, it is contemplated that a plurality of markers for desired traits are sequentially or simultaneously selected and/or introgressed. The combinations of markers that are selected for in a single plant are not limited, and can include any combination of markers disclosed herein or any marker linked to the markers disclosed herein, or any markers located within the QTL intervals defined herein.

In some embodiments, a first Cannabis plant or germplasm exhibiting a desired trait (the donor) can be crossed with a second Cannabis plant or germplasm (the recipient, e.g., an elite or exotic Cannabis, depending on characteristics that are desired in the progeny) to create an introgressed Cannabis plant or germplasm as part of a breeding program. In some aspects, the recipient plant can also contain one or more loci associated with one or more desired traits, which can be qualitative or quantitative trait loci. In another aspect, the recipient plant can contain a transgene.

MAS, as described herein, using additional markers flanking either side of the DNA locus provide further efficiency because an unlikely double recombination event would be needed to simultaneously break linkage between the locus and both markers. Moreover, using markers tightly flanking a locus, one skilled in the art of MAS can reduce linkage drag by more accurately selecting individuals that have less of the potentially deleterious donor parent DNA. Any marker linked to or among the chromosome intervals described herein can thus find use within the scope of this invention.

Similarly, by identifying plants lacking a desired marker locus, plants having unfavorable modified cannabinoids can be identified and eliminated from subsequent crosses. These marker loci can be introgressed into any desired genomic background, germplasm, plant, line, variety, etc., as part of an overall MAS breeding program designed to enhance modified cannabinoids. The invention also provides chromosome QTL intervals that can be used in MAS to select plants that demonstrate different modified cannabinoid traits. The QTL intervals can also be used to counter-select plants that have less favorable modified cannabinoids.

Thus, the invention permits one skilled in the art to detect the presence or absence of modified cannabinoid genotypes in the genomes of Cannabis plants as part of a MAS program, as described herein. In one embodiment, a breeder ascertains the genotype at one or more markers for a parent having favorable modified cannabinoid, which contains a favorable modified cannabinoid allele, and the genotype at one or more markers for a parent with unfavorable modified cannabinoid, which lacks the favorable modified cannabinoid allele. A breeder can then reliably track the inheritance of the modified cannabinoid alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the parent having modified cannabinoid alleles can be reliably predicted to express the desirable phenotype and progeny that share genotypes with the parent having unfavorable modified cannabinoid alleles can be reliably predicted to express the undesirable phenotype. Thus, the laborious, inefficient, and potentially inaccurate process of manually phenotyping the progeny for modified cannabinoid traits is avoided.

Closely linked markers flanking the locus of interest that have alleles in linkage disequilibrium with modified cannabinoid alleles at that locus may be effectively used to select for progeny plants with desirable modified cannabinoid traits. Thus, the markers described herein, such as those listed in Tables 3 through 5, as well as other markers genetically linked to the same chromosome interval, may be used to select for Cannabis plants with different modified cannabinoid traits. Often, a set of these markers will be used, (e.g., 2 or more, 3 or more, 4 or more, 5 or more) in the flanking regions of the locus. Optionally, as described above, a marker flanking or within the actual locus may also be used. The parents and their progeny may be screened for these sets of markers, and the markers that are polymorphic between the two parents used for selection. In an introgression program, this allows for selection of the gene or locus genotype at the more proximal polymorphic markers and selection for the recurrent parent genotype at the more distal polymorphic markers.

In an embodiment, MAS is used to select one or more cannabis plants comprising modified cannabinoids, the method comprising: i) obtaining nucleic acids from a sample plant or its germplasm; (ii) detecting one or more markers that indicate modified cannabinoids, and (iii) indicating modified cannabinoids.

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes may in some circumstances be more informative than single SNPs and can be more descriptive of any particular genotype. Haplotypes of the present invention are described in Table 5, and can be used for marker assisted selection.

The choice of markers actually used to practice the invention is not limited and can be any marker that is genetically linked to the intervals as described herein, which includes markers mapping within the intervals. In certain embodiments, the invention further provides markers closely genetically linked to, or within approximately 0.5 CM of, the markers provided herein and chromosome intervals whose borders fall between or include such markers, and including markers within approximately 0.4 CM, 0.3 CM, 0.2 cM, and about 0.1 cM of the markers provided herein.

In some embodiments the markers and haplotypes described above can be used for marker assisted selection to produce additional progeny plants comprising the indicated modified cannabinoids. In some embodiments, backcrossing may be used in conjunction with marker-assisted selection.

### Gene Editing

In some embodiments gene editing is used to develop plants having modified cannabinoids. In particular, methods for selecting one or more cannabis plants having modified cannabinoids, the method comprising: (i) replacing a nucleic acid sequence of a parent plant with a nucleic acid sequence conferring modified cannabinoids, (ii) crossing or selfing the parent plant, thereby producing a plurality of progeny seed, and (iii), selecting one or more progeny plants grown from the progeny seed that comprise the nucleic acid sequence conferring modified cannabinoids, thereby selecting modified plants having modified cannabinoids.

Gene editing is well known in the art, and many methods can be used with the present invention. For example, a skilled artisan will recognize that the ability to engineer a trait relies on the action of the genome editing proteins and various endogenous DNA repair pathways. These pathways may be normally present in a cell or may be induced by the action of the genome editing protein. Using genetic and chemical tools to over-express or suppress one or more genes or elements of these pathways can improve the efficiency and/or outcome of the methods of the invention. For example, it can be useful to over-express certain homologous recombination pathway genes or suppression of non-homologous pathway genes, depending upon the desired modification.

For example, gene function can be modified using antisense modulation using at least one antisense compound, including antisense DNA, antisense RNA, a ribozyme, DNAzyme, a locked nucleic acid (LNA) and an aptamer. In some embodiments the molecules are chemically modified. In other embodiments the antisense molecule is antisense DNA or an antisense DNA analog.

RNA interference (RNAi) is another method known in the art to reduce gene function in plants, which is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs. The dsRNA used to initiate RNAi, may be isolated from native source or produced by known means, e.g., transcribed from DNA. Plasmids and vectors for generating RNAi molecules against target sequence are now readily available from commercial sources.

DNAzyme molecules, enzymatic oligonucleotides, and mutagenesis are other commonly known methods for reducing gene function. Any available mutagenesis procedure can be used, including but not limited to, site-directed point mutagenesis, random point mutagenesis, in vitro or in vivo homologous recombination (DNA shuffling), uracil-containing templates, oligonucleotide-directed mutagenesis, phosphorothioate-modified DNA mutagenesis, mutagenesis using gapped duplex DNA, point mismatch repair, repair-deficient host strains, restriction-selection and restriction-purification, deletion mutagenesis, total gene synthesis, double-strand break repair, zinc-finger nucleases (ZFN), transcription activator-like effector nucleases (TALEN), any other mutagenesis procedure known to a person skilled in the art.

A skilled artisan would also appreciate that clustered regularly interspaced short palindromic repeats (CRISPR)/CRISPR associated protein (Cas) system comprises genome engineering tools based on the bacterial CRISPR/Cas prokaryotic adaptive immune system. This RNA-based technology is very specific and allows targeted cleavage of genomic DNA guided by a customizable small noncoding RNA, resulting in gene modifications by both non-homologous end joining (NHEJ) and homology-directed repair (HDR) mechanisms (Belhaj K. et al., 2013. Plant Methods 2013, 9:39). In some embodiments, a CRISPR/Cas system comprises a CRISPR/Cas9 system. CRISPR-based gene editing systems need not be limited to Cas9 systems, as those skilled in the art are aware of other analogous editing enzymes, e.g., MAD7.

Methods for transformation of plant cells required for gene editing are well known in the art, and the selection of the most appropriate transformation technique for a particular embodiment of the invention may be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens mediated transformation. Transformation means introducing a nucleotide sequence in a plant in a manner to cause stable or transient expression of the sequence.

In planta transformation techniques (e.g., vacuum-infiltration, floral spraying or floral dip procedures) are well known in the art and may be used to introduce expression cassettes of the invention (typically in an Agrobacterium vector) into meristematic or germline cells of a whole plant. Such methods provide a simple and reliable method of obtaining transformants at high efficiency while avoiding the use of tissue culture. (see, e.g., Bechtold et at. 1993 C. R. Acad. Sci. 316:1194-1199; Chung et at. 2000 Transgenic Res. 9:471-476; Clough et al. 1998 Plant J. 16:735-743; and Desfeux et at. 2000 Plant Physiol 123:895-904). In these embodiments, seed produced by the plant comprise the expression cassettes encoding the genome editing proteins of the invention. The seed can be selected based on the ability to germinate under conditions that inhibit germination of the untransformed seed.

If transformation techniques require use of tissue culture, transformed cells may be regenerated into plants in accordance with techniques well known to those of skill in the art. The regenerated plants may then be grown, and crossed with the same or different plant varieties using traditional breeding techniques to produce seed, which are then selected under the appropriate conditions.

The expression cassette can be integrated into the genome of the plant cells, in which case subsequent generations will express the genome editing proteins of the invention. Alternatively, the expression cassette is not integrated into the genome of the plant's cell, in which case the genome editing protein is transiently expressed in the transformed cells and is not expressed in subsequent generations.

A genome editing protein itself may be introduced into the plant cell. In these embodiments, the introduced genome editing protein is provided in sufficient quantity to modify the cell but does not persist after a contemplated period of time has passed or after one or more cell divisions. In such embodiments, no further steps are needed to remove or segregate away the genome editing protein and the modified cell. In these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

The genome editing protein can also be expressed in Agrobacterium as a fusion protein, fused to an appropriate domain of a virulence protein that is translocated into plants (e.g., VirD2, VirE2, VirE2 and VirF). The Vir protein fused with the genome editing protein travels to the plant cell's nucleus, where the genome editing protein would produce the desired double stranded break in the genome of the cell. (see Vergunst et at. 2000 Science 290:979-82).

### Kits for Use in Diagnostic Applications

Kits for use in diagnostic, research, and prognostic applications are also provided by the invention. Such kits may include any or all of the following: assay reagents, buffers, nucleic acids for detecting the target sequences and other hybridization probes and/or primers. The kits may include instructional materials containing directions (i.e., protocols) for the practice of the methods of this invention. While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), cloud-based media, and the like. Such media may include addresses to internet sites that provide such instructional materials.

### EXAMPLES

Aspects of the present teachings can be further understood in light of the following examples, which should not be construed as limiting the scope of the present teachings in any way.

The practice of the present teachings employ, unless otherwise indicated, conventional methods of protein chemistry, biochemistry, recombinant DNA techniques and pharmacology, within the skill of the art. Such techniques are explained fully in the literature. See, e.g., T. Creighton, Proteins: Structures and Molecular Properties, 1993, W. Freeman and Co.; A. Lehninger, Biochemistry, Worth Publishers, Inc. (current addition); J. Sambrook et al., Molecular Cloning: A Laboratory Manual, 2nd Edition, 1989; Methods In Enzymology, S. Colowick and N. Kaplan, eds., Academic Press, Inc.; Remington's Pharmaceutical Sciences, 18th Edition, 1990, Mack Publishing Company, Easton, Pa.; Carey and Sundberg, Advanced Organic Chemistry, Vols. A and B, 3rd Edition, 1992, Plenum Press.

The practice of the present teachings also employ, unless otherwise indicated, conventional methods of statistical analysis, within the skill of the art. Such techniques are explained fully in the literature. See, e.g., J. Little and D. Rubin, Statistical Analysis with Missing Data, 2nd Edition 2002, John Wiley and Sons, Inc., NJ; M. Pepe, The Statistical Evaluation of Medical Tests for Classification and Prediction (Oxford Statistical Science Series) 2003, Oxford University Press, Oxford, UK; X. Zhoue et al., Statistical Methods in Diagnostic Medicine 2002, John Wiley and Sons, Inc., NJ; T. Hastie et. al, The Elements of Statistical Learning: Data Mining, Inference, and Prediction, Second Edition 2009, Springer, N.Y.; W. Cooley and P. Lohnes, Multivariate procedures for the behavioral sciences 1962, John Wiley and Sons, Inc. NY; E. Jackson, A User's Guide to Principal Components 2003, John Wiley and Sons, Inc., NY.

### Example 1—Discovery of Markers Associated with Cannabinoid Levels

### Material

The germplasm used for QTL mapping was an F2 mapping population (n=294) between a cannabis and a hemp variety segregating for production of THC and/or CBD (Total THC/Total CBD ratio<0.33 for type III, 0.33-3 for type II, and >3 for type I; Tables 1 and 3). The 294 accessions were evaluated in two consecutive experiments performed in a greenhouse (n=198) and a growth room (n=96), respectively. All 294 accessions were used to create a linkage map, however, flower chemotype data were collected on 200 accessions. Chemotype data were not collected on plants in poor health and/or those that exhibited hermaphroditism. This resulted in 142 accessions with chemotype data collected in the greenhouse experiment (=averages of up to 3 clonal replicates per accession) and 58 accessions with chemotype data from the growth room experiment (Tables 1 and 3).

TABLE 1
F2 population used for QTL mapping. First column: seed lot ID of F2 population grown
in greenhouse (*) and growth room (**); second column: cannabinoid type; third column:
number of accessions per seed lot per type; fourth column: minimum value for Total
Cannabinoids (%) per seed lot per cannabinoid type; fifth column: maximum value
for Total Cannabinoids (%) per seed lot per cannabinoid type; sixth column: minimum
value for Total Cannabinoids to CBG Ratio per seed lot per cannabinoid type (NA =
no data because chemotyping method used for growth room samples was unable to
detect CBG levels); seventh column maximum value for Total Cannabinoids to CBG
Ratio per seed lot per cannabinoid type (NA = no data because chemotyping method
used for growth room samples was unable to detect CBG levels).

| | | | Total | Total | Total | Total |
|---|---|---|---|---|---|---|
| | | | Cannabinoids | Cannabinoids | Cannabinoids | Cannabinoids |
| | | | minimum | maximum | to CBG Ratio | to CBG Ratio |
| Seed lot | Type | Count | (%) | (%) | minimum | maximum |
| 19GAR2-1* | I | 27 | 6.70 | 15.79 | 6.39 | 13.47 |
| 19GAR2-1* | II | 74 | 5.57 | 17.72 | 5.75 | 14.71 |
| 19GAR2-1* | III | 41 | 7.21 | 18.77 | 6.50 | 10.89 |
| 19MND1-1** | I | 14 | 6.50 | 17.80 | NA | NA |
| 19MND1-1** | II | 29 | 4.80 | 18.80 | NA | NA |
| 19MND1-1** | III | 15 | 7.00 | 16.70 | NA | NA |

The germplasm used for nested association mapping (NAM) of all three cannabinoid types was a set of 144 diverse seed lots consisting of 1-56 accessions each; this data set included accessions with Total Varin up to 11.6% (n=895; Table 2; set 1 Table 3). NAM of type I made use of a set of 122 diverse seed lots consisting of 1-36 accessions each; this data set included accessions with Total Varin up to 2.4% (n=682; set 2 Table 3). NAM of type III made use of a set of 24 diverse seed lots consisting of 2-18 accessions each; this data set included accessions with Total Varin up to 2.9% (n=146; set 3 Table 3). Subsequently, additional NAM of type I was performed on a set of 38 diverse type I seed lots (>=3 accessions per seed lot) with Total Varin less than 0.5% (n=233; set 4 Table 3), a set of 18 diverse type I seed lots (>=3 accessions per seed lot) with Total Varin greater than 1.6% (n=266; set 5 Table 3), and a F2 population segregating for type I and IV (type IV is defined as mainly CBG with negligible amounts of THC and CBD; n=85; set 6 Table 3). Except for the F2 population segregating for type I, II, and III evaluated in a greenhouse (n=142; set 7 Table 3), which was grown at up to three clonal replicates per accession, each accession was grown as a single plant in all other experiments.

TABLE 2
Seed lots used for NAM analysis of sets 1-3.

| | | | Average Total | | |
|---|---|---|---|---|---|
| | | | Cannabinoids | | Average THC to |
| | Cannabinoid | #Accessions in | (%) in types I, II, | Average Total | CBG Ratio in |
| Seed lot | Type | seed lot | and III | THC (%) in type I | type I |
| 19GAR1-1 | I | 6 | 14.59 | 13.81 | 8.48 |
| 19GAR1-12 | I | 3 | 20.67 | 18.98 | 8.00 |
| 19GAR1-16 | I | 5 | 13.86 | 13.05 | 8.25 |
| 19GAR1-17 | I | 3 | 12.86 | 12.55 | 10.48 |
| 19GAR1-19 | I | 4 | 19.01 | 18.06 | 9.79 |
| 19GAR1-2 | I | 3 | 16.96 | 16.11 | 9.34 |
| 19GAR1-23 | I | 2 | 22.82 | 21.56 | 10.43 |
| 19GAR1-24 | I | 8 | 15.6 | 14.15 | 6.44 |
| 19GAR1-25 | I | 6 | 16.89 | 15.90 | 9.10 |
| 19GAR1-27 | I | 4 | 15.34 | 14.55 | 8.71 |
| 19GAR1-28 | I | 3 | 17.85 | 17.07 | 10.26 |
| 19GAR1-3 | I | 6 | 16.11 | 15.37 | 9.56 |
| 19GAR1-30 | I | 4 | 22.33 | 21.62 | 13.17 |
| 19GAR1-32 | I | 2 | 18 | 17.66 | 13.71 |
| 19GAR1-34 | I | 6 | 17.51 | 16.90 | 11.40 |
| 19GAR1-36 | I | 2 | 15.91 | 15.41 | 11.19 |
| 19GAR1-4 | I | 5 | 21.72 | 20.43 | 9.59 |
| 19GAR1-5 | I | 4 | 17.88 | 16.84 | 8.69 |
| 19GAR1-57 | I | 2 | 25.02 | 23.93 | 11.90 |
| 19GAR1-6 | I | 3 | 15.08 | 14.44 | 9.43 |
| 19GAR1-7 | I | 3 | 13.95 | 13.37 | 9.30 |
| 19GAR3-101 | I | 6 | 17.04 | 15.28 | 5.94 |
| 19GAR3-109 | I | 2 | 14.44 | 14.36 | 6.57 |
| 19GAR3-110 | I | 9 | 18.77 | 17.09 | 7.21 |
| 19GAR3-116 | I | 4 | 19.14 | 18.13 | 9.81 |
| 19GAR3-117 | I | 6 | 13.82 | 13.17 | 8.61 |
| 19GAR3-118 | I | 3 | 11.35 | 10.58 | 6.48 |
| 19GAR3-119 | I | 6 | 13.64 | 12.61 | 6.75 |
| 19GAR3-120 | I | 4 | 16.53 | 15.40 | 8.24 |
| 19GAR3-121 | I | 5 | 19.95 | 17.95 | 6.41 |
| 19GAR3-123 | I | 2 | 14.87 | 14.21 | 9.17 |
| 19GAR3-125 | I | 4 | 20.02 | 18.74 | 8.86 |
| 19GAR3-126 | I | 4 | 19.14 | 17.77 | 7.92 |
| 19GAR3-127 | I | 8 | 16.6 | 15.51 | 8.00 |
| 19GAR3-128 | I | 8 | 14.02 | 13.33 | 8.54 |
| 19GAR3-129 | I | 7 | 17.98 | 16.77 | 8.11 |
| 19GAR3-130 | I | 6 | 17.67 | 16.51 | 8.38 |
| 19GAR3-131 | I | 9 | 20.95 | 20.06 | 11.17 |
| 19GAR3-60 | I | 3 | 17.34 | 16.25 | 8.68 |
| 19GAR3-62 | I | 2 | 18.51 | 17.51 | 9.22 |
| 19GAR3-70 | I | 2 | 17.71 | 15.02 | 4.34 |
| 19GAR3-72 | I | 3 | 18 | 16.48 | 7.21 |
| 19GAR3-74 | I | 2 | 17.7 | 16.04 | 6.44 |
| 19GAR3-78 | I | 5 | 14.8 | 11.63 | 3.05 |
| 19GAR3-85 | I | 4 | 19.15 | 18.12 | 9.44 |
| 19GAR3-86 | I | 5 | 21.41 | 20.15 | 9.40 |
| 19GAR3-88 | I | 5 | 19.84 | 18.85 | 10.05 |
| 19GAR3-89 | I | 2 | 15.56 | 14.68 | 8.35 |
| 19GAR3-90 | I | 4 | 18.88 | 17.98 | 10.00 |
| 19GAR3-91 | I | 3 | 21.02 | 19.43 | 8.09 |
| 19GAR3-92 | I | 9 | 24.15 | 22.48 | 9.15 |
| 19GAR3-93 | I | 4 | 19.26 | 18.13 | 8.99 |
| 19GAR3-94 | I | 4 | 20.81 | 19.31 | 8.14 |
| 19GAR3-95 | I | 4 | 16.66 | 15.23 | 6.80 |
| 19GAR3-96 | I | 4 | 15.37 | 14.66 | 9.34 |
| 19GAR3-99 | I | 6 | 15.19 | 14.15 | 7.58 |
| 20TP1B-1001 | I | 10 | 24.11 | 22.89 | 12.21 |
| 20TP1B-1002 | I | 7 | 25.52 | 24.63 | 14.14 |
| 20TP1B-1003 | I | 3 | 23.34 | 22.54 | 13.48 |
| 20TP1B-1004 | I | 3 | 20.52 | 19.78 | 12.85 |
| 20TP1B-1005 | I | 2 | 18.37 | 17.92 | 13.25 |
| 20TP1B-1006 | I | 4 | 16.18 | 15.85 | 12.60 |
| 20TP1B-1007 | I | 7 | 15.5 | 15.38 | 14.05 |
| 20TP1B-1008 | I | 3 | 21.63 | 20.80 | 12.44 |
| 20TP1B-1009 | I | 13 | 20.56 | 20.30 | 16.96 |
| 20TP1B-1010 | I | 7 | 19.5 | 18.92 | 13.28 |
| 20TP1B-1011 | I | 11 | 17.82 | 17.24 | 11.87 |
| 20TP1B-1012 | I | 12 | 21.65 | 20.96 | 14.19 |
| 20TP1B-1013 | I | 7 | 18.62 | 18.08 | 12.66 |
| 20TP1B-1014 | I | 14 | 21.91 | 21.41 | 15.88 |
| 20TP1B-1015 | I | 10 | 21.59 | 21.00 | 15.26 |
| 20TP1B-1016 | I | 4 | 18.38 | 17.54 | 10.42 |
| 20TP1B-1017 | I | 9 | 19.21 | 18.40 | 11.67 |
| 20TP1B-1018 | I | 8 | 18.23 | 17.52 | 11.02 |
| 20TP1B-1019 | I | 13 | 23.45 | 22.65 | 11.48 |
| 20TP1B-1020 | I | 4 | 26 | 24.83 | 12.70 |
| 20TP1B-1021 | I | 7 | 24 | 23.04 | 14.27 |
| 20TP1B-1022 | I | 12 | 22.75 | 21.65 | 12.24 |
| 20TP1B-1023 | I | 15 | 23.16 | 21.88 | 12.29 |
| 20TP1B-1024 | I | 2 | 16.91 | 16.17 | 10.19 |
| 20TP1B-1025 | I | 10 | 29.02 | 27.07 | 10.72 |
| 20TP1B-1028 | I | 4 | 15.25 | 14.46 | 8.90 |
| 20TP1B-1029 | I | 8 | 18.65 | 17.89 | 11.14 |
| 20TP1B-1031 | I | 9 | 16.81 | 16.22 | 11.14 |
| 20TP1B-1032 | I | 10 | 20.58 | 20.16 | 15.22 |
| 20TP1B-1033 | I | 6 | 22.02 | 20.86 | 10.51 |
| 20TP1B-1035 | I | 4 | 19.02 | 18.44 | 12.98 |
| 20TP1B-1036 | I | 5 | 18.3 | 17.58 | 11.44 |
| 20TP1B-1037 | I | 10 | 16.6 | 16.16 | 13.34 |
| 20TP1B-1038 | I | 7 | 17.91 | 17.39 | 13.23 |
| 20TP1B-1039 | I | 10 | 17.51 | 16.84 | 12.26 |
| 20TP1B-1040 | I | 5 | 18.42 | 17.77 | 11.61 |
| 20TP1B-1041 | I | 13 | 18.17 | 17.42 | 12.24 |
| 20TP1B-1042 | I | 12 | 19.88 | 18.87 | 10.95 |
| 20TP1C-1022 | I | 4 | 21.59 | 20.23 | 10.63 |
| 20TP1C-1023 | I | 2 | 23.81 | 22.53 | 12.03 |
| 20TP1C-1025 | I | 5 | 27.36 | 25.53 | 10.71 |
| 20TP1C-1027 | I | 2 | 24.38 | 23.09 | 11.71 |
| 20TP1C-1028 | I | 7 | 24.15 | 22.82 | 11.97 |
| 20TP1C-1029 | I | 10 | 18.75 | 17.64 | 9.99 |
| 20TP1C-1030 | I | 7 | 16.72 | 15.68 | 8.50 |
| 20TP1C-1031 | I | 3 | 18.01 | 17.58 | 14.18 |
| 20TP1C-1032 | I | 2 | 18.72 | 18.51 | 15.70 |
| 20TP1C-1034 | I | 2 | 27.21 | 25.85 | 12.60 |
| 20TP1C-1036 | I | 4 | 23.7 | 22.39 | 12.08 |
| 20TP1C-1042 | I | 4 | 21.69 | 20.39 | 10.13 |
| 20TP1C-1050 | I | 4 | 27.2 | 29.38 | 9.04 |
| 20VLDY-1001 | I | 36 | 23.83 | 23.16 | 15.88 |
| 20VLP2-1 | I | 4 | 6.12 | 5.54 | 5.35 |
| 20VLP2-11 | I | 6 | 6.54 | 6.43 | 6.21 |
| 20VLP2-3 | I | 1 | 9.03 | 8.45 | 5.98 |
| 20VLP2-6 | I | 2 | 4.48 | 4.37 | 4.65 |
| 20VLP2-7 | I | 6 | 6.71 | 6.29 | 5.00 |
| 20VLP2-8 | I | 4 | 5.37 | 5.19 | 5.34 |
| 20VLP4-6 | I | 19 | 8.18 | 6.91 | 3.99 |
| 19GAR1-52 | II | 20 | 14.33 | NA | NA |
| 19GAR3-100 | I/II | 2 | 14.13 | 14.66 | 6.74 |
| 19GAR3-65 | II | 2 | 14.5 | NA | NA |
| 20TP1C-1053 | I/II | 5 | 21.18 | 25.12 | 15.30 |
| 21VLP5-1 | I/II/III | 56 | 5.53 | 5.80 | 6.13 |
| 19GAR1-38 | III | 2 | 18.1 | NA | NA |
| 19GAR1-44 | III | 11 | 13.93 | NA | NA |
| 19GAR1-45 | III | 13 | 16.25 | NA | NA |
| 19GAR1-47 | III | 9 | 16.18 | NA | NA |
| 19GAR1-48 | III | 9 | 13.29 | NA | NA |
| 19GAR1-49 | III | 7 | 17.66 | NA | NA |
| 19GAR1-50 | III | 13 | 14.45 | NA | NA |
| 19GAR1-53 | III | 2 | 15.38 | NA | NA |
| 19GAR1-54 | III | 5 | 17.57 | NA | NA |
| 19GAR1-56 | III | 4 | 9.34 | NA | NA |
| 19HAMT-3 | III | 8 | 10.23 | NA | NA |
| 19HAMT-4 | III | 7 | 15.03 | NA | NA |
| 19HAMT-5 | III | 4 | 4.09 | NA | NA |
| 19HAMT-6 | III | 10 | 11.34 | NA | NA |
| 19HAMT-7 | III | 9 | 11.97 | NA | NA |
| 19HAMT-8 | III | 15 | 13.46 | NA | NA |

First column: seed lot ID; second column: cannabinoid type; third column: number of accessions per seed lot; fourth column: average value of Total Cannabinoids (%) across all three cannabinoid types per seed lot; fifth column: average value of Total THC (%) for type I accessions per seed lot (NA = no data because all accessions in seed lot were of a type other than type I); sixth column: average value of Total THC to Total CBG Ratio for type I accessions per seed lot (NA = no data because all accessions in seed lot were of a type other than type I).

TABLE 3
Data sets used for cannabinoid mapping. First column: data set number;
second column: population type type; third column: cannabinoid
type; fourth column: Total Varin % range in data set; fifth column:
number of seed lots in data set; sixth column: number of accessions
in data set; seventh column: number of SNPs used in analysis after
application of filters for low quality SNPs, missing data and minor
allele frequency (and test for Hardy-Weinberg equilibrium for the
F2 segregating for type I, II, and III).

| | | | | | | Number |
|---|---|---|---|---|---|---|
| | | Canna- | | Number | Number | of SNPs |
| | | binoid | Total | of seed | of ac- | for |
| Set | Population | type | Varin | lots | cessions | analysis |
| 1 | Diversity panel | I, II, III | 0-11.8% | 144 | 895 | 37,761 |
| 2 | Diversity panel | I | 0-2.4% | 122 | 682 | 36,494 |
| 3 | Diversity panel | III | 0-3.0% | 24 | 146 | 34,432 |
| 4 | Diversity panel | I | <0.5% | 38 | 233 | 29,833 |
| 5 | Diversity panel | I | >1.6% | 18 | 266 | 28,211 |
| 6 | F2 | I, IV | <0.5% | 1 | 85 | 9,837 |
| 7 | F2 | I, II, III | <0.5% | 1 | 200 | 7,607 |

### Flower Cannabinoid Data Collection

Cannabinoid data were obtained via HPLC (except for the F2 population grown in a growth room which was chemotyped using an Orange Photonics Light Lab) of flower (cola) tissue which was dried for at least one week. Total THC was calculated as (0.877*THCA)+THC; Total CBD was calculated as (0.877*CBDA)+CBD; Total CBG was calculated as (0.878*CBGA)+CBG; Total CBC was calculated as (0.877*CBCA)+CBC; Total THCV was calculated as (0.877*THCVA)+THCV; Total CBDV was calculated as (0.877*CBDVA)+CBDV; Total CBGV was calculated as (0.878*CBGVA)+CBGV. Total Cannabinoids were calculated as Total THC+Total CBD+Total CBG+Total CBC+Total THCV+Total CBDV+Total CBGV. Total Cannabinoids to CBG Ratio was calculated as (Total Cannabinoids+1)/(Total CBG+1), THC to CBG Ratio was calculated as (Total THC+Total THCV+1)/(Total CBG+Total CBGV+1), and CBD to CBG Ratio was calculated as (Total CBD+Total CBDV+1)/(Total CBG+Total CBGV+1). CBC to Total Cannabinoids Ratio was calculated as (Total CBC+1)/(Total Cannabinoids+1). CBC to CBG Ratio was calculated as (Total CBC+1)/(Total CBG+Total CBGV+1). Total Varin was calculated as Total THCV+Total CBDV+Total CBGV.

Total CBG+Total CBGV constitutes the remaining CBG and CBGV after conversion to cannabinoids. This value together with THC to CBG Ratio is an indication of conversion efficiency in which a plant converts CBG to THC, whereas Total Cannabinoids is an indication of CBG and CBGV precursor production.

### Association Mapping

The set of 144 diverse seed lots (n=895; set 1 table 3) was genotyped with an Illumina bead array. After initial SNP QC, further filtering steps were performed to filter out known low quality SNPs, followed by filtering for missing data (<10%) and minor allele frequency (>1%) using vcftools (Danecek, Petr, et al. “The variant call format and VCFtools.” Bioinformatics 27.15 (2011): 2156-2158). This resulted in 37,761 array SNPs for input in nested association mapping (NAM) analysis. This set consisting of 682 type I accessions (set 2 Table 3), after application of the same filters for QC, low quality, missing data and minor allele frequency contained 36,494 SNPs for analysis. Application of the same filters on the set of 146 type III accessions (set 3 Table 3) resulted in 34,432 SNPs for analysis. Application of these filters on sets 4-6 resulted in 9,837-29,833 SNPs for analysis (Table 3). Missing data were subsequently imputed (R package NAM “snpQC” option; Xavier, Alencar, et al. “NAM: association studies in multiple populations.” Bioinformatics 31.23 (2015): 3862-3864). NAM was the R NAM performed using package (https://cran.r-project.org/web/packages/NAM/index.html) using seed lots as family structure and a kinship matrix to control for relatedness (GWAS2 function).

NAM of Total Cannabinoids for all three types in the set of 895 accessions (set 1 Table 3) identified three significant SNPs (p<1.32E-06 Bonferroni threshold; SEQ ID NOs: 1, 11, and 22, Table 4). SNP marker 90_707845 is located at position 1,605,949 on chromosome 1 (p=2.19E-07), SNP marker 141928_866974 is located at position 511,858 on chromosome 2 (p=1.02E-06), and SNP marker 194726_3613 is located at position 68,168,149 on chromosome 3 (p=5.62E-08). Two of these three SNP markers (90_707845 and 194726_3613) were significantly associated (p=3.24E-07 and 1.15E-07, respectively) with Total THC+Total THCV in the set of 682 type I accessions (set 2 Table 3; SEQ ID Nos: 1 and 22; Table 5). A second SNP ˜1 Mbp from the first SNP on chromosome 1 (SEQ ID NO: 1) was found significantly associated with Total THC+THCV in this set of type I accessions (SEQ ID NO: 2; 90_1205270 at position 2,187,135; p=3.98E-07; Table 5). NAM of Total Cannabinoids in the set of 682 type I accessions (set 2 Table 3) did not result in any significant associations. NAM of Total CBD+Total CBDV as well as Total Cannabinoids in the set of 146 type III accessions (set 3 Table 3) did not result in any significant associations.

TABLE 4
NAM results for Total Cannabinoids in a set of 895 accessions of all three cannabinoid types (accessions of type I, II,
and Ill; set 1 Table 3). Each respective marker is identified as a chromosome position within the CsaAba2 reference genome
as well as at position 51 of the sequence identifier described in column 1. Left and right flanking SNPs of the marker
haplotype are the first non-significant SNPs to the left and right of the SNP marker. First column, SNP marker number;
Second column, SNP marker name; Third column, NAM p-value; Fourth column, beneficial genotype (A = homozygous for
reference allele, B = homozygous for alternative allele, X = heterozygous); Fifth column, reference allele call;
Sixth column, alternative allele call; Seventh column, Abacus reference genome (version CsaAba2) chromosome. Eighth column,
Abacus reference genome (version CsaAba2) position in bp; Ninth column, left flanking SNP of haplotype surrounding SNP
marker; Tenth column, right flanking SNP of haplotype surrounding SNP marker; Eleventh column, Abacus reference genome
(version CsaAba2) position in bp for left flanking SNP of haplotype surrounding SNP marker; Twelfth column, Abacus reference
genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | Abacus | | | left | right |
| | | | | | | | reference | Left | Right | flanking | flanking |
| | | | | | | | genome | flanking | flanking | SNP of | SNP of |
| | SNP | | | | | | CsaAba2 | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 90_{—} | 2.19E−07 | A, X | T | A | 1 | 1,605,949 | 90_{—} | 90_{—} | 1,593,859 | 1,611,839 |
| NO: 1 | 707845 | | | | | | | 695755 | 713734 | | |
| SEQ ID | 141928_{—} | 1.02E−06 | B, X | G | A | 2 | 511,858 | 141928_{—} | 141928_{—} | 506,060 | 518,277 |
| NO: 11 | 866974 | | | | | | | 872772 | 860547 | | |
| SEQ ID | 194726_{—} | 5.62E−08 | B | G | A | 3 | 68,168,149 | 142044_{—} | 142044_{—} | 68,162,527 | 68,173,892 |
| NO: 22 | 3613 | | | | | | | 1814203 | 1802838 | | |

TABLE 5
NAM results for Total THC + Total THCV in a set of 682 type I accessions (set 2 Table 3). Each respective marker is identified
as a chromosome position within the CsaAba2 reference genome as well as at position 51 of the sequence identifier described
in column 1. Left and right flanking SNPs of the marker haplotype are the first non-significant SNPs to the left and right
of the SNP marker. First column, SNP marker number; Second column, SNP marker name; Third column, NAM p-value; Fourth column,
beneficial genotype (A = homozygous for reference allele, B = homozygous for alternative allele, X = heterozygous);
Fifth column, reference allele call; Sixth column, alternative allele call; Seventh column, Abacus reference genome (version
CsaAba2) chromosome. Eighth column, Abacus reference genome (version CsaAba2) position in bp; Ninth column, left flanking
SNP of haplotype surrounding SNP marker; Tenth column, right flanking SNP of haplotype surrounding SNP marker; Eleventh column,
Abacus reference genome (version CsaAba2) position in bp for left flanking SNP of haplotype surrounding SNP marker; Twelfth
column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | Abacus | | | left | right |
| | | | | | | | reference | Left | Right | flanking | flanking |
| | | | | | | | genome | flanking | flanking | SNP of | SNP of |
| | SNP | | | | | | CsaAba2 | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 90_{—} | 3.24E−07 | A, X | T | A | 1 | 1,605,949 | 90_{—} | 90_{—} | 1,593,859 | 1,611,839 |
| NO: 1 | 707845 | | | | | | | 695755 | 713734 | | |
| SEQ ID | 90_{—} | 3.98E−07 | B | G | A | 1 | 2,187,135 | 141820_{—} | 90_{—} | 2,183,741 | 2,194,270 |
| NO: 2 | 1205270 | | | | | | | 233661 | 1213012 | | |
| SEQ ID | 194726_{—} | 1.15E−07 | B | G | A | 3 | 68,168,149 | 142044_{—} | 142044_{—} | 68,162,527 | 68,173,892 |
| NO:22 | 3613 | | | | | | | 1814203 | 1802838 | | |

NAM of Total THC+Total THCV in the set of 233 type I low varin accessions (set 4 Table 3) identified one significant SNP (p<1.68E-06 Bonferroni threshold; Table 7; SEQ ID. 135): SNP marker 141928_166641 located at 1,981,515 bp on chromosome 7 (p=1.48E-06). The same SNP marker was also found to be significantly associated with Total Cannabinoids (p=8.13E-07; Table 6 and 7). Three additional SNP markers were significantly associated with Total Cannabinoids in this data set: SNP marker 141928_611188 at position 822,718 bp on chromosome 2, SNP marker 141928_604731 at position 829,175 bp on chromosome 2, and SNP marker 141928_547218 at position 889,775 on chromosome 2 (Table 6). All three SNP markers are part of the same locus for Total Cannabinoids on chromosome 2.

TABLE 6
NAM results for Total Cannabinoids in a set of 233 type I accessions (Total Varin <0.5%; set 4 Table 3). Each respective
marker is identified as a chromosome position within the CsaAba2 reference genome as well as at position 51 of the sequence
identifier described in column 1. Left and right flanking SNPs of the marker haplotype are the first non-significant
SNPs to the left and right of the SNP marker. First column, SNP marker number; Second column, SNP marker name; Third
column, NAM p-value; Fourth column, beneficial genotype (A = homozygous for reference allele, B = homozygous
for alternative allele, X = heterozygous), *= B inferred from segregation pattern; Fifth column, reference
allele call; Sixth column, alternative allele call; Seventh column, Abacus reference genome (version CsaAba2) chromosome.
Eighth column, Abacus reference genome (version CsaAba2) position in bp; Ninth column, left flanking SNP of haplotype
surrounding SNP marker; Tenth column, right flanking SNP of haplotype surrounding SNP marker; Eleventh column, Abacus
reference genome (version CsaAba2) position in bp for left flanking SNP of haplotype surrounding SNP marker; Twelfth
column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | Abacus | | | left | right |
| | | | | | | | reference | Left | Right | flanking | flanking |
| | | | | | | | genome | flanking | flanking | SNP of | SNP of |
| | SNP | | | | | | CsaAba2 | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 141928_{—} | 1.51E−06 | A, X | T | C | 2 | 822,718 | 141928_{—} | 141928_{—} | 812,504 | 850,793 |
| NO: 12 | 611188 | | | | | | | 621402 | 583114 | | |
| SEQ ID | 141928_{—} | 8.13E−07 | A, X | C | T | 2 | 829,175 | 141928_{—} | 141928_{—} | 812,504 | 850,793 |
| NO: 13 | 604731 | | | | | | | 621402 | 583114 | | |
| SEQ ID | 141928_{—} | 1.32E−06 | A, X | G | A | 2 | 889,775 | 141928_{—} | 141928_{—} | 887,683 | 891,812 |
| NO: 14 | 547218 | | | | | | | 549308 | 545181 | | |
| SEQ ID | 141928_{—} | 8.13E−07 | B, X* | A | T | 7 | 1,981,515 | 142713_{—} | 142713_{—} | 1,959,582 | 2,009,523 |
| NO: 135 | 166641 | | | | | | | 4053295 | 4012422 | | |

TABLE 7
NAM results for Total THC + Total THCV in a set of 233 type I accessions (Total Varin <0.5%; set 4 Table 3).
Each respective marker is identified as a chromosome position within the CsaAba2 reference genome as well as at position
51 of the sequence identifier described in column 1. Left and right flanking SNPs of the marker haplotype are the
first non-significant SNPs to the left and right of the SNP marker. First column, SNP marker number; Second column,
SNP marker name; Third column, NAM p-value; Fourth column, beneficial genotype (A = homozygous for reference
allele, B = homozygous for alternative allele, X = heterozygous); Fifth column, reference allele call;
Sixth column, alternative allele call; Seventh column, Abacus reference genome (version CsaAba2) chromosome. Eighth
column, Abacus reference genome (version CsaAba2) position in bp; Ninth column, left flanking SNP of haplotype surrounding
SNP marker; Tenth column, right flanking SNP of haplotype surrounding SNP marker; Eleventh column, Abacus reference
genome (version CsaAba2) position in bp for left flanking SNP of haplotype surrounding SNP marker; Twelfth column,
Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | Abacus | | | left | right |
| | | | | | | | reference | Left | Right | flanking | flanking |
| | | | | | | | genome | flanking | flanking | SNP of | SNP of |
| | SNP | | | | | | CsaAba2 | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 141928_{—} | 1.48E−06 | B, X | A | T | 7 | 1,981,515 | 142713_{—} | 142713_{—} | 1,959,582 | 2,009,523 |
| NO: 135 | 166641 | | | | | | | 4053295 | 4012422 | | |

NAM of Total THC+Total THCV in the set of 266 type I high varin accessions (set 5 Table 3) identified seven significant (p<1.77E-06 Bonferroni threshold) SNP markers on chromosome 6 and 7, and one significant SNP on chromosome 9 (Table 9). NAM of Total Cannabinoids in this data set identified the same seven SNP markers on chromosome 6 plus an additional significant SNP marker on this chromosome, four out of the seven SNP markers on chromosome 7 and the same SNP marker on chromosome 9 as was identified after NAM based on Total THC+Total THCV (Table 8).

TABLE 8
NAM results for Total Cannabinoids in a set of 266 type I high varin accessions (set 5 Table 3). Each respective marker is identified as a
chromosome position within the CsaAba2 reference genome as well as at position 51 of the sequence identifier described in column 1. Left
and right flanking SNPs of the marker haplotype are the first non-significant SNPs to the left and right of the SNP marker. First column,
SNP marker number; Second column, SNP marker name; Third column, NAM p-value; Fourth column, beneficial genotype (A = homozygous for
reference allele, B = homozygous for alternative allele, X = heterozygous); *= B inferred based on segregation patterns, **=
A inferred based on segregation patterns, ***= X inferred based on segregation patterns; Fifth column, reference allele call; Sixth column,
alternative allele call; Seventh column, Abacus reference genome (version CsaAba2) chromosome. Eighth column, Abacus reference genome (version
CsaAba2) position in bp; Ninth column, left flanking SNP of haplotype surrounding SNP marker; Tenth column, right flanking SNP of haplotype
surrounding SNP marker; Eleventh column, Abacus reference genome (version CsaAba2) position in bp for left flanking SNP of haplotype surrounding
SNP marker; Twelfth column, Abacus reference genome (version CsaAba2) position in bp for right flanking SNP of haplotype surrounding SNP marker.

| | | | | | | | | | | | Position | Position |
|---|---|---|---|---|---|---|---|---|---|---|---|---|
| | | | | | | | | Abacus | | | left | right |
| | | | P-value | | | | | reference | Left | Right | flanking | flanking |
| | | P-value | Total | | | | | genome | flanking | flanking | SNP of | SNP of |
| | SNP | Total | THC + | | | | Chro- | CsaAba2 | SNP of | SNP of | marker | marker |
| | marker | Canna- | Total | Geno- | Ref | Alt | mo- | position | marker | marker | haplotype | haplotype |
| SEQ ID | name | binoids | THCV | type | call | call | some | (bp) | haplotype | haplotype | (bp) | (bp) |
| SEQ ID | 323_{—} | 1.29E−06 | 7.08E−07 | B, X | A | C | 6 | 8,445,770 | 323_{—} | 142139_{—} | 8,437,332 | 8,460,311 |
| NO: 119 | 197605 | | | | | | | | 206044 | 2675 | | |
| SEQ ID | 142100_{—} | 1.17E−08 | 1.32E−07 | B, X* | C | G | 6 | 15,287,401 | 142100_{—} | 142100_{—} | 15,261,229 | 15,310,117 |
| NO: 121 | 734171 | | | | | | | | 760303 | 711291 | | |
| SEQ ID | 140390_{—} | 2.95E−07 | 7.08E−07 | A, X | G | A | 6 | 26,717,252 | 140390_{—} | 140390_{—} | 26,684,684 | 26,723,996 |
| NO: 122 | 247532 | | | | | | | | 224903 | 254278 | | |
| SEQ ID | 120908_{—} | 6.31E−08 | 1.95E−07 | A, X | C | T | 6 | 29,145,396 | 177373_{—} | 142034_{—} | 29,097,877 | 29,158,571 |
| NO: 123 | 131 | | | | | | | | 36440 | 12403 | | |
| SEQ ID | 140896_{—} | 2.88E−13 | 3.02E−12 | A, X | T | G | 6 | 43,665,911 | 168334_{—} | 135981_{—} | 43,662,000 | 44,089,274 |
| NO: 124 | 7749 | | | | | | | | 13697 | 8533 | | |
| SEQ ID | 128460_{—} | 5.13E−07 | 1.91E−06 | B, X | A | G | 6 | 45,567,480 | 140885_{—} | 140885_{—} | 45,546,384 | 45,612,738 |
| NO: 125 | 3743 | | | | | | | | 7279 | 14407 | | |
| SEQ ID | 139926_{—} | 1.02E−07 | 3.63E−07 | B, X | T | C | 6 | 57,867,261 | 139926_{—} | 139926_{—} | 57,860,326 | 57,877,559 |
| NO: 126 | 79630 | | | | | | | | 154351 | 89892 | | |
| SEQ ID | 104456_{—} | 3.09E−07 | 6.92E−07 | A, X ** | T | A | 6 | 67,382,064 | 339_{—} | 339_{—} | 67,379,156 | 67,421,842 |
| NO: 127 | 347 | | | | | | | | 87955 | 126377 | | |
| SEQ ID | 122295_{—} | 3.31E−06 | 5.01E−07 | B, X* | T | C | 7 | 60,624 | 113435_{—} | 125685_{—} | 15,258 | 115,293 |
| NO: 128 | 716 | | | | | | | | 5260 | 2461 | | |
| SEQ ID | 142713_{—} | 8.51E−09 | 4.57E−09 | A, X | A | G | 7 | 3,441,223 | 142713_{—} | 142713_{—} | 3,412,885 | 3,477,646 |
| NO: 138 | 2757703 | | | | | | | | 2783763 | 2723486 | | |
| SEQ ID | 142713_{—} | 4.37E−06 | 1.51E−06 | B, X | T | G | 7 | 5,752,776 | 142713_{—} | 136103_{—} | 5,746,452 | 5,845,317 |
| NO: 140 | 697174 | | | | | | | | 703498 | 1788 | | |
| SEQ ID | 142713_{—} | 4.37E−06 | 1.38E−06 | B, X | A | T | 7 | 6,225,622 | 142713_{—} | 142713_{—} | 6,221,113 | 6,256,430 |
| NO: 145 | 275962 | | | | | | | | 280471 | 259973 | | |
| SEQ ID | 61055_{—} | 1.55E−06 | 5.37E−07 | B, X | A | T | 7 | 6,340,996 | 124644_{—} | 142713_{—} | 6,337,560 | 6,410,830 |
| NO: 146 | 4286 | | | | | | | | 11023 | 157782 | | |
| SEQ ID | 141963_{—} | 1.15E−06 | 3.55E−07 | B, X | G | T | 7 | 7,643,910 | 141963_{—} | 141963_{—} | 7,639,988 | 7,658,574 |
| NO: 150 | 1527311 | | | | | | | | 1531282 | 1522118 | | |
| SEQ ID | 141246_{—} | 1.07E−07 | 2.19E−08 | A, X | C | T | 7 | 23,406,044 | 139024_{—} | 141246_{—} | 23,372,911 | 23,432,690 |
| NO: 188 | 683086 | | | | | | | | 22776 | 709730 | | |
| SEQ ID | 424_{—} | 9.12E−08 | 2.09E−07 | A, X*** | A | G | 9 | 7,624,628 | 424_{—} | 424_{—} | 7,596,844 | 7,632,863 |
| NO: 360 | 3044082 | | | | | | | | 3016300 | 3052317 | | |

TABLE 9
NAM results for Total THC + Total THCV in a set of 266 type I high varin accessions (set 5 Table 3). Each respective marker is identified
as a chromosome position within the CsaAba2 reference genome as well as at position 51 of the sequence identifier described in column 1. Left
and right flanking SNPs of the marker haplotype are the first non-significant SNPs to the left and right of the SNP marker. First column,
SNP marker number; Second column, SNP marker name; Third column, NAM p-value; Fourth column, beneficial genotype (A = homozygous for
reference allele, B = homozygous for alternative allele, X = heterozygous); Fifth column, reference allele call; Sixth column, alternative
allele call; Seventh column, Abacus reference genome (version CsaAba2) chromosome. Eighth column, Abacus reference genome (version CsaAba2)
position in bp; Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding SNP marker;
Twelfth column, Abacus reference genome (version CsaAba2) position in bp for right flanking SNP of haplotype surrounding SNP marker.

| | | | | | | | Abacus | | | Position | Position |
|---|---|---|---|---|---|---|---|---|---|---|---|
| | | | | | | | reference | Left | Right | left | right |
| | | | | | | | genome | flanking | flanking | SNP of | SNP of |
| | SNP | | | | | | CsaAba2 | SNP of | SNP of | marker | marker |
| | marker | | Geno- | Ref | Alt | | position | marker | marker | haplotype | haplotype |
| SEQ ID | name | P-value | type | call | call | Chromosome | (bp) | haplotype | haplotype | (bp) | (bp) |
| SEQ ID | 323_{—} | 7.08E−07 | B, X | A | C | 6 | 8,445,770 | 323_{—} | 142139_{—} | 8,437,332 | 8,460,311 |
| NO: 119 | 197605 | | | | | | | 206044 | 2675 | | |
| SEQ ID | 142100_{—} | 1.32E−07 | B, X | C | G | 6 | 15,287,401 | 142100_{—} | 142100_{—} | 15,261,229 | 15,310,117 |
| NO: 121 | 734171 | | | | | | | 760303 | 711291 | | |
| SEQ ID | 140390_{—} | 7.08E−07 | A, X | G | A | 6 | 26,717,252 | 140390_{—} | 140390_{—} | 26,684,684 | 26,723,996 |
| NO: 122 | 247532 | | | | | | | 224903 | 254278 | | |
| SEQ ID | 120908_{—} | 1.95E−07 | A, X | C | T | 6 | 29,145,396 | 177373_{—} | 142034_{—} | 29,097,877 | 29,158,571 |
| NO: 123 | 131 | | | | | | | 36440 | 12403 | | |
| SEQ ID | 140896_{—} | 3.02E−12 | A, X | T | G | 6 | 43,665,911 | 168334_{—} | 135981_{—} | 43,662,000 | 44,089,274 |
| NO: 124 | 7749 | | | | | | | 13697 | 8533 | | |
| SEQ ID | 139926_{—} | 3.63E−07 | B, X | T | C | 6 | 57,867,261 | 139926_{—} | 139926_{—} | 57,860,326 | 57,877,559 |
| NO: 126 | 79630 | | | | | | | 154351 | 89892 | | |
| SEQ ID | 104456_{—} | 6.92E−07 | A, X | T | A | 6 | 67,382,064 | 339_{—} | 339_{—} | 67,379,156 | 67,421,842 |
| NO: 127 | 347 | | | | | | | 87955 | 126377 | | |
| SEQ ID | 122295_{—} | 5.01E−07 | B, X | T | C | 7 | 60,624 | 113435_{—} | 125685_{—} | 15,258 | 115,293 |
| NO: 128 | 716 | | | | | | | 5260 | 2461 | | |
| SEQ ID | 142713_{—} | 4.57E−09 | A, X | A | G | 7 | 3,441,223 | 142713_{—} | 142713_{—} | 3,412,885 | 3,477,646 |
| NO: 138 | 2757703 | | | | | | | 2783763 | 2723486 | | |
| SEQ ID | 142713_{—} | 1.51E−06 | B, X | T | G | 7 | 5,752,776 | 142713_{—} | 136103_{—} | 5,746,452 | 5,845,317 |
| NO: 140 | 697174 | | | | | | | 703498 | 1788 | | |
| SEQ ID | 142713_{—} | 1.38E−06 | B, X | A | T | 7 | 6,225,622 | 142713_{—} | 142713_{—} | 6,221,113 | 6,256,430 |
| NO: 145 | 275962 | | | | | | | 280471 | 259973 | | |
| SEQ ID | 61055_{—} | 5.37E−07 | B, X | A | T | 7 | 6,340,996 | 124644_{—} | 142713_{—} | 6,337,560 | 6,410,830 |
| NO: 146 | 4286 | | | | | | | 11023 | 157782 | | |
| SEQ ID | 141963_{—} | 3.55E−07 | B, X | G | T | 7 | 7,643,910 | 141963_{—} | 141963_{—} | 7,639,988 | 7,658,574 |
| NO: 150 | 1527311 | | | | | | | 1531282 | 1522118 | | |
| SEQ ID | 141246_{—} | 2.19E−08 | A, X | C | T | 7 | 23,406,044 | 139024_{—} | 141246_{—} | 23,372,911 | 23,432,690 |
| NO: 188 | 683086 | | | | | | | 22776 | 709730 | | |
| SEQ ID | 424_{—} | 2.09E−07 | A, X | A | G | 9 | 7,624,628 | 424_{—} | 424_{—} | 7,596,844 | 7,632,863 |
| NO: 360 | 3044082 | | | | | | | 3016300 | 3052317 | | |

NAM of Total THC+Total THCV in the set of 85 F2 accessions segregating for type I and IV (set 6 Table 3) identified two significant (p<5.08E-06 Bonferroni threshold) SNP markers on chromosome 1 and 3, one significant SNP marker on chromosome 4, 18 significant SNP markers on chromosome 7 (6 of these SNPs are part of a locus between positions 52,923,743 and 58,538,433 on chromosome 7), and one significant SNP marker on chromosome 9 (Table 10).

TABLE 10
NAM results for Total THC + Total THCV in a set of 85 type I and IV segregating F2 accessions (set 6 Table 3). Each respective marker
is identified as a chromosome position within the CsaAba2 reference genome as well as at position 51 of the sequence identifier described
in column 1. Left and right flanking SNPs of the marker haplotype are the first non-significant SNPs to the left and right of the SNP
marker. First column, SNP marker number; Second column, SNP marker name; Third column, NAM p-value; Fourth column, beneficial genotype
(A = homozygous for reference allele, B = homozygous for alternative allele, X = heterozygous), *= B inferred based
on segregation patterns, **= X inferred based on segregation patterns; Fifth column, reference allele call; Sixth column, alternative
allele call; Seventh column, Abacus reference genome (version CsaAba2) chromosome. Eighth column, Abacus reference genome (version CsaAba2)
position in bp; Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding SNP
marker; Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | Abacus | | | left | right |
| | | | | | | | reference | Left | Right | flanking | flanking |
| | | | | | | | genome | flanking | flanking | SNP of | SNP of |
| | SNP | | | | | | CsaAba2 | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 123886_{—} | 5.13E−12 | B, X | G | A | 1 | 38,978,759 | 335_{—} | 335_{—} | 38,944,628 | 39,073,783 |
| NO: 4 | 2182 | | | | | | | 10251 | 146423 | | |
| SEQ ID | 211_{—} | 2.75E−09 | B, X* | G | A | 1 | 67,769,631 | 177642_{—} | 211_{—} | 67,761,686 | 67,892,254 |
| NO: 8 | 137879 | | | | | | | 4242 | 40813 | | |
| SEQ ID | 142169_{—} | 1.29E−07 | B, X* | T | A | 3 | 16,010,588 | 142169_{—} | 142169_{—} | 15,652,280 | 16,182,525 |
| NO: 20 | 3375917 | | | | | | | 3070479 | 3507807 | | |
| SEQ ID | 192305_{—} | 1.70E−11 | B, X* | G | A | 3 | 39,837,146 | 141681_{—} | 141681_{—} | 39,699,406 | 40,350,847 |
| NO: 21 | 372 | | | | | | | 308495 | 865243 | | |
| SEQ ID | 142603_{—} | 9.55E−08 | B, X* | A | G | 4 | 2,127,802 | 142603_{—} | 142603_{—} | 2,082,401 | 2,155,110 |
| NO: 23 | 10585838 | | | | | | | 10626237 | 10558530 | | |
| SEQ ID | 133563_{—} | 2.00E−09 | A, X | C | T | 7 | 6,705,244 | 126388_{—} | 141963_{—} | 6,622,471 | 6,808,016 |
| NO: 147 | 14816 | | | | | | | 2166 | 2224316 | | |
| SEQ ID | 121207_{—} | 3.02E−06 | B, X* | C | T | 7 | 11,063,067 | 141801_{—} | 141801_{—} | 10,965,365 | 11,193,689 |
| NO: 164 | 1396 | | | | | | | 189282 | 349680 | | |
| SEQ ID | 141801_{—} | 2.69E−08 | B, X* | G | A | 7 | 11,067,412 | 141801_{—} | 141801_{—} | 10,965,365 | 11,193,689 |
| NO: 165 | 250754 | | | | | | | 189282 | 349680 | | |
| SEQ ID | 142257_{—} | 3.02E−06 | B, X* | G | T | 7 | 17,000,256 | 142257_{—} | 140408_{—} | 16,992,324 | 17,419,297 |
| NO: 179 | 3011452 | | | | | | | 3003533 | 52694 | | |
| SEQ ID | 142257_{—} | 3.02E−06 | B, X* | T | C | 7 | 17,008,461 | 142257_{—} | 140408_{—} | 16,992,324 | 17,419,297 |
| NO: 180 | 3019506 | | | | | | | 3003533 | 52694 | | |
| SEQ ID | 141356_{—} | 6.17E−11 | B, X** | G | C | 7 | 28,081,703 | 141356_{—} | 141356_{—} | 27,759,260 | 28,263,307 |
| NO: 191 | 607414 | | | | | | | 889685 | 457676 | | |
| SEQ ID | 141356_{—} | 6.17E−11 | B, X** | C | T | 7 | 28,685,688 | 160678_{—} | Cannabis.v1_{—} | 28,594,408 | 29,061,134 |
| NO: 192 | 120636 | | | | | | | 5015 | scf2920- | | |
| | | | | | | | | | 38973_127 | | |
| SEQ ID | 141405_{—} | 1.58E−08 | B, X* | C | A | 7 | 30,520,237 | 141405_{—} | 163837_{—} | 29,891,019 | 30,608,774 |
| NO: 200 | 11474 | | | | | | | 624010 | 179 | | |
| SEQ ID | 134489_{—} | 2.00E−06 | B, X | T | C | 7 | 32,259,550 | 163837_{—} | 134489_{—} | 30,608,774 | 32,279,982 |
| NO: 202 | 82446 | | | | | | | 179 | 69901 | | |
| SEQ ID | 141673_{—} | 1.12E−07 | B | C | T | 7 | 36,589,991 | 141673_{—} | 141673_{—} | 36,579,046 | 36,880,336 |
| NO: 212 | 555374 | | | | | | | 544430 | 797742 | | |
| SEQ ID | 128722_{—} | 2.51E−08 | B, X | A | T | 7 | 37,130,207 | 141673_{—} | 141673_{—} | 37,049,888 | 37,211,605 |
| NO: 213 | 7519 | | | | | | | 952580 | 985209 | | |
| SEQ ID | 91474_{—} | 6.31E−07 | A, X | C | T | 7 | 47,324,655 | 142465_{—} | 134245_{—} | 46,967,630 | 48,198,578 |
| NO: 231 | 12377 | | | | | | | 1389141 | 2769 | | |
| SEQ ID | 141735_{—} | 5.75E−18 | B, X* | C | A | 7 | 52,923,743 | 140726_{—} | 132241_{—} | 52,544,592 | 53,396,185 |
| NO: 288 | 113420 | | | | | | | 109620 | 2253 | | |
| SEQ ID | 170870_{—} | 2.40E−06 | A, X | T | C | 7 | 54,375,898 | 141735_{—} | 140997_{—} | 53,505,022 | 54,400,345 |
| NO: 293 | 14856 | | | | | | | 492163 | 363509 | | |
| SEQ ID | 142086_{—} | 5.75E−18 | B, X* | G | A | 7 | 56,032,988 | 142086_{—} | 142086_{—} | 56,018,989 | 56,076,209 |
| NO: 310 | 826368 | | | | | | | 818053 | 869588 | | |
| SEQ ID | 142086_{—} | 5.75E−18 | B, X* | G | A | 7 | 56,301,604 | 142086_{—} | 142086_{—} | 56,171,548 | 56,426,824 |
| NO: 314 | 1049946 | | | | | | | 948791 | 1157635 | | |
| SEQ ID | 141318_{—} | 5.75E−18 | B, X* | A | T | 7 | 56,967,275 | 141318_{—} | 141318_{—} | 56,910,768 | 57,069,404 |
| NO: 328 | 522267 | | | | | | | 572197 | 415904 | | |
| SEQ ID | 142593_{—} | 5.75E−18 | B, X* | C | T | 7 | 58,538,433 | 142593_{—} | 142593_{—} | 58,428,139 | 58,607,780 |
| NO: 348 | 660489 | | | | | | | 559015 | 696671 | | |
| SEQ ID | 80270_{—} | 9.55E−08 | B, X* | G | A | 9 | 46,475,498 | 141524_{—} | 141524_{—} | 46,392,138 | 46,584,908 |
| NO: 361 | 136 | | | | | | | 1180960 | 1033824 | | |

NAM of CBC to Total Cannabinoids Ratio in the set of 85 type I and IV segregating F2 accessions (set 6 Table 3) identified 29 significant SNP markers on chromosome 7, locus consisting of 26 SNPs between positions 56.0-57.2 Mbp (Table 11).

TABLE 11
NAM results for CBC to Total Cannabinoids Ratio (=(Total CBC + 1)/(Total Cannabinoids + 1)) in a set of 85 type I and type
IV segregating F2 accessions (set 6 Table 3). Each respective marker is identified as a chromosome position within the CsaAba2 reference
genome as well as at position 51 of the sequence identifier described in column 1. Left and right flanking SNPs of the marker haplotype
are the first non-significant SNPs to the left and right of the SNP marker. First column, SNP marker number; Second column, SNP marker
name; Third column, NAM p-value; Fourth column, beneficial genotype (A = homozygous for reference allele, B = homozygous
for alternative allele, X = heterozygous); Fifth column, reference allele call; Sixth column, alternative allele call; Seventh
column, Abacus reference genome (version CsaAba2) chromosome. Eighth column, Abacus reference genome (version CsaAba2) position in
bp; Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding
SNP marker; Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | Abacus | | | left | right |
| | | | | | | | reference | Left | Right | flanking | flanking |
| | | | | | | | genome | flanking | flanking | SNP of | SNP of |
| | SNP | | | | | | CsaAba2 | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 141735_{—} | 9.77E−08 | A, X | C | A | 7 | 52,923,743 | 140726_{—} | 132241_{—} | 52,544,592 | 53,396,185 |
| NO: 288 | 113420 | | | | | | | 109620 | 2253 | | |
| SEQ ID | 142086_{—} | 4.07E−06 | A, X | A | T | 7 | 55,970,630 | 142086_{—} | 142086_{—} | 55,939,712 | 56,081,903 |
| NO: 307 | 769641 | | | | | | | 738926 | 875282 | | |
| SEQ ID | 142086_{—} | 4.07E−06 | A, X | A | T | 7 | 55,984,088 | 142086_{—} | 142086_{—} | 55,939,712 | 56,081,903 |
| NO: 308 | 783150 | | | | | | | 738926 | 875282 | | |
| SEQ ID | 142086_{—} | 4.07E−06 | A, X | G | A | 7 | 56,018,989 | 142086_{—} | 142086_{—} | 55,939,712 | 56,081,903 |
| NO: 309 | 818053 | | | | | | | 738926 | 875282 | | |
| SEQ ID | 142086_{—} | 9.77E−08 | A, X | G | A | 7 | 56,032,988 | 142086_{—} | 142086_{—} | 55,939,712 | 56,081,903 |
| NO: 310 | 826368 | | | | | | | 738926 | 875282 | | |
| SEQ ID | 142086_{—} | 4.07E−06 | A, X | C | T | 7 | 56,076,209 | 142086_{—} | 142086_{—} | 55,939,712 | 56,081,903 |
| NO: 311 | 869588 | | | | | | | 738926 | 875282 | | |
| SEQ ID | 142086_{—} | 4.07E−06 | A, X | A | G | 7 | 56,171,548 | 142086_{—} | 142086_{—} | 56,166,983 | 56,434,732 |
| NO: 313 | 948791 | | | | | | | 944226 | 1165543 | | |
| SEQ ID | 142086_{—} | 9.77E−08 | A, X | G | A | 7 | 56,301,604 | 142086_{—} | 142086_{—} | 56,166,983 | 56,434,732 |
| NO: 314 | 1049946 | | | | | | | 944226 | 1165543 | | |
| SEQ ID | 142086_{—} | 4.07E−06 | A, X | T | G | 7 | 56,426,824 | 142086_{—} | 142086_{—} | 56,166,983 | 56,434,732 |
| NO: 316 | 1157635 | | | | | | | 944226 | 1165543 | | |
| SEQ ID | 142086_{—} | 4.07E−06 | B, X | A | G | 7 | 56,430,375 | 142086_{—} | 142086_{—} | 56,166,983 | 56,434,732 |
| NO: 317 | 1161186 | | | | | | | 944226 | 1165543 | | |
| SEQ ID | 142086_{—} | 4.07E−06 | A, X | C | T | 7 | 56,440,283 | 142086_{—} | 134458_{—} | 56,434,732 | 56,593,122 |
| NO: 318 | 1171094 | | | | | | | 1165543 | 15032 | | |
| SEQ ID | 142086_{—} | 4.07E−06 | B, X | A | G | 7 | 56,488,125 | 142086_{—} | 134458_{—} | 56,434,732 | 56,593,122 |
| NO: 320 | 1211275 | | | | | | | 1165543 | 15032 | | |
| SEQ ID | 142086_{—} | 4.07E−06 | B, X | T | G | 7 | 56,492,081 | 142086_{—} | 134458_{—} | 56,434,732 | 56,593,122 |
| NO: 321 | 1215230 | | | | | | | 1165543 | 15032 | | |
| SEQ ID | 142086_{—} | 4.07E−06 | A, X | C | A | 7 | 56,538,007 | 142086_{—} | 134458_{—} | 56,434,732 | 56,593,122 |
| NO: 322 | 1258351 | | | | | | | 1165543 | 15032 | | |
| SEQ ID | 102958_{—} | 4.07E−06 | B, X | T | C | 7 | 56,700,085 | 100708_{—} | 141318_{—} | 56,620,519 | 56,809,638 |
| NO: 323 | 690 | | | | | | | 6525 | 673231 | | |
| SEQ ID | 141318_{—} | 4.07E−06 | A, X | G | C | 7 | 56,782,341 | 100708_{—} | 141318_{—} | 56,620,519 | 56,809,638 |
| NO: 324 | 699916 | | | | | | | 6525 | 673231 | | |
| SEQ ID | 141318_{—} | 4.07E−06 | A, X | C | A | 7 | 56,872,960 | 141318_{—} | 141318_{—} | 56,809,638 | 57,276,534 |
| NO: 326 | 610006 | | | | | | | 673231 | 174028 | | |
| SEQ ID | 141318_{—} | 4.07E−06 | A, X | T | C | 7 | 56,910,768 | 141318_{—} | 141318_{—} | 56,809,638 | 57,276,534 |
| NO: 327 | 572197 | | | | | | | 673231 | 174028 | | |
| SEQ ID | 141318_{—} | 9.77E−08 | A, X | A | T | 7 | 56,967,275 | 141318_{—} | 141318_{—} | 56,809,638 | 57,276,534 |
| NO: 328 | 522267 | | | | | | | 673231 | 174028 | | |
| SEQ ID | 141318_{—} | 4.07E−06 | A, X | C | T | 7 | 57,069,404 | 141318_{—} | 141318_{—} | 56,809,638 | 57,276,534 |
| NO: 329 | 415904 | | | | | | | 673231 | 174028 | | |
| SEQ ID | 141318_{—} | 4.07E−06 | A, X | C | T | 7 | 57,080,583 | 141318_{—} | 141318_{—} | 56,809,638 | 57,276,534 |
| NO: 330 | 404726 | | | | | | | 673231 | 174028 | | |
| SEQ ID | 141318_{—} | 4.07E−06 | B, X | T | A | 7 | 57,089,709 | 141318_{—} | 141318_{—} | 56,809,638 | 57,276,534 |
| NO: 331 | 395600 | | | | | | | 673231 | 174028 | | |
| SEQ ID | 141318_{—} | 4.07E−06 | A, X | C | T | 7 | 57,104,188 | 141318_{—} | 141318_{—} | 56,809,638 | 57,276,534 |
| NO: 332 | 381127 | | | | | | | 673231 | 174028 | | |
| SEQ ID | 124011_{—} | 4.07E−06 | B, X | C | T | 7 | 57,120,122 | 141318_{—} | 141318_{—} | 56,809,638 | 57,276,534 |
| NO: 333 | 4556 | | | | | | | 673231 | 174028 | | |
| SEQ ID | 141318_{—} | 4.07E−06 | B, X | G | A | 7 | 57,152,672 | 141318_{—} | 141318_{—} | 56,809,638 | 57,276,534 |
| NO: 334 | 337651 | | | | | | | 673231 | 174028 | | |
| SEQ ID | 141318_{—} | 4.07E−06 | B, X | A | G | 7 | 57,228,643 | 141318_{—} | 141318_{—} | 56,809,638 | 57,276,534 |
| NO: 335 | 264113 | | | | | | | 673231 | 174028 | | |
| SEQ ID | 141318_{—} | 4.07E−06 | A, X | A | G | 7 | 57,233,796 | 141318_{—} | 141318_{—} | 56,809,638 | 57,276,534 |
| NO: 336 | 269266 | | | | | | | 673231 | 174028 | | |
| SEQ ID | 142593_{—} | 3.24E−06 | B, X | T | A | 7 | 58,275,951 | 142593_{—} | 139328_{—} | 58,244,327 | 58,353,998 |
| NO: 343 | 437322 | | | | | | | 405698 | 269 | | |
| SEQ ID | 142593_{—} | 9.77E−08 | A, X | C | T | 7 | 58,538,433 | 142593_{—} | 142593_{—} | 58,428,139 | 58,607,780 |
| NO: 348 | 660489 | | | | | | | 559015 | 696671 | | |

NAM of Total CBG+Total CBGV in the set of 895 type I, II, and Ill accessions (set 1 Table 3) identified 83 significant SNPs on all chromosomes; chromosome 7 contains a locus consisting of 10 SNPs between positions 49.6-52.6 Mbp (Table 12). NAM of Total CBG+Total CBGV in the set of 682 type I accessions (set 2 Table 3) identified 32 significant SNPs on chromosomes 1, 5, 6, 7, 8, 9, and X; 11 SNPs are part of a locus between positions 50.7-52.6 Mbp on chromosome 7 (Table 13). NAM of Total CBG+Total CBGV in the set of 233 low varin type I accessions (set 4 Table 3) identified one locus on chromosome 2 (three significant SNP markers; Table 14). NAM of Total CBG+Total CBGV in the set of 266 high varin type I accessions (set 5 Table 3) identified three significant SNPs on chromosome 1, one locus (consisting of 11 significant SNPs) between positions 2.1-4.4 Mbp, another locus (three significant SNPs) between positions 20.0-20.1 Mbp, as well as two additional significant SNPs on chromosome 5 and one significant SNP on chromosome X (Table 15). NAM of Total CBG+Total CBGV in the set of 85 segregating type I and IV F2 accessions (set 6 Table 3) identified 24 significant SNPs on chromosomes 1, 3, 4, 6, 7, and 9; one locus consisting of 5 SNPs between positions 52.9-59.5 Mbp on chromosome 7 (Table 16).

TABLE 12
NAM results for Total CBG + Total CBGV in a set of 895 type I, II, and III accessions (set 1 Table 3). Each respective marker is
identified as a chromosome position within the CsaAba2 reference genome as well as at position 51of the sequence identifier described
in column 1. Left and right flanking SNPs of the marker haplotype are the first non-significant SNPs to the left and right of the SNP
marker. First column, SNP marker number; Second column, SNP marker name; Third column, NAM p-value; Fourth column, beneficial genotype
(A = homozygous for reference allele, B = homozygous for alternative allele, X = heterozygous), *= B inferred based
on segregation patterns, **= A inferred based on segregation patterns; Fifth column, reference allele call; Sixth column, alternative
allele call; Seventh column, Abacus reference genome (version CsaAba2) chromosome. Eighth column, Abacus reference genome (version CsaAba2)
position in bp; Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding
SNP marker; Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | Chro- | genome | SNP of | SNP of | marker | marker |
| | marker | | Geno- | Ref | Alt | mo- | position | marker | marker | haplotype | haplotype |
| SEQ ID | name | p-value | type | call | call | some | (bp) | haplotype | haplotype | (bp) | (bp) |
| SEQ ID | 139707_{—} | 7.08E−07 | A, X | C | T | 1 | 32,398,208 | 271_{—} | Cannabis.v1_{—} | 32,414,393 | 32,414,393 |
| NO: 3 | 9823 | | | | | | | 284600 | scf1708- | | |
| | | | | | | | | | 94356_101 | | |
| SEQ ID | 141928_1 | 8.13E−07 | B, X | T | A | 2 | 1,961,209 | 141928_{—} | 141928_{—} | 1,966,129 | 1,966,129 |
| NO: 16 | 706518 | | | | | | | 1690100 | 1711436 | | |
| SEQ ID | Cannabis.v1_{—} | 7.41E−07 | B, X | T | G | 3 | 166,141 | 142372_{—} | 142372_{—} | 168,861 | 168,861 |
| NO: 17 | scf211- | | | | | | | 3580712 | 3576149 | | |
| | 243477_101 | | | | | | | | | | |
| SEQ ID | Cannabis.v1_{—} | 1.02E−06 | B, X* | C | T | 3 | 174,381 | 142372_{—} | 142372_{—} | 175,515 | 175,515 |
| NO: 18 | scf211- | | | | | | | 3572536 | 3569494 | | |
| | 234052_100 | | | | | | | | | | |
| SEQ ID | 142603_{—} | 8.13E−08 | B, X | C | T | 4 | 9,387,181 | 142603_{—} | 142603_{—} | 9,395,794 | 9,395,794 |
| NO: 25 | 4009627 | | | | | | | 4012253 | 4000816 | | |
| SEQ ID | 142050_{—} | 6.61E−07 | A, X | T | C | 5 | 1,173,474 | 142050_{—} | 142050_{—} | 1,194,306 | 1,194,306 |
| NO: 28 | 3752784 | | | | | | | 3763725 | 3735942 | | |
| SEQ ID | 142050_{—} | 1.07E−09 | B, X* | T | C | 5 | 1,181,979 | 142050_{—} | 142050_{—} | 1,194,306 | 1,194,306 |
| NO: 29 | 3748265 | | | | | | | 3763725 | 3735942 | | |
| SEQ ID | 142050_{—} | 7.94E−09 | B, X | T | C | 5 | 2,038,965 | 142050_{—} | 142050_{—} | 2,065,182 | 2,065,182 |
| NO: 31 | 2956805 | | | | | | | 2983781 | 2930662 | | |
| SEQ ID | 142050_{—} | 6.76E−08 | A, X | G | C | 5 | 2,208,629 | 142050_{—} | 142050_{—} | 2,288,919 | 2,288,919 |
| NO: 34 | 2787250 | | | | | | | 2818430 | 2714906 | | |
| SEQ ID | 140767_{—} | 2.09E−07 | A, X | G | A | 6 | 4,023,145 | 140767_{—} | 140767_{—} | 4,037,935 | 4,037,935 |
| NO: 118 | 20769 | | | | | | | 41489 | 5964 | | |
| SEQ ID | Cannabis.v1_{—} | 4.47E−07 | A, X | C | G | 7 | 858,349 | 142713_{—} | 142713_{—} | 866,330 | 866,330 |
| NO: 129 | scf2008- | | | | | | | 5032015 | 5005698 | | |
| | 17655_101 | | | | | | | | | | |
| SEQ ID | 142713_{—} | 6.03E−07 | A, X | G | A | 7 | 948,328 | 142713_{—} | 142713_{—} | 975,143 | 975,143 |
| NO: 130 | 4924636 | | | | | | | 4936245 | 4898311 | | |
| SEQ ID | 142713_{—} | 2.40E−08 | A, X | T | C | 7 | 954,115 | 142713_{—} | 142713_{—} | 975,143 | 975,143 |
| NO: 131 | 4919331 | | | | | | | 4936245 | 4898311 | | |
| SEQ ID | 142713_{—} | 3.24E−07 | A, X | G | A | 7 | 1,155,154 | 142713_{—} | 142713_{—} | 1,170,814 | 1,170,814 |
| NO: 132 | 4720731 | | | | | | | 4724950 | 4705487 | | |
| SEQ ID | 142713_{—} | 2.14E−07 | B, X | C | A | 7 | 1,928,237 | Cannabis.v1_{—} | 142713_{—} | 1,932,407 | 1,932,407 |
| NO: 134 | 4084703 | | | | | | | scf25- | 4080533 | | |
| | | | | | | | | 161410_100 | | | |
| SEQ ID | 123596_{—} | 4.27E−07 | B, X | C | T | 7 | 2,243,260 | 142713_{—} | 142713_{—} | 2,262,669 | 2,262,669 |
| NO: 136 | 6054 | | | | | | | 3822857 | 3776797 | | |
| SEQ ID | 142713_{—} | 8.13E−07 | B, X | A | G | 7 | 2,612,238 | 142713_{—} | 142713_{—} | 2,670,280 | 2,670,280 |
| NO: 137 | 3450569 | | | | | | | 3452575 | 3417887 | | |
| SEQ ID | 142713_{—} | 2.00E−07 | A, X | A | G | 7 | 5,348,707 | 142713_{—} | Cannabis.v1_{—} | 5,389,490 | 5,389,490 |
| NO: 139 | 1054639 | | | | | | | 1079517 | scf90- | | |
| | | | | | | | | | 237785_100 | | |
| SEQ ID | 136103_{—} | 8.51E−07 | A, X | A | G | 7 | 5,851,251 | 136103_{—} | 116926_{—} | 5,871,508 | 5,871,508 |
| NO: 141 | 7722 | | | | | | | 1788 | 2349 | | |
| SEQ ID | 142713_{—} | 8.51E−08 | A, X | A | C | 7 | 5,900,788 | 116926_{—} | 142713_{—} | 5,908,268 | 5,908,268 |
| NO: 142 | 576343 | | | | | | | 2349 | 568710 | | |
| SEQ ID | 141963_{—} | 1.51E−07 | A, X | C | G | 7 | 9,079,344 | 141963_{—} | 141963_{—} | 9,082,114 | 9,082,114 |
| NO: 153 | 295684 | | | | | | | 318536 | 292862 | | |
| SEQ ID | 141963_{—} | 1.32E−07 | A, X** | T | C | 7 | 9,272,731 | 141963_{—} | 139018_{—} | 9,295,164 | 9,295,164 |
| NO: 154 | 145177 | | | | | | | 151147 | 8959 | | |
| SEQ ID | 141963_{—} | 5.50E−10 | A, X | T | C | 7 | 9,421,290 | 141963_{—} | 141963_{—} | 9,432,681 | 9,432,681 |
| NO: 155 | 21605 | | | | | | | 30010 | 10162 | | |
| SEQ ID | 141501_{—} | 1.23E−08 | A, X | A | G | 7 | 9,595,805 | 141501_{—} | 141501_{—} | 9,609,554 | 9,609,554 |
| NO: 156 | 36079 | | | | | | | 26176 | 49801 | | |
| SEQ ID | 141501_{—} | 2.24E−10 | A, X | A | T | 7 | 9,598,562 | 141501_{—} | 141501_{—} | 9,609,554 | 9,609,554 |
| NO: 157 | 38812 | | | | | | | 26176 | 49801 | | |
| SEQ ID | 141501_{—} | 4.57E−08 | A, X | G | A | 7 | 9,619,898 | 141501_{—} | Cannabis.v1_{—} | 9,625,781 | 9,625,781 |
| NO: 158 | 60143 | | | | | | | 54360 | scf8348- | | |
| | | | | | | | | | 7318_101 | | |
| SEQ ID | 141501_{—} | 3.16E−12 | A, X | T | C | 7 | 9,668,980 | 141501_{—} | 141501_{—} | 9,677,657 | 9,677,657 |
| NO: 160 | 109163 | | | | | | | 103072 | 117840 | | |
| SEQ ID | Cannabis.v1_{—} | 2.45E−08 | A, X | A | G | 7 | 9,716,257 | 141501_{—} | 141501_{—} | 9,716,591 | 9,716,591 |
| NO: 161 | scf3004- | | | | | | | 131864 | 147147 | | |
| | 35184_100 | | | | | | | | | | |
| SEQ ID | 141501_{—} | 1.35E−07 | A, X | G | T | 7 | 9,868,420 | 141501_{—} | 141501_{—} | 9,891,604 | 9,891,604 |
| NO: 162 | 283981 | | | | | | | 271358 | 307928 | | |
| SEQ ID | 141501_{—} | 3.80E−08 | A, X | G | A | 7 | 9,922,946 | 141501_{—} | 141501_{—} | 9,951,332 | 9,951,332 |
| NO: 163 | 338551 | | | | | | | 307928 | 358005 | | |
| SEQ ID | 141801_{—} | 1.91E−07 | A, X | G | A | 7 | 12,851,206 | 141801_{—} | 141801_{—} | 12,855,919 | 12,855,919 |
| NO: 167 | 1808917 | | | | | | | 1803353 | 1813629 | | |
| SEQ ID | 142257_{—} | 4.07E−07 | B, X | C | T | 7 | 13,928,304 | 142257_{—} | 193_{—} | 14,002,688 | 14,002,688 |
| NO: 169 | 302302 | | | | | | | 297018 | 99207 | | |
| SEQ ID | 142257_{—} | 2.75E−07 | B, X | G | A | 7 | 13,933,289 | 142257_{—} | 193_{—} | 14,002,688 | 14,002,688 |
| NO: 170 | 307287 | | | | | | | 297018 | 99207 | | |
| SEQ ID | 142257_{—} | 1.23E−06 | A, X | C | A | 7 | 14,271,073 | 142257_{—} | 142257_{—} | 14,288,814 | 14,288,814 |
| NO: 171 | 641820 | | | | | | | 613939 | 659561 | | |
| SEQ ID | 142257_{—} | 2.75E−07 | B, X | C | T | 7 | 14,579,677 | 142257_{—} | 142257_{—} | 14,592,996 | 14,592,996 |
| NO: 172 | 921419 | | | | | | | 896528 | 934794 | | |
| SEQ ID | 142257_{—} | 5.25E−07 | A, X | T | C | 7 | 14,940,971 | 142257_{—} | 142257_{—} | 14,945,873 | 14,945,873 |
| NO: 173 | 1207521 | | | | | | | 1202934 | 1212423 | | |
| SEQ ID | 142257_{—} | 8.91E−07 | A, X | G | A | 7 | 15,036,406 | 142257_{—} | 142257_{—} | 15,048,022 | 15,048,022 |
| NO: 174 | 1294752 | | | | | | | 1224103 | 1306368 | | |
| SEQ ID | 142257_{—} | 1.55E−07 | B, X | C | T | 7 | 15,091,266 | 142257_{—} | 142257_{—} | 15,110,059 | 15,110,059 |
| NO: 175 | 1345842 | | | | | | | 1315886 | 1364635 | | |
| SEQ ID | 142257_{—} | 6.31E−11 | B, X | A | C | 7 | 15,105,660 | 142257_{—} | 142257_{—} | 15,110,059 | 15,110,059 |
| NO: 176 | 1360236 | | | | | | | 1315886 | 1364635 | | |
| SEQ ID | 113379_{—} | 1.55E−07 | B, X | C | T | 7 | 15,187,940 | 132546_{—} | 113379_{—} | 15,223,104 | 15,223,104 |
| NO: 177 | 27494 | | | | | | | 5913 | 3032 | | |
| SEQ ID | 142257_{—} | 8.13E−07 | B, X | T | C | 7 | 16,603,812 | 113268_{—} | 142257_{—} | 16,609,331 | 16,609,331 |
| NO: 178 | 2622256 | | | | | | | 2339 | 2627775 | | |
| SEQ ID | 112589_{—} | 1.02E−07 | B, X | C | G | 7 | 17,355,416 | 144949_{—} | 140408_{—} | 17,363,692 | 17,363,692 |
| NO: 181 | 1106 | | | | | | | 1104 | 7684 | | |
| SEQ ID | 140408_{—} | 1.10E−07 | B, X | C | T | 7 | 17,450,215 | 140408_{—} | 140408_{—} | 17,459,892 | 17,459,892 |
| NO: 182 | 80987 | | | | | | | 68797 | 90665 | | |
| SEQ ID | 142254_{—} | 1.10E−07 | B, X | G | A | 7 | 18,095,653 | 142254_{—} | 142254_{—} | 18,109,800 | 18,109,800 |
| NO: 183 | 8625765 | | | | | | | 6864308 | 8611876 | | |
| SEQ ID | 141820_{—} | 1.32E−09 | A, X | T | C | 7 | 21,035,025 | 141820_{—} | 141820_{—} | 21,047,228 | 21,047,228 |
| NO: 186 | 1679571 | | | | | | | 1626886 | 1691546 | | |
| SEQ ID | 141246_{—} | 1.10E−06 | A, X | C | T | 7 | 22,964,819 | 141246_{—} | 141246_{—} | 22,984,143 | 22,984,143 |
| NO: 187 | 298026 | | | | | | | 226793 | 310131 | | |
| SEQ ID | 102314_{—} | 3.24E−07 | A, X | T | A | 7 | 24,653,415 | 140899_{—} | 141203_{—} | 24,673,734 | 24,673,734 |
| NO: 189 | 4697 | | | | | | | 3945 | 688463 | | |
| SEQ ID | 141673_{—} | 1.12E−08 | B, X | G | A | 7 | 36,579,046 | 141673_{—} | 141673_{—} | 36,584,469 | 36,584,469 |
| NO: 211 | 544430 | | | | | | | 532615 | 549852 | | |
| SEQ ID | 142000_{—} | 3.72E−07 | B, X | G | A | 7 | 42,289,736 | 141440_{—} | 129064_{—} | 42,379,636 | 42,379,636 |
| NO: 223 | 50342 | | | | | | | 14502 | 9654 | | |
| SEQ ID | 142587_{—} | 1.26E−09 | A, X | A | G | 7 | 44,227,026 | 142587_{—} | 142587_{—} | 44,231,810 | 44,231,810 |
| NO: 226 | 52570 | | | | | | | 39525 | 57354 | | |
| SEQ ID | 142465_{—} | 0.000001 | B, X | A | G | 7 | 48,089,881 | 142465_{—} | 142465_{—} | 48,095,767 | 48,095,767 |
| NO: 232 | 459658 | | | | | | | 568559 | 453772 | | |
| SEQ ID | 141293_{—} | 1.02E−06 | A, X | T | G | 7 | 49,618,132 | 141293_{—} | 141293_{—} | 49,628,859 | 49,628,859 |
| NO: 252 | 22467 | | | | | | | 44740 | 11669 | | |
| SEQ ID | 140888_{—} | 1.26E−06 | A, X | T | C | 7 | 50,213,053 | 140888_{—} | 140888_{—} | 50,302,149 | 50,302,149 |
| NO: 260 | 324605 | | | | | | | 309099 | 394798 | | |
| SEQ ID | 140888_{—} | 2.19E−08 | A, X | C | T | 7 | 50,221,231 | 140888_{—} | 140888_{—} | 50,302,149 | 50,302,149 |
| NO: 261 | 332783 | | | | | | | 309099 | 394798 | | |
| SEQ ID | 142269_{—} | 1.55E−07 | A, X | A | C | 7 | 50,854,826 | 61402_{—} | 142269_{—} | 50,861,059 | 50,861,059 |
| NO: 273 | 135398 | | | | | | | 592 | 141638 | | |
| SEQ ID | 142269_{—} | 1.55E−07 | A, X | C | T | 7 | 51,054,719 | 142269_{—} | 142269_{—} | 51,062,076 | 51,062,076 |
| NO: 274 | 279954 | | | | | | | 264262 | 287311 | | |
| SEQ ID | 142269_{—} | 2.40E−08 | A, X | G | A | 7 | 51,173,524 | 138948_{—} | 142269_{—} | 51,226,826 | 51,226,826 |
| NO: 275 | 389457 | | | | | | | 776 | 435176 | | |
| SEQ ID | 140250_{—} | 1.66E−08 | A, X | G | A | 7 | 52,285,032 | 102540_{—} | 140250_{—} | 52,291,349 | 52,291,349 |
| NO: 276 | 15736 | | | | | | | 5394 | 9419 | | |
| SEQ ID | 140250_{—} | 4.68E−08 | A, X | T | C | 7 | 52,296,271 | 140250_{—} | 140726_{—} | 52,322,834 | 52,322,834 |
| NO: 277 | 4497 | | | | | | | 9419 | 298459 | | |
| SEQ ID | 140726_{—} | 3.31E−07 | A, X | T | A | 7 | 52,554,676 | 140726_{—} | 129407_{—} | 52,760,459 | 52,760,459 |
| NO: 286 | 99547 | | | | | | | 102031 | 1096 | | |
| SEQ ID | 140726_{—} | 2.82E−07 | A, X | A | G | 7 | 52,561,249 | 140726_{—} | 129407_{—} | 52,760,459 | 52,760,459 |
| NO: 287 | 92974 | | | | | | | 102031 | 1096 | | |
| SEQ ID | 141735_{—} | 2.29E−10 | B, X | C | A | 7 | 53,231,544 | 141735_{—} | 141735_{—} | 53,283,191 | 53,283,191 |
| NO: 290 | 269885 | | | | | | | 261623 | 320834 | | |
| SEQ ID | 142086_{—} | 3.09E−07 | A, X | A | G | 7 | 55,716,705 | 142086_{—} | 142086_{—} | 55,731,905 | 55,731,905 |
| NO: 304 | 537884 | | | | | | | 502220 | 553084 | | |
| SEQ ID | 142086_{—} | 3.24E−12 | A, X | G | A | 7 | 56,018,989 | 142086_{—} | 142086_{—} | 56,032,988 | 56,032,988 |
| NO: 309 | 818053 | | | | | | | 800563 | 826368 | | |
| SEQ ID | 142086_{—} | 1.10E−11 | A, X | C | T | 7 | 56,076,209 | 142086_{—} | 142086_{—} | 56,081,903 | 56,081,903 |
| NO: 311 | 869588 | | | | | | | 860739 | 875282 | | |
| SEQ ID | 123168_{—} | 5.89E−07 | A, X | T | A | 7 | 56,309,021 | 142086_{—} | 142086_{—} | 56,329,245 | 56,329,245 |
| NO: 315 | 1814 | | | | | | | 1042502 | 1076113 | | |
| SEQ ID | 141318_{—} | 3.16E−07 | B, X | C | T | 7 | 56,802,628 | 141318_{—} | 141318_{—} | 56,806,750 | 56,806,750 |
| NO: 325 | 680241 | | | | | | | 686896 | 676119 | | |
| SEQ ID | 142593_{—} | 7.94E−07 | B, X | C | T | 7 | 58,933,090 | 142593_{—} | 142593_{—} | 58,943,140 | 58,943,140 |
| NO: 349 | 985262 | | | | | | | 978320 | 995090 | | |
| SEQ ID | 171_{—} | 6.17E−08 | B, X | A | G | 8 | 8,502,077 | 171_{—} | 171_{—} | 8,507,767 | 8,507,767 |
| NO: 352 | 7957663 | | | | | | | 7952747 | 7963353 | | |
| SEQ ID | 171_{—} | 5.89E−09 | B, X | C | T | 8 | 8,679,838 | 171_{—} | 171_{—} | 8,717,782 | 8,717,782 |
| NO: 353 | 8121469 | | | | | | | 8112594 | 8159679 | | |
| SEQ ID | 171_{—} | 5.01E−08 | B, X | C | T | 8 | 9,091,526 | 171_{—} | 171_{—} | 9,094,186 | 9,094,186 |
| NO: 354 | 8499463 | | | | | | | 8482342 | 8502123 | | |
| SEQ ID | 171_{—} | 5.01E−08 | B, X | C | T | 8 | 9,196,738 | 171_{—} | 171_{—} | 9,199,573 | 9,199,573 |
| NO: 355 | 8589723 | | | | | | | 8587359 | 8592558 | | |
| SEQ ID | 171_{—} | 3.39E−10 | B, X | C | T | 8 | 9,309,715 | 171_{—} | 171_{—} | 9,316,329 | 9,316,329 |
| NO: 356 | 8689494 | | | | | | | 8684912 | 8696108 | | |
| SEQ ID | 171_{—} | 6.76E−07 | B, X | T | C | 8 | 21,389,309 | 171_{—} | 171_{—} | 21,404,217 | 21,404,217 |
| NO: 357 | 14699246 | | | | | | | 14732118 | 14684338 | | |
| SEQ ID | 424_{—} | 7.08E−07 | A, X | C | T | 9 | 4,684,198 | 134460_{—} | 424_{—} | 4,692,766 | 4,692,766 |
| NO: 359 | 524674 | | | | | | | 3810 | 533240 | | |
| SEQ ID | 142293_{—} | 1.12E−07 | B, X | A | G | X | 55,358,400 | 142293_{—} | 142293_{—} | 55,391,085 | 55,391,085 |
| NO: 363 | 8670653 | | | | | | | 8666416 | 8685959 | | |
| SEQ ID | 142293_{—} | 1.12E−07 | B, X | C | T | X | 55,956,503 | 142293_{—} | 142293_{—} | 55,962,857 | 55,962,857 |
| NO: 364 | 9139942 | | | | | | | 9128282 | 9146296 | | |
| SEQ ID | 142293_{—} | 8.13E−07 | B, X | T | C | X | 56,164,045 | 142293_{—} | 142293_{—} | 56,169,204 | 56,169,204 |
| NO: 365 | 9300135 | | | | | | | 9282283 | 9305295 | | |
| SEQ ID | 142293_{—} | 1.12E−07 | B, X | G | C | X | 56,268,500 | 142293_{—} | 142293_{—} | 56,305,767 | 56,305,767 |
| NO: 366 | 9329697 | | | | | | | 9320817 | 9366772 | | |
| SEQ ID | 142293_{—} | 1.12E−07 | B, X | C | A | X | 56,281,906 | 142293_{—} | 142293_{—} | 56,305,767 | 56,305,767 |
| NO: 367 | 9343100 | | | | | | | 9320817 | 9366772 | | |
| SEQ ID | 141076_{—} | 5.01E−07 | B, X* | C | T | X | 56,610,941 | 141076_{—} | 141076_{—} | 56,652,118 | 56,652,118 |
| NO: 368 | 384537 | | | | | | | 388565 | 343360 | | |
| SEQ ID | un5002_{—} | 6.46E−08 | B, X | T | C | X | 78,814,483 | 170_{—} | 170_{—} | 78,823,688 | 78,823,688 |
| NO: 369 | 64_65 | | | | | | | 803173 | 814355 | | |
| SEQ ID | 170_{—} | 1.26E−06 | B, X | C | T | X | 79,583,866 | 170_{—} | 137565_{—} | 79,599,466 | 79,599,466 |
| NO: 370 | 1217515 | | | | | | | 1212922 | 17266 | | |

TABLE 13
NAM results for Total CBG + Total CBGV in a set of 682 type I accessions (set 2 Table 3). Each respective marker is identified
as a chromosome position within the CsaAba2 reference genome as well as at position 51 of the sequence identifier described in
column 1. Left and right flanking SNPs of the marker haplotype are the first non-significant SNPs to the left and right of the
SNP marker. First column, SNP marker number; Second column, SNP marker name; Third column, NAM p-value; Fourth column, beneficial
genotype (A = homozygous for reference allele, B = homozygous for alternative allele, X = heterozygous), *=
B inferred based on segregation patterns; Fifth column, reference allele call; Sixth column, alternative allele call; Seventh column,
Abacus reference genome (version CsaAba2) chromosome. Eighth column, Abacus reference genome (version CsaAba2) position in bp;
Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding
SNP marker; Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 139707_{—} | 1.35E−06 | A, X | C | T | 1 | 32,398,208 | 271_{—} | 110109_{—} | 32,379,705 | 32,422,316 |
| NO: 3 | 9823 | | | | | | | 287512 | 781 | | |
| SEQ ID | 331_{—} | 1.20E−06 | B, X | T | A | 5 | 166,081 | 331_{—} | 331_{—} | 158,841 | 176,716 |
| NO: 26 | 143425 | | | | | | | 136181 | 154060 | | |
| SEQ ID | 142050_{—} | 4.79E−08 | A, X | G | A | 5 | 796,653 | 142050_{—} | 142050_{—} | 790,537 | 801,459 |
| NO: 27 | 4065320 | | | | | | | 4071436 | 4060514 | | |
| SEQ ID | 142050_{—} | 1.91E−08 | B, X | T | C | 5 | 1,181,979 | 142050_{—} | 80041_{—} | 1,173,474 | 1,210,033 |
| NO: 29 | 3748265 | | | | | | | 3752784 | 1050 | | |
| SEQ ID | 142050_{—} | 1.45E−08 | B, X* | T | C | 5 | 2,038,965 | 142050_{—} | 142050_{—} | 2,011,766 | 2,065,182 |
| NO: 31 | 2956805 | | | | | | | 2983781 | 2930662 | | |
| SEQ ID | 142050_{—} | 1.35E−07 | A, X | G | C | 5 | 2,208,629 | 142050_{—} | 142050_{—} | 2,177,531 | 2,288,919 |
| NO: 34 | 2787250 | | | | | | | 2818430 | 2714906 | | |
| SEQ ID | 140767_{—} | 1.62E−08 | A, X | G | A | 6 | 4,023,145 | 140767_{—} | 140767_{—} | 4,002,406 | 4,037,935 |
| NO: 118 | 20769 | | | | | | | 41489 | 5964 | | |
| SEQ ID | 142713_{—} | 3.98E−07 | A, X | G | A | 7 | 1,755,387 | 142713_{—} | 142713_{—} | 1,739,821 | 1,760,293 |
| NO: 133 | 4248039 | | | | | | | 4263724 | 4243133 | | |
| SEQ ID | 141963_{—} | 9.55E−08 | A, X | T | C | 7 | 9,421,290 | 141963_{—} | 141963_{—} | 9,404,565 | 9,432,681 |
| NO: 155 | 21605 | | | | | | | 30010 | 10162 | | |
| SEQ ID | 141501_{—} | 3.89E−07 | A, X | A | T | 7 | 9,598,562 | 141501_{—} | 141501_{—} | 9,595,805 | 9,609,554 |
| NO: 157 | 38812 | | | | | | | 36079 | 49801 | | |
| SEQ ID | 141501_{—} | 2.00E−07 | A, X | G | A | 7 | 9,619,898 | 141501_{—} | Cannabis.v1_{—} | 9,609,554 | 9,625,781 |
| NO: 158 | 60143 | | | | | | | 49801 | scf8348- | | |
| | | | | | | | | | 7318_101 | | |
| SEQ ID | 141501_{—} | 6.92E−09 | A, X | T | C | 7 | 9,668,980 | 141501_{—} | 141501_{—} | 9,662,890 | 9,677,657 |
| NO: 160 | 109163 | | | | | | | 103072 | 117840 | | |
| SEQ ID | 142257_{—} | 5.62E−07 | B, X | A | C | 7 | 15,105,660 | 142257_{—} | 142257_{—} | 15,091,266 | 15,110,059 |
| NO: 176 | 1360236 | | | | | | | 1345842 | 1364635 | | |
| SEQ ID | 142587_{—} | 9.33E−07 | A, X | A | G | 7 | 44,227,026 | Cannabis.v1_{—} | 142587_{—} | 44,203,159 | 44,231,810 |
| NO: 226 | 52570 | | | | | | | scf1712- | 57354 | | |
| | | | | | | | | 15390_100 | | | |
| SEQ ID | 142269_{—} | 1.70E−07 | A | T | C | 7 | 50,745,259 | 196496_{—} | 142269_{—} | 50,724,154 | 50,747,700 |
| NO: 268 | 27530 | | | | | | | 1078 | 29971 | | |
| SEQ ID | 142269_{—} | 6.46E−08 | A, X | A | C | 7 | 50,854,826 | 142269_{—} | 142269_{—} | 50,822,516 | 50,861,059 |
| NO: 273 | 135398 | | | | | | | 104610 | 141638 | | |
| SEQ ID | 142269_{—} | 6.46E−08 | A, X | C | T | 7 | 51,054,719 | 142269_{—} | 140726_{—} | 51,022,401 | 52,322,834 |
| NO: 274 | 279954 | | | | | | | 264262 | 298459 | | |
| SEQ ID | 142269_{—} | 1.05E−07 | A, X | G | A | 7 | 51,173,524 | 142269_{—} | 140726_{—} | 51,022,401 | 52,322,834 |
| NO: 275 | 389457 | | | | | | | 264262 | 298459 | | |
| SEQ ID | 140250_{—} | 7.24E−08 | A, X | G | A | 7 | 52,285,032 | 142269_{—} | 140726_{—} | 51,022,401 | 52,322,834 |
| NO: 276 | 15736 | | | | | | | 264262 | 298459 | | |
| SEQ ID | 140250_{—} | 1.05E−07 | A, X | T | C | 7 | 52,296,271 | 142269_{—} | 140726_{—} | 51,022,401 | 52,322,834 |
| NO: 277 | 4497 | | | | | | | 264262 | 298459 | | |
| SEQ ID | 140726_{—} | 1.07E−06 | A, X | A | G | 7 | 52,322,834 | 140250_{—} | 140726_{—} | 52,296,271 | 52,332,628 |
| NO: 278 | 298459 | | | | | | | 4497 | 288665 | | |
| SEQ ID | 140726_{—} | 1.07E−06 | A, X | G | A | 7 | 52,332,628 | 140726_{—} | un105509_{—} | 52,322,834 | 52,441,872 |
| NO: 279 | 288665 | | | | | | | 298459 | 43_89 | | |
| SEQ ID | 126812_{—} | 2.88E−07 | A, X | G | A | 7 | 52,439,705 | 140726_{—} | un105509_{—} | 52,322,834 | 52,441,872 |
| NO: 281 | 8470 | | | | | | | 298459 | 43_89 | | |
| SEQ ID | 140726_{—} | 3.72E−07 | A, X | T | A | 7 | 52,554,676 | 140726_{—} | 140726_{—} | 52,552,192 | 52,561,249 |
| NO: 286 | 99547 | | | | | | | 102031 | 92974 | | |
| SEQ ID | 140726_{—} | 3.24E−07 | A, X | A | G | 7 | 52,561,249 | 140726_{—} | 129407_{—} | 52,554,676 | 52,760,459 |
| NO: 287 | 92974 | | | | | | | 99547 | 1096 | | |
| SEQ ID | 141735_{—} | 1.95E−07 | B, X | C | A | 7 | 53,231,544 | 141735_{—} | 141735_{—} | 53,210,452 | 53,283,191 |
| NO: 290 | 269885 | | | | | | | 292604 | 320834 | | |
| SEQ ID | 142086_{—} | 3.89E−09 | A, X | G | A | 7 | 56,018,989 | 142086_{—} | 142086_{—} | 56,001,500 | 56,032,988 |
| NO: 309 | 818053 | | | | | | | 800563 | 826368 | | |
| SEQ ID | 142086_{—} | 2.95E−09 | A, X | C | T | 7 | 56,076,209 | 142086_{—} | 142086_{—} | 56,067,360 | 56,081,903 |
| NO: 311 | 869588 | | | | | | | 860739 | 875282 | | |
| SEQ ID | 171_{—} | 9.33E−07 | B, X | C | T | 8 | 8,679,838 | 171_{—} | 171_{—} | 8,670,961 | 8,717,782 |
| NO: 353 | 8121469 | | | | | | | 8112594 | 8159679 | | |
| SEQ ID | 171_{—} | 4.47E−08 | B, X | C | T | 8 | 9,309,715 | 171_{—} | 171_{—} | 9,305,133 | 9,316,329 |
| NO: 356 | 8689494 | | | | | | | 8684912 | 8696108 | | |
| SEQ ID | 142415_{—} | 9.33E−07 | A, X | G | T | 9 | 2,690,026 | 142415_{—} | 142415_{—} | 2,680,294 | 2,697,330 |
| NO: 358 | 2566909 | | | | | | | 2557682 | 2574213 | | |
| SEQ ID | un5002_{—} | 9.77E−07 | B, X | T | C | X | 78,814,483 | 170_{—} | 170_{—} | 78,812,503 | 78,823,688 |
| NO: 369 | 64_65 | | | | | | | 803173 | 814355 | | |

TABLE 14
NAM results for Total CBG + Total CBGV in a set of 233 type I accessions (set 4 Table 3). Each respective marker
is identified as a chromosome position within the CsaAba2 reference genome as well as at position 51 of the sequence
identifier described in column 1. Left and right flanking SNPs of the marker haplotype are the first non-significant
SNPs to the left and right of the SNP marker. First column, SNP marker number; Second column, SNP marker name;
Third column, NAM p-value; Fourth column, beneficial genotype (A = homozygous for reference allele, B =
homozygous for alternative allele, X = heterozygous); Fifth column, reference allele call; Sixth column, alternative
allele call; Seventh column, Abacus reference genome (version CsaAba2) chromosome. Eighth column, Abacus reference
genome (version CsaAba2) position in bp; Ninth column, left flanking SNP of haplotype surrounding SNP marker; Tenth
column, right flanking SNP of haplotype surrounding SNP marker; Eleventh column, Abacus reference genome (version
CsaAba2) position in bp for left flanking SNP of haplotype surrounding SNP marker; Twelfth column, Abacus reference
genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 141928_{—} | 1.23E−06 | B, X | G | A | 2 | 122,735 | 141928_{—} | 141928_{—} | 119,953 | 136,319 |
| NO: 9 | 1221494 | | | | | | | 1224277 | 1208195 | | |
| SEQ ID | 141928_{—} | 1.05E−07 | B, X | T | C | 2 | 188,542 | 141928_{—} | 141928_{—} | 181,346 | 196,868 |
| NO: 10 | 1159985 | | | | | | | 1163175 | 1151659 | | |
| SEQ ID | 141928_{—} | 1.02E−07 | B, X | T | C | 2 | 1,785,269 | 141928_{—} | 141928_{—} | 1,767,164 | 1,791,640 |
| NO: 15 | 1533875 | | | | | | | 1515774 | 1540244 | | |

TABLE 15
NAM results for Total CBG + Total CBGV in a set of 266 high varin type I accessions (set 5 Table 3). Each respective marker
is identified as a chromosome position within the CsaAba2 reference genome as well as at position 51 of the sequence identifier
described in column 1. Left and right flanking SNPs of the marker haplotype are the first non-significant SNPs to the left and right
of the SNP marker. First column, SNP marker number; Second column, SNP marker name; Third column, NAM p-value; Fourth column, beneficial
genotype (A = homozygous for reference allele, B = homozygous for alternative allele, X = heterozygous), *=
B inferred based on segregation patterns; Fifth column, reference allele call; Sixth column, alternative allele call; Seventh column,
Abacus reference genome (version CsaAba2) chromosome. Eighth column, Abacus reference genome (version CsaAba2) position in bp; Ninth
column, left flanking SNP of haplotype surrounding SNP marker; Tenth column, right flanking SNP of haplotype surrounding SNP marker;
Eleventh column, Abacus reference genome (version CsaAba2) position in bp for left flanking SNP of haplotype surrounding SNP marker;
Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 300_{—} | 8.32E−07 | B, X | T | C | 1 | 48,670,446 | 138706_{—} | 300_{—} | 48,665,164 | 48,727,602 |
| NO: 5 | 56425 | | | | | | | 11040 | 84463 | | |
| SEQ ID | 166_{—} | 1.12E−06 | B, X | G | C | 1 | 64,341,256 | 166_{—} | 166_{—} | 64,338,238 | 64,349,232 |
| NO: 6 | 982294 | | | | | | | 985249 | 976188 | | |
| SEQ ID | 142264_{—} | 7.59E−07 | B, X | C | T | 1 | 67,666,507 | 140222_{—} | 211_{—} | 67,660,656 | 67,691,271 |
| NO: 7 | 961 | | | | | | | 573 | 217708 | | |
| SEQ ID | 142050_{—} | 1.62E−08 | B, X* | A | G | 5 | 2,132,683 | 142050_{—} | 142050_{—} | 2,120,881 | 2,167,503 |
| NO: 32 | 2863275 | | | | | | | 2874978 | 2828458 | | |
| SEQ ID | 142050_{—} | 5.89E−08 | B, X* | A | C | 5 | 2,177,531 | 142050_{—} | 142050_{—} | 2,167,503 | 2,208,629 |
| NO: 33 | 2818430 | | | | | | | 2828458 | 2787250 | | |
| SEQ ID | 142050_{—} | 3.72E−09 | B, X | C | T | 5 | 2,366,529 | 142050_{—} | 142050_{—} | 2,364,964 | 2,534,579 |
| NO: 36 | 2637918 | | | | | | | 2639484 | 2550524 | | |
| SEQ ID | 142050_{—} | 5.89E−09 | B, X | T | C | 5 | 2,899,343 | 142050_{—} | 142050_{—} | 2,844,078 | 2,908,474 |
| NO: 37 | 2212118 | | | | | | | 2261596 | 2202989 | | |
| SEQ ID | 142050_{—} | 4.07E−08 | B, X | C | T | 5 | 3,074,649 | 142050_{—} | 142050_{—} | 3,061,958 | 3,081,773 |
| NO: 38 | 2052266 | | | | | | | 2064957 | 2045142 | | |
| SEQ ID | 142050_{—} | 9.12E−08 | B, X | T | C | 5 | 3,086,874 | 142050_{—} | 142050_{—} | 3,081,773 | 3,089,662 |
| NO: 39 | 2040041 | | | | | | | 2045142 | 2037253 | | |
| SEQ ID | 138186_{—} | 5.25E−10 | B, X | T | C | 5 | 3,485,895 | 142050_{—} | 138186_{—} | 3,454,995 | 3,493,107 |
| NO: 41 | 4346 | | | | | | | 1726442 | 11557 | | |
| SEQ ID | 138186_{—} | 1.70E−08 | B, X | A | G | 5 | 3,535,297 | 138186_{—} | 138186_{—} | 3,526,980 | 3,541,316 |
| NO: 42 | 53738 | | | | | | | 45421 | 59758 | | |
| SEQ ID | 142050_{—} | 1.91E−07 | B, X* | G | A | 5 | 3,599,637 | 121249_{—} | Cannabis.v1_{—} | 3,585,965 | 3,604,863 |
| NO: 43 | 1719103 | | | | | | | 123 | scf876- | | |
| | | | | | | | | | 151191_101 | | |
| SEQ ID | 142050_{—} | 6.17E−07 | B, X | C | G | 5 | 3,961,139 | 142050_{—} | 142050_{—} | 3,945,751 | 3,965,771 |
| NO: 45 | 1411714 | | | | | | | 1427098 | 1407082 | | |
| SEQ ID | 142050_{—} | 1.66E−06 | B, X | A | T | 5 | 4,384,123 | 142050_{—} | 142050_{—} | 4,376,633 | 4,391,586 |
| NO: 47 | 1011763 | | | | | | | 1019253 | 1003919 | | |
| SEQ ID | 135428_{—} | 1.51E−06 | B, X | C | T | 5 | 19,988,534 | 141363_{—} | 141363_{—} | 19,976,402 | 20,026,964 |
| NO: 77 | 7370 | | | | | | | 474561 | 514500 | | |
| SEQ ID | 141363_{—} | 8.32E−07 | B, X* | T | G | 5 | 20,017,410 | 141363_{—} | 141363_{—} | 19,976,402 | 20,026,964 |
| NO: 78 | 504946 | | | | | | | 474561 | 514500 | | |
| SEQ ID | 141363_{—} | 8.32E−07 | B, X | G | A | 5 | 20,148,519 | 141363_{—} | 82188_{—} | 20,145,940 | 20,173,122 |
| NO: 79 | 624738 | | | | | | | 622159 | 1691 | | |
| SEQ ID | 140360_{—} | 1.10E−06 | B, X | C | T | 5 | 36,148,442 | 140360_{—} | 140360_{—} | 36,089,359 | 36,288,826 |
| NO: 95 | 367283 | | | | | | | 401088 | 247867 | | |
| SEQ ID | 419_{—} | 1.48E−06 | B, X* | C | A | 5 | 53,489,757 | 419_{—} | 419_{—} | 53,299,267 | 53,496,045 |
| NO: 104 | 678771 | | | | | | | 547859 | 685059 | | |
| SEQ ID | 170_{—} | 6.61E−07 | B | A | C | X | 81,104,593 | 170_{—} | 170_{—} | 81,098,479 | 81,109,058 |
| NO: 371 | 3873246 | | | | | | | 3879360 | 3868781 | | |

TABLE 16
NAM results for Total CBG + Total CBGV in a set of 85 segregating type I and IV F2 accessions (set 6 Table 3). Each respective marker
is identified as a chromosome position within the CsaAba2 reference genome as well as at position 51 of the sequence identifier described
in column 1. Left and right flanking SNPs of the marker haplotype are the first non-significant SNPs to the left and right of the SNP
marker. First column, SNP marker number; Second column, SNP marker name; Third column, NAM p-value; Fourth column, beneficial genotype
(A = homozygous for reference allele, B = homozygous for alternative allele, X = heterozygous), *= B inferred based
on segregation patterns, **= X inferred based on segregation patterns; Fifth column, reference allele call; Sixth column, alternative
allele call; Seventh column, Abacus reference genome (version CsaAba2) chromosome. Eighth column, Abacus reference genome (version CsaAba2)
position in bp; Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding SNP
marker; Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 123886_ | 3.63E−14 | A, X | G | A | 1 | 38,978,759 | 335_ | 335_ | 38,944,628 | 39,073,783 |
| NO: 4 | 2182 | | | | | | | 10251 | 146423 | | |
| SEQ ID | 211_ | 5.01E−13 | A, X | G | A | 1 | 67,769,631 | 177642_ | 211_ | 67,761,686 | 67,892,254 |
| NO: 8 | 137879 | | | | | | | 4242 | 40813 | | |
| SEQ ID | 142169_ | 4.68E−13 | A, X | T | A | 3 | 16,010,588 | 142169_ | 142169_ | 15,652,280 | 16,182,525 |
| NO: 20 | 3375917 | | | | | | | 3070479 | 3507807 | | |
| SEQ ID | 192305_ | 3.63E−14 | A, X | G | A | 3 | 39,837,146 | 141681_ | 141681_ | 39,699,406 | 40,350,847 |
| NO: 21 | 372 | | | | | | | 308495 | 865243 | | |
| SEQ ID | 142603_ | 6.76E−12 | A, X | A | G | 4 | 2,127,802 | 142603_ | 142603_ | 2,082,401 | 2,155,110 |
| NO: 23 | 10585838 | | | | | | | 10626237 | 10558530 | | |
| SEQ ID | 142100_ | 3.02E−10 | A, X | A | T | 6 | 12,788,565 | 142100_ | 142100_ | 12,779,880 | 12,825,612 |
| NO: 120 | 2816765 | | | | | | | 2825507 | 2788033 | | |
| SEQ ID | 133563_ | 7.59E−12 | B, X* | C | T | 7 | 6,705,244 | 126388_ | 141963_ | 6,622,471 | 6,808,016 |
| NO: 147 | 14816 | | | | | | | 2166 | 2224316 | | |
| SEQ ID | 121207_ | 3.98E−11 | A, X | C | T | 7 | 11,063,067 | 141801_ | 141801_ | 10,965,365 | 11,193,689 |
| NO: 164 | 1396 | | | | | | | 189282 | 349680 | | |
| SEQ ID | 141801_ | 2.14E−13 | A, X | G | A | 7 | 11,067,412 | 141801_ | 141801_ | 10,965,365 | 11,193,689 |
| NO: 165 | 250754 | | | | | | | 189282 | 349680 | | |
| SEQ ID | 141801_ | 3.02E−10 | B, X | G | A | 7 | 12,636,172 | 141801_ | 141801_ | 12,588,065 | 12,851,206 |
| NO: 166 | 1619833 | | | | | | | 1574227 | 1808917 | | |
| SEQ ID | 142257_ | 3.98E−11 | A, X | G | T | 7 | 17,000,256 | 142257_ | 140408_ | 16,992,324 | 17,419,297 |
| NO: 179 | 3011452 | | | | | | | 3003533 | 52694 | | |
| SEQ ID | 142257_ | 3.98E−11 | A, X | T | C | 7 | 17,008,461 | 142257_ | 140408_ | 16,992,324 | 17,419,297 |
| NO: 180 | 3019506 | | | | | | | 3003533 | 52694 | | |
| SEQ ID | 141356_ | 1.00E−14 | B, X** | G | C | 7 | 28,081,703 | 141356_ | 141356_ | 27,759,260 | 28,263,307 |
| NO: 191 | 607414 | | | | | | | 889685 | 457676 | | |
| SEQ ID | 141356_ | 1.00E−14 | B, X ** | C | T | 7 | 28,685,688 | 160678_ | Cannabis.v1_ | 28,594,408 | 29,061,134 |
| NO: 192 | 120636 | | | | | | | 5015 | scf2920- | | |
| | | | | | | | | | 38973_127 | | |
| SEQ ID | 141405_ | 9.77E−13 | A, X | C | A | 7 | 30,520,237 | 141405_ | 163837_ | 29,891,019 | 30,608,774 |
| NO: 200 | 11474 | | | | | | | 624010 | 179 | | |
| SEQ ID | 134489_ | 6.92E−09 | A, X | T | C | 7 | 32,259,550 | 163837_ | 134489_ | 30,608,774 | 32,279,982 |
| NO: 202 | 82446 | | | | | | | 179 | 69901 | | |
| SEQ ID | 141673_ | 5.89E−12 | A, X | C | T | 7 | 36,589,991 | 141673_ | 141673_ | 36,579,046 | 36,880,336 |
| NO: 212 | 555374 | | | | | | | 544430 | 797742 | | |
| SEQ ID | 128722_ | 6.31E−11 | A, X | A | T | 7 | 37,130,207 | 141673_ | 141673_ | 37,049,888 | 37,211,605 |
| NO: 213 | 7519 | | | | | | | 952580 | 985209 | | |
| SEQ ID | 141735_ | 6.76E−20 | A, X | C | A | 7 | 52,923,743 | 140726_ | 132241_ | 52,544,592 | 53,396,185 |
| NO: 288 | 113420 | | | | | | | 109620 | 2253 | | |
| SEQ ID | 142086_ | 6.76E−20 | A, X | G | A | 7 | 56,032,988 | 142086_ | 142086_ | 56,018,989 | 56,076,209 |
| NO: 310 | 826368 | | | | | | | 818053 | 869588 | | |
| SEQ ID | 142086_ | 6.76E−20 | A, X | G | A | 7 | 56,301,604 | 142086_ | 142086_ | 56,171,548 | 56,426,824 |
| NO: 314 | 1049946 | | | | | | | 948791 | 1157635 | | |
| SEQ ID | 141318_ | 6.76E−20 | A, X | A | T | 7 | 56,967,275 | 141318_ | 141318_ | 56,910,768 | 57,069,404 |
| NO: 328 | 522267 | | | | | | | 572197 | 415904 | | |
| SEQ ID | 142593_ | 6.76E−20 | A, X | C | T | 7 | 58,538,433 | 142593_ | 142593_ | 58,428,139 | 58,607,780 |
| NO: 348 | 660489 | | | | | | | 559015 | 696671 | | |
| SEQ ID | 80270_ | 6.76E−12 | A, X | G | A | 9 | 46,475,498 | 141524_ | 141524_ | 46,392,138 | 46,584,908 |
| NO: 361 | 136 | | | | | | | 1180960 | 1033824 | | |

NAM of Total Cannabinoids to CBG Ratio in the set of 895 type I, II, and Ill accessions (set 1 Table 3) identified 44 significant SNPs on chromosomes 1, 3, 6, and 7; one locus consisting of 5 SNPs between positions 50.9-52.3 Mbp on chromosome 7 (Table 17).

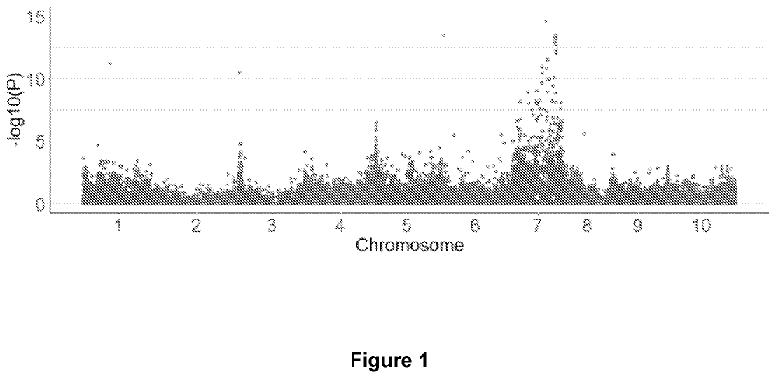

NAM of THC to CBG Ratio in the set of 682 type I accessions (set 2 Table 3) resulted in 80 significantly associated SNPs on chromosomes 1, 3, 5, 6, and 7; one locus consisting of 10 SNPs between position 49.3-52.3 Mbp on chromosome 7 (Table 18; FIG. 1). NAM of THC to CBG Ratio in the set of 233 low varin type I accessions (set 4 Table 3) identified 25 significant SNPs on chromosomes 1, 6, and 7; one locus consisting of 10 SNPs between positions 50.8-52.6 Mbp on chromosome 7 (Table 19). NAM of the THC to CBG Ratio in the set of 266 high varin type I accessions (set 5 Table 3) identified 83 significantly associated SNP markers on chromosomes 5 and 7 (Table 20). NAM of THC to CBG Ratio in the set of 85 segregating type I and IV F2 accessions (set 6 Table 3) identified 80 significant SNPs on chromosomes 1 and 7 (Table 21).

TABLE 17
NAM results for Total Cannabinoids to Total CBG Ratio (=Total Cannabinoids + 1)/(Total CBG + Total CBGV + 1)
in a set of 895 type I, II, and III accessions (set 1 Table 3). Each respective marker is identified as a chromosome position
within the CsaAba2 reference genome as well as at position 51 of the sequence identifier described in column 1. Left and right
flanking SNPs of the marker haplotype are the first non-significant SNPs to the left and right of the SNP marker. First column,
SNP marker number; Second column, SNP marker name; Third column, NAM p-value; Fourth column, beneficial genotype (A =
homozygous for reference allele, B = homozygous for alternative allele, X = heterozygous), *= B inferred based on
segregation patterns, **= A inferred based on segregation patterns; Fifth column, reference allele call; Sixth column,
alternative allele call; Seventh column, Abacus reference genome (version CsaAba2) chromosome. Eighth column, Abacus reference
genome (version CsaAba2) position in bp; Ninth column, left flanking SNP of haplotype surrounding SNP marker; Tenth column,
right flanking SNP of haplotype surrounding SNP marker; Eleventh column, Abacus reference genome (version CsaAba2) position
in bp for left flanking SNP of haplotype surrounding SNP marker; Twelfth column, Abacus reference genome (version CsaAba2)
position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 139707_ | 2.57E−10 | A, X | C | T | 1 | 32,398,208 | 271_ | Cannabis.v1_ | 32,382,605 | 32,414,393 |
| NO: 3 | 9823 | | | | | | | 284600 | scf1708- | | |
| | | | | | | | | | 94356_101 | | |
| SEQ ID | 140726_ | 1.95E−08 | A, X | G | T | 3 | 1,276,491 | 80520_ | 80520_ | 1,276,073 | 1,284,641 |
| NO: 19 | 292253 | | | | | | | 5454 | 14022 | | |
| SEQ ID | 140767_ | 8.13E−12 | B, X* | G | A | 6 | 4,023,145 | 140767_ | 140767_ | 4,002,406 | 4,037,935 |
| NO: 118 | 20769 | | | | | | | 41489 | 5964 | | |
| SEQ ID | 142713_ | 5.89E−08 | B, X | C | T | 7 | 6,147,814 | 142713_ | 142713_ | 6,125,186 | 6,165,666 |
| NO: 144 | 342957 | | | | | | | 368644 | 331562 | | |
| SEQ ID | 141963_ | 2.00E−07 | A, X | A | T | 7 | 7,857,512 | 141963_ | 141963_ | 7,822,322 | 7,864,334 |
| NO: 151 | 1357946 | | | | | | | 1375821 | 1351173 | | |
| SEQ ID | 141501_ | 1.17E−06 | B, X | A | G | 7 | 9,595,805 | 141501_ | 141501_ | 9,585,902 | 9,598,562 |
| NO: 156 | 36079 | | | | | | | 26176 | 38812 | | |
| SEQ ID | 141501_ | 3.16E−08 | B | T | C | 7 | 9,668,980 | 141501_ | 141501_ | 9,662,890 | 9,677,657 |
| NO: 160 | 109163 | | | | | | | 103072 | 117840 | | |
| SEQ ID | 142254_ | 1.32E−10 | B, X* | C | G | 7 | 18,616,969 | 79267_ | 142254_ | 18,581,032 | 18,640,383 |
| NO: 184 | 8167871 | | | | | | | 1301 | 8153669 | | |
| SEQ ID | 169366_ | 6.31E−09 | B, X | T | A | 7 | 20,028,244 | 141820_ | 141820_ | 20,022,570 | 20,050,272 |
| NO: 185 | 808 | | | | | | | 785266 | 798805 | | |
| SEQ ID | Cannabis.v1_ | 4.57E−07 | B, X* | T | C | 7 | 29,686,440 | 141405_ | 141405_ | 29,661,512 | 29,686,508 |
| NO: 194 | scf2492- | | | | | | | 872197 | 891141 | | |
| | 7828_101 | | | | | | | | | | |
| SEQ ID | 141366_ | 1.86E−07 | B, X | G | A | 7 | 34,956,997 | 141366_ | 141366_ | 34,941,497 | 34,997,619 |
| NO: 207 | 479351 | | | | | | | 463851 | 519971 | | |
| SEQ ID | 141366_ | 1.07E−09 | A, X | G | T | 7 | 35,637,532 | 141366_ | 141366_ | 35,592,132 | 35,695,775 |
| NO: 208 | 1038384 | | | | | | | 1007895 | 1086187 | | |
| SEQ ID | 141366_ | 5.01E−09 | A, X | T | A | 7 | 35,653,590 | 141366_ | 141366_ | 35,592,132 | 35,695,775 |
| NO: 209 | 1054439 | | | | | | | 1007895 | 1086187 | | |
| SEQ ID | 141673_ | 5.89E−09 | B, X | A | G | 7 | 36,003,907 | 141366_ | 141673_ | 35,799,742 | 36,153,842 |
| NO: 210 | 28623 | | | | | | | 1164229 | 162635 | | |
| SEQ ID | 141673_ | 7.59E−07 | B, X | A | T | 7 | 37,994,590 | 141673_ | 141673_ | 37,898,780 | 38,009,095 |
| NO: 215 | 1587680 | | | | | | | 1517781 | 1602182 | | |
| SEQ ID | 103034_ | 3.09E−07 | B, X | C | A | 7 | 38,736,896 | 103034_ | 140833_ | 38,729,407 | 38,794,976 |
| NO: 216 | 15764 | | | | | | | 23253 | 343246 | | |
| SEQ ID | 141440_ | 1.20E−11 | B, X | T | G | 7 | 41,296,609 | 141440_ | 141440_ | 41,248,971 | 41,343,647 |
| NO: 217 | 750937 | | | | | | | 798575 | 705988 | | |
| SEQ ID | 141440_ | 1.26E−07 | B, X | A | G | 7 | 41,986,329 | 141440_ | Cannabis.v1_ | 41,982,953 | 41,988,552 |
| NO: 220 | 188532 | | | | | | | 191907 | scf357- | | |
| | | | | | | | | | 238502_101 | | |
| SEQ ID | 125191_ | 1.05E−11 | A, X | T | C | 7 | 42,051,322 | 141440_ | 141440_ | 42,043,493 | 42,096,029 |
| NO: 221 | 848 | | | | | | | 131293 | 83423 | | |
| SEQ ID | 141440_ | 3.63E−07 | B, X | T | C | 7 | 42,152,092 | 141440_ | 141440_ | 42,096,029 | 42,156,112 |
| NO: 222 | 38014 | | | | | | | 83423 | 33994 | | |
| SEQ ID | 142000_ | 1.51E−12 | A, X** | G | A | 7 | 42,289,736 | 141440_ | 129064_ | 42,175,605 | 42,379,636 |
| NO: 223 | 50342 | | | | | | | 14502 | 9654 | | |
| SEQ ID | 142000_ | 1.05E−10 | A, X | A | G | 7 | 42,415,983 | 129064_ | 142000_ | 42,379,636 | 42,418,406 |
| NO: 224 | 155277 | | | | | | | 9654 | 157700 | | |
| SEQ ID | 142000_ | 7.41E−10 | B, X | C | T | 7 | 42,793,823 | 142000_ | 142000_ | 42,758,132 | 42,819,971 |
| NO: 225 | 474918 | | | | | | | 447709 | 501060 | | |
| SEQ ID | 142587_ | 9.33E−11 | B, X | G | A | 7 | 44,942,488 | 142587_ | 142587_ | 44,924,842 | 44,959,410 |
| NO: 229 | 645410 | | | | | | | 627764 | 662308 | | |
| SEQ ID | Cannabis.v1_ | 1.12E−06 | A, X | C | A | 7 | 46,093,742 | 142465_ | 142465_ | 46,090,347 | 46,097,781 |
| NO: 230 | scf874- | | | | | | | 2027803 | 2020369 | | |
| | 145989_100 | | | | | | | | | | |
| SEQ ID | 142465_ | 1.35E−09 | B, X | C | T | 7 | 48,399,881 | 142465_ | 142465_ | 48,393,114 | 48,443,534 |
| NO: 235 | 198069 | | | | | | | 204856 | 162626 | | |
| SEQ ID | 141293_ | 2.04E−07 | B, X | A | G | 7 | 48,949,721 | 141293_ | 141293_ | 48,862,965 | 49,016,897 |
| NO: 239 | 620463 | | | | | | | 707501 | 562032 | | |
| SEQ ID | Cannabis.v1_ | 9.77E−09 | A, X | G | C | 7 | 49,294,749 | 141293_ | 141293_ | 49,288,761 | 49,297,082 |
| NO: 246 | scf2913- | | | | | | | 331017 | 322698 | | |
| | 43590_99 | | | | | | | | | | |
| SEQ ID | 199432_ | 9.33E−07 | B, X | T | C | 7 | 50,582,962 | 140888_ | 129796_ | 50,541,967 | 50,605,020 |
| NO: 264 | 1874 | | | | | | | 589904 | 924 | | |
| SEQ ID | 142269_ | 7.24E−08 | A, X | T | C | 7 | 50,745,259 | 142269_ | 142269_ | 50,737,827 | 50,747,700 |
| NO: 268 | 27530 | | | | | | | 20098 | 29971 | | |
| SEQ ID | 142269_ | 1.07E−07 | B, X | G | C | 7 | 50,820,763 | 142269_ | 142269_ | 50,818,092 | 50,822,516 |
| NO: 271 | 102855 | | | | | | | 100184 | 104610 | | |
| SEQ ID | 142269_ | 2.24E−13 | B, X | A | C | 7 | 50,854,826 | 61402_ | 142269_ | 50,832,568 | 50,861,059 |
| NO: 273 | 135398 | | | | | | | 592 | 141638 | | |
| SEQ ID | 142269_ | 2.24E−13 | B, X | C | T | 7 | 51,054,719 | 142269_ | 142269_ | 51,022,401 | 51,062,076 |
| NO: 274 | 279954 | | | | | | | 264262 | 287311 | | |
| SEQ ID | 142269_ | 2.82E−14 | B, X | G | A | 7 | 51,173,524 | 138948_ | 142269_ | 51,141,514 | 51,226,826 |
| NO: 275 | 389457 | | | | | | | 776 | 435176 | | |
| SEQ ID | 140250_ | 1.45E−14 | B, X | G | A | 7 | 52,285,032 | 102540_ | 140250_ | 51,909,282 | 52,291,349 |
| NO: 276 | 15736 | | | | | | | 5394 | 9419 | | |
| SEQ ID | 140250_ | 5.25E−13 | B, X | T | C | 7 | 52,296,271 | 140250_ | 107140_ | 52,291,349 | 52,412,182 |
| NO: 277 | 4497 | | | | | | | 9419 | 596 | | |
| SEQ ID | 140726_ | 5.13E−10 | B, X | A | G | 7 | 52,322,834 | 140250_ | 107140_ | 52,291,349 | 52,412,182 |
| NO: 278 | 298459 | | | | | | | 9419 | 596 | | |
| SEQ ID | 140726_ | 8.32E−10 | B, X | G | A | 7 | 52,332,628 | 140250_ | 107140_ | 52,291,349 | 52,412,182 |
| NO: 279 | 288665 | | | | | | | 9419 | 596 | | |
| SEQ ID | 126812_ | 4.90E−07 | B, X | G | A | 7 | 52,439,705 | 126812_ | un105509_ | 52,435,669 | 52,441,872 |
| NO: 281 | 8470 | | | | | | | 12506 | 43_89 | | |
| SEQ ID | 140726_ | 7.76E−12 | B, X | T | A | 7 | 52,554,676 | 140726_ | 129407_ | 52,552,192 | 52,760,459 |
| NO: 286 | 99547 | | | | | | | 102031 | 1096 | | |
| SEQ ID | 140726_ | 2.40E−11 | B, X | A | G | 7 | 52,561,249 | 140726_ | 129407_ | 52,552,192 | 52,760,459 |
| NO: 287 | 92974 | | | | | | | 102031 | 1096 | | |
| SEQ ID | 140997_ | 2.82E−07 | B, X | A | G | 7 | 54,400,345 | 140997_ | 140997_ | 54,394,199 | 54,410,973 |
| NO: 294 | 363509 | | | | | | | 357365 | 374142 | | |
| SEQ ID | 142593_ | 2.24E−07 | B, X | C | T | 7 | 58,233,054 | 142593_ | Cannabis.v1_ | 58,220,261 | 58,237,329 |
| NO: 340 | 394425 | | | | | | | 381654 | scf10647- | | |
| | | | | | | | | | 281_100 | | |
| SEQ ID | 142593_ | 1.12E−06 | B, X | A | G | 7 | 59,740,097 | 142593_ | 142593_ | 59,728,563 | 59,762,135 |
| NO: 350 | 1664077 | | | | | | | 1652544 | 1686117 | | |

TABLE 18
NAM results for THC to CBG Ratio (=(Total THC + Total THCV + 1)/(Total CBG + Total CBGV + 1) in a set of 682
type I accessions (set 2 Table 3). Each respective marker is identified as a chromosome position within the CsaAba2 reference genome
as well as at position 51 of the sequence identifier described in column 1. Left and right flanking SNPs of the marker haplotype
are the first non-significant SNPs to the left and right of the SNP marker. First column, SNP marker number; Second column, SNP
marker name; Third column, NAM p-value; Fourth column, beneficial genotype (A = homozygous for reference allele, B = homozygous
for alternative allele, X = heterozygous); Fifth column, reference allele call; Sixth column, alternative allele call; Seventh
column, Abacus reference genome (version CsaAba2) chromosome. Eighth column, Abacus reference genome (version CsaAba2) position
in bp; Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding
SNP marker; Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | Chro- | genome | SNP of | SNP of | marker | marker |
| | marker | | Geno- | Ref | Alt | mo- | position | marker | marker | haplotype | haplotype |
| SEQ ID | name | p-value | type | call | call | some | (bp) | haplotype | haplotype | (bp) | (bp) |
| SEQ ID | 139707_ | 6.03E−12 | B, X | C | T | 1 | 32,398,208 | 271_ | 110109_ | 32,379,705 | 32,422,316 |
| NO: 3 | 9823 | | | | | | | 287512 | 781 | | |
| SEQ ID | 140726_ | 3.47E−11 | B, X | G | T | 3 | 1,276,491 | 80520_ | 80520_ | 1,271,584 | 1,284,641 |
| NO: 19 | 292253 | | | | | | | 965 | 14022 | | |
| SEQ ID | un71484_ | 1.29E−06 | A, X | T | C | 5 | 1,845,149 | 142050_ | 142050_ | 1,840,325 | 1,848,408 |
| NO: 30 | 59_60 | | | | | | | 3133579 | 3125495 | | |
| SEQ ID | 142050_ | 3.31E−07 | A | T | C | 5 | 2,038,965 | 142050_ | 142050_ | 2,011,766 | 2,065,182 |
| NO: 31 | 2956805 | | | | | | | 2983781 | 2930662 | | |
| SEQ ID | 142050_ | 5.89E−07 | B, X | T | C | 5 | 2,364,964 | 142050_ | 142050_ | 2,360,380 | 2,366,529 |
| NO: 35 | 2639484 | | | | | | | 2644069 | 2637918 | | |
| SEQ ID | 140767_ | 2.75E−14 | B, X | G | A | 6 | 4,023,145 | 140767_ | 140767_ | 4,002,406 | 4,037,935 |
| NO: 118 | 20769* | | | | | | | 41489 | 5964 | | |
| SEQ ID | 142713_ | 2.40E−07 | B, X | C | T | 7 | 6,147,814 | 142713_ | 142713_ | 6,125,186 | 6,165,666 |
| NO: 144 | 342957 | | | | | | | 368644 | 331562 | | |
| SEQ ID | 141963_ | 7.08E−07 | B, X | G | A | 7 | 7,085,058 | Cannabis.v1_ | 141963_ | 7,083,154 | 7,103,045 |
| NO: 148 | 1974061 | | | | | | | scf219- | 1960371 | | |
| | | | | | | | | 8150_101 | | | |
| SEQ ID | 141963_ | 8.32E−07 | A, X | A | T | 7 | 7,857,512 | 141963_ | 141963_ | 7,822,322 | 7,864,334 |
| NO: 151 | 1357946 | | | | | | | 1375821 | 1351173 | | |
| SEQ ID | 141963_ | 2.14E−07 | B, X | T | C | 7 | 8,656,466 | 141963_ | 141963_ | 8,652,992 | 8,669,729 |
| NO: 152 | 654218 | | | | | | | 657690 | 640967 | | |
| SEQ ID | 141501_ | 1.29E−06 | B, X | A | G | 7 | 9,595,805 | 141501_ | 141501_ | 9,585,902 | 9,598,562 |
| NO: 156 | 36079 | | | | | | | 26176 | 38812 | | |
| SEQ ID | Cannabis.v1_ | 1.02E−06 | B, X | C | A | 7 | 9,625,781 | 141501_ | 141501_ | 9,619,898 | 9,646,812 |
| NO: 159 | scf8348- | | | | | | | 60143 | 86994 | | |
| | 7318_101 | | | | | | | | | | |
| SEQ ID | 141501_ | 6.92E−09 | B, X | T | C | 7 | 9,668,980 | 141501_ | 141501_ | 9,662,890 | 9,677,657 |
| NO: 160 | 109163 | | | | | | | 103072 | 117840 | | |
| SEQ ID | Cannabis.v1_ | 1.62E−07 | B, X | A | G | 7 | 9,716,257 | 141501_ | 141501_ | 9,701,317 | 9,716,591 |
| NO: 161 | scf3004- | | | | | | | 131864 | 147147 | | |
| | 35184_100 | | | | | | | | | | |
| SEQ ID | 142254_ | 1.17E−09 | B, X | C | G | 7 | 18,616,969 | 79267_ | 142254_ | 18,581,032 | 18,640,383 |
| NO: 184 | 8167871 | | | | | | | 1301 | 8153669 | | |
| SEQ ID | 169366_ | 8.71E−09 | B, X | T | A | 7 | 20,028,244 | 141820_ | 141820_ | 20,022,570 | 20,050,272 |
| NO: 185 | 808 | | | | | | | 785266 | 798805 | | |
| SEQ ID | 102314_ | 3.09E−08 | B, X | T | A | 7 | 24,653,415 | 140899_ | 141203_ | 24,593,497 | 24,673,734 |
| NO: 189 | 4697 | | | | | | | 3945 | 688463 | | |
| SEQ ID | 140923_ | 7.76E−08 | B, X | G | A | 7 | 25,703,806 | 141203_ | 140923_ | 25,478,783 | 25,784,004 |
| NO: 190 | 39555 | | | | | | | 59754 | 95154 | | |
| SEQ ID | 141405_ | 6.76E−09 | B, X | T | G | 7 | 29,420,823 | 141405_ | Cannabis.v1_ | 29,416,077 | 29,482,333 |
| NO: 193 | 1135170 | | | | | | | 1139916 | scf1602.41093_ | | |
| | | | | | | | | | 100 | | |
| SEQ ID | Cannabis.v1_ | 1.74E−07 | B, X | T | C | 7 | 29,686,440 | 141405_ | 141405_ | 29,661,512 | 29,686,508 |
| NO: 194 | scf2492- | | | | | | | 872197 | 891141 | | |
| | 7828_101* | | | | | | | | | | |
| SEQ ID | 141405_ | 1.00E−08 | B, X | T | C | 7 | 30,033,193 | 141405_ | 123741_ | 29,985,750 | 30,047,823 |
| NO: 195 | 510744 | | | | | | | 1548967 | 1366 | | |
| SEQ ID | 141405_ | 6.46E−09 | B, X | T | C | 7 | 30,196,905 | 141405_ | 141405_ | 30,115,726 | 30,346,994 |
| NO: 196 | 363961 | | | | | | | 437885 | 247883 | | |
| SEQ ID | 141405_ | 2.29E−07 | B, X | T | C | 7 | 30,306,362 | 141405_ | 141405_ | 30,115,726 | 30,346,994 |
| NO: 197 | 280237 | | | | | | | 437885 | 247883 | | |
| SEQ ID | 141405_ | 8.13E−10 | B, X | C | A | 7 | 30,318,939 | 141405_ | 141405_ | 30,115,726 | 30,346,994 |
| NO: 198 | 267660 | | | | | | | 437885 | 247883 | | |
| SEQ ID | 141405_ | 9.55E−09 | B, X | G | C | 7 | 30,411,594 | 141405_ | 108628_ | 30,346,994 | 30,489,245 |
| NO: 199 | 197268 | | | | | | | 247883 | 7313 | | |
| SEQ ID | 141405_ | 1.38E−07 | B, X | T | G | 7 | 30,696,835 | 104259_ | 138450_ | 30,670,182 | 30,816,363 |
| NO: 201 | 150427 | | | | | | | 4288 | 25011 | | |
| SEQ ID | 111559_ | 2.82E−08 | B, X | A | G | 7 | 33,474,698 | 263_ | 140414_ | 33,455,822 | 33,524,411 |
| NO: 203 | 3929 | | | | | | | 499450 | 158077 | | |
| SEQ ID | 263_ | 5.13E−09 | B, X | A | C | 7 | 33,580,732 | 263_ | 263_ | 33,577,092 | 33,700,189 |
| NO: 204 | 598117 | | | | | | | 594477 | 698199 | | |
| SEQ ID | 263_ | 2.51E−08 | B, X | T | A | 7 | 33,990,610 | 142186_ | 263_ | 33,969,097 | 34,028,788 |
| NO: 205 | 949233 | | | | | | | 8487 | 970530 | | |
| SEQ ID | Cannabis.v1_ | 2.51E−08 | B, X | C | G | 7 | 34,059,134 | 263_ | 263_ | 34,028,788 | 34,136,591 |
| NO: 206 | scf1779- | | | | | | | 970530 | 1026719 | | |
| | 30705_100 | | | | | | | | | | |
| SEQ ID | 141366_ | 6.92E−08 | B, X | G | A | 7 | 34,956,997 | 141366_ | 141366_ | 34,941,497 | 34,997,619 |
| NO: 207 | 479351 | | | | | | | 463851 | 519971 | | |
| SEQ ID | 141366_ | 1.05E−11 | A, X | G | T | 7 | 35,637,532 | 141366_ | 141366_ | 35,592,132 | 35,695,775 |
| NO: 208 | 1038384 | | | | | | | 1007895 | 1086187 | | |
| SEQ ID | 141366_ | 2.09E−10 | A, X | T | A | 7 | 35,653,590 | 141366_ | 141366_ | 35,592,132 | 35,695,775 |
| NO: 209 | 1054439 | | | | | | | 1007895 | 1086187 | | |
| SEQ ID | 141673_ | 3.24E−11 | A, X | A | G | 7 | 36,003,907 | 141366_ | 141673_ | 35,799,742 | 36,153,842 |
| NO: 210 | 28623 | | | | | | | 1164229 | 162635 | | |
| SEQ ID | 103034_ | 2.51E−08 | B, X | C | A | 7 | 38,736,896 | 103034_ | 140833_ | 38,729,407 | 38,794,976 |
| NO: 216 | 15764 | | | | | | | 23253 | 343246 | | |
| SEQ ID | 141440_ | 2.51E−15 | B, X | T | G | 7 | 41,296,609 | 141440_ | 141440_ | 41,248,971 | 41,343,647 |
| NO: 217 | 750937 | | | | | | | 798575 | 705988 | | |
| SEQ ID | 141440_ | 2.24E−07 | B, X | G | A | 7 | 41,852,842 | 141440_ | 141440_ | 41,820,799 | 41,931,751 |
| NO: 219 | 311917 | | | | | | | 337223 | 274189 | | |
| SEQ ID | 141440_ | 9.77E−08 | B, X | A | G | 7 | 41,986,329 | 141440_ | Cannabis.v1_ | 41,982,953 | 41,988,552 |
| NO: 220 | 188532 | | | | | | | 191907 | scf357_ | | |
| | | | | | | | | | 238502_101 | | |
| SEQ ID | 125191_ | 1.35E−11 | A, X | T | C | 7 | 42,051,322 | 141440_ | 141440_ | 42,043,493 | 42,096,029 |
| NO: 221 | 848 | | | | | | | 131293 | 83423 | | |
| SEQ ID | 141440_ | 3.80E−08 | A, X | T | C | 7 | 42,152,092 | 141440_ | 141440_ | 42,096,029 | 42,156,112 |
| NO: 222 | 38014 | | | | | | | 83423 | 33994 | | |
| SEQ ID | 142000_ | 2.75E−12 | A, X | G | A | 7 | 42,289,736 | 141440_ | 129064_ | 42,175,605 | 42,379,636 |
| NO: 223 | 50342* | | | | | | | 14502 | 9654 | | |
| SEQ ID | 142000_ | 1.02E−10 | A, X | A | G | 7 | 42,415,983 | 129064_ | 142000_ | 42,379,636 | 42,418,406 |
| NO: 224 | 155277 | | | | | | | 9654 | 157700 | | |
| SEQ ID | 142000_ | 1.15E−09 | B, X | C | T | 7 | 42,793,823 | 142000_ | 142000_ | 42,758,132 | 42,819,971 |
| NO: 225 | 474918 | | | | | | | 447709 | 501060 | | |
| SEQ ID | 142587_ | 9.33E−07 | B, X | C | T | 7 | 44,700,019 | 142587_ | 142587_ | 44,692,277 | 44,723,890 |
| NO: 227 | 425040 | | | | | | | 417298 | 448915 | | |
| SEQ ID | 142587_ | 9.77E−11 | B, X | G | A | 7 | 44,942,488 | 142587_ | 142587_ | 44,924,842 | 44,959,410 |
| NO: 229 | 645410 | | | | | | | 627764 | 662308 | | |
| SEQ ID | Cannabis.v1_ | 4.68E−07 | A, X | C | A | 7 | 46,093,742 | 142465_ | 142465_ | 46,090,347 | 46,097,781 |
| NO: 230 | scf874- | | | | | | | 2027803 | 2020369 | | |
| | 145989_100 | | | | | | | | | | |
| SEQ ID | 142465_ | 6.03E−09 | B, X | C | T | 7 | 48,399,881 | 142465_ | 142465_ | 48,393,114 | 48,443,534 |
| NO: 235 | 198069 | | | | | | | 204856 | 162626 | | |
| SEQ ID | 141293_ | 1.38E−07 | A, X | A | G | 7 | 48,949,721 | 699733_ | 141293_ | 48,870,733 | 49,016,897 |
| NO: 239 | 620463 | | | | | | | 141293 | 562032 | | |
| SEQ ID | Cannabis.v1_ | 4.17E−10 | B, X | G | C | 7 | 49,294,749 | 141293_ | 141293_ | 49,288,761 | 49,297,082 |
| NO: 246 | scf2913- | | | | | | | 331017 | 322698 | | |
| | 43590_99 | | | | | | | | | | |
| SEQ ID | 142269_ | 8.32E−11 | B, X | T | C | 7 | 50,745,259 | 196496_ | 142269_ | 50,724,154 | 50,747,700 |
| NO: 268 | 27530 | | | | | | | 1078 | 29971 | | |
| SEQ ID | 142269_ | 3.63E−07 | B, X | G | C | 7 | 50,820,763 | 142269_ | 142269_ | 50,818,092 | 50,822,516 |
| NO: 271 | 102855 | | | | | | | 100184 | 104610 | | |
| SEQ ID | 142269_ | 1.15E−13 | B, X | A | C | 7 | 50,854,826 | 142269_ | 142269_ | 50,822,516 | 50,861,059 |
| NO: 273 | 135398 | | | | | | | 104610 | 141638 | | |
| SEQ ID | 142269_ | 1.15E−13 | B, X | C | T | 7 | 51,054,719 | 142269_ | 142269_ | 51,022,401 | 51,062,076 |
| NO: 274 | 279954 | | | | | | | 264262 | 287311 | | |
| SEQ ID | 142269_ | 5.37E−14 | B, X | G | A | 7 | 51,173,524 | 138948_ | 142269_ | 51,141,514 | 51,226,826 |
| NO: 275 | 389457 | | | | | | | 776 | 435176 | | |
| SEQ ID | 140250_ | 2.75E−14 | B, X | G | A | 7 | 52,285,032 | 102540_ | 126812_ | 51,909,282 | 52,435,669 |
| NO: 276 | 15736 | | | | | | | 5394 | 12506 | | |
| SEQ ID | 140250_ | 5.37E−14 | B, X | T | C | 7 | 52,296,271 | 102540_ | 126812_ | 51,909,282 | 52,435,669 |
| NO: 277 | 4497 | | | | | | | 5394 | 12506 | | |
| SEQ ID | 140726_ | 8.13E−13 | B, X | A | G | 7 | 52,322,834 | 102540_ | 126812_ | 51,909,282 | 52,435,669 |
| NO: 278 | 298459 | | | | | | | 5394 | 12506 | | |
| SEQ ID | 140726_ | 8.13E−13 | B, X | G | A | 7 | 52,332,628 | 102540_ | 126812_ | 51,909,282 | 52,435,669 |
| NO: 279 | 288665 | | | | | | | 5394 | 12506 | | |
| SEQ ID | 107140_ | 7.76E−09 | A, X | G | T | 7 | 52,412,182 | 102540_ | 126812_ | 51,909,282 | 52,435,669 |
| NO: 280 | 596 | | | | | | | 5394 | 12506 | | |
| SEQ ID | 126812_ | 1.35E−09 | B, X | G | A | 7 | 52,439,705 | 126812_ | un105509_ | 52,435,669 | 52,441,872 |
| NO: 281 | 8470 | | | | | | | 12506 | 43_89 | | |
| SEQ ID | 140726_ | 1.95E−13 | B, X | T | A | 7 | 52,554,676 | 140726_ | 129407_ | 52,552,192 | 52,760,459 |
| NO: 286 | 99547 | | | | | | | 102031 | 1096 | | |
| SEQ ID | 140726_ | 5.13E−13 | B, X | A | G | 7 | 52,561,249 | 140726_ | 129407_ | 52,552,192 | 52,760,459 |
| NO: 287 | 92974 | | | | | | | 102031 | 1096 | | |
| SEQ ID | 138184_ | 4.47E−07 | B, X | T | G | 7 | 52,932,343 | 142415_ | 138061_ | 52,926,238 | 53,035,545 |
| NO: 289 | 13409* | | | | | | | 1947679 | 1796 | | |
| SEQ ID | 140997_ | 1.38E−07 | B, X | A | G | 7 | 54,400,345 | 140997_ | 140997_ | 54,394,199 | 54,410,973 |
| NO: 294 | 363509 | | | | | | | 357365 | 374142 | | |
| SEQ ID | 142086_ | 1.00E−06 | B, X | G | A | 7 | 56,018,989 | 142086_ | 142086_ | 56,001,500 | 56,032,988 |
| NO: 309 | 818053 | | | | | | | 800563 | 826368 | | |
| SEQ ID | 142086_ | 4.27E−07 | B, X | C | T | 7 | 56,076,209 | 142086_ | 142086_ | 56,067,360 | 56,102,656 |
| NO: 311 | 869588 | | | | | | | 860739 | 896032 | | |
| SEQ ID | 142086_ | 9.77E−07 | B, X | T | C | 7 | 56,081,903 | 142086_ | 142086_ | 56,067,360 | 56,102,656 |
| NO: 312 | 875282 | | | | | | | 860739 | 896032 | | |
| SEQ ID | 142086_ | 5.89E−07 | B, X | T | C | 7 | 56,448,133 | 142086_ | 142086_ | 56,440,283 | 56,450,990 |
| NO: 319 | 1178755 | | | | | | | 1171094 | 1181612 | | |
| SEQ ID | 142593_ | 5.37E−07 | B, X | C | A | 7 | 57,770,425 | 142593_ | 142593_ | 57,760,733 | 57,805,989 |
| NO: 337 | 25086 | | | | | | | 15395 | 50946 | | |
| SEQ ID | 142593_ | 1.05E−06 | A, X | A | G | 7 | 58,178,218 | 59957_ | 142593_ | 58,146,715 | 58,181,665 |
| NO: 338 | 339238 | | | | | | | 545 | 342684 | | |
| SEQ ID | 142593_ | 2.51E−07 | A, X | T | C | 7 | 58,190,719 | 142593_ | 142593_ | 58,186,525 | 58,197,878 |
| NO: 339 | 351738 | | | | | | | 347544 | 358897 | | |
| SEQ ID | 142593_ | 1.32E−06 | B, X | C | T | 7 | 58,233,054 | 381654_ | Cannabis.v1_ | 58,220,261 | 58,237,329 |
| NO: 340 | 394425 | | | | | | | 142593 | scf10647- | | |
| | | | | | | | | | 281_100 | | |
| SEQ ID | 142593_ | 8.91E−07 | A, X | T | G | 7 | 58,244,327 | 142593_ | 142593_ | 58,237,682 | 58,252,232 |
| NO: 341 | 405698 | | | | | | | 399053 | 413605 | | |
| SEQ ID | 142593_ | 3.89E−07 | A, X | A | G | 7 | 58,271,366 | 142593_ | 142593_ | 58,266,320 | 58,275,951 |
| NO: 342 | 432737 | | | | | | | 427693 | 437322 | | |
| SEQ ID | 142593_ | 2.40E−08 | A, X | G | A | 7 | 58,317,539 | 142593_ | 142593_ | 58,305,695 | 58,324,561 |
| NO: 344 | 461393 | | | | | | | 44636 | 48415 | | |
| SEQ ID | 142593_ | 7.41E−09 | B, X | T | C | 7 | 58,450,928 | un32563_ | 142593_ | 58,450,413 | 58,456,572 |
| NO: 345 | 581741 | | | | | | | 47_48 | 587385 | | |
| SEQ ID | 142593_ | 1.23E−06 | B, X | T | C | 7 | 58,481,147 | 142593_ | 142593_ | 58,467,957 | 58,522,091 |
| NO: 346 | 611955 | | | | | | | 598766 | 644146 | | |
| SEQ ID | 142593_ | 2.51E−07 | B, X | A | G | 7 | 59,740,097 | 142593_ | 142593_ | 59,728,563 | 59,762,135 |
| NO: 350 | 1664077 | | | | | | | 162544 | 1686117 | | |
| SEQ ID | Cannabis.v1_ | 7.94E−07 | B | T | A | 7 | 60,366,254 | 142593_ | 142593_ | 60,363,767 | 60,366,472 |
| NO: 351 | scf7184- | | | | | | | 2223616 | 2226321 | | |
| | 962_100 | | | | | | | | | | |

TABLE 19
NAM results for THC to CBG Ratio (=(Total THC + Total THCV + 1)/(Total CBG + Total CBGV + 1) in the set of 233
type I accessions (set 4 Table 3). Each respective marker is identified as a chromosome position within the CsaAba2 reference genome
as well as at position 51 of the sequence identifier described in column 1. Left and right flanking SNPs of the marker haplotype are
the first non-significant SNPs to the left and right of the SNP marker. First column, SNP marker number; Second column, SNP marker name;
Third column, NAM p-value; Fourth column, beneficial genotype (A = homozygous for reference allele, B = homozygous for alternative
allele, X = heterozygous, *B inferred based on segregation patterns; Fifth column, reference allele call; Sixth column, alternative
allele call; Seventh column, Abacus reference genome (version CsaAba2) chromosome. Eighth column, Abacus reference genome (version CsaAba2)
position in bp; Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding
SNP marker; Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 139707_ | 3.98E−08 | B, X | C | T | 1 | 32,398,208 | 271_ | 110109_ | 32,363,260 | 32,422,316 |
| NO: 3 | 9823 | | | | | | | 304048 | 781 | | |
| SEQ ID | 140767_ | 2.82E−10 | B, X* | G | A | 6 | 4,023,145 | 140767_ | 155782_ | 4,002,406 | 4,087,346 |
| NO: 118 | 20769 | | | | | | | 41489 | 7231 | | |
| SEQ ID | 142713_ | 2.19E−08 | B, X | T | C | 7 | 5,908,268 | 142713_ | 142713_ | 5,900,788 | 5,921,575 |
| NO: 143 | 568710 | | | | | | | 576343 | 555748 | | |
| SEQ ID | 141963_ | 7.94E−09 | B, X | G | A | 7 | 7,639,988 | 141963_ | 141963_ | 7,630,020 | 7,643,910 |
| NO: 149 | 1531282 | | | | | | | 1541249 | 1527311 | | |
| SEQ ID | 142257_ | 9.12E−07 | X | C | A | 7 | 13,731,414 | 142257_ | 123717_ | 13,715,325 | 13,740,309 |
| NO: 168 | 131770 | | | | | | | 115678 | 1052 | | |
| SEQ ID | 142254_ | 1.62E−07 | B, X* | C | G | 7 | 18,616,969 | 79267_ | 142254_ | 18,581,032 | 18,640,383 |
| NO: 184 | 8167871 | | | | | | | 1301 | 8153669 | | |
| SEQ ID | 141405_ | 2.75E−08 | B, X | G | C | 7 | 30,411,594 | 141405_ | 108628_ | 30,318,939 | 30,489,245 |
| NO: 199 | 197268 | | | | | | | 267660 | 7313 | | |
| SEQ ID | 141673_ | 1.51E−07 | A, X | A | G | 7 | 37,505,904 | 141673_ | 141673_ | 37,495,060 | 37,639,835 |
| NO: 214 | 1231418 | | | | | | | 1220738 | 1339456 | | |
| SEQ ID | 141440_ | 1.62E−06 | B, X | A | G | 7 | 41,659,351 | 141440_ | 141440_ | 41,651,577 | 41,675,164 |
| NO: 218 | 450292 | | | | | | | 458068 | 440631 | | |
| SEQ ID | 142587_ | 1.58E−06 | A, X | G | A | 7 | 44,916,971 | 142587_ | 142587_ | 44,911,280 | 44,924,842 |
| NO: 228 | 619893 | | | | | | | 614202 | 627764 | | |
| SEQ ID | 141293_ | 7.59E−08 | B, X | G | T | 7 | 49,114,154 | 141293_ | 141293_ | 49,085,607 | 49,134,977 |
| NO: 242 | 474861 | | | | | | | 503430 | 454000 | | |
| SEQ ID | 142269_ | 3.24E−07 | B, X | T | C | 7 | 50,745,259 | 196496_ | 142269_ | 50,724,154 | 50,765,250 |
| NO: 268 | 27530 | | | | | | | 1078 | 47480 | | |
| SEQ ID | 142269_ | 7.59E−07 | A, X | G | C | 7 | 50,767,962 | 142269_ | 142269_ | 50,765,250 | 50,777,808 |
| NO: 269 | 50192 | | | | | | | 47480 | 60092 | | |
| SEQ ID | 142269_ | 4.07E−09 | B, X | G | C | 7 | 50,820,763 | 142269_ | 142269_ | 50,818,092 | 50,822,516 |
| NO: 271 | 102855 | | | | | | | 100184 | 104610 | | |
| SEQ ID | 142269_ | 6.31E−12 | B, X | A | C | 7 | 50,854,826 | 142269_ | 142269_ | 50,822,516 | 50,959,554 |
| NO: 273 | 135398 | | | | | | | 104610 | 204126 | | |
| SEQ ID | 142269_ | 2.95E−12 | B, X | C | T | 7 | 51,054,719 | 142269_ | 142269_ | 51,022,401 | 51,062,076 |
| NO: 274 | 279954 | | | | | | | 264262 | 287311 | | |
| SEQ ID | 142269_ | 2.95E−12 | B, X | G | A | 7 | 51,173,524 | 138948_ | 142269_ | 51,141,514 | 51,226,826 |
| NO: 275 | 389457 | | | | | | | 776 | 435176 | | |
| SEQ ID | 140250_ | 1.62E−09 | B, X | G | A | 7 | 52,285,032 | 142269_ | 140250_ | 51,262,584 | 52,291,349 |
| NO: 276 | 15736 | | | | | | | 462851 | 9419 | | |
| SEQ ID | 140250_ | 1.29E−10 | B, X | T | C | 7 | 52,296,271 | 140250_ | 107140_ | 52,291,349 | 52,412,182 |
| NO: 277 | 4497 | | | | | | | 9419 | 596 | | |
| SEQ ID | 140726_ | 2.34E−08 | B, X | A | G | 7 | 52,322,834 | 140250_ | 107140_ | 52,291,349 | 52,412,182 |
| NO: 278 | 298459 | | | | | | | 9419 | 596 | | |
| SEQ ID | 140726_ | 2.34E−08 | B, X | G | A | 7 | 52,332,628 | 140250_ | 107140_ | 52,291,349 | 52,412,182 |
| NO: 279 | 288665 | | | | | | | 9419 | 596 | | |
| SEQ ID | 140726_ | 4.27E−10 | B, X | T | A | 7 | 52,554,676 | 140726_ | 129407_ | 52,552,192 | 52,760,459 |
| NO: 286 | 99547 | | | | | | | 102031 | 1096 | | |
| SEQ ID | 140726_ | 4.27E−10 | B, X | A | G | 7 | 52,561,249 | 140726_ | 129407_ | 52,552,192 | 52,760,459 |
| NO: 287 | 92974 | | | | | | | 102031 | 1096 | | |
| SEQ ID | 142593_ | 4.17E−07 | A, X | G | A | 7 | 58,317,539 | 142593_ | 142593_ | 58,305,695 | 58,328,372 |
| NO: 344 | 461393 | | | | | | | 449636 | 472226 | | |
| SEQ ID | 142593_ | 6.61E−07 | B, X | C | T | 7 | 58,528,791 | 142593_ | 142593_ | 58,524,187 | 58,538,433 |
| NO: 347 | 650845 | | | | | | | 646242 | 660489 | | |

TABLE 20
NAM results for THC to CBG Ratio (=(Total THC + Total THCV + 1)/(Total CBG + Total CBGV + 1)) in a set of 266
high varin type I accesions (set 5 Table 3). Each respective marker is identified as a chromosome position within the CsaAba2 reference
genome as well as at position 51 of the sequence identifier described in column 1. Left and right flanking SNPs of the marker haplotype
are the first non-significant SNPs to the left and right of the SNP marker. First column, SNP marker number; Second column, SNP marker
name; Third column, NAM p-value; Fourth column, beneficial genotype (A = homozygous for reference allele, B = homozygous
for alternative allele, X = heterozygous); Fifth column, reference allele call; Sixth column, alternative allele call; Seventh
column, Abacus reference genome (version CsaAba2) chromosome. Eighth column, Abacus reference genome (version CsaAba2) position in
bp; Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding
SNP marker; Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | Chro- | genome | SNP of | SNP of | marker | marker |
| | marker | | Geno- | Ref | Alt | mo- | position | marker | marker | haplotype | haplotype |
| SEQ ID | name | p-value | type | call | call | some | (bp) | haplotype | haplotype | (bp) | (bp) |
| SEQ ID | 142050_ | 7.08E−09 | A, X | C | T | 5 | 2,366,529 | 142050_ | 142050_ | 2,364,964 | 2,534,579 |
| NO: 36 | 2637918 | | | | | | | 2639484 | 2550524 | | |
| SEQ ID | 142050_ | 9.55E−09 | A | T | C | 5 | 2,899,343 | 142050_ | 142050_ | 2,844,078 | 2,908,474 |
| NO: 37 | 2212118 | | | | | | | 2261596 | 2202989 | | |
| SEQ ID | 142050_ | 2.63E−08 | A, X | C | T | 5 | 3,074,649 | 142050_ | 142050_ | 3,061,958 | 3,081,773 |
| NO: 38 | 2052266 | | | | | | | 2064957 | 2045142 | | |
| SEQ ID | 142050_ | 5.50E−08 | A, X | T | C | 5 | 3,086,874 | 142050_ | 142050_ | 3,081,773 | 3,089,662 |
| NO: 39 | 2040041 | | | | | | | 2045142 | 2037253 | | |
| SEQ ID | 142050_ | 7.76E−07 | A, X | G | A | 5 | 3,432,405 | 142050_ | 142050_ | 3,395,357 | 3,454,995 |
| NO: 40 | 1748980 | | | | | | | 1783530 | 1726442 | | |
| SEQ ID | 138186_ | 2.57E−10 | A | T | C | 5 | 3,485,895 | 142050_ | 138186_ | 3,454,995 | 3,493,107 |
| NO: 41 | 4346 | | | | | | | 1726442 | 11557 | | |
| SEQ ID | 138186_ | 1.07E−08 | A, X | A | G | 5 | 3,535,297 | 138186_ | 138186_ | 3,526,980 | 3,541,316 |
| NO: 42 | 53738 | | | | | | | 45421 | 59758 | | |
| SEQ ID | 142050_ | 1.12E−06 | A, X | G | A | 5 | 3,599,637 | 121249_ | Cannabis.v1_ | 3,585,965 | 3,604,863 |
| NO: 43 | 1719103 | | | | | | | 123 | scf876- | | |
| | | | | | | | | | 151191_101 | | |
| SEQ ID | 128680_ | 7.59E−07 | A, X | G | A | 5 | 3,673,686 | 142050_ | 142050_ | 3,656,543 | 3,680,073 |
| NO: 44 | 3420 | | | | | | | 1662261 | 1642364 | | |
| SEQ ID | 142050_ | 2.29E−07 | A, X | C | G | 5 | 3,961,139 | 142050_ | 142050_ | 3,945,751 | 3,965,771 |
| NO: 45 | 1411714 | | | | | | | 1427098 | 1407082 | | |
| SEQ ID | 142050_ | 4.07E−08 | A, X | A | G | 5 | 4,118,245 | 142050_ | 142050_ | 4,109,676 | 4,130,254 |
| NO: 46 | 1254508 | | | | | | | 1263077 | 1242499 | | |
| SEQ ID | 142050_ | 7.94E−07 | A | A | T | 5 | 4,384,123 | 142050_ | 142050_ | 4,376,633 | 4,391,586 |
| NO: 47 | 1011763 | | | | | | | 1019253 | 1003919 | | |
| SEQ ID | 142254_ | 1.95E−07 | A, X | C | T | 5 | 6,491,377 | Cannabis.v1_ | 142254_ | 6,489,377 | 6,516,042 |
| NO: 48 | 6896550 | | | | | | | scf676- | 6871885 | | |
| | | | | | | | | 73596_100 | | | |
| SEQ ID | 142254_ | 1.58E−07 | A, X | T | G | 5 | 6,666,604 | 142254_ | 142254_ | 6,636,230 | 6,668,100 |
| NO: 49 | 6738322 | | | | | | | 6768574 | 6736826 | | |
| SEQ ID | 142254_ | 2.14E−07 | A, X | A | C | 5 | 7,129,622 | 142254_ | 139365_ | 7,067,915 | 7,147,487 |
| NO: 50 | 6394766 | | | | | | | 6446672 | 5134 | | |
| SEQ ID | 142254_ | 1.66E−07 | A, X | T | C | 5 | 7,245,930 | 142254_ | 142254_ | 7,226,971 | 7,257,339 |
| NO: 51 | 6291758 | | | | | | | 6310320 | 6287538 | | |
| SEQ ID | 142254_ | 6.17E−07 | A, X | A | G | 5 | 7,904,873 | 142254_ | 142254_ | 7,897,923 | 7,933,626 |
| NO: 52 | 5690149 | | | | | | | 5697099 | 5663294 | | |
| SEQ ID | 142254_ | 7.59E−07 | A, X | G | A | 5 | 8,078,921 | 123919_ | 142254_ | 8,074,202 | 8,089,196 |
| NO: 53 | 5526537 | | | | | | | 8973 | 5516261 | | |
| SEQ ID | Cannabis.v1_ | 8.13E−07 | A, X | G | A | 5 | 9,111,167 | un55928_ | 142254_ | 9,091,191 | 9,164,971 |
| NO: 54 | scf5269- | | | | | | | 60_61 | 4629725 | | |
| | 5524_101 | | | | | | | | | | |
| SEQ ID | 142254_ | 8.13E−07 | A, X | T | C | 5 | 9,113,994 | un55928_ | 142254_ | 9,091,191 | 9,164,971 |
| NO: 55 | 4671720 | | | | | | | 60_61 | 4629725 | | |
| SEQ ID | 142254_ | 9.77E−08 | A, X | T | C | 5 | 9,472,805 | 142254_ | 142254_ | 9,453,509 | 9,478,633 |
| NO: 56 | 4412683 | | | | | | | 4430076 | 4406855 | | |
| SEQ ID | 142254_ | 4.79E−07 | A, X | C | T | 5 | 9,565,471 | 142254_ | 142254_ | 9,542,524 | 9,570,961 |
| NO: 57 | 4329494 | | | | | | | 4343189 | 4324001 | | |
| SEQ ID | 142254_ | 9.55E−07 | A, X | G | C | 5 | 9,734,723 | 142254_ | 142254_ | 9,718,369 | 9,771,730 |
| NO: 58 | 4191858 | | | | | | | 4203025 | 4177651 | | |
| SEQ ID | 142254_ | 4.79E−07 | A, X | G | T | 5 | 9,752,109 | 142254_ | 142254_ | 9,718,369 | 9,771,730 |
| NO: 59 | 4187450 | | | | | | | 4203025 | 4177651 | | |
| SEQ ID | 142254_ | 1.58E−07 | A, X | C | T | 5 | 10,155,920 | 142254_ | 142254_ | 10,143,433 | 10,200,275 |
| NO: 60 | 3855623 | | | | | | | 3868110 | 3820494 | | |
| SEQ ID | 142254_ | 2.57E−07 | A, X | C | T | 5 | 10,439,554 | 142254_ | 142254_ | 10,402,308 | 10,486,467 |
| NO: 61 | 3554355 | | | | | | | 3589351 | 3508586 | | |
| SEQ ID | 142254_ | 4.37E−07 | A, X | A | G | 5 | 10,451,559 | 142254_ | 142254_ | 10,402,308 | 10,486,467 |
| NO: 62 | 3540209 | | | | | | | 3589351 | 3508586 | | |
| SEQ ID | 142254_ | 9.77E−08 | A, X | A | G | 5 | 10,469,451 | 142254_ | 142254_ | 10,402,308 | 10,486,467 |
| NO: 63 | 3525755 | | | | | | | 3589351 | 3508586 | | |
| SEQ ID | 142254_ | 9.77E−08 | A, X | G | A | 5 | 10,548,740 | 142254_ | 142254_ | 10,531,323 | 10,586,049 |
| NO: 64 | 3458512 | | | | | | | 3475925 | 3421161 | | |
| SEQ ID | 123613_ | 1.35E−06 | A, X | T | C | 5 | 11,161,216 | 142254_ | 76613_ | 11,131,783 | 11,193,898 |
| NO: 65 | 11373 | | | | | | | 2906337 | 2309 | | |
| SEQ ID | 142254_ | 3.31E−07 | A, X | G | A | 5 | 11,245,818 | 142254_ | 142254_ | 11,217,176 | 11,252,809 |
| NO: 66 | 2808940 | | | | | | | 2832338 | 2801948 | | |
| SEQ ID | 142254_ | 1.10E−06 | A, X | C | A | 5 | 11,257,676 | 142254_ | 142254_ | 11,252,809 | 11,322,876 |
| NO: 67 | 2797082 | | | | | | | 2801948 | 2742141 | | |
| SEQ ID | 142254_ | 1.74E−06 | A, X | A | G | 5 | 11,310,169 | 142254_ | 142254_ | 11,252,809 | 11,322,876 |
| NO: 68 | 2754905 | | | | | | | 2801948 | 2742141 | | |
| SEQ ID | 142254_ | 1.38E−06 | A, X | A | T | 5 | 12,446,524 | 142254_ | 142254_ | 12,444,383 | 12,472,642 |
| NO: 69 | 1798647 | | | | | | | 1800788 | 1772523 | | |
| SEQ ID | 142254_ | 2.29E−07 | A, X | T | G | 5 | 12,932,181 | 142254_ | 113928_ | 12,829,839 | 12,944,302 |
| NO: 70 | 1356204 | | | | | | | 1448029 | 192 | | |
| SEQ ID | 142254_ | 5.13E−07 | A, X | C | T | 5 | 13,378,138 | 142254_ | 142254_ | 13,375,598 | 13,383,733 |
| NO: 71 | 945759 | | | | | | | 948299 | 940164 | | |
| SEQ ID | 142254_ | 1.55E−06 | A, X | A | G | 5 | 13,758,916 | 142254_ | 131195_ | 13,754,147 | 13,766,692 |
| NO: 72 | 618972 | | | | | | | 623740 | 3896 | | |
| SEQ ID | 109654_ | 1.26E−06 | A, X | T | A | 5 | 14,847,788 | 78705_ | 139496_ | 14,759,093 | 14,860,844 |
| NO: 73 | 9870 | | | | | | | 5431 | 12193 | | |
| SEQ ID | 140930_ | 5.75E−07 | A, X | C | G | 5 | 15,194,741 | 140930_ | 140930_ | 15,085,497 | 15,201,574 |
| NO: 74 | 95958 | | | | | | | 12013 | 102791 | | |
| SEQ ID | 103043_ | 8.51E−07 | A, X | G | A | 5 | 16,033,894 | 140930_ | 103043_ | 15,975,544 | 16,037,097 |
| NO: 75 | 12945 | | | | | | | 750030 | 9742 | | |
| SEQ ID | 141234_ | 7.41E−07 | A, X | G | A | 5 | 18,706,160 | 141234_ | 141234_ | 18,699,991 | 18,716,207 |
| NO: 76 | 646804 | | | | | | | 652973 | 636582 | | |
| SEQ ID | 135428_ | 8.91E−07 | A, X | C | T | 5 | 19,988,534 | 141363_ | 141363_ | 19,976,402 | 20,026,964 |
| NO: 77 | 7370 | | | | | | | 474561 | 514500 | | |
| SEQ ID | 141363_ | 2.45E−07 | A, X | T | G | 5 | 20,017,410 | 141363_ | 141363_ | 19,976,402 | 20,026,964 |
| NO: 78 | 504946 | | | | | | | 474561 | 514500 | | |
| SEQ ID | 141363_ | 1.23E−07 | A, X | G | A | 5 | 20,148,519 | 141363_ | 82188_ | 20,145,940 | 20,173,122 |
| NO: 79 | 624738 | | | | | | | 622159 | 1691 | | |
| SEQ ID | 141363_ | 1.55E−06 | A, X | A | T | 5 | 20,537,946 | 141363_ | 167_ | 20,483,019 | 20,546,290 |
| NO: 80 | 961227 | | | | | | | 906338 | 2512371 | | |
| SEQ ID | 125598_ | 1.10E−06 | A, X | C | A | 5 | 20,790,022 | 110596_ | 142351_ | 20,721,287 | 20,835,904 |
| NO: 81 | 9975 | | | | | | | 5073 | 264927 | | |
| SEQ ID | 141416_ | 0.000001 | A, X | T | C | 5 | 21,524,826 | 141416_ | 141416_ | 21,516,323 | 21,554,970 |
| NO: 82 | 227999 | | | | | | | 220078 | 258124 | | |
| SEQ ID | 124185_ | 7.76E−07 | A, X | C | T | 5 | 21,563,573 | 141416_ | 141416_ | 21,554,970 | 21,626,778 |
| NO: 83 | 3188 | | | | | | | 258124 | 323219 | | |
| SEQ ID | 141632_ | 5.50E−07 | A, X | T | G | 5 | 23,962,791 | 141632_ | Cannabis.v1_ | 23,872,960 | 24,016,565 |
| NO: 84 | 409539 | | | | | | | 488116 | scf523- | | |
| | | | | | | | | | 1286_100 | | |
| SEQ ID | 141632_ | 0.000001 | A, X | A | G | 5 | 23,968,827 | 141632_ | Cannabis.v1_ | 23,872,960 | 24,016,565 |
| NO: 85 | 403504 | | | | | | | 488116 | scf523- | | |
| | | | | | | | | | 1286_100 | | |
| SEQ ID | 141632_ | 0.000001 | A, X | A | T | 5 | 23,975,759 | 141632_ | Cannabis.v1_ | 23,872,960 | 24,016,565 |
| NO: 86 | 396575 | | | | | | | 488116 | scf523- | | |
| | | | | | | | | | 1286_100 | | |
| SEQ ID | 141287_ | 9.77E−07 | A, X | A | C | 5 | 24,897,787 | 141287_ | 141287_ | 24,799,598 | 24,906,306 |
| NO: 87 | 286298 | | | | | | | 370216 | 277779 | | |
| SEQ ID | 140600_ | 2.24E−07 | A, X | C | T | 5 | 25,356,228 | 140600_ | Cannabis.v1_ | 25,342,928 | 25,387,614 |
| NO: 88 | 466642 | | | | | | | 479942 | scf55- | | |
| | | | | | | | | | 46739_100 | | |
| SEQ ID | 140600_ | 2.24E−07 | A, X | T | C | 5 | 25,364,836 | 140600_ | Cannabis.v1_ | 25,342,928 | 25,387,614 |
| NO: 89 | 458034 | | | | | | | 479942 | scf55- | | |
| | | | | | | | | | 46739_100 | | |
| SEQ ID | 112336_ | 2.24E−07 | A, X | G | C | 5 | 25,387,741 | Cannabis.v1_ | 112336_ | 25,387,614 | 25,391,623 |
| NO: 90 | 826 | | | | | | | scf55- | 4708 | | |
| | | | | | | | | 46739_100 | | | |
| SEQ ID | 140600_ | 2.24E−07 | A, X | G | A | 5 | 25,536,887 | 112336_ | 140600_ | 25,391,623 | 25,548,921 |
| NO: 91 | 301818 | | | | | | | 4708 | 289787 | | |
| SEQ ID | 140106_ | 1.17E−06 | A, X | G | T | 5 | 28,880,563 | Cannabis.v1_ | 111161_ | 28,847,049 | 28,956,941 |
| NO: 92 | 450343 | | | | | | | scf2781- | 8163 | | |
| | | | | | | | | 47404_101 | | | |
| SEQ ID | 140641_ | 9.77E−07 | A, X | T | C | 5 | 32,311,818 | 140641_ | 140641_ | 32,296,352 | 32,322,620 |
| NO: 93 | 457525 | | | | | | | 442114 | 468303 | | |
| SEQ ID | 140641_ | 9.77E−07 | A, X | C | T | 5 | 32,387,532 | 140641_ | 140641_ | 32,322,620 | 32,390,660 |
| NO: 94 | 525818 | | | | | | | 468303 | 528950 | | |
| SEQ ID | 140360_ | 1.15E−06 | A, X | C | T | 5 | 36,148,442 | 140360_ | 140360_ | 36,089,359 | 36,288,826 |
| NO: 95 | 367283 | | | | | | | 401088 | 247867 | | |
| SEQ ID | 140776_ | 1.17E−06 | A, X | C | T | 5 | 40,532,113 | 79574_ | 140776_ | 40,385,082 | 40,557,005 |
| NO: 96 | 59092 | | | | | | | 11339 | 83984 | | |
| SEQ ID | 140951_ | 1.17E−06 | A, X | C | T | 5 | 42,410,724 | 140951_ | 140951_ | 42,375,856 | 42,528,615 |
| NO: 97 | 116615 | | | | | | | 172782 | 78820 | | |
| SEQ ID | 140951_ | 6.76E−07 | A, X | A | G | 5 | 42,557,566 | 140951_ | 140951_ | 42,550,360 | 42,563,140 |
| NO: 98 | 49477 | | | | | | | 56876 | 43908 | | |
| SEQ ID | 141161_ | 1.17E−06 | A, X | A | C | 5 | 43,499,675 | 140951_ | 141161_ | 42,604,248 | 43,518,393 |
| NO: 99 | 142548 | | | | | | | 2843 | 131500 | | |
| SEQ ID | 140777_ | 1.17E−06 | A, X | G | A | 5 | 44,062,719 | 140777_ | 105410_ | 44,026,725 | 44,095,222 |
| NO: 100 | 165625 | | | | | | | 136854 | 15189 | | |
| SEQ ID | 142674_ | 2.24E−07 | A, X | T | C | 5 | 45,687,721 | 142674_ | 109556_ | 45,678,680 | 45,734,373 |
| NO: 101 | 1045013 | | | | | | | 1057546 | 14093 | | |
| SEQ ID | 141905_ | 1.74E−06 | A, X | T | A | 5 | 48,977,178 | 141905_ | 141905_ | 48,970,067 | 48,984,121 |
| NO: 102 | 1834674 | | | | | | | 1841785 | 1827731 | | |
| SEQ ID | 419_ | 6.31E−07 | A, X | C | T | 5 | 52,819,178 | 419_ | 419_ | 52,798,015 | 52,858,896 |
| NO: 103 | 166991 | | | | | | | 155127 | 206708 | | |
| SEQ ID | 419_ | 7.59E−07 | A, X | C | A | 5 | 53,489,757 | 419_ | 419_ | 53,299,267 | 53,496,045 |
| NO: 104 | 678771 | | | | | | | 547859 | 685059 | | |
| SEQ ID | 419_ | 7.24E−07 | A, X | C | T | 5 | 53,719,271 | 419_ | 132777_ | 53,705,956 | 53,815,660 |
| NO: 105 | 884190 | | | | | | | 873901 | 26261 | | |
| SEQ ID | 132_ | 9.55E−07 | A, X | C | T | 5 | 54,339,213 | 419_ | 122648_ | 54,304,731 | 54,406,395 |
| NO: 106 | 07915905 | | | | | | | 1415013 | 7050 | | |
| SEQ ID | 419_ | 6.31E−07 | A, X | C | T | 5 | 56,258,287 | 419_ | 419_ | 56,185,902 | 56,294,577 |
| NO: 107 | 3122991 | | | | | | | 3056817 | 3144559 | | |
| SEQ ID | 419_ | 6.31E−07 | A, X | C | A | 5 | 57,537,902 | 419_ | 419_ | 57,493,670 | 57,540,049 |
| NO: 108 | 4202255 | | | | | | | 4171991 | 4204402 | | |
| SEQ ID | 419_ | 1.48E−06 | A, X | T | A | 5 | 57,827,101 | 419_ | 419_ | 57,816,878 | 57,845,773 |
| NO: 109 | 4445987 | | | | | | | 4435764 | 4464665 | | |
| SEQ ID | 419_ | 1.48E−06 | A, X | C | T | 5 | 58,026,390 | 419_ | 419_ | 57,996,384 | 58,043,891 |
| NO: 110 | 4603364 | | | | | | | 4576513 | 4620866 | | |
| SEQ ID | Cannabis.v1_ | 1.74E−06 | A, X | C | T | 5 | 58,596,336 | 136872_ | 123617_ | 58,582,581 | 58,598,359 |
| NO: 111 | scf125- | | | | | | | 13081 | 11858 | | |
| | 78737_100 | | | | | | | | | | |
| SEQ ID | 141566_ | 3.63E−07 | A, X | T | A | 5 | 59,820,696 | 141566_ | 141566_ | 59,813,998 | 59,876,892 |
| NO: 112 | 980231 | | | | | | | 973534 | 1020606 | | |
| SEQ ID | 141610_ | 3.63E−07 | A, X | T | C | 5 | 60,280,385 | 101158_ | 141610_ | 60,254,151 | 60,285,659 |
| NO: 113 | 5634 | | | | | | | 997 | 10974 | | |
| SEQ ID | 102762_ | 3.63E−07 | A, X | A | G | 5 | 60,295,701 | 141610_ | 59034_ | 60,285,659 | 60,313,853 |
| NO: 114 | 325 | | | | | | | 10974 | 2802 | | |
| SEQ ID | 141610_ | 1.38E−06 | A, X | A | G | 5 | 61,249,054 | Cannabis.v1_ | 141610_ | 61,225,099 | 61,253,927 |
| NO: 115 | 733919 | | | | | | | scf35- | 738792 | | |
| | | | | | | | | 312504_101 | | | |
| SEQ ID | 418_ | 5.37E−07 | A | C | T | 5 | 64,186,528 | 418_ | 418_ | 64,168,576 | 64,227,025 |
| NO: 116 | 238459 | | | | | | | 250346 | 203609 | | |
| SEQ ID | 126818_ | 1.17E−07 | A, X | A | G | 5 | 64,884,077 | 126818_ | 126818_ | 64,881,518 | 64,888,996 |
| NO: 117 | 5537 | | | | | | | 2978 | 10455 | | |
| SEQ ID | 141246_ | 1.51E−06 | A, X | C | T | 7 | 23,406,044 | 139024_ | 141246_ | 23,372,911 | 23,432,690 |
| NO: 188 | 683086 | | | | | | | 22776 | 709730 | | |

TABLE 21
NAM results for THC to CBG Ratio (=(Total THC + Total THCV + 1)/(Total CBG + Total CBGV + 1)) in a set of 85 segregating
type I and IV accesions (set 6 Table 3). Each respective marker is identified as a chromosome position within the CsaAba2 reference genome
as well as at position 51 of the sequence identifier described in column 1. Left and right flanking SNPs of the marker haplotype are the
first non-significant SNPs to the left and right of the SNP marker. First column, SNP marker number; Second column, SNP marker name; Third
column, NAM p-value; Fourth column, beneficial genotype (A = homozygous for reference allele, B = homozygous for alternative
allele, X = heterozygous, *= B inferred based on segregation patterns; Fifth column, reference allele call; Sixth column, alternative
allele call; Seventh column, Abacus reference genome (version CsaAba2) chromosome. Eighth column, Abacus reference genome (version CsaAba2)
position in bp; Ninth column, left flanking SNP of haplotype surrounding SNP marker; Tenth column, right flanking SNP of haplotype surrounding
SNP marker; Eleventh column, Abacus reference genome (version CsaAba2) position in bp for left flanking SNP of haplotype surrounding SNP
marker; Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 123886_ | 3.02E−06 | B, X | G | A | 1 | 38,978,759 | 335_ | 335_ | 38,944,628 | 39,073,783 |
| NO: 4 | 2182 | | | | | | | 10251 | 146423 | | |
| SEQ ID | 142465_ | 2.82E−06 | B, X | C | T | 7 | 48,365,472 | 134245_ | 361_ | 48,198,578 | 49,711,589 |
| NO: 233 | 223976 | | | | | | | 2769 | 37055 | | |
| SEQ ID | 142465_ | 2.82E−06 | B, X | G | A | 7 | 48,366,628 | 134245_ | 361_ | 48,198,578 | 49,711,589 |
| NO: 234 | 222820 | | | | | | | 2769 | 37055 | | |
| SEQ ID | 142465_ | 2.82E−06 | A, X | T | C | 7 | 48,587,800 | 134245_ | 361_ | 48,198,578 | 49,711,589 |
| NO: 236 | 44455 | | | | | | | 2769 | 37055 | | |
| SEQ ID | 141293_ | 2.82E−06 | A, X | T | A | 7 | 48,688,297 | 134245_ | 361_ | 48,198,578 | 49,711,589 |
| NO: 237 | 830644 | | | | | | | 2769 | 37055 | | |
| SEQ ID | 141293_ | 2.82E−06 | B, X | G | T | 7 | 48,694,159 | 134245_ | 361_ | 48,198,578 | 49,711,589 |
| NO: 238 | 824822 | | | | | | | 2769 | 37055 | | |
| SEQ ID | 135018_ | 2.82E−06 | A, X | C | G | 7 | 49,044,262 | 134245_ | 361_ | 48,198,578 | 49,711,589 |
| NO: 240 | 30043 | | | | | | | 2769 | 37055 | | |
| SEQ ID | 135018_ | 2.82E−06 | A, X | A | T | 7 | 49,065,141 | 134245_ | 361_ | 48,198,578 | 49,711,589 |
| NO: 241 | 9167 | | | | | | | 2769 | 37055 | | |
| SEQ ID | 141293_ | 2.82E−06 | A, X | C | A | 7 | 49,134,977 | 134245_ | 361_ | 48,198,578 | 49,711,589 |
| NO: 243 | 454000 | | | | | | | 2769 | 37055 | | |
| SEQ ID | 141293_ | 2.82E−06 | A, X | T | C | 7 | 49,139,036 | 134245_ | 361_ | 48,198,578 | 49,711,589 |
| NO: 244 | 449941 | | | | | | | 2769 | 37055 | | |
| SEQ ID | 141293_ | 2.82E−06 | A, X | T | A | 7 | 49,272,668 | 134245_ | 361_ | 48,198,578 | 49,711,589 |
| NO: 245 | 345180 | | | | | | | 2769 | 37055 | | |
| SEQ ID | 141293_ | 2.82E−06 | A, X | G | T | 7 | 49,297,082 | 134245_ | 361_ | 48,198,578 | 49,711,589 |
| NO: 247 | 322698 | | | | | | | 2769 | 37055 | | |
| SEQ ID | 141293_ | 2.82E−06 | B, X | G | A | 7 | 49,304,484 | 134245_ | 361_ | 48,198,578 | 49,711,589 |
| NO: 248 | 315297 | | | | | | | 2769 | 37055 | | |
| SEQ ID | 141293_ | 2.82E−06 | B, X | T | G | 7 | 49,331,724 | 134245_ | 361_ | 48,198,578 | 49,711,589 |
| NO: 249 | 289619 | | | | | | | 2769 | 37055 | | |
| SEQ ID | 141293_ | 2.82E−06 | B, X | T | C | 7 | 49,344,499 | 134245_ | 361_ | 48,198,578 | 49,711,589 |
| NO: 250 | 277076 | | | | | | | 2769 | 37055 | | |
| SEQ ID | 141293_ | 2.82E−06 | A, X | T | A | 7 | 49,394,880 | 134245_ | 361_ | 48,198,578 | 49,711,589 |
| NO: 251 | 234853 | | | | | | | 2769 | 37055 | | |
| SEQ ID | 141293_ | 2.82E−06 | B, X | A | G | 7 | 49,628,859 | 134245_ | 361_ | 48,198,578 | 49,711,589 |
| NO: 253 | 11669 | | | | | | | 2769 | 37055 | | |
| SEQ ID | 141293_ | 2.82E−06 | B, X | G | T | 7 | 49,634,834 | 134245_ | 361_ | 48,198,578 | 49,711,589 |
| NO: 254 | 5695 | | | | | | | 2769 | 37055 | | |
| SEQ ID | 137284_ | 2.82E−06 | B, X | T | G | 7 | 49,664,112 | 134245_ | 361_ | 48,198,578 | 49,711,589 |
| NO: 255 | 22272 | | | | | | | 2769 | 37055 | | |
| SEQ ID | Cannabis.v1_ | 2.82E−06 | A, X | A | T | 7 | 49,666,319 | 134245_ | 361_ | 48,198,578 | 49,711,589 |
| NO: 256 | scf2253- | | | | | | | 2769 | 37055 | | |
| | 57653_100 | | | | | | | | | | |
| SEQ ID | 140888_ | 2.82E−06 | A, X | C | G | 7 | 49,843,878 | 361_ | 130363_ | 49,711,589 | 50,350,437 |
| NO: 257 | 17485 | | | | | | | 37055 | 10672 | | |
| SEQ ID | 140888_ | 3.47E−06 | A, X | G | T | 7 | 49,979,803 | 361_ | 130363_ | 49,711,589 | 50,350,437 |
| NO: 258 | 133719 | | | | | | | 37055 | 10672 | | |
| SEQ ID | 140888_ | 2.82E−06 | A, X | T | G | 7 | 50,084,463 | 361_ | 130363_ | 49,711,589 | 50,350,437 |
| NO: 259 | 207098 | | | | | | | 37055 | 10672 | | |
| SEQ ID | 140888_ | 2.82E−06 | A, X | A | G | 7 | 50,302,149 | 361_ | 130363_ | 49,711,589 | 50,350,437 |
| NO: 262 | 394798 | | | | | | | 37055 | 10672 | | |
| SEQ ID | 140888_ | 2.82E−06 | B, X | T | A | 7 | 50,515,764 | 130363_ | 140888_ | 50,350,437 | 50,528,030 |
| NO: 263 | 563703 | | | | | | | 10672 | 575969 | | |
| SEQ ID | 199432_ | 2.82E−06 | B, X | T | C | 7 | 50,582,962 | 140888_ | 142269_ | 50,528,030 | 50,822,516 |
| NO: 264 | 1874 | | | | | | | 575969 | 104610 | | |
| SEQ ID | 140888_ | 2.82E−06 | B, X | T | C | 7 | 50,630,217 | 140888_ | 142269_ | 50,528,030 | 50,822,516 |
| NO: 265 | 669626 | | | | | | | 575969 | 104610 | | |
| SEQ ID | 140888_ | 2.82E−06 | A, X | G | A | 7 | 50,662,578 | 140888_ | 142269_ | 50,528,030 | 50,822,516 |
| NO: 266 | 701986 | | | | | | | 575969 | 104610 | | |
| SEQ ID | 142269_ | 2.82E−06 | B, X | T | G | 7 | 50,715,700 | 140888_ | 142269_ | 50,528,030 | 50,822,516 |
| NO: 267 | 6486 | | | | | | | 575969 | 104610 | | |
| SEQ ID | 142269_ | 2.82E−06 | A, X | G | C | 7 | 50,767,962 | 140888_ | 142269_ | 50,528,030 | 50,822,516 |
| NO: 269 | 50192 | | | | | | | 575969 | 104610 | | |
| SEQ ID | 142269_ | 2.82E−06 | A, X | C | A | 7 | 50,818,092 | 140888_ | 142269_ | 50,528,030 | 50,822,516 |
| NO: 270 | 100184 | | | | | | | 575969 | 104610 | | |
| SEQ ID | 126812_ | 2.82E−06 | B, X | G | A | 7 | 52,439,705 | 142269_ | 140726_ | 50,822,516 | 52,523,987 |
| NO: 281 | 8470 | | | | | | | 104610 | 130225 | | |
| SEQ ID | un105509_ | 2.82E−06 | B, X | C | G | 7 | 52,441,872 | 142269_ | 140726_ | 50,822,516 | 52,523,987 |
| NO: 282 | 43_89 | | | | | | | 104610 | 130225 | | |
| SEQ ID | 140726_ | 2.82E−06 | B, X | A | T | 7 | 52,507,913 | 142269_ | 140726_ | 50,822,516 | 52,523,987 |
| NO: 283 | 146295 | | | | | | | 104610 | 130225 | | |
| SEQ ID | 140726_ | 2.82E−06 | B, X | A | C | 7 | 52,510,830 | 142269_ | 140726_ | 50,822,516 | 52,523,987 |
| NO: 284 | 143382 | | | | | | | 104610 | 130225 | | |
| SEQ ID | 140726_ | 2.82E−06 | B, X | C | A | 7 | 52,517,301 | 142269_ | 140726_ | 50,822,516 | 52,523,987 |
| NO: 285 | 136911 | | | | | | | 104610 | 130225 | | |
| SEQ ID | 141735_ | 3.89E−06 | B, X* | C | A | 7 | 52,923,743 | 140726_ | 132241_ | 52,544,592 | 53,396,185 |
| NO: 288 | 113420 | | | | | | | 109620 | 2253 | | |
| SEQ ID | 141735_ | 2.82E−06 | A, X | C | T | 7 | 53,449,873 | 132241_ | 170870_ | 53,396,185 | 54,375,898 |
| NO: 291 | 437148 | | | | | | | 2253 | 14856 | | |
| SEQ ID | 141735_ | 1.82E−06 | B, X | A | G | 7 | 53,505,022 | 132241_ | 170870_ | 53,396,185 | 54,375,898 |
| NO: 292 | 492163 | | | | | | | 2253 | 14856 | | |
| SEQ ID | 140997_ | 1.82E−06 | B, X | A | G | 7 | 54,400,345 | 170870_ | 140997_ | 54,375,898 | 54,452,078 |
| NO: 294 | 363509 | | | | | | | 14856 | 415246 | | |
| SEQ ID | 140997_ | 1.82E−06 | A, X | G | A | 7 | 54,464,358 | 140997_ | 140997_ | 54,452,078 | 54,646,726 |
| NO: 295 | 427582 | | | | | | | 415246 | 589675 | | |
| SEQ ID | 140997_ | 1.82E−06 | A, X | T | G | 7 | 54,509,399 | 140997_ | 140997_ | 54,452,078 | 54,646,726 |
| NO: 296 | 464201 | | | | | | | 415246 | 589675 | | |
| SEQ ID | 140997_ | 1.82E−06 | B, X | A | G | 7 | 54,513,057 | 140997_ | 140997_ | 54,452,078 | 54,646,726 |
| NO: 297 | 467859 | | | | | | | 415246 | 589675 | | |
| SEQ ID | 133080_ | 1.82E−06 | B, X | C | T | 7 | 54,555,051 | 140997_ | 140997_ | 54,452,078 | 54,646,726 |
| NO: 298 | 7333 | | | | | | | 415246 | 589675 | | |
| SEQ ID | 140997_ | 1.82E−06 | B, X | G | A | 7 | 54,693,540 | 140997_ | 140997_ | 54,683,300 | 54,714,058 |
| NO: 299 | 628227 | | | | | | | 617987 | 648743 | | |
| SEQ ID | 140997_ | 1.82E−06 | B, X | C | A | 7 | 54,722,302 | 140997_ | 131855_ | 54,714,058 | 54,860,264 |
| NO: 300 | 656987 | | | | | | | 648743 | 1935 | | |
| SEQ ID | 140997_ | 1.82E−06 | B, X | G | A | 7 | 54,915,189 | 131855_ | 137156_ | 54,860,264 | 55,029,197 |
| NO: 301 | 859069 | | | | | | | 1935 | 6696 | | |
| SEQ ID | 142086_ | 1.82E−06 | B, X | G | A | 7 | 55,277,957 | 142086_ | 142086_ | 55,255,666 | 55,316,629 |
| NO: 302 | 207043 | | | | | | | 184758 | 231920 | | |
| SEQ ID | 142086_ | 1.82E−06 | A, X | A | T | 7 | 55,452,244 | 142086_ | 142086_ | 55,316,629 | 55,939,712 |
| NO: 303 | 326496 | | | | | | | 231920 | 738926 | | |
| SEQ ID | 142086_ | 4.27E−06 | B, X | A | G | 7 | 55,716,705 | 142086_ | 142086_ | 55,316,629 | 55,939,712 |
| NO: 304 | 537884 | | | | | | | 231920 | 738926 | | |
| SEQ ID | 142086_ | 4.27E−06 | B, X | T | A | 7 | 55,735,556 | 142086_ | 142086_ | 55,316,629 | 55,939,712 |
| NO: 305 | 556735 | | | | | | | 231920 | 738926 | | |
| SEQ ID | 142086_ | 4.37E−06 | B, X | C | T | 7 | 55,836,464 | 142086_ | 142086_ | 55,316,629 | 55,939,712 |
| NO: 306 | 642992 | | | | | | | 231920 | 738926 | | |
| SEQ ID | 142086_ | 2.57E−06 | B, X | A | T | 7 | 55,970,630 | 142086_ | 142086_ | 55,939,712 | 56,102,656 |
| NO: 307 | 769641 | | | | | | | 738926 | 896032 | | |
| SEQ ID | 142086_ | 2.57E−06 | B, X | A | T | 7 | 55,984,088 | 142086_ | 142086_ | 55,939,712 | 56,102,656 |
| NO: 308 | 783150 | | | | | | | 738926 | 896032 | | |
| SEQ ID | 142086_ | 2.57E−06 | B, X | G | A | 7 | 56,018,989 | 142086_ | 142086_ | 55,939,712 | 56,102,656 |
| NO: 309 | 818053 | | | | | | | 738926 | 896032 | | |
| SEQ ID | 142086_ | 3.89E−06 | B, X* | G | A | 7 | 56,032,988 | 142086_ | 142086_ | 55,939,712 | 56,102,656 |
| NO: 310 | 826368 | | | | | | | 738926 | 896032 | | |
| SEQ ID | 142086_ | 2.57E−06 | B, X | C | T | 7 | 56,076,209 | 142086_ | 142086_ | 55,939,712 | 56,102,656 |
| NO: 311 | 869588 | | | | | | | 738926 | 896032 | | |
| SEQ ID | 142086_ | 4.47E−06 | B, X | T | C | 7 | 56,081,903 | 142086_ | 142086_ | 55,939,712 | 56,102,656 |
| NO: 312 | 875282 | | | | | | | 738926 | 896032 | | |
| SEQ ID | 142086_ | 2.57E−06 | B, X | A | G | 7 | 56,171,548 | 142086_ | 142086_ | 56,166,983 | 56,434,732 |
| NO: 313 | 948791 | | | | | | | 944226 | 1165543 | | |
| SEQ ID | 142086_ | 3.89E−06 | B, X* | G | A | 7 | 56,301,604 | 142086_ | 142086_ | 56,166,983 | 56,434,732 |
| NO: 314 | 1049946 | | | | | | | 944226 | 1165543 | | |
| SEQ ID | 142086_ | 2.57E−06 | B, X | T | G | 7 | 56,426,824 | 142086_ | 142086_ | 56,166,983 | 56,434,732 |
| NO: 316 | 1157635 | | | | | | | 944226 | 1165543 | | |
| SEQ ID | 142086_ | 2.57E−06 | A, X | A | G | 7 | 56,430,375 | 142086_ | 142086_ | 56,166,983 | 56,434,732 |
| NO: 317 | 1161186 | | | | | | | 944226 | 1165543 | | |
| SEQ ID | 142086_ | 2.57E−06 | B, X | C | T | 7 | 56,440,283 | 142086_ | 134458_ | 56,434,732 | 56,593,122 |
| NO: 318 | 1171094 | | | | | | | 1165543 | 15032 | | |
| SEQ ID | 142086_ | 2.57E−06 | A, X | A | G | 7 | 56,488,125 | 142086_ | 134458_ | 56,434,732 | 56,593,122 |
| NO: 320 | 1211275 | | | | | | | 1165543 | 15032 | | |
| SEQ ID | 142086_ | 2.57E−06 | A, X | T | G | 7 | 56,492,081 | 142086_ | 134458_ | 56,434,732 | 56,593,122 |
| NO: 321 | 1215230 | | | | | | | 1165543 | 15032 | | |
| SEQ ID | 142086_ | 2.57E−06 | B, X | C | A | 7 | 56,538,007 | 142086_ | 134458_ | 56,434,732 | 56,593,122 |
| NO: 322 | 1258351 | | | | | | | 1165543 | 15032 | | |
| SEQ ID | 102958_ | 2.57E−06 | A, X | T | C | 7 | 56,700,085 | 100708_ | 141318_ | 56,620,519 | 56,809,638 |
| NO: 323 | 690 | | | | | | | 6525 | 673231 | | |
| SEQ ID | 141318_ | 2.57E−06 | B, X | G | C | 7 | 56,782,341 | 100708_ | 141318_ | 56,620,519 | 56,809,638 |
| NO: 324 | 699916 | | | | | | | 6525 | 673231 | | |
| SEQ ID | 141318_ | 2.57E−06 | B, X | C | A | 7 | 56,872,960 | 141318_ | 141318_ | 56,809,638 | 57,276,534 |
| NO: 326 | 610006 | | | | | | | 673231 | 174028 | | |
| SEQ ID | 141318_ | 2.57E−06 | B, X | T | C | 7 | 56,910,768 | 141318_ | 141318_ | 56,809,638 | 57,276,534 |
| NO: 327 | 572197 | | | | | | | 673231 | 174028 | | |
| SEQ ID | 141318_ | 3.89E−06 | B, X* | A | T | 7 | 56,967,275 | 141318_ | 141318_ | 56,809,638 | 57,276,534 |
| NO: 328 | 522267 | | | | | | | 673231 | 174028 | | |
| SEQ ID | 141318_ | 2.57E−06 | B, X | C | T | 7 | 57,069,404 | 141318_ | 141318_ | 56,809,638 | 57,276,534 |
| NO: 329 | 415904 | | | | | | | 673231 | 174028 | | |
| SEQ ID | 141318_ | 2.57E−06 | B, X | C | T | 7 | 57,080,583 | 141318_ | 141318_ | 56,809,638 | 57,276,534 |
| NO: 330 | 404726 | | | | | | | 673231 | 174028 | | |
| SEQ ID | 141318_ | 2.57E−06 | A, X | T | A | 7 | 57,089,709 | 141318_ | 141318_ | 56,809,638 | 57,276,534 |
| NO: 331 | 395600 | | | | | | | 673231 | 174028 | | |
| SEQ ID | 141318_ | 2.57E−06 | B, X | C | T | 7 | 57,104,188 | 141318_ | 141318_ | 56,809,638 | 57,276,534 |
| NO: 332 | 381127 | | | | | | | 673231 | 174028 | | |
| SEQ ID | 124011_ | 2.57E−06 | A, X | C | T | 7 | 57,120,122 | 141318_ | 141318_ | 56,809,638 | 57,276,534 |
| NO: 333 | 4556 | | | | | | | 673231 | 174028 | | |
| SEQ ID | 141318_ | 2.57E−06 | A, X | G | A | 7 | 57,152,672 | 141318_ | 141318_ | 56,809,638 | 57,276,534 |
| NO: 334 | 337651 | | | | | | | 673231 | 174028 | | |
| SEQ ID | 141318_ | 2.57E−06 | A, X | A | G | 7 | 57,228,643 | 141318_ | 141318_ | 56,809,638 | 57,276,534 |
| NO: 335 | 264113 | | | | | | | 673231 | 174028 | | |
| SEQ ID | 141318_ | 2.57E−06 | B, X | A | G | 7 | 57,233,796 | 141318_ | 141318_ | 56,809,638 | 57,276,534 |
| NO: 336 | 269266 | | | | | | | 673231 | 174028 | | |
| SEQ ID | 142593_ | 3.89E−06 | B, X* | C | T | 7 | 58,538,433 | 142593_ | 142593_ | 58,428,139 | 58,607,780 |
| NO: 348 | 660489 | | | | | | | 559015 | 696671 | | |

NAM of CBC to CBG Ratio in the set of 233 low varin type I accessions (set 4 Table 3) identified 10 significant SNPs on chromosomes 6 and 7; one locus consisting of 3 SNPs between position 50.1-51.2 Mbp on chromosome 7 (Table 22). NAM of CBC to CBG Ratio in the set of 266 high varin type I accessions (set 5 Table 3) identified two significant SNPs on chromosome 5 (Table 23). NAM of CBC to CBG ratio in the set of 85 segregating type I and IV F2 accessions (set 6 Table 3) identified 21 significant SNPs on chromosome 7; one locus consisting of 6 SNPs between positions 54.5-55.3 Mbp on chromosome 7 (Table 24).

In total, 369 SNP markers were significantly associated with one or more cannabinoid traits (Table 27). For most traits SNP markers were found in one or more loci on chromosome 7 in a region with low levels of recombination near the centromere, which is supported by internal (see next section for more background on the linkage map; Table 28) as well as external linkage map comparisons with physical positions (Laverty, Kaitlin U., et al. “A physical and genetic map of Cannabis sativa identifies extensive rearrangements at the THC/CBD acid synthase loci.” Genome research 29.1 (2019): 146-156).

TABLE 22
NAM results for CBC to CBG Ratio (=(Total CBC + 1)/(Total CBG + Total CBGV + 1)) in a set of 233 type I accesions (set
4 Table 3). Each respective marker is identified as a chromosome position within the CsaAba2 reference genome as well as at position
51 of the sequence identifier described in column 1. Left and right flanking SNPs of the marker haplotype are the first non-significant
SNPs to the left and right of the SNP marker. First column, SNP marker number; Second column, SNP marker name; Third column, NAM p-value;
Fourth column, beneficial genotype (A = homozygous for reference allele, B = homozygous for alternative allele, X = heterozygous),
*= B inferred based on segregation patterns; Fifth column, reference allele call; Sixth column, alternative allele call; Seventh
column, Abacus reference genome (version CsaAba2) chromosome. Eighth column, Abacus reference genome (version CsaAba2) position in bp;
Ninth column, left flanking SNP of haplotype surrounding SNP marker; Tenth column, right flanking SNP of haplotype surrounding SNP marker;
Eleventh column, Abacus reference genome (version CsaAba2) position in bp for left flanking SNP of haplotype surrounding SNP marker;
Twelfth column, Abacus reference genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 140767_ | 2.75E−08 | B, X* | G | A | 6 | 4,023,145 | 140767_ | 155782_ | 4,002,406 | 4,087,346 |
| NO: 118 | 20769 | | | | | | | 41489 | 7231 | | |
| SEQ ID | 141963_ | 1.17E−07 | B, X | G | A | 7 | 7,639,988 | 141963_ | 141963_ | 7,630,020 | 7,643,910 |
| NO: 149 | 1531282 | | | | | | | 1541249 | 1527311 | | |
| SEQ ID | 141440_ | 4.57E−07 | B, X | A | G | 7 | 41,659,351 | 141440_ | 141440_ | 41,651,577 | 41,675,164 |
| NO: 218 | 450292 | | | | | | | 458068 | 440631 | | |
| SEQ ID | 142269_ | 4.68E−09 | B, X | A | C | 7 | 50,854,826 | 142269_ | 142269_ | 50,822,516 | 50,959,554 |
| NO: 273 | 135398 | | | | | | | 104610 | 204126 | | |
| SEQ ID | 142269_ | 8.91E−09 | B, X | C | T | 7 | 51,054,719 | 142269_ | 142269_ | 51,022,401 | 51,062,076 |
| NO: 274 | 279954 | | | | | | | 264262 | 287311 | | |
| SEQ ID | 142269_ | 8.91E−09 | B, X | G | A | 7 | 51,173,524 | 138948_ | 142269_ | 51,141,514 | 51,226,826 |
| NO: 275 | 389457 | | | | | | | 776 | 435176 | | |
| SEQ ID | 140250_ | 1.35E−08 | B, X | T | C | 7 | 52,296,271 | 140250_ | 140726_ | 52,291,349 | 52,322,834 |
| NO: 277 | 4497 | | | | | | | 9419 | 298459 | | |
| SEQ ID | 140726_ | 7.76E−07 | B, X | T | A | 7 | 52,554,676 | 140726_ | 129407_ | 52,552,192 | 52,760,459 |
| NO: 286 | 99547 | | | | | | | 102031 | 1096 | | |
| SEQ ID | 140726_ | 7.76E−07 | B, X | A | G | 7 | 52,561,249 | 140726_ | 129407_ | 52,552,192 | 52,760,459 |
| NO: 287 | 92974 | | | | | | | 102031 | 1096 | | |
| SEQ ID | 142593_ | 3.02E−08 | B, X | C | T | 7 | 58,528,791 | 142593_ | 142593_ | 58,524,187 | 58,538,433 |
| NO: 347 | 650845 | | | | | | | 646242 | 660489 | | |

TABLE 23
NAM results for CBC to CBG Ratio (=(Total CBC + 1)/(Total CBG + Total CBGV + 1)) in a set of 266 type
I high varin accesions (set 5 Table 3). Each respective marker is identified as a chromosome position within the CsaAba2
reference genome as well as at position 51 of the sequence identifier described in column 1. Left and right flanking SNPs
of the marker haplotype are the first non-significant SNPs to the left and right of the SNP marker. First column, SNP marker
number; Second column, SNP marker name; Third column, NAM p-value; Fourth column, beneficial genotype (A = homozygous
for reference allele, B = homozygous for alternative allele, X = heterozygous; Fifth column, reference allele
call; Sixth column, alternative allele call; Seventh column, Abacus reference genome (version CsaAba2) chromosome. Eighth
column, Abacus reference genome (version CsaAba2) position in bp; Ninth column, left flanking SNP of haplotype surrounding
SNP marker; Tenth column, right flanking SNP of haplotype surrounding SNP marker; Eleventh column, Abacus reference genome
(version CsaAba2) position in bp for left flanking SNP of haplotype surrounding SNP marker; Twelfth column, Abacus reference
genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 141363_ | 1.66E−06 | A, X | T | G | 5 | 20,017,410 | 135428_ | 141363_ | 19,988,534 | 20,026,964 |
| NO: 78 | 504946 | | | | | | | 7370 | 514500 | | |
| SEQ ID | 140360_ | 1.26E−06 | A, X | C | T | 5 | 36,148,442 | 140360_ | 140360_ | 36,089,359 | 36,288,826 |
| NO: 95 | 367283 | | | | | | | 401088 | 247867 | | |

TABLE 24
NAM results for CBC to CBG Ratio (=(Total CBC + 1)/(Total CBG + Total CBGV + 1)) in a set of 85 segregating type
I and IV accesions (set 6 Table 3). Each respective marker is identified as a chromosome position within the CsaAba2 reference genome
as well as at position 51 of the sequence identifier described in column 1. Left and right flanking SNPs of the marker haplotype
are the first non-significant SNPs to the left and right of the SNP marker. First column, SNP marker number; Second column, SNP marker
name; Third column, NAM p-value; Fourth column, beneficial genotype (A = homozygous for reference allele, B = homozygous
for alternative allele, X=heterozygous), *= B inferred based on segregation patterns, **= X inferred based on segregation
patterns; Fifth column, reference allele call; Sixth column, alternative allele call; Seventh column, Abacus reference genome (version
CsaAba2) chromosome. Eighth column, Abacus reference genome (version CsaAba2) position in bp; Ninth column, left flanking SNP of
haplotype surrounding SNP marker; Tenth column, right flanking SNP of haplotype surrounding SNP marker; Eleventh column, Abacus reference
genome (version CsaAba2) position in bp for left flanking SNP of haplotype surrounding SNP marker; Twelfth column, Abacus reference
genome (version CsaAba2) position in bp for right flanking SNP of haplotype surrounding SNP marker.

| | | | | | | | | | | Position | Position |
|---|---|---|---|---|---|---|---|---|---|---|---|
| | | | | | | | | | | left | right |
| | | | | | | | Abacus | Left | Right | flanking | flanking |
| | | | | | | | reference | flanking | flanking | SNP of | SNP of |
| | SNP | | | | | | genome | SNP of | SNP of | marker | marker |
| | marker | | Geno- | Ref | Alt | | position | marker | marker | haplotype | haplotype |
| SEQ ID | name | p-value | type | call | call | Chromosome | (bp) | haplotype | haplotype | (bp) | (bp) |
| SEQ ID | 123886_ | 6.76E−09 | B, X | G | A | 1 | 38,978,759 | 335_ | 335_ | 38,944,628 | 39,073,783 |
| NO: 4 | 2182 | | | | | | | 10251 | 146423 | | |
| SEQ ID | 211_ | 2.00E−06 | B, X** | G | A | 1 | 67,769,631 | 177642_ | 211_ | 67,761,686 | 67,892,254 |
| NO: 8 | 137879 | | | | | | | 4242 | 40813 | | |
| SEQ ID | 141356_ | 5.01E−06 | B, X** | G | C | 7 | 28,081,703 | 141356_ | 141356_ | 27,759,260 | 28,263,307 |
| NO: 191 | 607414 | | | | | | | 889685 | 457676 | | |
| SEQ ID | 141356_ | 5.01E−06 | B, X** | C | T | 7 | 28,685,688 | 160678_ | Cannabis.v1_ | 28,594,408 | 29,061,134 |
| NO: 192 | 120636 | | | | | | | 5015 | scf2920- | | |
| | | | | | | | | | 38973_127 | | |
| SEQ ID | 142269_ | 4.79E−06 | A, X | T | A | 7 | 50,822,516 | 142269_ | 126812_ | 50,818,092 | 52,439,705 |
| NO: 272 | 104610 | | | | | | | 100184 | 8470 | | |
| SEQ ID | 141735_ | 5.89E−09 | B, X* | C | A | 7 | 52,923,743 | 140726_ | 132241_ | 52,544,592 | 53,396,185 |
| NO: 288 | 113420 | | | | | | | 109620 | 2253 | | |
| SEQ ID | 141735_ | 3.63E−06 | B, X | A | G | 7 | 53,505,022 | 141735_ | 170870_ | 53,449,873 | 54,375,898 |
| NO: 292 | 492163 | | | | | | | 437148 | 14856 | | |
| SEQ ID | 140997_ | 3.63E−06 | B, X | A | G | 7 | 54,400,345 | 170870_ | 140997_ | 54,375,898 | 54,452,078 |
| NO: 294 | 363509 | | | | | | | 14856 | 415246 | | |
| SEQ ID | 140997_ | 3.63E−06 | A, X | G | A | 7 | 54,464,358 | 140997_ | 140997_ | 54,452,078 | 54,646,726 |
| NO: 295 | 427582 | | | | | | | 415246 | 589675 | | |
| SEQ ID | 140997_ | 3.63E−06 | A, X | T | G | 7 | 54,509,399 | 140997_ | 140997_ | 54,452,078 | 54,646,726 |
| NO: 296 | 464201 | | | | | | | 415246 | 589675 | | |
| SEQ ID | 140997_ | 3.63E−06 | B, X | A | G | 7 | 54,513,057 | 140997_ | 140997_ | 54,452,078 | 54,646,726 |
| NO: 297 | 467859 | | | | | | | 415246 | 589675 | | |
| SEQ ID | 133080_ | 3.63E−06 | B, X | C | T | 7 | 54,555,051 | 140997_ | 140997_ | 54,452,078 | 54,646,726 |
| NO: 298 | 7333 | | | | | | | 415246 | 589675 | | |
| SEQ ID | 140997_ | 3.63E−06 | B, X | G | A | 7 | 54,693,540 | 140997_ | 140997_ | 54,683,300 | 54,714,058 |
| NO: 299 | 628227 | | | | | | | 617987 | 648743 | | |
| SEQ ID | 140997_ | 3.63E−06 | B, X | C | A | 7 | 54,722,302 | 140997_ | 131855_ | 54,714,058 | 54,860,264 |
| NO: 300 | 656987 | | | | | | | 648743 | 1935 | | |
| SEQ ID | 140997_ | 3.63E−06 | B, X | G | A | 7 | 54,915,189 | 131855_ | 137156_ | 54,860,264 | 55,029,197 |
| NO: 301 | 859069 | | | | | | | 1935 | 6696 | | |
| SEQ ID | 142086_ | 3.63E−06 | B, X | G | A | 7 | 55,277,957 | 142086_ | 142086_ | 55,255,666 | 55,316,629 |
| NO: 302 | 207043 | | | | | | | 184758 | 231920 | | |
| SEQ ID | 142086_ | 3.63E−06 | A, X | A | T | 7 | 55,452,244 | 142086_ | 142086_ | 55,316,629 | 55,716,705 |
| NO: 303 | 326496 | | | | | | | 231920 | 537884 | | |
| SEQ ID | 142086_ | 5.89E−09 | B, X* | G | A | 7 | 56,032,988 | 142086_ | 142086_ | 56,018,989 | 56,076,209 |
| NO: 310 | 826368 | | | | | | | 818053 | 869588 | | |
| SEQ ID | 142086_ | 5.89E−09 | B, X* | G | A | 7 | 56,301,604 | 142086_ | 142086_ | 56,171,548 | 56,426,824 |
| NO: 314 | 1049946 | | | | | | | 948791 | 1157635 | | |
| SEQ ID | 141318_ | 5.89E−09 | B, X* | A | T | 7 | 56,967,275 | 141318_ | 141318_ | 56,910,768 | 57,069,404 |
| NO: 328 | 522267 | | | | | | | 572197 | 415904 | | |
| SEQ ID | 142593_ | 5.89E−09 | B, X* | C | T | 7 | 58,538,433 | 142593_ | 142593_ | 58,428,139 | 58,607,780 |
| NO: 348 | 660489 | | | | | | | 559015 | 696671 | | |

### QTL Mapping

The F2 mapping population segregating for types I, II, and III (n=294; set 7, Table 3) was genotyped with an Illumina bead array. After initial SNP QC, further filtering steps were performed to filter out known low quality SNPs, SNPs with large numbers of missing values (>50%), linked SNPs (SNPs in 5 kb regions evaluated for LD>0.2) and SNPs with a minimum allele frequency<1% using vcftools (Danecek, Petr, et al. “The variant call format and VCFtools.” Bioinformatics 27.15 (2011): 2156-2158). Subsequently, SNPs deviating from Hardy-Weinberg equilibrium were removed based on a threshold of 1E-06 using plink (Purcell, Shaun, et al. “PLINK: a tool set for whole-genome association and population-based linkage analyses.” The American journal of human genetics 81.3 (2007): 559-575.). After these filtering steps, 7607 array SNPs remained for map construction and QTL analysis. A linkage map was constructed using the F2 mapping population SNP data using the package MSTmap (http://mstmap.org/). QTLs were mapped on this linkage map using the R package QTL (https://rqtl.org/). QTL mapping was performed separately for the greenhouse and growth room data for Total Cannabinoids (the growth room data did not include Total THCV, Total CBDV, Total CBG, and Total CBGV because of inability to detect these with the chemotyping method). Total Cannabinoids to CBG Ratio QTL mapping was only performed using the greenhouse data since the chemotyping method used for the growth room data was not able to detect low quantities of Total CBG.

Significant QTLs were detected for Total Cannabinoids in greenhouse data on chromosomes 1 (LOD=4.40; p=0.008), 4 (LOD-5.09; p=0.001) and 10 (LOD-6.67; p<0.001; chromosome 10=X chromosome). A significant QTL was detected for Total Cannabinoids growth room data on chromosome 1 (LOD=4.79; p=0.01). Significant QTLs were detected for Total Cannabinoids to Total CBG Ratio greenhouse data on chromosomes 4 (LOD=7.80; p<0.001), 7 (LOD=8.44; p<0.001), and 10 (LOD=5.14; p=0.001).

TABLE 25
Most significant NAM SNP markers within QTL 1.5 LOD support intervals with Fisher's combined
probability (which includes QTL p-value) below the Bonferroni multi-test threshold. First column, SNP
marker number; second column, SNP marker name; third column, mapped trait for NAM (for QTL mapping
Total Cannabinoids was mapped instead of Total THC, Total Cannabinoids to CBG Ratio was mapped
instead of THC to CBG Ratio); fourth column: Abacus reference genome (CsaAba2 version) chromosome;
fifth column: Abacus reference genome (CsaAba2 version) position of SNP marker in bp; sixth column:
position of the SNP marker on the F2 population linkage map in cM; sixth column (*Not part of the
linkage map, two flanking SNPs 90_683036 and 90_794889 at positions 1,581,181 and 1,705,516 were
located on the linkage map at 8.07 and 10.37 cM, respectively. **Not part of the linkage map, two
flanking SNPs 142293_8648325 and 142293_8666416 at positions 55,309,422 and 55,354,218 both were
located on the linkage map at 47.48 cM.); seventh column: position of the QTL on the F2 population
linkage map in cM; eight column: LOD value of the QTL; ninth column: start and end positions in
cM of the 1.5 LOD support interval surrounding the QTL; tenth column: NAM p-value for mapped
trait; eleventh column: QTL p-value for mapped trait; twelfth column: Fisher's combined
p-value based on NAM and QTL p-values for mapped trait.

| | | | | | | | | Positions | | | |
|---|---|---|---|---|---|---|---|---|---|---|---|
| | | | | Position | Position | | | 1.5 LOD | | | |
| | | | | SNP | SNP | Position | LOD | support | | | |
| | SNP | Mapped | | marker | marker | QTL | value | interval | NAM | QTL | Combined |
| SEQ ID | marker | trait | Chromosome | (bp) | (CM) | (CM) | QTL | (CM) | p-value | p-value | p-value* |
| SEQ ID | 90_ | Total | 1 | 1,605,949 | 8.07-10.37* | 7.56 | 4.79 | 6.00- | 3.24E−07 | 1.00E−02 | 6.66E−08 |
| NO: 1 | 708457 | THC in | | | | | | 12.00 | | | |
| | | type I | | | | | | | | | |
| SEQ ID | 142603_ | Total | 4 | 8,478,160 | 46.39 | 53.89 | 5.09 | 30.00- | 3.02E−05 | 1.00E−03 | 5.53E−07 |
| NO: 24 | 4839736 | Cannabinoids | | | | | | 77.22 | | | |
| | | in types I, | | | | | | | | | |
| | | II, and III | | | | | | | | | |
| SEQ ID | 141440_ | THC to CBG | 7 | 41,296,609 | 39.56 | 36.627 | 8.43 | 34.239- | 2.51E−15 | 1.00E−03 | 1.04E−16 |
| NO: 217 | 750937 | ratio in | | | | | | 39.56 | | | |
| | | type I | | | | | | | | | |
| SEQ ID | 142293_ | Total | 10 | 55,334,163 | 47.48** | 46.79 | 6.67 | 41.49- | 2.24E−05 | 1.00E−03 | 4.17E−07 |
| NO: 362 | 8653733 | Cannabinoids | | | | | | 52.081 | | | |
| | | in types I, | | | | | | | | | |
| | | II, and III | | | | | | | | | |

Validation of SNP Markers with QTL Results

Validation of SNP markers was performed by comparing NAM and QTL mapping results. Table 25 shows within each QTL's 1.5 LOD support interval the most significant SNP (as identified through NAM of data sets 1-3) with a Fisher's combined probability with its corresponding QTL p-value below the Bonferroni multi-test threshold. This resulted in two additional significant SNP (SEQ IDs 24 and 362 Table 27). The beneficial genotype for the mapped traits corresponded well between the F2 and NAM populations (Table 26). For two SNP markers, the homozygous alternative allele genotype, which was the beneficial genotype in the F2 population, was absent from the NAM population. For those two SNP markers, the heterozygous genotype was identified as the beneficial genotype since average mapped trait values were higher for the heterozygous as compared to the homozygous allele genotype.

TABLE 26
Beneficial genotypes for most significant NAM SNP markers within QTL 1.5 LOD support intervals with Fisher's
combined probability (which includes QTL p-value) below the Bonferroni multi-test threshold. First column: SNP
marker number; second column: SNP marker name; third column: mapped trait (for QTL mapping Total Cannabinoids
was mapped instead of Total THC, Total Cannabinoids to CBG Ratio was mapped instead of THC to CBG Ratio); fourth
column Abacus reference genome (CsaAba2 version) allele; fifth column: alternate allele; sixth column: beneficial
genotype for mapped trait (*The NAM population segregates T/T and A/T with A/T significantly associated with
Total THC in type I. Since in the F2 population the beneficial genotype was A/A, it is assumed that the beneficial
genotype for this marker is A/T as well as A/A. **The NAM population segregated C/C and T/C with T/C significantly
associated with Total Cannabinoids in types I, II, and III. Since in the F2 population the beneficial genotype
was T/T, it is assumed that the beneficial genotype for this marker is T/C as well as T/T.)

| | | | | | | | | Position | Position |
|---|---|---|---|---|---|---|---|---|---|
| | | | | | | | | left | right |
| | | | | | | Left | Right | flanking | flanking |
| | | | | | | flanking | flanking | SNP of | SNP of |
| | | | | | | SNP of | SNP of | marker | marker |
| | SNP | Mapped | Reference | Alternate | Beneficial | marker | marker | haplotype | haplotype |
| SEQ ID | marker | trait | allele | allele | genotype | haplotype | haplotype | (bp) | (bp) |
| SEQ ID | 90_ | THC in | T | A | A/T and | 90_ | 90_ | 1,593,859 | 1,611,839 |
| NO: 1 | 707845 | type I | | | A/A* | 695755 | 713734 | | |
| SEQ ID | 142603_ | Total | G | C | C/C | 129260_ | 142603_ | 8,461,995 | 8,498,792 |
| NO: 24 | 4839736 | Cannabinoids | | | | 2903 | 4819405 | | |
| | | in types I, | | | | | | | |
| | | II, and III | | | | | | | |
| SEQ ID | 141440_ | THC to | T | G | G/G | 141440_ | 141440_ | 41,248,971 | 41,343,647 |
| NO: 217 | 750937 | CBG Ratio | | | | 798575 | 705988 | | |
| | | in type I | | | | | | | |
| SEQ ID | 142293_ | Total | T | C | T/C and | 142293_ | 142293_ | 55,309,422 | 55,349,111 |
| NO: 362 | 8653733 | Cannabinoids | | | T/T** | 8648325 | 8661311 | | |
| | | in types | | | | | | | |
| | | I, II, and III | | | | | | | |

Table 27 provides a listing of the sequences of the present invention, which are located at position 51 of each respective sequence:

TABLE 27
50 bp flanking sequences surrounding SNP markers.
First column: SNP marker number;
second column: SNP marker name;
third column: 101 bp sequence with the SNP marker at position 51 bp,
sequence is from the Abacus reference genome (version CsaAba2).

| | | |
|---|---|---|
| SEQ ID NO: 1 | 90_707845 | AAATAAATAAAACTAAAGAAAGCTGGCACGAGGCT |
| | | TTTTCTTGTGTACTATAAATTAGTTAAATTTGATA |
| | | TATAAATTTTCTCTCTCTCTTTTCAAGTTGA |
| SEQ ID NO: 2 | 90_1205270 | TGGCTTTAATATCGTACATTACAATATTACATTGT |
| | | CCAAACTGCCCCCGAGACCTTTTAGCACATTGCCC |
| | | TGAATTTGGTAATCGACCACCATTACTGAGC |
| SEQ ID NO: 3 | 139707_9823 | GGATCGAAATGATGAGTTAGTTGGACGATCCAAAA |
| | | GCGAAGGAGCACGTTCGAGTTGGTTCACTCGTCCT |
| | | GCTACGTACTCACCTCCGACAGCTCAAAGTA |
| SEQ ID NO: 4 | 123886_2182 | TCAGCCAGCAGCTCAGTCACGGCTCCACCCGAGAA |
| | | GAACCAAACAACAATGCAGCAGCTTCTAACATGGC |
| | | TGGTAACACCTCTTTTGTTCAAAAATTATCT |
| SEQ ID NO: 5 | 300_56425 | TTATGTTCAGAAGTTGAATTGAGGTGATTTAAGTT |
| | | GAATTATGTTAAGTTTGTGGATTTTGGTGATTTTT |
| | | GGGCAAGGTTTGAGTTTAAGAACTCAAGCTT |
| SEQ ID NO: 6 | 166_982294 | ACTTGTTGTATGAAAAATGAATGAGCTTTGGAAAG |
| | | AGGAATCAGTTGTAAGAAGATGTGACTGAGGTTGT |
| | | AGAAAGAATATTTCAAAAAGACAATTACATT |
| SEQ ID NO: 7 | 142264_961 | CATCGCTCGGCGCAAGGTACGTAAAATCTTGAGCC |
| | | CATTCGAGTAATAATCACTTTGAGCATTTATACTT |
| | | CAAATTAATTCAGCATTGTCTTGCCCAATCG |
| SEQ ID NO: 8 | 211_137879 | TCCCCGAATCATGCGCCATTAAATTATCACATAGA |
| | | GATCTTACCATGTCTGTCAAGTGGTTAATTTCTTC |
| | | TGGAAGTGATAGTATATTTTTCACTTCTTCA |
| SEQ ID NO: 9 | 141928_1221494 | TGATATTATTTTACTATCTTTTTGGGATAACATGT |
| | | GAGCTTCAATTATTTGTAACAAGAAGAGTTAATGA |
| | | GGTCGAATTATTTTAAAAAATAAATTTATTT |
| SEQ ID NO: 10 | 141928_1159985 | AGAGAATTAAATCTCTTTGTTGGAGTCATGCTCTG |
| | | TAGTTCTCCGTTATGTATGGCTCACAAAGTGGCAA |
| | | GAATCACGCGCTTGAGCAGAGCGTGGAAGGA |
| SEQ ID NO: 11 | 141928_866974 | AACCCGAGCATGCGCAGTCTCAGTCTTAGTGTTAA |
| | | GCTGTAAAACCAGTTGTTATTATCTTGTTCTTCAT |
| | | CTTATCTGAGCAACAAATTCTTGCTCCATTG |
| SEQ ID NO: 12 | 141928_611188 | CTCTCCACTCCTCTCTCTCTTAAATTTTCTTTCCT |
| | | TTAATTTTTCCACCATACCAAACATAGGGTAAGTA |
| | | TATTTTTTTTTTTGTCACAAAGCATTCTTTT |
| SEQ ID NO: 13 | 141928_604731 | AAGAAAAAGATGAGTACCCTTTTAGAGGCTGATGA |
| | | TAAAGCCTCGAACTTCGTAGAGAGATGAAGAATTA |
| | | GTACATGAAAAATTAAAGCTTTGATGATTTT |
| SEQ ID NO: 14 | 141928_547218 | TTTCTTGCTACTCACTACAAGAATGTTAAGAGATT |
| | | TGAAGAAGCAGAGGTGTATTGTACCCGTCTTCTGG |
| | | ATTACAATGGCCCTGTAAGTTCTAATATTGT |
| SEQ ID NO: 15 | 141928_1533875 | TCGGTAGTGACAATACTCAGGGATCCAGTTGACCG |
| | | TGTCTTTAGTACTTATGAATTTTCAATAGAAGTAG |
| | | CAGCTAGGTTCTTGGTGCATCCTAATTTAAC |
| SEQ ID NO: 16 | 141928_1706518 | TGAGAAGGGCATTAGTTCAAAATTTGTGTATATAG |
| | | TCTTAACCTTGAAAATTTGGATTGGATTGGACCCC |
| | | AACCCAACACCCTTACTTGCAAGCCAGTTAT |
| SEQ ID NO: 17 | Cannabis.v1_ | AGCAACTGCAGAACCACTCCCACCCCAAAACAAGT |
| | scf211- | CACCAAACAAGGAGATGGCCCTTAAATATTCTTAG |
| | 243477_101 | AGGAAAAATGATAAGTGAATAATTAATTATA |
| SEQ ID NO: 18 | Cannabis.v1_ | ATTATAAACAATAGGAAAAACCAACACCAGAGATA |
| | scf211- | ACACAAAAACATGTACACAACTTTATAATGCCGCT |
| | 234052_100 | CTACCATGAATATGTCCAGTCTTCTCGCAAA |
| SEQ ID NO: 19 | 140726_292253 | ATTTTCCAGGTGTTTCTTTGGACTTCAAATCTGAT |
| | | GGCAGCAATGAAATTGCTACAGCAACTATTTCAAT |
| | | CTCATCCCATTTTGACAGAGGTGTTAATGAG |
| SEQ ID NO: 20 | 142169_3375917 | AAAATGCATTTCTGAGTTATTATGAGAAACTGTTG |
| | | GGAACTAGTATGCATTATAGAAGGAAGGTTCTGCG |
| | | ATCTGTAGTCAATCTTGGCCCCAAGATCACA |
| SEQ ID NO: 21 | 192305_372 | AGTTCTCATCTAATGATTGTTGTGTGAAGGACAAC |
| | | CAAACAAAGAAGAAAGTGCTTCAAGGGAAGCTTAA |
| | | GGAAGGTCTCTATCAATTCAACTCTCACTGT |
| SEQ ID NO: 22 | 194726_3613 | TGATATGGAATTTTTCTCAGGTTGGGGTTATTTGT |
| | | GGATAATCTCAAACGGAGATGTTCATTTGACTTTG |
| | | CTGATGTGAATTTTCTTTCTTTTGGGATCTT |
| SEQ ID NO: 23 | 142603_10585838 | TCTAGGTGATGCACTTGAGGGCTCTCGAAGAAGGA |
| | | TTCCCAATGATTGTTAATGAAGACCCAATCAATTC |
| | | GTTCTTTGATAGTATTGGCAGCAGCTCGATT |
| SEQ ID NO: 24 | 142603_4839736 | AGGAAGAATGTCGCAGAAAAAGTAGATTTCGTAGG |
| | | ACAATATAGGGTACAGTAATAAAATTTATAGGGTT |
| | | ATTCTTGTTAAAATTATTGGGAAATTTACAT |
| SEQ ID NO: 25 | 142603_4009627 | TTATATGGAGACCATTCAAGACCTTCTTGATCCAT |
| | | CAAATGATAACATCCCCTTTGTGGAAGATCCAAGA |
| | | ACTGGTGATGTTTCAGTACCTGGTGCGACTC |
| SEQ ID NO: 26 | 331_143425 | AAAACCCTACCGTACAATGTGAGTGTCTTCGCTGT |
| | | TAATTCTCTTCTCGTTCCGTACGGATTCGATCTAA |
| | | TGGCTTCCGAGACTCGACCTCCATTAGGTTT |
| SEQ ID NO: 27 | 142050_4065320 | AATTTAAGTTCTGGGTTTTCATGGATTTGCCTCCT |
| | | TATTGAGACCAACCAGTCTTTTTCCTGTTTGGCTG |
| | | CCTGGAAAATCTCATTGGCGTAATGGGTATT |
| SEQ ID NO: 28 | 142050_3752784 | TGATTATCATTGTTAATTATTGTTTTACCTATATC |
| | | TATATATGTAATTGTTAACTATATCCATTTTAATA |
| | | GAGACCCAATAACTAACCAAATCTTCTAATT |
| SEQ ID NO: 29 | 142050_3748265 | GTGTGTTGTTAGGCCTTGGACCAAATAAAGGGCAA |
| | | TAAAGGTAAATTGTCTATTGAAGATCTAAACATCT |
| | | CTATGAGACGAAAGAATTATACTTAAATAAA |
| SEQ ID NO: 30 | un71484_59_60 | TTGTTTGAGCTACTCCTTTCAGTCTCAAATCAGGA |
| | | TTGCTTTTGTCTAGATATTCGGGTCGCAGCAAGCT |
| | | CGAATTAAAGCATTGTGAAAACATCAGGCTT |
| SEQ ID NO: 31 | 142050_2956805 | CTCTGGATGATTACCGTCATGTGGGTTGTTATCAG |
| | | CCATTTAATATGGGTTTGTATTTGTTTTATAAGGA |
| | | AGGATTTTCCAAGTTTTTATCACCAAGACTC |
| SEQ ID NO: 32 | 142050_2863275 | CGAGGTAGACAAACAGCTCTTCAAGGCTAGCCAAC |
| | | GAACCTGTGTTTAGGAGTGAAAAATCTATCCCAAA |
| | | TGCCATCATAACGCCATCTAACAGCACTTCC |
| SEQ ID NO: 33 | 142050_2818430 | TTTCTTTGGGCTATCTCCACTGCAAACTTTACTAA |
| | | AGGGCTCGTAATGTCATCTCCTTCAAATTTCAAGA |
| | | ATGTTGCATTCATCCCATTTTCATTACTCTC |
| SEQ ID NO: 34 | 142050_2787250 | TAGTCTTCATTGGGTCTCTGGTCTTTCTTCACTAG |
| | | TATATCTAGAGATGAGAGCAAACCTTAGTGCAACA |
| | | TCTGATCATTGGCTACTCTCTTTAAACAAAA |
| SEQ ID NO: 35 | 142050_2639484 | TTGCTCTTGCTAATTTTGTAGTACTAAGGATTAAT |
| | | GAAAGTGGAGAAAATTGATGAACTCCCAAGCAAAA |
| | | CATTATTATAATTTTGGCTTATTAATTACAA |
| SEQ ID NO: 36 | 142050_2637918 | TTTGCAGTATAGATATTTAAATGATTAGTTATATT |
| | | AATAATTTAATATGGCCACTTGATTTATTTTAGAT |
| | | TCATACATAGGTAAGCATCCATTAAATTTTC |
| SEQ ID NO: 37 | 142050_2212118 | ATTTATACATAATAACAGTCTTTTACAAACAGCAA |
| | | GAACACGAAACGCAATAGGCCGCCCTACCCTAAAG |
| | | ACGGGCATAACACAAAACAATCCTTCTTAAT |
| SEQ ID NO: 38 | 142050_2052266 | TATGAAGCAGAAGAACAACAACCAAAACAATAATA |
| | | TTCAGCCCTTCATACCTGGTTCAGTTATTATTTTG |
| | | TTTTGTTATTATTTTCAGTTATGCTTGGTAT |
| SEQ ID NO: 39 | 142050_2040041 | TCCTCTCCTTCCTCAAGATCTCTATGACACCATGT |
| | | CACTATTATGATTCTTCCTCCTGGGGCTGCAACTC |
| | | TAACCAATTCACTCACAAACTGCACACTCAC |
| SEQ ID NO: 40 | 142050_1748980 | TTGGGAGTTGAAGAATTGATTCCCATACTCAGTTA |
| | | TCTTCATCCTGGAGAGCTCTCCTATGCTAAATATT |
| | | ATGCTGGTTACTTAATGAAGGAGGTAATTAA |
| SEQ ID NO: 41 | 138186_4346 | TCATTGTAGAGCTTATATTAATAGTGGCTGAGTCA |
| | | AGTTCCACTCTTCAATATTTGACTCAAAATATTGA |
| | | CAATATTATTCAATTAATTATCTGCATTAAA |
| SEQ ID NO: 42 | 138186_53738 | TGAATTGATTTGTGTAATTTTAGTTTTTGTTGCCT |
| | | TACTTTTACTTGATTATGGAATCAGGTCATTTTTG |
| | | GATGATAATAATCATAGGGGCTCCCCAAATG |
| SEQ ID NO: 43 | 142050_1719103 | TCTCTGTCTTGGGCAGAGGATCATTTTCAACATAT |
| | | TATGCTATTTTTTCTGCTTATCTTCTAAAAACGGT |
| | | TGGATCTTGGGTGTCCTCCTCAGCCTCTGCA |
| SEQ ID NO: 44 | 128680_3420 | TGAGTCAACTGAAGTCTACGAGCTCTGACTCTTGC |
| | | TTGGACTCTAACCAAGGCTTGCATACATCTCATTG |
| | | TCATTTGTGCTTGTTTTCTCACATTGTGGCC |
| SEQ ID NO: 45 | 142050_1411714 | CTCTTTGTCGATCTCTATAAAAATTAAATAAAAAT |
| | | CAAGAAGAAAAAACTCTCACGTACTTCATCAATTA |
| | | ATTAATTCTTCTCCATATTATATATCCTAGA |
| SEQ ID NO: 46 | 142050_1254508 | ATCTCTTTCACCTTTGTTATTTTCTCTCACTACAT |
| | | TCCTAAACATTTCCCATATGCACCAAGGGCCACAA |
| | | CAACAACAACAATCCCTTCATTTAGTTTGAC |
| SEQ ID NO: 47 | 142050_1011763 | GGAAAAAAAATATTCATGTGTTTTCAGATGCATAA |
| | | ATTTAGAAAGAAGTAATAAAAACTCCTCTTTTTTG |
| | | TAACTCTGAATTTGAACGATTTGTGTCTAAA |
| SEQ ID NO: 48 | 142254_6896550 | ACTAAACTATTGTGGCTACAAAGACAAATAATTAA |
| | | TCCTTGTAGAATAAACAAAGCTTCAAGATAATTAC |
| | | AAGCTGATTAAGAAATAAAAAAAATAGGACT |
| SEQ ID NO: 49 | 142254_6738322 | GAACTGGCCAAAGTTTTCCACAAAATGGCTTGGCT |
| | | AGTCACACCAAGGCATATACCAAATGAAGAGATGG |
| | | GAAACCTTAGAAGAAATGGCCATCTCTTGTC |
| SEQ ID NO: 50 | 142254_6394766 | TACAGTACTAACATATAAGATAATTGCGTCATTAC |
| | | ATTCGTAATTTTGTGAGTTAGAATTTTAGTTTTCA |
| | | AGGAATTATTTCGTCCGTGGCTTGTTTTAAT |
| SEQ ID NO: 51 | 142254_6291758 | GATGACGATTCTTTCCCTTAGGAAAATTGCTCCTT |
| | | TGTTGACCTTTCCCTTAAAGGAGATCAGAGTGAAC |
| | | TTTACGTGGATCTTGAGCCTTTTTATTCCTT |
| SEQ ID NO: 52 | 142254_5690149 | TTCCATCCCCATTCTCTATCGATATCCATTCAAAA |
| | | TGAGTTTCTTCTCCCAAATCAAGCTTTGTCACATG |
| | | AAGGATGCATTACTTCCGCACTTCGCAGCCA |
| SEQ ID NO: 53 | 142254_5526537 | TTGGTGGCCCGGCCCAGAGATGCATGGAGGAAAAT |
| | | GTTTAGTATGAATGAGATTGATGTGGCAACAAATG |
| | | GGTTCTCAAGAGCAAATGTCATTGGTAGTGG |
| SEQ ID NO: 54 | Cannabis.v1_ | ATATTTTCCATATCTTATTTGGCAACAGTTATCAT |
| | scf5269-5524_101 | ATCAGCACGCATGCTGCATTTCGTATTTGGTACCA |
| | | TAGGACATACATAGTCACACTTAAGTTTTGA |
| SEQ ID NO: 55 | 142254_4671720 | CAAACAGGTTTGGATCGGAGATAGTGGTATACTGT |
| | | TAGAATATTTTACGTTGAGATTACCCAGCAGCAGC |
| | | GTTGAGCGAATTGGAAAGATGCTATCGTGCA |
| SEQ ID NO: 56 | 142254_4412683 | ATTTTCATTATGTTTCTTTGACAGCTAGATGAGGA |
| | | CAAAGGGAAAGTTTCTGCTCCAACTTGTACAAAAT |
| | | CTGTCTCTGAACGTATAGAAGAGGAGGTGTG |
| SEQ ID NO: 57 | 142254_4329494 | CCTTGTATTTTAGCCAAAGCCCAATGTATTTTAGA |
| | | TGAGTGGGTTATATTCACTAACTTGGTCTTGAAGG |
| | | ATTGTAATTTCAAATTGGGTCGTAAGAGGCA |
| SEQ ID NO: 58 | 142254_4191858 | AGTAGTGAAGTTCAAACCAGTTTCAAAAGTTCCAA |
| | | ACACAAAGTTTGAATGAGATCACTATTTGGTAATA |
| | | TAATAAGGAACTTTCTCAAGTAAGTCCACAT |
| SEQ ID NO: 59 | 142254_4187450 | CCATTGCCAAAAAAAAAAGATAACTTGTGGGTGAA |
| | | GTAGGTACATAATGTGTACGTGAAGGAGGAAGATT |
| | | GGTGGAATTAGTATTGGCACAAACTGGTTGC |
| SEQ ID NO: 60 | 142254_3855623 | ATGAATTGATCCAAGCCAAAGAATACTTCTTTCTT |
| | | TTGTTGCAGAAATTCCACTAAATAACACACCGCAG |
| | | GATCTGAACCACGCGAAACGCAGGCTGTTGA |
| SEQ ID NO: 61 | 142254_3554355 | TCTACAACACACCCTGTGTATACAGACCACAAAGG |
| | | CAAGTAGAAAAATTTCCAAACCAGCATACTTCAAT |
| | | GACTAATCCTTTTCTGTTTCCAACAGTGCAA |
| SEQ ID NO: 62 | 142254_3540209 | ATGTAGAATGTTCCTCAGCGACAATGGTTAGGGCT |
| | | CACTTCATAGATTTAAATTTTGTGCTCAGATTACC |
| | | TCCATTTTTTATCTTGTGTATGCGAATGAAA |
| SEQ ID NO: 63 | 142254_3525755 | TTTAGGGTTACACCATCGTTATCTTGGAAGATGAA |
| | | AAGGTTGAAGATGTGAAATATTATTCAATTCAACA |
| | | CGTGCTAACTCAACAAAAGTTTAATCATTTT |
| SEQ ID NO: 64 | 142254_3458512 | AAAATATGAATCGAGTCAGCACGACATGCACAAAC |
| | | TTAATTAGAACACTAGCTGGTAGTGAGGTAAACAT |
| | | ATTATATATTATTTCATTTTTCTCTCTCTCT |
| SEQ ID NO: 65 | 123613_11373 | GGATGTCCAGAGGATCTAACCTAACAATATGAATG |
| | | CGTTCCTAGACAGAGTTGACGACTTTATCAAGCTA |
| | | GAGGAAGTTTTCCAGAAAGCCCAGATAGGGA |
| SEQ ID NO: 66 | 142254_2808940 | ACTTGTCAATGGGTGAACGAAGTCACCTTACACAT |
| | | CGTGCCAATTGTTAAGGGTTCCAAGCAATCAAGCA |
| | | AGATTAAGTAAGGAGAAAATGACCAGCCAGG |
| SEQ ID NO: 67 | 142254_2797082 | ACGAAACTAAATATGTGTGGCAGGGGCACCACAAC |
| | | ATTCTCATAAAAACTCCCACAGAGCCCAAATAAGA |
| | | CACTTGACAGAGCTAATGACCAAAATTTATG |
| SEQ ID NO: 68 | 142254_2754905 | AATTTTCTGGATCATTAGTTTCTCCTTCTCTTTCA |
| | | GCATCCACCCTAAAAACTACTTCTTCCTCTTTAGG |
| | | ATCCTTTGGGTCATCTGGTTGTTGTCAATGT |
| SEQ ID NO: 69 | 142254_1798647 | GGTTGCATTGCACTGGTTGGCATGGTTAACTAACT |
| | | AGTGATGAGAAGTAAAGTTCATCTCTATAATCGAG |
| | | AGGTAATGTGTGTTTATTGTATATTTTTTTA |
| SEQ ID NO: 70 | 142254_1356204 | AGGTGCAAGTGGTTACGATGTTGCTCATAGTACTA |
| | | GTTTCGCTGTGAGGTTACTTGAATTCTAATTGGGA |
| | | ACATGCCTTCAGTTTCATATGTGAGTGAGAA |
| SEQ ID NO: 71 | 142254_945759 | ATATTTATGGCAAAAATAAGGAATGGACTGGCCTA |
| | | GCTATGGCTCAACAACAACTACCAGACTCACTACA |
| | | ATCACTGAAAGTATATGAAAAGATATATGTA |
| SEQ ID NO: 72 | 142254_618972 | CTCCAGGACCCAGAGTTGTCCAAGATAAAAGGTTG |
| | | CCCAAGTGATACCCAAATGGGTACTCAAACGGCCT |
| | | ATCACGGACGCTAACCACCCAAAAGGATCGC |
| SEQ ID NO: 73 | 109654_9870 | TGAATTCGCTCAACAGCTCAGGCAGAAATGTCCAA |
| | | AGCCAAACAATGACATAACCGCAGGACAGTTTTTG |
| | | GACTCAACTTCGTCCAGCTTCGACAACGATT |
| SEQ ID NO: 74 | 140930_95958 | TCTATATTCTCAGTCTCTAGTTTTTAATGATAAAC |
| | | TGAAGACTCTATTTTCGTATTCATCAACCCTTGTC |
| | | ATATGTTCTCAACTGTTCCTGTTACTGCAGG |
| SEQ ID NO: 75 | 103043_12945 | GTCATATATAGCTTTCCATAATTTCCTAATTTTAG |
| | | CCCGTATGACATATAGGCATCATGAAAGATGTCGA |
| | | ACCATTTCTATGGTTTAAGCGTACTATATAT |
| SEQ ID NO: 76 | 141234_646804 | TGGTTAACTTTATTGAAGCTTGACCGTTGGTGTGG |
| | | GTCTCATTTTGTTGCGGATTAAGTATGTGAAGCTT |
| | | AGTTGGCTTTTGGTCTACCAATATATATATA |
| SEQ ID NO: 77 | 135428_7370 | AATCAAGTGGCTCCTAAAAGTAACAGGGACTTCAA |
| | | GAATATCCTGTATAACAGACTCATCAAAATGATTT |
| | | CTCAGATTGCCTATATCCCAATACCCAGAGT |
| SEQ ID NO: 78 | 141363_504946 | TTCAAAATATGGGTGAGTGTAATGTGTTTGGGTTA |
| | | GGTGGAATTATTCATTCACGTGTTTCTTATGATGG |
| | | AAAGGAATTGTAATATATATATATATATTAT |
| SEQ ID NO: 79 | 141363_624738 | TATCAAACTTAAATTTAAAGCTTGCAGTGAGAAGC |
| | | CCACTATAATCATGAGGTTAGAAATGAACCCAAAA |
| | | TTAAGGAACAAGATAAGATCCCTTAATTTTT |
| SEQ ID NO: 80 | 141363_961227 | TTCCAAGGAAAACAAACAAGCAGTACGTTAAAGTC |
| | | TAAAGGGCCGCACCAACGTATTCCACCTTTTTACA |
| | | GATAATTGTTATTAATTTCTCACCATTACCA |
| SEQ ID NO: 81 | 125598_9975 | ATCCCAAGTGATAGCGGAATCACATTTAATGTCAA |
| | | ACCATTAATGAAGGCCGTAGAAATTGCTGAGAAGG |
| | | CAAGGGATGCCATACTTAGCCGGAAATTCCA |
| SEQ ID NO: 82 | 141416_227999 | AGTCTCTGTATATAGAATATACAAATTTATATTCT |
| | | TTCCAACGGATTAGATGACTGTTGAACAATGAGTT |
| | | TTGAACTCGTAACCATCCTCATCTACAATAC |
| SEQ ID NO: 83 | 124185_3188 | CAGGTAACCCAAACAATTCGAAACCCCAACCAAAT |
| | | CAAATCTAAACCTCACCAAACCTTCCATACCTGCT |
| | | TAAAATACGCCAATAAACATAACCGAAGCAA |
| SEQ ID NO: 84 | 141632_409539 | TTTTGATTTGCCGTTGAACAATGTGATTTTCAATT |
| | | TCTTTATAGTTTTATTATTGTTTGGTGCTAGTTTA |
| | | TTAATCTTTATTTTTTTCAAATATTTTTGTT |
| SEQ ID NO: 85 | 141632_403504 | GTGATCCACGGCTGAAGGACTAGGGAATGCCATTG |
| | | GCAAAAATCAAGGCAATCGCCCGAACCCATCCAAA |
| | | GTCGCACCACTCATCCATATTTTCAAGAGAT |
| SEQ ID NO: 86 | 141632_396575 | CAAAAAATCCCGATGGCTGTAAAGGAAATAAGGTG |
| | | AATTAGATCAATTAAATTTTGTGGCTAGCCGTATA |
| | | AAACCATCCCCTTCACCACCACTCAGACATA |
| SEQ ID NO: 87 | 141287_286298 | TACTCTATCTTTTGCGTGCAGCTCTAATCTCAGGC |
| | | CCTGATTACATGTATAATATAAACCATTGTACTTC |
| | | CATGAGATTATATTTCCATTTGCATTTGTTA |
| SEQ ID NO: 88 | 140600_466642 | AGCCAGTCATGGAGTCACCAGTCTTAACGGAGCCT |
| | | CAAATAACAAAGGGTCCAGAGTAGGAGTCATACTG |
| | | ATCTCCCCACATGGACATTAGCTCTAGAGTT |
| SEQ ID NO: 89 | 140600_458034 | TAGTATCCTGCATGAACAGAAGCAGTTTTCCTTCT |
| | | GGTCCATCTCCTAAATAGAGTTGTTGAATTTTTAA |
| | | GCAGGAGGCAAGATTTTTGCCTTCCCTTTCC |
| SEQ ID NO: 90 | 112336_826 | CATGTTACGAACTTTCCTTAAGAGATCTTGTAGAG |
| | | CAGCCGGTAGTGGCCGAAGCTCGGCGAAACGAAGA |
| | | CAAGGATGCAAATATAAGTAGGAACGATAGT |
| SEQ ID NO: 91 | 140600_301818 | CCTGCACCGTGAAACATGGCTTGCCCTTTCTATAC |
| | | GAATGCTCTACAAATGGATTCAATTGCCTTCAGAA |
| | | CTTTCTTTGGCAAAATCACTATTTGACTCCA |
| SEQ ID NO: 92 | 140106_450343 | TATGGATACGACATAGTCCGTTTACTAACGTGACT |
| | | CCAGTGTAACTAATTGGGCGACAACACTAGACAAA |
| | | AAAGAATTAAATCTGACCTTCGTTGTGATTC |
| SEQ ID NO: 93 | 140641_457525 | AAAACTCTTCCGCTGCATACCTGATTTAGTAGCAT |
| | | AAAACTAACAGATTTTGCTTTAGGTGTAGTTCCAA |
| | | CTCGTGAAGTTGTAGAGAAAACTTGAATTCT |
| SEQ ID NO: 94 | 140641_525818 | AAAAGTCAGCATTTAAAACCCTAACGAGAAAACAT |
| | | AAGAAATTATGCCTTCTGAATAATGATACTTCAGT |
| | | TGGTGGCTAATGAGGTAGTGTAATAAAGAGC |
| SEQ ID NO: 95 | 140360_367283 | GTGTAGCCTTTGAGCAAACTTAACAAGTCTTTGAT |
| | | TTTTCTCAAGTAGGCCACCATTCTATCAGCTCGAG |
| | | CTGTGTATTCACTCATGATTTGATTCACAAC |
| SEQ ID NO: 96 | 140776_59092 | TTGATCAGGTAAGATATCCACACTCTCGATGGAAG |
| | | CACCAGACCGTTAACCTCCAAAAGTAATCAACCGA |
| | | CAACAAAGTTGTATGGATCTGATGCTTGCCA |
| SEQ ID NO: 97 | 140951_116615 | TTCACAATTAAGCCATTGCTTATTGAAAATTCATA |
| | | AATAAGGACCTTTTTCCTAAAGGGGATAACTTATG |
| | | AGGGGTAGCTCCTCAACTTGAGATTCGACCC |
| SEQ ID NO: 98 | 140951_49477 | AGTTAAAATAATATTTGCTACTACTAGAGGCCAAA |
| | | CTAGATATCCTCTTTAATTTATTATAACTTATTAC |
| | | AAAAAGAAGATAGGCAAACAGGGGTGTCCTA |
| SEQ ID NO: 99 | 141161_142548 | AACACCGGAAGAAGAAATGGCATATGATGATCACA |
| | | TTCATCACATGTGAAAATTTTGATCATTAAAGCGT |
| | | GATGAGCGCTGATTCCCTCGTCATTAAGCCG |
| SEQ ID NO: 100 | 140777_165625 | CCAGACTGTATTTCATGGAGTCGCGGCTCGATTAA |
| | | GTGAAGAAGAAGCTAGCAAGCTCGAGAAAGAAGAA |
| | | GGTGTGCTGGCCATATTCCCGGAAAAGAAGT |
| SEQ ID NO: 101 | 142674_1045013 | CTTCTCCTTTATTTTTCTTTTCCGACCTTTGACTT |
| | | TCCTGTAGGAACCTCTACAGTTTGAATACTAACGA |
| | | AAACTTTGATTTCTCAGGTACAGTAATTAAT |
| SEQ ID NO: 102 | 141905_1834674 | GAAGAACTGGCAACTCATCTCCTGTTTAATGGAAT |
| | | CGCTATTGTCGACCTTGGTCTTTGTGGTTTTTGGG |
| | | TTCTTGATTTCTGTTGTCACCCTTCGTGTCG |
| SEQ ID NO: 103 | 419_166991 | AAGCTGCCTCCAAAAGTGAAAAAGTTGATGTGATG |
| | | TGTTGGTATAAGGTGCCTTCTTACTATGATTCAAA |
| | | TTTTGACTAAAAGGGTGACTGCCAAGTGGTT |
| SEQ ID NO: 104 | 419_678771 | GTAGTCTTTGCAGTGAACATTGATTGGACCTTTAT |
| | | GTTCATACATATAAGCTCGGGCCACAATGTTGTTG |
| | | ATATTGTCGGAGCAGAGATATACTAGCTGAT |
| SEQ ID NO: 105 | 419_884190 | ATCCTTCAGATGACACTTATAAAGATTAAATTCAA |
| | | AGAAAACTTGATTGTCCATGACAAACTTTGACACA |
| | | CTAAGCAATTTCTTAGTTATCTCAGGAAGAT |
| SEQ ID NO: 106 | 132079_15905 | GCTAATGCAAAAAAACTTGCTTTCAACACAACAAT |
| | | GGCAACAATTTAGTACCTGAAGATACCTAACCACT |
| | | TTTTAATTTCAACTAGGCATCTTTTACTATA |
| SEQ ID NO: 107 | 419_3122991 | TGGATAGCTTCTGCCAAAACTCCACCAGTAAGTCA |
| | | AACATGGAGTTTCCTCTACTATCTCTAATAAGCTT |
| | | GTAACAAGGCATTAACTTCCACACTACTTTA |
| SEQ ID NO: 108 | 419_4202255 | AGTTTGAAATTATATGTCATGGTCTACTTTTTGTG |
| | | AAACTATGTGATTTGCGCAGGTCACGCATTCACCA |
| | | GAAGGAATGGAAACACTTCGTTACTTTCTCT |
| SEQ ID NO: 109 | 419_4445987 | GTATAAACATGGGGATCCAATCCATAAACCTACTA |
| | | CTTTGACACAAGACGTGTGAGAAATGACAATATAC |
| | | TCATGGTAATGCAGGGCCCTTAAGCCTATGA |
| SEQ ID NO: 110 | 419_4603364 | CGTTATCAATGATATCTTCTTTGTGGATGCATCTT |
| | | GGAAAGAGGGTGTCGCGGGCTTAGCAGCAATGTGG |
| | | AGGTCTCAAGATAGGGTCTGCTGGTATTCAA |
| SEQ ID NO: 111 | Cannabis.v1_ | TCTACGGGAATGGCAATGTTGTGGTTCAGCATCAA |
| | scf125- | CATCTTGTTCCCAACCATTATTTCTTTGTTTCGGA |
| | 78737_100 | CAGTGGCTTTCACTCCATGGCCAGTAATGGA |
| SEQ ID NO: 112 | 141566_980231 | TCCCAAAAGAGATGGAAACCCCATCACGTGTCCTA |
| | | AAATTACAATACCATTAACCATTTCCACTCATTAA |
| | | TCTATTTCATTTTGAGCCTTTAATCGTTATT |
| SEQ ID NO: 113 | 141610_5634 | CGGCGTAATATAGCCGGAGACCGCGAAAATTTGCA |
| | | TTTCAGCAATACGAATGGCAATAACGTTAGGGTCA |
| | | GAAATTATTCAGTGCAGCGGGGTCGATGGAA |
| SEQ ID NO: 114 | 102762_325 | CAGCAGACAGATGATGACAAGTGGGGATTTTGGTT |
| | | TGTATATACATCAATAAGTTTTTGTATAAGAGATT |
| | | TGTATTTGGACTCTAACCAAGTTGTCAAAGA |
| SEQ ID NO: 115 | 141610_733919 | AGCCTAAGTATCTGTATGGTGGCTTAGTAGTTGTT |
| | | ACTTTTGAGTTAGACATATGCCAAAGTTGTATTTC |
| | | TTTTTCCTTTTCCATGGATATAGATGTCATT |
| SEQ ID NO: 116 | 418_238459 | ATATAGGTATGTATCAAGACAAACTTTTATCTTGA |
| | | GAATTTTTGTGCTTGCTTTTCTCACCTGAAGTATA |
| | | GAGAAATGTTTTGGGGAAGTACTTTTTATTT |
| SEQ ID NO: 117 | 126818_5537 | GGAAATAAGTGGCTCTACAAAATGAAATTTAATGC |
| | | AAATGGTACATTTTAAAGGTACAAGGCTCGTCTTG |
| | | TAGCTAAGGGCTTCGCCAAATACTTGGGGTT |
| SEQ ID NO: 118 | 140767_20769 | GCGATCGATGTTTACATAGCCGGTGAATACTGTCA |
| | | TTGGGCAAAAGGAAAGTTTTGCTGTGGAGGATTAG |
| | | TGTGTCAAGGTCCTGCAATATATTGTTACTA |
| SEQ ID NO: 119 | 323_197605 | TAGATCCAAGTGGATCAAGATCAAGGACCCGCGAA |
| | | CATAACAGATCAACTAAAACTCATGCCCATGACAG |
| | | GCGAACATGAGAGGAAGGTGGATTGCGACGT |
| SEQ ID NO: 120 | 142100_2816765 | ATTCCCCGCAAATAGACCCACAGTGATATTTATTT |
| | | AGTTATGTTTCCCAGAGTTATATATCTAAGTGCTC |
| | | AAATATCTGAAACAACAAGAGCAAAAAGAGC |
| SEQ ID NO: 121 | 142100_734171 | ATTTGGGGCACAAAAACCCAACAACTACACAGTTG |
| | | AAGAGAGATTAAATTCATTGATTGGTACTACCTAT |
| | | ATCAACAAGGTGCATCTTGTTTTCTAGTAGC |
| SEQ ID NO: 122 | 140390_247532 | GAGTAATGCATATAAAAAATGAGGTTGCATAATGT |
| | | TTTATGACCACAAAGGAAGTAATTGTGGAGAAAGT |
| | | AGTTGCTTACCAACAGACTTACAATTCCAAT |
| SEQ ID NO: 123 | 120908_131 | CTTCTCGGCAATAAGAAGCTTTTTGTTAACTTCAT |
| | | CCAATTCCATTCGGACGGCTCCAAGTTGAACCTGT |
| | | TGGGTTTTATGCCCTAAATA |
| | | ATACTCTTTAC |
| SEQ ID NO: 124 | 140896_7749 | TTTGATCTGATTTTACCCTGAATAAGCATTCATGA |
| | | AGCTTAATATTTCATTCCCTGCTATTGCATCCACA |
| | | ACCTGGTCGATTCTTGGAAGCGGAAAACAGT |
| SEQ ID NO: 125 | 128460_3743 | TTGTATATGCTTACTTTAGGCAAATTAGACTTAGA |
| | | GTTAGATTAGTAGCGATGGTCCAAGCTAGAATCAT |
| | | TACTCATTACTCATTACTCATTGTATACAGT |
| SEQ ID NO: 126 | 139926_79630 | GAGTTTTGTTTTTTGGCAGCGATGTTAATAGAGGT |
| | | TGCATGCTTCCATGATTGGAAATTTTCTTGACCAG |
| | | TCAAGAGAAATGAAGCGATGAGGTTATTTGG |
| SEQ ID NO: 127 | 104456_347 | GTTAATGTCACCCAAATGTGATGGTAATGGGTCAC |
| | | TAGAGTTTAGTTCTGTTATTGTCTTTTATTGTGAA |
| | | GTTGGGTGTTCGGTGAGCTAGATAGCATAGC |
| SEQ ID NO: 128 | 122295_716 | CTGTTTCAATCATTTTTCTTTTACTGCTGCTCTCT |
| | | GCTCTTTATTAGTTTTTCTTTACAGATTTTGTTTT |
| | | CTGATTTCATTTGCTAAGTATTTCCCAAAGC |
| SEQ ID NO: 129 | Cannabis.v1_ | ATCTAGTTGATCATTCTCATTTTTTATGTGTACAG |
| | scf2008- | ACTGCAGTGATGCCCCTAGCAGGGGAGGAACTTCA |
| | 17655_101 | TTTGGTAGCAATGTATTCCAGGAAAAATGAT |
| SEQ ID NO: 130 | 142713_4924636 | TAGTAATATGTCTCTTATGATTGATTATTCCTATA |
| | | CACACCATTGCTACTGTACTAGTGTTGTTCTTGCT |
| | | TGATTGATTTGATTAATCAATGCTAGATGGT |
| SEQ ID NO: 131 | 142713_4919331 | AATTCTAGCTACAGTTCTCAAGATGTTTACAAAGA |
| | | ATTCAGCAAAGTGCTTTTCAATTGAGATTATGATT |
| | | TTAGGTCCCTTCCTTCTGTGAATTCATTCAC |
| SEQ ID NO: 132 | 142713_4720731 | AGCTTAAGCAAGAAATTTAACTTTTCGGTTCAGAG |
| | | TTATCGCCTTATTGCGGTGGCATTCATGATAGCTT |
| | | TCGGTTTAGCTCATGCGCGGTTATGAATTCG |
| SEQ ID NO: 133 | 142713_4248039 | TTATTACAGGTTTGGAACTGGTTTCAAAATAGGCG |
| | | ATATGCTATAAGGGCGAAATTAAGTAGGAACCCTG |
| | | GAAAGTTAAGTGTCTCATCTATGCCTCGGGA |
| SEQ ID NO: 134 | 142713_4084703 | CTGATTGAGAGAGATGTATGGAAGGATAGATGGAT |
| | | TTGATTGCTGACTTTCCTCTATTAATCATTTTATT |
| | | TCTTTCTCACTTTTCTCTCTTTATATATATA |
| SEQ ID NO: 135 | 141928_166641 | TTTGATCAGGCCCCAAACCATGTCTTTCCATCTCA |
| | | TTCCAAGTTTGTTCAACTCTTTCTTGACAATCCCA |
| | | ATCCATAAACAACTTCAACAACAAATTATAT |
| SEQ ID NO: 136 | 123596_6054 | CTAGGCTAGGTGGGCCCTAGGCACAGGCGTAGCCC |
| | | GCCTATGCCCAGGGCCGGGCCTAGGTGCAGAGAAT |
| | | TCTAGATACCAAAAATAAAAAGCAGTTACAT |
| SEQ ID NO: 137 | 142713_3450569 | GGGTTTCAACGGAAACATTTGAATGGGCCTTAAAG |
| | | GGCCCAGTAATGATGAAAAAGTTGGGGGTGTTGTG |
| | | GTCCACTAGTGGGTTTGGGCCGTTGGGACTT |
| SEQ ID NO: 138 | 142713_2757703 | AAAGTTGAGCCAAAACAAGCCAGAATAACAAACAA |
| | | ATTAAACACAACTTGAAAGGCTAGGCCACTAAATT |
| | | TTAACGCTCTCTGAAGAAAATGTAAGTTTGG |
| SEQ ID NO: 139 | 142713_1054639 | AAAGAAATATGTGCATGTGATTGTCCACTAAATTC |
| | | TGCTCAACTTTTACTAAATTGTTTTGATCTTATTG |
| | | AAAGTTACAATTTTCAAATATATATTATATA |
| SEQ ID NO: 140 | 142713_697174 | CAACATATTACATAAAACAATTGATCTTAAATCTC |
| | | CCATATCCATAGGCTTCTTCTTCTTAGGAATTAAG |
| | | ATTAAAATAGTTTTGTTAAGATCTATAGGAA |
| SEQ ID NO: 141 | 136103_7722 | ACTAGTATTCTAATGGAGATCCCATTAAGAAGTAC |
| | | ATTGAAATACATAAAACATTATTGTCTTTTTGGCA |
| | | TCAAAAAGCAAAGGTAGCTTGGCTAAAAGAA |
| SEQ ID NO: 142 | 142713_576343 | TACTACGGTTTAGGGCCATCTCTAATAGGAATAGC |
| | | TCCATATATTGCTGTAAACTTTTGTGTTTTCGACT |
| | | TGTGAGTTCACTTATACTTTCATCTGTTATA |
| SEQ ID NO: 143 | 142713_568710 | AAATCTCAAGCGTGCTCGCATACTAGTAAAATCAC |
| | | ATGTATTACACTCTATTGTTCCAGGTAGCATTCTA |
| | | AAGTTCTGTTCTTAAAATATTTTTTGGTTAC |
| SEQ ID NO: 144 | 142713_342957 | AAGAACCAGTGAAGCTAGAGAGACCACTGACGGTA |
| | | CAGGTCTTAGTGATGCTAGAGGGACCAATTATGTA |
| | | AATTAACCATGAAGCCACTCCTCCTGATCAT |
| SEQ ID NO: 145 | 142713_275962 | GAAGATGAAGGATATGAACTTGCTGATGAGCTAGG |
| | | CTTGCTGGAAGACATAATTATCTTTTGTTGGGTTT |
| | | TCTTTCTTTTCTGACTACCTTTCTCTATCTG |
| SEQ ID NO: 146 | 61055_4286 | AAAAATAACCAAGCTTTCTCCATTGGAGGATGGCT |
| | | TTTCAAAACTTGATTAACTGTGTATGAATCCCATC |
| | | TTATAGGGAGGCCCTGAAGCTGATCTCGAGC |
| SEQ ID NO: 147 | 133563_14816 | CCCATCTACTAACAACAATTTTTGGCGAGTTATCA |
| | | AAGAACAAAGAAGAACTAACAATGAAACAAAACTA |
| | | TGTCACACACACAAAACTAAAACACTCAAAA |
| SEQ ID NO: 148 | 141963_1974061 | GGCCAGAACCTAGCTATATAATATCTAATAGGCAT |
| | | AGTGATGAGTCATGAGCCATGTATCTACAAATATT |
| | | CTTGGTGCATTCCCCCTACCTATCATTCATT |
| SEQ ID NO: 149 | 141963_1531282 | CATCTTGGCAATGTTGCAGGGTTCCTACTTACTCC |
| | | AATAATGCTGTCATCGATGGGGATGTCAGGTCCAT |
| | | TCATTCTCTTCTCGTCGTTTGGGTTAGTCTG |
| SEQ ID NO: 150 | 141963_1527311 | TCTCTTTGTAGACCTACTTACATTCTATGGCTTTT |
| | | CTACCAGGAGGTAAGGGAACCTTCACCCAAGTTTT |
| | | CTTTTGAGTAAGAGCTTTGATTTCAATTTGC |
| SEQ ID NO: 151 | 141963_1357946 | AAAGTGGGTTGTGGAGAAGAGAAAACTCAGTTAAT |
| | | ATCATCATCTTACAAAATATTTTTTGTACACTTTT |
| | | ATATTTTTTTGGGGGGTGGGAAGTGTGAGAT |
| SEQ ID NO: 152 | 141963_654218 | TGTTGGTCAGATGAAGGTTTCGGCATCTGATTTCT |
| | | CTTGTACGAGTGAACTGGACAATGCTGAGTGCGTG |
| | | TTGAGAGAGTCTGTTCTCTTTGGGGTTGAAC |
| SEQ ID NO: 153 | 141963_295684 | GAGGCTTTAAGTTGGATAAAAACCTCTAATCTTTC |
| | | GAACATTGTTATTGACACTCATTGTCTTATTGCTA |
| | | TACAAGCTTTGCATAGCTCGGAGGATATGCC |
| SEQ ID NO: 154 | 141963_145177 | TTTCAACTCTTTCTCTAAAATTTCAAGATACTCAC |
| | | TTACTTCTTCTAATGTCTTTTTCCGCTCCTCCTTA |
| | | ACCCAGAAAGCTTTCCATGCAGTTGGCACGA |
| SEQ ID NO: 155 | 141963_21605 | GAAGGACTTGTTAAATACAACTCAAGATGTCTAAG |
| | | TAGAGAAGTGCCACATGACTACTGACATAAACATA |
| | | GCGTCAACACACACCCAAAAGGTGCCTTAAA |
| SEQ ID NO: 156 | 141501_36079 | ATATAATTTTCTGATTACAGTGTGTACTCAACTCA |
| | | TGAAATATTATGATTAAGCTAAGAAAGAACAAACT |
| | | CACACACTACCACCACAAGAGAGAGAGAGAG |
| SEQ ID NO: 157 | 141501_38812 | ACTGATAAACACAAGTCAACCAATCAAACCCAATT |
| | | GGGGCTTTGTAAGTCATCCCTTCCCACTATTTAAA |
| | | TAGCCTCATTATTAGTTTATTAGAAAATTTT |
| SEQ ID NO: 158 | 141501_60143 | AAATTCCTTTAAGGAGATGAGCATCTTGTCTAGCT |
| | | CGAAGAAGAGAAGACGAACCCTCAGCACAAAGAAT |
| | | GAAAAGAAAGGGGATAAATGATCCCCTTGTC |
| SEQ ID NO: 159 | Cannabis.v1_ | ATCGGAAGAATGACCAGTTGGAACGCGAGGCTGCG |
| | scf8348- | ACTACAAGAGTTGGACGCAATGATTCACCTCCTAA |
| | 7318_101 | CCAACCGAACACATCCGCTAGGCGACCTCGA |
| SEQ ID NO: 160 | 141501_109163 | CATCAATATGGTTCTGGCTATAAGTATTCAACATG |
| | | ACATCATCTTTATTTTTCCATAATAAAGTGATACC |
| | | TCCACTATGTCCTTGAGAGTCCACAGTGAAC |
| SEQ ID NO: 161 | Cannabis.v1_ | CCACTCTTTTTAGTGAATAATTACATTAATTTAGT |
| | scf3004- | TTTCCAAAACAATTTAAAGATAAAATGGGAAACTA |
| | 35184_100 | CTTACTTTCCCATAATACAATAATGACATGT |
| SEQ ID NO: 162 | 141501_283981 | GTCCCTGAAAAAAAAATTATTTTTAGGACAGTCCC |
| | | AAATTCTTATATTTGGAGTAGTGAGTTTTCGGGAG |
| | | TTAAAACAGAGATTGATTACAGCTTTGGTAT |
| SEQ ID NO: 163 | 141501_338551 | TTAGAGAAAGGATAGTGACTTTTTCAAAAATTTGC |
| | | GTGATGCTGATGTTGGTAATGGATTGCAGTTTGCT |
| | | ACCAAAATTTTACAACTAATGATTGCTTTTC |
| SEQ ID NO: 164 | 121207_1396 | TTGATCTTTTTTTTTCTCCGAATCTCTGGGCATTT |
| | | TGCTTTCAATCTGTACTCATCTAAAGCTTTCCACC |
| | | CGATTAGTAGCCAGTTCTTTGGATTTCTACT |
| SEQ ID NO: 165 | 141801_250754 | GTTTAATATAATTATAAAAAAAGCCCTTTAATTAT |
| | | TTTTTAATATAAAAAGTATTTTACTTATTTTCTAT |
| | | GCAGGTGATTGGTGAACTGTCCATTAAATTT |
| SEQ ID NO: 166 | 141801_1619833 | ACAAGCCTTTACCCAGTCGGTGAAAGGAGCAGTGA |
| | | ATCCAAGTCTCAATAGGACCAATATAATGAAATCC |
| | | CAATCCATCTTATCATACGCCTTTTCGATGT |
| SEQ ID NO: 167 | 141801_1808917 | TCTTACCTTTTATATTCACCATTGGATTTCAGTTA |
| | | GAATTAGTCAACAATGGTTGAAGAAATCTTTGGTT |
| | | TTCTTCTCGTTTGCTTCAGCCGAAGCTAAGA |
| SEQ ID NO: 168 | 142257_131770 | CCTCCAAATACTGGCATATCCAAAAATTAAAAATT |
| | | TCTTATACTATGCACCAGGTGGTGGTCACACCATG |
| | | TTCAAGATAAAACGACACAAGGTTAAAAAAA |
| SEQ ID NO: 169 | 142257_302302 | TATTTTCATCCACCGGATCTCGATTCTCTTTTCGT |
| | | AATTCCTTCTAGAGGCCAAGCCTGTGCTTAGTTAA |
| | | TAGTAGTTAGCCTCCTTTTTTCAGTAGAATT |
| SEQ ID NO: 170 | 142257_307287 | ATGCCTTGATGCCAGTGGGAAAAATCCAACTACTT |
| | | GTCACCCTAAAAGGAGAAAATAGTGCCAACGCCTT |
| | | CAAGCATTGTACATTTGCAGTAGTGAATTGC |
| SEQ ID NO: 171 | 142257_641820 | CAAATTTTAGATTCCATCTATTTTCGTTATTATTT |
| | | TTCTGTTATATGTTGCGTGTGTTCATTAAAGTAAC |
| | | AAATATCATATTTTCATTTGTCCATCCATAT |
| SEQ ID NO: 172 | 142257_921419 | AATAATTGCTCCGCGGACCCTCAAAGTGTTGGATT |
| | | TTATTTTACCACGATCTAGATTTACTAACATGTAT |
| | | GTTTGATTAACACCCTAAATATGAATTCACT |
| SEQ ID NO: 173 | 142257_1207521 | GCATTACAAAAATAAATATATAAATAAATACAGCA |
| | | GTATCAATGACTAAATATTTATCTTCATAAACCGA |
| | | AAACATACTTTCAGCTCAGTCTCGGGGTGGC |
| SEQ ID NO: 174 | 142257_1294752 | CTCAAAGATCACCTAAAAACTATAACTAATCCTAC |
| | | TGCATTTCAAGTGCAGAAATATTGTATTTCAGCAG |
| | | GATATTGCTTGCTTAGTCCCAGTCAAAATCG |
| SEQ ID NO: 175 | 142257_1345842 | ATTATCTATAACTTCAGTATTTAATAGACTTTATA |
| | | TATTAAAACTATAATCATGAACATACCAAGTAACA |
| | | TAAAATTCACTTCTATATCATTCAATGATCT |
| SEQ ID NO: 176 | 142257_1360236 | CAAGTAGGTTATCATGGACATGCTGATTTGGTTTC |
| | | ATCGATGCATTTGTAAAGTTTCTAGTAGCATCAAA |
| | | ACAAGCTTGATCTGATGTCTTTCCAATCAAT |
| SEQ ID NO: 177 | 113379_27494 | CAACAGCCTCCCTCAGTTTCTCCTTGTGCTCATCA |
| | | AGGTTAGTATGAAGCCCACAGATGAAGGAAACTTA |
| | | TGCGATGTTCTCTGTGTTTCTAGCACCAAGC |
| SEQ ID NO: 178 | 142257_2622256 | CTTACATTGCAGAGAAGAGACATACAAAAATAAAT |
| | | AAGAAAAATGAAAACTGGTCCCAAGTGAAAATCAA |
| | | ATCAATTGGAACATGACAATTGTATTCGTCA |
| SEQ ID NO: 179 | 142257_3011452 | TAGAACCCTTAGGATTACAAGGTATAAATCAATAA |
| | | TGGCAAAACTCAAGTGTTGAGATGATGCTAGTGTA |
| | | GGCGTGTGAGAGAGATGAAGAGAAGAGAAGC |
| SEQ ID NO: 180 | 142257_3019506 | ATCTGACAGTGCTTTTCAAATGCAAAACTTTGGTG |
| | | AATGGATCAACTAGGTTGTCTTCTGTGTCCACTTT |
| | | CTCGACCAGTACATCCCCTCTTGCCACATAC |
| SEQ ID NO: 181 | 112589_1106 | ACATCACAAATGCTGATTTTAGGGCATTCCATGTA |
| | | GGAGCCTGAAAATTGCAGGTGAAACCCAATCAGTC |
| | | AAAAATAGATGTTAGGAAAACTTTGTGAGAA |
| SEQ ID NO: 182 | 140408_80987 | CGTATTGCCACGTCAAGGTGCAAAAAATAGGATAA |
| | | TAGTAAGCTTTGTATCTTTACTGGACCAAGAAGTT |
| | | AGGCCTTCAATTTTCGACCCAAGTGTGATAT |
| SEQ ID NO: 183 | 142254_8625765 | TGATTTGAGGCGATCAAGAATTTTATCAATGCACA |
| | | GATTAAGCCTCATCAGAACGTGGGTGATCACCTGC |
| | | TCTAGATAGCTAGTTTTTTCCAAGAAGCTAA |
| SEQ ID NO: 184 | 142254_8167871 | ATTTACCATTACATCCTTCAATGTATTTTATCCTT |
| | | GAAATACATAGCTACCTATAAATGATATTTTAGTA |
| | | AACTAATATAATTACTGAAATGAGTACTCAA |
| SEQ ID NO: 185 | 169366_808 | GTTAGAACTATAGTTGGTGTATATATGATTGCATC |
| | | TTGATATTGTGAATATATGTGTTTGGTAATTCAGA |
| | | AAGTATGAACCAATGCTTCAAA |
| | | CACTAGTAT |
| SEQ ID NO: 186 | 141820_1679571 | ATGGAAGAAGACATTATATTCTGCTACCATCATTG |
| | | TAAGATTTTCCATAATTTTATATGTATTTATTTCA |
| | | TTATTTTAAGAATTTTATGTTTGGGGTGGTA |
| SEQ ID NO: 187 | 141246_298026 | ATAGACAAATTAAAGGCCAGAGAAAGCTGATGGTG |
| | | AGGTGACATCTAATTCGGAAAATGTTTACAGCTTG |
| | | TTGCCTTAAGTTGTGCCACCGAAAGCGACGA |
| SEQ ID NO: 188 | 141246_683086 | AGTCAGTCTGAAGTTGGCATACACTTACTCCCGAG |
| | | CTAAAACTACTGCTTCCAAGAACATGGCTCTATCC |
| | | GACACCTTAAAGGCAGAGGCGGGCTTGGCCA |
| SEQ ID NO: 189 | 102314_4697 | TTAAAAAAATTAAAAAATTGAGAAATCAAAGAAAA |
| | | TGAGGAAAAACTCATTCAAATTGCCACCTCAATTC |
| | | GTTAATGTTTTTCTTTATATATATTAGGAAA |
| SEQ ID NO: 190 | 140923_39555 | CCAAGATGGTCCTTTCTGGGGGGATTTTGCTGGAC |
| | | AAGTTTTGGACTTAAGCCTTGATCCTATGACTGAT |
| | | TAGTTCACGAGCTTCCTAGGGTCCTCTTCGG |
| SEQ ID NO: 191 | 141356_607414 | AACCCTAGCCAGGCATACAAGATCACCTAGCCAGC |
| | | CTATTATATCCCCTTGGCCTACTAGACATGTTGGT |
| | | ACAACTACTTGATGGTGTGTAACTACTTTCG |
| SEQ ID NO: 192 | 141356_120636 | ATATTGCTCTACCCATATACTGCATTATGCATTTT |
| | | TTATCATTGCTACGACGAGTGTCTTCTCTTCCTTG |
| | | CATTGATTGAGTATGAGATACTGTACGCCTC |
| SEQ ID NO: 193 | 141405_1135170 | TCGACCCTTTTTACTTATATTATTTTAAAGAATTA |
| | | ATAAGATCATAAGGATTTGGGCAAGTGAGTTTTGT |
| | | TATCCTGATTTTTGGCACTGACGTGGCATGT |
| SEQ ID NO: 194 | Cannabis.v1_ | TCTCTCTCAGTTTTATATATGATTAAAAAGATTAA |
| | scf2492- | CCACTCCACTTGCCCTCTAATATAAAGACTCACTC |
| | 7828_101 | TATTTATATTAAAAAATAAACCGCCCGCCAA |
| SEQ ID NO: 195 | 141405_510744 | TCCCTATCCCAAAAACAGACTTGGACCTATATAGT |
| | | GATTAGGATACGAACTTGGACTCGGACTAAGACCC |
| | | AGACTGGGATCGAAATGCTGGACCAAGATCT |
| SEQ ID NO: 196 | 141405_363961 | ACTGAAATCGGGACCCGAACCCAAACTCAGACTAG |
| | | ATGTCGAACTTTGACTCTAACAAAAACCCAAACTT |
| | | GATCTCAGAATAACTAGAACCGAACTAGGAC |
| SEQ ID NO: 197 | 141405_280237 | CAAACTCAAACACATACACCTAGAATCAGACCCAA |
| | | AGTTAGATCCGTACCTGGACCCAAACTCGAACCCA |
| | | GACCAGGATCAGAACCAAGGACTTAGACCCA |
| SEQ ID NO: 198 | 141405_267660 | GACCTAGACTCAAGATGCGAACCCGAACTTAGACC |
| | | TAGACTAGGATCTACCAGCCGGACTAGGATGGGGG |
| | | ACCTAGACCAGGGACCTAGGCCTTGGAACCA |
| SEQ ID NO: 199 | 141405_197268 | AGGCCGAGACCCGTGATAGTGATCCAAATAAAGAT |
| | | ACGTGACTCGGGTAAGGGGGCTGGAACAGACCAGT |
| | | AGTCGGTGTCTAGATCTGGGACTCAGACTAG |
| SEQ ID NO: 200 | 141405_11474 | TGGTCTTCAGACAGCCCTCCTCAGACCGAGAAAAC |
| | | TCGGACAAAACCTGTCTGAAACCACACGCGTCCGA |
| | | GGAAAAAGAAGGTTCCGCGCGCGTCCGGGTG |
| SEQ ID NO: 201 | 141405_150427 | GGTCACGGTCCTAGTCTTGTTCTAGGGCCTAGTTT |
| | | GGCAATTCTTGTTCATCTTCTAATTTTTCGTCCTA |
| | | GGTCTAGGTTTGTGTTCGGGTTTGGTTCATA |
| SEQ ID NO: 202 | 134489_82446 | TTTCTACAGACACAACTCACAAAAAGAGATAGATA |
| | | GAGAAACACAATACATATATCAACTTGGAAATAGG |
| | | GATCATGTGAGCGGGTTGTTCCCAATTTAAA |
| SEQ ID NO: 203 | 111559_3929 | TCACATCGAGTCGCGAATAGACGCGCCTGACACGT |
| | | GTCACCCATTCAAACACAAGGAGAGCTTGGATGAA |
| | | GTCTACACGCAACTTTGAGAGTCTCGTACAT |
| SEQ ID NO: 204 | 263_598117 | TTAGAATTGCGTTAATTAAATAAGATAAGACTCGT |
| | | TGAAAATTTTGTTTTAGTTGATGTCACTTTGGGTC |
| | | GACATTTTCTTATTCTACACACAAGCTTCAC |
| SEQ ID NO: 205 | 263_949233 | ACATTGGCAATTGTAACACGATCAACTATTTGTAA |
| | | GGTTATATTCATGTATCTTAGACAAATCTAAAACA |
| | | TGTTTGATTATGTAATCATACACATCATACA |
| SEQ ID NO: 206 | Cannabis.v1_ | GAATTTGCCAATATAAGAAATTATTTACAGAAAAA |
| | scf1779- | CCATCCCAGAAGAGACTACAGATAGAAAGTGTGTA |
| | 30705_100 | AACTATTTACATTGCCTCAATGATGAAACAC |
| SEQ ID NO: 207 | 141366_479351 | ATTTGGGAAAAGAGATTGAACCGCTCCAACAGCGA |
| | | GAATCAAAGCTCATCGTCCACCACAAGCCGGTCAG |
| | | ATTACGGGCTGAGCTGTAACGAGAATTCAGC |
| SEQ ID NO: 208 | 141366_1038384 | ATTGGAAACAATCTTCTATTCATTTAAAATTTCAG |
| | | ATTTTACTCTTATAAGGTTTCAATTTTTGGTTTTG |
| | | TCAATATTATTCTTGTTCCAAAATGAAGGCT |
| SEQ ID NO: 209 | 141366_1054439 | CTATTCAAATGGAAACTTTCCACAATGGTCTTAAT |
| | | GTTTCTACTCACAAATTATTGAATGCTTTACCTAA |
| | | GGGGGTATTCTTTCTAAGTCCTACAATGAGC |
| SEQ ID NO: 210 | 141673_28623 | CGATCATGGGGTCTAGGTTTAGTCCCATATCTTCC |
| | | CAGTGTACTCCCCTCAAAAGGGTCCGCCATGGGCC |
| | | TCCCTTAACCATTTCTTCAAAGCCTCAGCAT |
| SEQ ID NO: 211 | 141673_544430 | ATTTCTAAAAGCACACTAAAAACTAATTTAGGCAG |
| | | ATTAAATGAAGGCCAGGGACAGGTAACATTATAAA |
| | | CTCTGAGCTCACCTGATGTATGTGATCCAAT |
| SEQ ID NO: 212 | 141673_555374 | TTGATCAGGCATGAGATTGAGGGTTTTGCGATGTA |
| | | TTGTATATGTTCAGGCCAAGCTAGACCACAAGGGT |
| | | TGGGTCTACTGTTAAATGCCAAAATATTACA |
| SEQ ID NO: 213 | 128722_7519 | GATTTATATAAAATGTAAAAGACTTGCTCCTAAGC |
| | | CAGGAAGTCTTTATTAATTGGATATACAAATGTGT |
| | | TGGTTCGAAAGTCAAAAGAATAACCACACTT |
| SEQ ID NO: 214 | 141673_1231418 | TTGTTGCTTGGATCAAAGTTTGATTTAGTGATCTT |
| | | TATGCTTGTGTTGAGATAGCTATTTTAATTGGTGT |
| | | AATTTAATTTCAGCTGCTGAAAAGGATTGGA |
| SEQ ID NO: 215 | 141673_1587680 | GATGTGATTGGTGCTCTGTCATTGATAATATACTC |
| | | TCTCACTCTTGTTCCACTTCTCAAGTATGTTATCG |
| | | TTGTGTGCAAGGCAAATGACAATGGTCAAGG |
| SEQ ID NO: 216 | 103034_15764 | CCAGTTCCAAGCATTTTTCTCAAGAAGTCTCTTGG |
| | | AATTTTATCCATTTTCATCTATAACGAAGATACAA |
| | | GTGCTCTCGGGAAGAAAACGAGACCATTTGA |
| SEQ ID NO: 217 | 141440_750937 | GAGTGGTGTGATGATGAAAAGCGGTTTTGGTGTAG |
| | | AAGGAGAAGAAAATCTTGGGTTTATTATGGTGAAA |
| | | TTTAAAACAGAGAAGTAGGAAGAGTTTGTGG |
| SEQ ID NO: 218 | 141440_450292 | AGTGAACTGTGTTAATATCTTCTGCCCATAGACTT |
| | | GTTGCTAAAGAGGTGAAACCTTTTGTTTTTCCATA |
| | | AACTTTCTATTTTAATTTTATAAAGATTGTT |
| SEQ ID NO: 219 | 141440_311917 | TTAAGTTTGTTTGGTATTCATTTCTTGTAATTTTG |
| | | CTACATCTGGTTGTAGTGGTCTCCATTAGTGGAAG |
| | | CAATATCTTGAGTAACGTCTCCAACAGCCCT |
| SEQ ID NO: 220 | 141440_188532 | AGATTTAGACGGTCTGTAACATCTCCAACTGCCCA |
| | | AATGGAAGGGACTGAAGTTCGAGAGTATTCATCAA |
| | | CCTAAAGAAACAAGTGAAGGAGAAAAGCATT |
| SEQ ID NO: 221 | 125191_848 | TGTGAAAATTAAAGGAAAAAAAATCATTTTCTGGT |
| | | TGTCTACCTCTTAAATTGACCCCCTCTCCCTCTCT |
| | | CAATCACTTTTGTAATTTTTTAGAGAATCTT |
| SEQ ID NO: 222 | 141440_38014 | AGGAGCGTCTCTTTTGTAGAAAATTTCACGATCCT |
| | | AAGGAAGTAGGGACTTAACTTGATTATTAGATTTA |
| | | ATTTTATTTATGTTGGATAACATTAGACATA |
| SEQ ID NO: 223 | 142000_50342 | CTACTAATGAACCTGTTGTTGCATCTGCTTCTTTA |
| | | GAAATGGCTGAATTCGACTTGCATTTGGGAAGAAT |
| | | GGAGACAATGTTTGTTACAACTCTGCACTCT |
| SEQ ID NO: 224 | 142000_155277 | GAGGATTCATGCAACAAGCATGCGAGTGATTAGAA |
| | | AAGTCTAAATCTAGGAAGTTTTGTGACAGGGACGA |
| | | TGGTGAAGAAGTTAATAAATCCCCCTGATAA |
| SEQ ID NO: 225 | 142000_474918 | ATAAGAATCATGGCAATATCTATGCTTCCGCTGAT |
| | | AGTAGCATAACAAAACGCAGTGTTTCCTTTGCAAT |
| | | CCTGCAAAGCTAGATCGTTTTCTTCCATCAA |
| SEQ ID NO: 226 | 142587_52570 | TTAACCGTTTCCCAACTAGTTTATCGTTTCTTGGA |
| | | ATTTCGAGGAAATTAAGAATCGTATTTTTGTCATA |
| | | ACTTTTAACTCGGGTGTTTGTTTTGGACGTT |
| SEQ ID NO: 227 | 142587_425040 | TTAATTCAACATACTGTTAGTAAATCTAGATCTTT |
| | | GTAAAATATTCCCAACAGACACTTGATGTACAACT |
| | | TGCATGCAAGACAACAATTCAAGCTGGGAAT |
| SEQ ID NO: 228 | 142587_619893 | GTGGATTGCTGGCTGTGAGGTAGTTAATGATGATA |
| | | GAGTAGTTTGTGGTTGTTGTTGGGTGAAATAGGAA |
| | | TTGAATGGGCGTGGGGGTGGAGGTTGAAAGC |
| SEQ ID NO: 229 | 142587_645410 | TTTGACGGGGCAGTTCTGATCCAAACAAATGGATT |
| | | TCTTATAAGATCTCGGATTGGGAACCCGATCTGGT |
| | | ACAAAATTATCCAAATCTAATCAGACCCGTC |
| SEQ ID NO: 230 | Cannabis.v1_ | TTTTGACATTACTTAGTCGTTCACCAGTTCTCGGA |
| | scf874- | TATGTTGTTGTTTCGCAAAATTGAGAGACTCGAAA |
| | 145989_100 | ATACTCAATTTGTGACTAGTTCACTTTGCCC |
| SEQ ID NO: 231 | 91474_12377 | TCTACCTCTACCTTGGTTTCCACGAAACCATTTAC |
| | | TTTGCCCTGAACCACCAGATGTCGAGGTAACCTTC |
| | | CTTTTTAGATCAATGGTGGAGTCACCACCAT |
| SEQ ID NO: 232 | 142465_459658 | TAAAGCCACTGAATAATGATCACTCTCTTCTAATC |
| | | CATTATTCTCTCCTTATTTGTCTAACATGGGTCCT |
| | | CAAAGTGGTTTTGCTAGGGAGCAACAGGTAA |
| SEQ ID NO: 233 | 142465_223976 | TACAGTAATTATAATAACTTTTCCTAGCTAATTAG |
| | | CTACACCAACCAAAACGCCAAGTTTCAGTTGATAG |
| | | TTGGACCACCTAAAGAAAAACCAAAAAACTA |
| SEQ ID NO: 234 | 142465_222820 | ATTCATTATTTAATAATTCATGGCATATGATTATG |
| | | GGTACAGTTGATAGTGGCATTAGCAGCCGCGGGAG |
| | | ACGGTGCAGCTACCTTCATGGCAGAGTTAGG |
| SEQ ID NO: 235 | 142465_198069 | CTAATTTTTTTTGCACATTTTTTGTAACTAATTAA |
| | | GAAAATGTATACATTCATGTAAAATGCCCTAATTT |
| | | TTAAGCTTACTTTAGCATTTTTTTGCCTTTG |
| SEQ ID NO: 236 | 142465_44455 | TACGTCATCGTATCGTGATGGGTCGAATTTCTCTG |
| | | GCTCTGGAAAATATTTTGGGCTTTTGTTTGTTGTA |
| | | CTCACTGTCCAGTATACCTGCAAATTTATAT |
| SEQ ID NO: 237 | 141293_830644 | CAATTCCTTCCCCGGCCGATTCATATCCACCTTGT |
| | | TGGGGCCAAGCTCCTTGTGGCAAACCCAAAAACTG |
| | | AGGTGTATGTGTTGTCGCCGTTCTAACGGAA |
| SEQ ID NO: 238 | 141293_824822 | GGAAAAAGAAACCTACTGTTGACAAAGAGAACAAG |
| | | AAAGATCAGCTGAATGAGCAAAGTGAAACAGGGGG |
| | | AATTGTTGGGATGACAGAGGATAAAGAAGCT |
| SEQ ID NO: 239 | 141293_620463 | TTACAAAATTCAAATGGAAATATAATAGCATGTCT |
| | | TTCAAAACTAATATTAGGTATGTATTTTATTTCTA |
| | | TACATAGATCACTAGATACAATATATATACT |
| SEQ ID NO: 240 | 135018_30043 | AACCTAAAAAGACATCTTTCCACTCCCCAGAATCA |
| | | ACCATCTTTTAGATGCCATAATTGGCATGAAATAA |
| | | TGAGTTTCATGGACACGTATTTAGGTTACAA |
| SEQ ID NO: 241 | 135018_9167 | CATTATCCTTGAGATTATATCTTTCAAGCATCTTT |
| | | TTCTATGCTTCGATAAATTCATCTTCCTCCTCATG |
| | | TTCCTATATACAATCACCAAAGTCTGTTGAG |
| SEQ ID NO: 242 | 141293_474861 | ATTGCAAGTCTAAAGGAGTGGTTGGCAGATCCAGC |
| | | CATTGGAAACAACCCGATCCTCAGACTGATTGCAG |
| | | GAACTATATTCTTTCATGAACAAGACTATAA |
| SEQ ID NO: 243 | 141293_454000 | TATTAGTTTAGCCCAAGGTTCAGCTAAAGGATGTG |
| | | CAAACATATCTGAATCAGCCACAAGCACCTATAAG |
| | | AAATAACAACAGTTGCCACCAACCAATCAGA |
| SEQ ID NO: 244 | 141293_449941 | TAGTCACCAGGGCGGCGGTCAAGAAAATCGACTTT |
| | | GATATTCACCCCAGTTGCCTTCTTACATGCTTCCA |
| | | CAAATTCCTTCACTGATCTACCTGAAAAAAG |
| SEQ ID NO: 245 | 141293_345180 | CCAAGTGGGGACTGGGCTCTGCTTACTGAGGAACA |
| | | GTCGTTGTGCTGTAATGTTATCTGATCTAAGTACT |
| | | AGGGGTGTTCAACAAAATTTACAAACCGCCC |
| SEQ ID NO: 246 | Cannabis.v1_ | GGTTAGATTGGTGCATTGTCAATTCATATTGGAAA |
| | scf2913- | GACCTCTTCCCAAGTGCATCCCATTTTTACCTCCC |
| | 43590_99 | TTTCCATGGATCTAATCATAGACCTCTTAAG |
| SEQ ID NO: 247 | 141293_322698 | CTGACTACCAAGTAGAATCTACTCTCCAAGTTCCT |
| | | ATTTATTCAACTAGAGAGGATAGTCTCATTTGGGG |
| | | TCACCATAATTCAGGTTTGTT |
| | | TACTGTCAAC |
| SEQ ID NO: 248 | 141293_315297 | AACTTACATTAGTACGGTGCTGGAGCATTTTTCTA |
| | | ATGGCTAATTCTATAGGAAGATTTTTGGCTCCTAG |
| | | ATATAGATTTTTTTTATCTAAGGAACAGTTT |
| SEQ ID NO: 249 | 141293_289619 | AATTAACTTTCCTTTGAACCTCTTGTGAATCTATT |
| | | TCCATTTCAAAAATTTGAAGTTTACTGTCTCACAC |
| | | CATTGTAGAGCTTTCAATAAAGCTATAGGTT |
| SEQ ID NO: 250 | 141293_277076 | ACATTTAAGCCCACTTTAAAGTATATTGAAGAATT |
| | | AGAGATGATGTTGTATATTTAGGGGTATTAATAGA |
| | | AAAAATCATTAGGTTTAAAGCTTCAGAAATC |
| SEQ ID NO: 251 | 141293_234853 | TAAAATAATTTCCAACACATTAAAATACTTATTCT |
| | | GCTCCTGCATTCTAATAGCAGCACACAAGCCCTTA |
| | | GAAACAAGAAGAAAGGTGTAAGAAGAGAGAG |
| SEQ ID NO: 252 | 141293_22467 | GTTCCAACGAATTTTCTTACTGCTGCAAATTCAAA |
| | | GTCTGTCGCTACAAATGCAGTATCTGTAGAAGCAA |
| | | AGTCTCTAGTAATTACAAAAAGTGTAAGATC |
| SEQ ID NO: 253 | 141293_11669 | AAAGTCACTTGAAGTATAACAAAGTTCACCGCTAT |
| | | TACAGGCCACCACCCAACCGAGGAAGTTTCAATTG |
| | | GAGCTGAGTTGTATATTCATTGATAAGCAAG |
| SEQ ID NO: 254 | 141293_5695 | TCTAGATGGCTCGCTAGTACTATAACTTTCTTCTC |
| | | GAATCACACGTTCAGGAGGAAAACAACTGAATTCT |
| | | CTTAGAGCGACAGACCTAACTCGGCATCCTC |
| SEQ ID NO: 255 | 137284_22272 | CGAGCTCCACAAGTAGTTGAGTTTTATCATTCACT |
| | | AATGAAGAGAGATTCTAGGAAGGATTCTTCAAATG |
| | | GAGGGATATGTGATGTTCCAGATGTTGCCAA |
| SEQ ID NO: 256 | Cannabis.v1_ | TTTTTTTTTTAGTTTTAGCTTTCTTTGGCACTTCT |
| | scf2253- | AGTTGAGCTAATAATAGTTTGACTTATTGAAGATA |
| | 57653_100 | GTACTTTGGTGAATGGTTCAATTGAAGCAAG |
| SEQ ID NO: 257 | 140888_17485 | AAAGTAGAACCCGTTTCGAAAGCACCATGTAGAAT |
| | | GGCTCCATCAGAACTCAAGGAGCTAGAAATACAAC |
| | | TTCAAGGAATGCTTGATTTAGGCTTTATCCG |
| SEQ ID NO: 258 | 140888_133719 | AAAGCTCTAAAACTAACACGAATTGAAAACATGAA |
| | | AAGTGCACGAAACTGGTTTTTGATCTGATTAACCG |
| | | GTTTTACCAGGCCTGTAAAACTGGTTAACCG |
| SEQ ID NO: 259 | 140888_207098 | AATCTTCTAATTTACTAAAAAAAAATATAGTGGAT |
| | | ATTGTTACAGTAGATTAGAGAATATATTGTGGGTA |
| | | TATATTGTTAGACATCATCAAGGAGGTATTG |
| SEQ ID NO: 260 | 140888_324605 | AAGTATTGTGTTGGCTGCTGGAGCTGTTCCATGGG |
| | | TTTCTCTTCATATAATATTCTTTTTGTGTTTTATG |
| | | TGTGACTATAAAAACTCATGCTGTTTTGTGG |
| SEQ ID NO: 261 | 140888_332783 | TTAGTCCCTAATTAATACAAGAATCTTGTATGTAT |
| | | TTTGCAAGTTATTTTCCCACTTGCATGGAAATTGG |
| | | AATAGTCATTTTGGAGGGGAGCACAAGAGAA |
| SEQ ID NO: 262 | 140888_394798 | ATAAATGAATGGGTTGCTTTCACATGGGGAAGGAA |
| | | GGATTCTCTTTTTGTAAAAGAGAGGAAGTGGTCAG |
| | | AGAATCCCAGTTAAGTTTTGTTGTTTTGTGA |
| SEQ ID NO: 263 | 140888_563703 | ACTTTATTCTAAAATATATCCCATAACCCCTGTTA |
| | | CAGTTCCATTCCTTATTATAATTAGAAAAATGATT |
| | | CACTAAGCAACAACAAATTATGTAACAAAAA |
| SEQ ID NO: 264 | 199432_1874 | GATTATGGTTTTATTACACACGTACAGATGGAATT |
| | | GATAGTTCATATGGATTGTGGTGGTTGCGAAAGCA |
| | | AGATAAAGAGAGCTCTTCTAAACTTAGAAGG |
| SEQ ID NO: 265 | 140888_669626 | TCTATGGAATTCATGGCAGGATTATCTTCAACGTG |
| | | AAATCCAGGAGCTTTTGTTTCTGCCTGGCTCTTTA |
| | | TAGATACTTCTTCGGAAAGGCTTTTCCATGC |
| SEQ ID NO: 266 | 140888_701986 | CGAGGAATGAATGACACAACACAATTGGAAAACAA |
| | | AGGTATCAAAGCACGGCATTCATGGAACACAGGGA |
| | | AAGTACTCCAAGCAAATGGTAGAACACCCGA |
| SEQ ID NO: 267 | 142269_6486 | GTCGATGATATGTTGGTAAAGTCGGTATAATGTGA |
| | | AGACCATAGTCGCCATACAAAGGACCTCGCAGAAT |
| | | GTTTCGAAATTCTAAAAAAGTACAATATGAA |
| SEQ ID NO: 268 | 142269_27530 | ACATGTTTCAAGTGCTGCAATTCTTGTACAAGTAA |
| | | TTTATTACCTCCACATTGAACTTGACCCATCACTA |
| | | TGTTTTCGGACACTCCACATCCAAACATGTT |
| SEQ ID NO: 269 | 142269_50192 | TATGACGACATCAGTAAAGATGCAAGGAAATTCTT |
| | | GACTCCCGATGTCCCGGAAGATGAGATTTATTAAA |
| | | GCTTTTGGTGTTGATACAAGGGTACATTATT |
| SEQ ID NO: 270 | 142269_100184 | ATCAATGATCTTAAGAACTCGCCTGGACCCTCTAA |
| | | GAAGGAGCTTCGAATCGCAACAAAGCAAGTTGTGG |
| | | GGCTATTACAGGACAATTACCCTGAGTTGGT |
| SEQ ID NO: 271 | 142269_102855 | AACTTAAATTAGTGAGGCAAGGAGGAATCATCCCA |
| | | GTGAAGCTATTATTGGCCAAATCTAGTACACTGAT |
| | | TGTTTCTAGCTTGCAAATCAAAGAAGGTAGC |
| SEQ ID NO: 272 | 142269_104610 | TTGCAGAGGAGGTAATGAAAAAGTTGGATTGAGAA |
| | | CAAAATAGGAGAAGGTGAAAACATAATAGGCTAAA |
| | | TGATATAACTTCATATTAATCTTTTAAAATT |
| SEQ ID NO: 273 | 142269_135398 | ACTATTAAAATATGGATACATATATTCCAATCTTC |
| | | CAATTTTCTTTCAAAATTACCTCTCTTTTCAATAC |
| | | CCCCATTCTCTCTAGCTCCCTTTTTCTATCC |
| SEQ ID NO: 274 | 142269_279954 | GCCAAGAGGTCATCTAGTCAGATCATGATCAAAGA |
| | | AAAACTCACTAATGCCGCTTATTTGAAAATTAAAA |
| | | ACATGCGCCTATTCTTCTATCAGGCTAATAT |
| SEQ ID NO: 275 | 142269_389457 | AAAGTCCAAAGAGGAGAAAAAGGCAGACACCTTTT |
| | | TCCTTTCTTTTAATTGTTTCTTTCCTTTGTCTTTT |
| | | ATTCCCTCATCATTGTACCTCCCCCACACAC |
| SEQ ID NO: 276 | 140250_15736 | CCCCAAGGGTGTGGGATATGTGCAAGGAGTCTAAT |
| | | CAAACTCACGAGAGTGCTATAATATAGCACTGAAG |
| | | CTCGCAAAAAAGAAGAAATCAATAAATGTTA |
| SEQ ID NO: 277 | 140250_4497 | AGCATTTCGGATTTTGGATTTTGTTTAATTAATTA |
| | | ATATTATTTTTCTCTTTCTGGTCAGATTCTCCATC |
| | | ACTGATGACCTACCCCCTCTTATCAGCTTTA |
| SEQ ID NO: 278 | 140726_298459 | TTTGAGTTCCCTCTTGGAAACTTGAACTTGTTCCA |
| | | ATTCTATCATTTGTGACTGTCTTTACAGTCAACTG |
| | | GAACTAGGCACAAAGTGCTAACAAGCTTCTC |
| SEQ ID NO: 279 | 140726_288665 | AAAACCTAACATTGAGAAGAGCTTGATCAGACACA |
| | | CCACAAGCCTTCTAAGTACAACACTCTGAGAAGAA |
| | | CTAGTCTCTCCTTCTAAAGATATATACCTAA |
| SEQ ID NO: 280 | 107140_596 | ACTACTTTGCGGCTTTTCTCACTTGGTTTTGATGT |
| | | CTTGATGATAAAAAAGTATATTTGCAAAGAATCTC |
| | | ATTCCCCTTACTTGTGGCCGACCTGTGCCAC |
| SEQ ID NO: 281 | 126812_8470 | TGGGCGGGTTTGAGAAAGTCCCACCCAAGTACTCA |
| | | GTACACACTCTCCCAGTTTCCCCTCTGGAAGTAAC |
| | | CCAACAATCATTCCTATCTAGAATACTACCA |
| SEQ ID NO: 282 | un105509_43_89 | CTTGTAGGTATGCATCTTGAACTTTCGAGTGATGA |
| | | GGAGGTCATGGATTTCTTATGAGAAGGCAGACCCT |
| | | CTCCTCCACTTCAATTCTAAGTCAGTTAGCT |
| SEQ ID NO: 283 | 140726_146295 | GCACAAGATTATCTTCTACATGTGTCTTTTTCTTG |
| | | CTATATGGATTTTTAACTATTAACATATATTTTGT |
| | | ATTCCTTTTAATAAATTTTTTCTTTTTACCA |
| SEQ ID NO: 284 | 140726_143382 | ATATTTTTCATTCTTTCATTCATTTTTCTCTCACA |
| | | ACTTTGTTCATCAACACCTTTAAACGAGTTCCTAA |
| | | AATGCTTCCAAAAAAACTCTAATAATAACTC |
| SEQ ID NO: 285 | 140726_136911 | TTAATGTAACAAATTATTACATGAATCTTTCAAGA |
| | | CAATTGTACAAGAAGCTGGCTCCCTATGTTTCAAC |
| | | CAATCCTAGAGAAACTTTCTTGAATTATCGA |
| SEQ ID NO: 286 | 140726_99547 | AAGGAAGATAAGATGTTAGTTCCCTATTAATATTC |
| | | CCAGTGGAGCAGACTTTTTTTGGTGATCTAAGTTG |
| | | AGTGTTCAGTTATTTGAACATCAGTATCTTA |
| SEQ ID NO: 287 | 140726_92974 | ATTTGGGGATGAAGGAGGGTACGAGTATTGACACC |
| | | ACTACTAGTACCAGTACCACCAAAACATGTGGATT |
| | | TACTGGATTTACTATTGTCTGCATTTGAGCG |
| SEQ ID NO: 288 | 141735_113420 | TCAAGCGGCCATAATATGGACTCCATATGGTGGAA |
| | | AGATGAGCGTGATTCCGGAATCTGATTCCCCTTTC |
| | | CCTCATAGGAAAGGAATCATTTTCATGAGTC |
| SEQ ID NO: 289 | 138184_13409 | TATCAATGCCCCCACATGATTCAAAATTGACATAA |
| | | TGTATGTTCTCTTATTTATTTTCCATGTCTGAATG |
| | | TAAGAGATAAAATAAGTTTTTATTTTAACAT |
| SEQ ID NO: 290 | 141735_269885 | AGTAGTGAGGGTATGCAATAGGGCCTAAGGGCTCC |
| | | CCACAATTGTAATTGCTGTACTCATACATATAATA |
| | | CATACATTTTTAGTGTTTGGCTACTATTTTG |
| SEQ ID NO: 291 | 141735_437148 | GTATTGACTAATTGTAAAATAAATTGTTATTTTTA |
| | | GTGTCATCATCATATCGTGTTTGTGTGGTGTGGAG |
| | | TTTTTAAATGTATTTTAGATGGGCAACAAAA |
| SEQ ID NO: 292 | 141735_492163 | TCAAATATAAAATTTTATAAAAAAAGTTACTATAC |
| | | GATGCAACATGATAAATACAAAACTATACAAAAAC |
| | | ACGAAAGAAGCACAAACATACCTATGAATAA |
| SEQ ID NO: 293 | 170870_14856 | TAAGAAAATGGAGGTTGGTTATTTAACCTTATATC |
| | | ACTATATAAGATTTATATATTTCTTTTCTATAAAG |
| | | TCTTCACACCAATTGATACGTTGGAGCATAT |
| SEQ ID NO: 294 | 140997_363509 | TACACAACTGATCAGCACGAGACTTTTCTTCCAGT |
| | | GCTTTCTTTCGGTTCATTTCTGAAAGCTTTTTATG |
| | | TTGTTCTGAATTCAACTTCTCCCTGTCAGCC |
| SEQ ID NO: 295 | 140997_427582 | ATTGATTTTTTCCTTGTTGCGATGAAGCCACCTTA |
| | | GATTTTTGTGGCTCAGCATGGCTTGACTAGTGAAA |
| | | CTAAAGAACAAATAAATGTAACTCATAACAT |
| SEQ ID NO: 296 | 140997_464201 | TTAAGGTTTGTTTTAAATTAAATTTCATTATTGAA |
| | | TTTATTCTTGTCATTTGCATCTCCTTCCTTTGGAG |
| | | CATATTTCAACCTTTCATGTTACCATGTACA |
| SEQ ID NO: 297 | 140997_467859 | GTTAATGGCTTCCCGTGATGTAAGTAGTAGTGAGT |
| | | AACATCATACATAACATATCCTAATAAAATCCCTC |
| | | CGAACAATGCTGGAGCAACTGATGGTGATGA |
| SEQ ID NO: 298 | 133080_7333 | AAATCGTCAGAAATTGTAACTCAGTCCTAATTAAT |
| | | TACGGATATAGATAACAGATGTAATACTCATCACA |
| | | ATTGCCGTACAGTGTTTGAGTGTCTTTTACG |
| SEQ ID NO: 299 | 140997_628227 | GCAAACCTGATCCATGGCCAACCAAAATTTGTTCT |
| | | TGGCCCAGGTACTCTGAATTGTTGATCAGATTTTA |
| | | AGCATCAGGTGTAAGGTGATTTGTTGCACCT |
| SEQ ID NO: 300 | 140997_656987 | ATATAGGGGCATCACCCTTCAAGTATTTTCACTGC |
| | | AAATTCTACTTATCACTTAGCTAAATCTCGTTTCT |
| | | TTTCACATGTTCCTTAATCTTCAAACGCTTC |
| SEQ ID NO: 301 | 140997_859069 | GCTGCCATTGATTCTCTTAATGCTTATTGTGGTCT |
| | | AAGTATGGTTATCAGGGGACTCTTTAGTCGCTTGG |
| | | AAAGTAGAAGAAAATTGCATTCCAGAATGGG |
| SEQ ID NO: 302 | 142086_207043 | AAAGACTCTCTCTTCCTTCTCACCTATTTGAACCC |
| | | TAGAAACCAGCAACAGCAAAGAAGATTTCTCCACC |
| | | AATTTCACAATTTCTTAGCTGAATTGACAAG |
| SEQ ID NO: 303 | 142086_326496 | GATGTTGCATCCATGCTTGACTATTCCCATCGTCA |
| | | AACATGCCTCGTAATAAGCCACTAAGAGCTCGCCA |
| | | TTGCTAGACCTTCACAACCCCTATTATTGAA |
| SEQ ID NO: 304 | 142086_537884 | CTTTAAGGTAGGCTATGTTCTGTTTGAGTGGGGAT |
| | | CCTATCCTTTGTTTCAAGATTGTGTTAATTGTTTC |
| | | AAGGAGTTTGTGTTGGGTTTTATGCCCTAAA |
| SEQ ID NO: 305 | 142086_556735 | AGTTAGTTAGTTAGTATTTTAGCTCGATTTTCTCA |
| | | CTTCTTTGGAATCCTTGTTTAGGTGTCGATCTGGA |
| | | GAGAGATTCAAATGAAAAGATTAAGATTGAG |
| SEQ ID NO: 306 | 142086_642992 | AGTCCTCATTGGTTTTTGATGCAAATCTGCTATGA |
| | | GAGCTGAGTTGCAGCCTCCATATCATCAATTCCCC |
| | | TTGATGTTGTAGACTTTTCTATGGAGCTGCC |
| SEQ ID NO: 307 | 142086_769641 | TGGGTTAATGCTTATTTATTTGCAGTCCGTTTGGT |
| | | TGCCGAGAAAGTGCTATAAAGATAAAGATTCTGCT |
| | | TTCTTTTTTTGCTTAGCAAGAAGAATACCCT |
| SEQ ID NO: 308 | 142086_783150 | AAATTCTGGTAAAAAAAATTCCATCAAGCATTGTT |
| | | TACTACTTTTGCTAAATTTTTTGCTTCTATTAATG |
| | | ATTTCTATGATTTCTTTTTTATTGGGGTTCC |
| SEQ ID NO: 309 | 142086_818053 | TATTTTTGTAAAGATTGGACCAAGAATCAGTATTT |
| | | TTGTAAATTTCCTTTGATATATTGTTTTAAACCTA |
| | | CCACTAATACTATTAATGCATATAAGACTTC |
| SEQ ID NO: 310 | 142086_826368 | AATTTTTTTGAACAACCCTAATTTGTGTGTGTTGT |
| | | GTTTTGGTTCTTAGGGTTTCTAAACTTTTCTTGAG |
| | | AAGTCTTTTAAGCCGTGGGTTCAAACGACAC |
| SEQ ID NO: 311 | 142086_869588 | TGAAGCAGTGAAGTCTTTTTGAAAAGCACGTGATA |
| | | GTTTCCTTTTCTCCACGAGGAGATTACTACTTCAT |
| | | TTTGGCCTTTGCTTAATGAGGTTGGAAACTT |
| SEQ ID NO: 312 | 142086_875282 | CTTAATATTCTAGCAAGTATCATGAAGATACATAT |
| | | AGGTAACTGGTTATATGTAAGATAGTAACAAGTTA |
| | | CATTGATGGTAATCGGGTTACCTTATATAAT |
| SEQ ID NO: 313 | 142086_948791 | AAAGCATTTGGTAGCTGGGGATTTTGTATGTGGTC |
| | | CACCAACACATTACAATCAGTTCCTATTTGGTAGC |
| | | TGGGCATAGTGTTGTTGCTCAGTTTTGATAT |
| SEQ ID NO: 314 | 142086_1049946 | TTCTACCTAGCCCCTTGTTCGAAGTAGTAGCCATG |
| | | TCTGAGATAGTGAACGAATTTTGGCAAAAGTATCT |
| | | TAACCAACTTCATCTTCATCCTCTCAGGACC |
| SEQ ID NO: 315 | 123168_1814 | CCCTACCTTATTTCCTTCTGGGTATGATAATGATG |
| | | ATGTTGTCGATGATGTTGATGTTGCTGCCAATGAA |
| | | GAAGAAGAAGAAAATGATGATGATGATGATG |
| SEQ ID NO: 316 | 142086_1157635 | ATGATTTTTACGTGGTTTGAGTGAATGAACTCTAG |
| | | TCCACGAATTAATAGTATTAAGCTAGAAAAGTTTT |
| | | GAAATATTTCATACAAGTATTTACGCCATAG |
| SEQ ID NO: 317 | 142086_1161186 | ACCAATGGAGGGCTCTAACTCTTGGCAATCTCTAC |
| | | TAATCTGCAATTATGAAAACATAAGAATGAGATAA |
| | | TGTATTTGTGAGTGTGTAACAGTGAACCAGA |
| SEQ ID NO: 318 | 142086_1171094 | ATGCTTGCTCGATGGGTGCAATAATTTTCATCATA |
| | | ACCCCATGCAAGGGTCGGAGAACCATCAAACTCAC |
| | | ATCGAACTAACATCGGGCCTGTGCTTTTTTT |
| SEQ ID NO: 319 | 142086_1178755 | ACTTCACATAACCTCTAAATAGATAAGTTTTGTGC |
| | | TAGCCCAAAGCCTACTCCTCCAAGCACCACCAACA |
| | | AAGTCAGAAGTCAAAACCTTGTTAAACTCTA |
| SEQ ID NO: 320 | 142086_1211275 | CAAGAACACATTGTTTAGATTCAAGTTTATTTATT |
| | | TTCAATATTTTCTTTATAGCGAATAGAGTAAACTT |
| | | TAACATCACCAATCAAAGATTTGGTAGCACG |
| SEQ ID NO: 321 | 142086_1215230 | AGGAAAACGGGTACTCCTGATAGCAGGGATTCCAA |
| | | GTTCATTGTTCTTCCTCAGGTGGAGATAAAAGCTA |
| | | GTGATGATGTGGTGAGTACTACGGTTATCTA |
| SEQ ID NO: 322 | 142086_1258351 | CTCTCTCTCGCTTCAGCTCTCTTTCTCGATAACTT |
| | | CATCAAGATGAAGATCATCGCCGCTTACTTGTTAG |
| | | CCGTTTTGGGTGGAAACGCTGCTCCTTCCGC |
| SEQ ID NO: 323 | 102958_690 | CCATTTTTTGGCTAGTTTGCAGTATGTTATTGGCT |
| | | CATTTGTGCATGGATTCAACTTCAAAGAGTGTTAT |
| | | TATAAATTTTCACAGATAAGTTTTAATCTTG |
| SEQ ID NO: 324 | 141318_699916 | AAAAACCAAAGTGGAACTGTAATTGCATACCCATA |
| | | CCAACCATCGCTGGTGCCCCAATAAGCAGAAGTAC |
| | | CAATAGAAGACCCCAAAATCACTACAAGGAA |
| SEQ ID NO: 325 | 141318_680241 | GTGAGAGTTGCAGCAAGTGTTCCAATAGGAATTGA |
| | | ACGCTGAGTGTCTTTCAGTGAGGCTGATCGATTAG |
| | | AACCTGCCATAATTCCTGTTACAGCAGGGAA |
| SEQ ID NO: 326 | 141318_610006 | AGAATTCTCCGAAGGAGAACCCCTATCCTGGGATG |
| | | TATCTTCGAATTCCACAACCATTCCCATAAAATTT |
| | | TCTTTGGCTGAAAACGAAGCTCTTGATCTAA |
| SEQ ID NO: 327 | 141318_572197 | GCAGCCTTACAGAAACAATTGGAAATTGAAAAGAC |
| | | TCAATATCAAAATTCTGTGCAGGCAACCAAAGCCA |
| | | TGACTTTAAACAATCTGAAAACAAGTCTCCC |
| SEQ ID NO: 328 | 141318_522267 | TCGCCAAGTTTATTTTGTTCACTGAAATTTTTGGT |
| | | TGCATGTTTCAAATCATTGTACTTAAAATTTACTG |
| | | GACCTTGTAATTCTGTACCTCCCAATATGTT |
| SEQ ID NO: 329 | 141318_415904 | GGGATGCTAAGGTCAGCTTTGGACTTGCCTCTTAT |
| | | TTTCTTACTCCATCACTATAAGATCTATAAGTTAA |
| | | GGAGTCCATATTTTGATACTCTCCATTATAT |
| SEQ ID NO: 330 | 141318_404726 | GGGGGATGAAATAATAAATTTTTTGATGATACAGA |
| | | GGACAAATAAAGTTTCGGGGTAGCAACTTGGATTT |
| | | AACGAGCTAAAGGATTTAAGGGTGTATCATT |
| SEQ ID NO: 331 | 141318_395600 | GAGTTCACTACAGTCCTCACAACAACAAGTCACAT |
| | | GCATGGATTGGATACTGTATAATATACAATAATAT |
| | | AAAGACGACCAGACCAGACTTTGCATACTGA |
| SEQ ID NO: 332 | 141318_381127 | ACTAGACATGATTAGCCAAAAAGAATTTGCCAGTG |
| | | TTGCAAACACCTAAACCGAATAGAGGAGTCAGCAA |
| | | TTAAATCTTGTGAAATTTGTTTCTACTTATG |
| SEQ ID NO: 333 | 124011_4556 | ATTAGAGACTACTTTGCTCGGAATAACTTCGATAT |
| | | TTCACCTTATGAAATCATTATCAATCTTGCTTGTT |
| | | AAGGCCTCGTCTTGCTCATTAGATGAGACAC |
| SEQ ID NO: 334 | 141318_337651 | GTCCTCCAAATGACAGCATTGGGCTTTAATGGCAT |
| | | GTTTAGTATGTACTCGTATGCTTCCTTAACAAGAC |
| | | CAGCTCTGCCCAACAAATCAATCATGCAACC |
| SEQ ID NO: 335 | 141318_264113 | ACATATGCAGTTCCTATACAGTAGTATACAGTATT |
| | | ATCATCTGAGAAAGAACAGCTAAGTACGGAGCAGC |
| | | CGTACTCAAATGTATCTAGGGGATAAGTTGA |
| SEQ ID NO: 336 | 141318_269266 | CTAAGATAGCAATATAATACTAGTTCAATGTCAAA |
| | | CTTAAACCTTGTAACAAGCTCAGATGCTTCAGCAT |
| | | CCCATTGGAGAACACAGAATTTGTATCTTTC |
| SEQ ID NO: 337 | 142593_25086 | GTAACCAAGAATGACAGACCTTCCAAACAAAATGT |
| | | TTAATTTTAGGAGGCCCCATTACCTTCCACAAAGA |
| | | AGTCCACCATGAATTCATCCGTCGAGGATCA |
| SEQ ID NO: 338 | 142593_339238 | TCAACAAGCTTTTTCACTTCTTTCATTACTTGTTC |
| | | CGAAGCACTAGACTTATTGTACCTAGTGGATTCAC |
| | | TCTTAGAAGAGTAAATGCTCAAAAACCCATG |
| SEQ ID NO: 339 | 142593_351738 | GAGCAAGGGAAAAGAAGAACATGGTGTGTTTTAAG |
| | | TTATACCCCAGGGAATTCACAGTTAGGTAGCTCGG |
| | | AAATCAAGGAAGAGGCGCAGCTTCTGCGGAA |
| SEQ ID NO: 340 | 142593_394425 | CGGAAAGGAGACCATTTTCTAGGCAAGGGCAGGCC |
| | | CAACCCAACCCCCAACGTTGACGACAAATAGCAAT |
| | | TTCGACCTGACGACTGAGTAGTACGAACTTA |
| SEQ ID NO: 341 | 142593_405698 | ACTAGGAGTGGTGTGTACACAAATTCCTTCACTCC |
| | | AAAGTAACCAAACATTGTCACTTGGTATAATATCA |
| | | TAGCTGCAAGGATTCGAAATACCATGTGTGG |
| SEQ ID NO: 342 | 142593_432737 | CAGACAAAAGAATGTGCTGACTTCAATGTTGTAAC |
| | | ACAATTGAAAGTATTAGTTTAGTGATCTAATTCAC |
| | | TAACAATTATACTTTAGAAAAGATTAAATAT |
| SEQ ID NO: 343 | 142593_437322 | CGGTTGAAGAAATTAACAAGAAATTACTGAAATTG |
| | | AGGTAAGTTTTTCCTTACTTTCTATCGGTTCTCTT |
| | | TCAACTGAAGAGAAAAGATAGATAGGATAAT |
| SEQ ID NO: 344 | 142593_461393 | AACCTTACCCATTCCAATTCCATTCAAGTTTTCAT |
| | | TCCCAGAAAGGTAATGAACTTCTAAGCTTTCAAAA |
| | | TTCTCATGTTTTATCCTTTTGTCTAGAAAGA |
| SEQ ID NO: 345 | 142593_581741 | GATGCTGTTATGTAGGTTGCTGCATCAACAAACAG |
| | | GGAAGTGGATGCACATATTCCTAACTACCCTAGCT |
| | | TACCGCCACAGCTTATTTGTCAACTTCACAA |
| SEQ ID NO: 346 | 142593_611955 | AAAAAATATAATAAATATATTATTACAATATTAAA |
| | | TGTTGTAGATATATGTTTTTAAATAAAAAATGTTA |
| | | CCTAGGGATGCACACGGGGGGGGAAATGGGC |
| SEQ ID NO: 347 | 142593_650845 | CAACTGTAATAAAAGAAAGCAGTTCCAGCGATCAA |
| | | GAAGAATTGCACAAACAATAAAATCACGAGCAACT |
| | | AAGAAGGCACGATACTCCTACGCTGGGCAGC |
| SEQ ID NO: 348 | 142593_660489 | GGTTGTTTAGGTTGAGGTTGATCAAATATTTGATT |
| | | AGTTAACATGGAAGCCACTTGTGCCATGGAAGGTC |
| | | TATCAGAAGCAAAGTCTTGAACACAAAGAAG |
| SEQ ID NO: 349 | 142593_985262 | GAAAGTAACGTAATATCAGCCTTCAAATATTGTAC |
| | | ATTTGTGGTAGTGGACTGTCCCACTGCATACAATG |
| | | TTATCTTGGGACGACCTGCTCTAGTAGACTT |
| SEQ ID NO: 350 | 142593_1664077 | ACACAAGCACGATAGAAGAAAATCTTAAACTATCT |
| | | CAAGCCGATCACTGCAAACACAAAATACCATGTCA |
| | | CAAAACAAGACGCTTATATTATATTTTCCAC |
| SEQ ID NO: 351 | Cannabis.v1_ | TCGGTCCACTGGTTTTACAACAAACAAACCAATCA |
| | scf7184- | AACATCAAGACAAAATGTACAAAAGAAACATTTCA |
| | 962_100 | ACACAAATGAAAAGCATTCAAATACCTGTTA |
| SEQ ID NO: 352 | 171_7957663 | CCATTTATGCCACTAATATCTCTTATTTTAATACT |
| | | ATGAAATGGGAACTCATGTGGCTTCGATACAAATT |
| | | ATTCATAGTTGCTGTAATATGCAAATGCAGT |
| SEQ ID NO: 353 | 171_8121469 | CCTTTTCTTTCTTCTCTAGCTATGTAGGTTGTTGC |
| | | ACACACCGAAAGGAGCATGAGATTTTAAAGTAATA |
| | | AAAGGGTTTGAATATTTTAATATCAGACCCT |
| SEQ ID NO: 354 | 171_8499463 | TTATTTATTTTGAGGATTTGTGATTGATATTAATT |
| | | CAATCAACAAATCAACTAGGAAATGCATTATGTCC |
| | | ATTGTCCATGTCGAACTTGATCCTTCCATGT |
| SEQ ID NO: 355 | 171_8589723 | TCAAGTTTAATTCAAAGGTTATGGAAAAATGGAGA |
| | | AATGTAAACGAGTTTCGAGATCAATGCAATACCTC |
| | | TAGATACATGTCCACAGCACGATAAACCTCA |
| SEQ ID NO: 356 | 171_8689494 | AGAGCTCGTCTTTTGGTTGATGACAGTGAGGGAGA |
| | | AAATGAAATGGAGGACGATGAACAGACTCTTTCGA |
| | | AAACCGAATCTACATTTGAGGATCTGTGTGG |
| SEQ ID NO: 357 | 171_14699246 | AATGAGTCGAGACAGTTCAGAATCTATGAAAATGG |
| | | TGAGTTTTGGAATGGTGATGACAAACCTTTTACTC |
| | | CTAAGTATTTGCAAACCACTACTTTATCCAG |
| SEQ ID NO: 358 | 142415_2566909 | TTTACCCAAAAAAAAACATGATAAGAAAAGAGAGG |
| | | AAGAATTGGTTGAGTGTCAATCTAAACACATGACA |
| | | TCATCAATGCATGCAATGTGTGTGTTACATG |
| SEQ ID NO: 359 | 424_524674 | TGTAAGGCTTGTGGTGGCATTTTCTTTGGGTTACA |
| | | GATGGACAAAAAGGTCGCTGAGAGAACCTTAAGGG |
| | | AGCACAAAGGTGATGCTGTTGCTGCTATTAG |
| SEQ ID NO: 360 | 424_3044082 | TACCACACATGGTCGAATCTGGGCTAAGACGGATT |
| | | AGACCTGCGCCTAAGATCCACCAAACTATGGGGCT |
| | | CAAAAAATATATTTTTTAATTAAAATTATAC |
| SEQ ID NO: 361 | 80270_136 | GACGCTGGCAGGGAATTTCGGAAGCACGATACATC |
| | | TGTCAAGAAGGGGCAGAGATCTTGATTGGAAAGGG |
| | | TGATATTGATTTGTGGGATAGGCCTTGGAAA |
| SEQ ID NO: 362 | 142293_8653733 | AGTGCTGCCAGCAAGGAAGTCGTGAGAGCGAGCCC |
| | | TGAGCTGCCCTCAATTCAAGATGATGCTTCGGAGG |
| | | TTTTCTTGGACCCGTGCGATGTACTGGAACC |
| SEQ ID NO: 363 | 142293_8670653 | ACCATAAAATAACTGGAAAATGCGGTATTAGGATT |
| | | TTGCTTCCATCTAAAAGGTGGAACAGAATAGAGAG |
| | | CTCCAACCAAAATACTAACACAATACAGAGA |
| SEQ ID NO: 364 | 142293_9139942 | AACACTAAAGCGATGCCTTCATCAATGGCTGCTCT |
| | | TCCAACTTGAGGCGACCCTTCAAAGGAACTTTGAC |
| | | CGTCCGGTTCTTTGAAATAGGCTACAACATT |
| SEQ ID NO: 365 | 142293_9300135 | AATTCAACTTTAATGGTTTTTGTTATTTATGGTTC |
| | | TAGAATTACCAAATCTTGTTTGAATTTGTGTCCCC |
| | | AAAACAGAAATTATATATATGTGCTACATAG |
| SEQ ID NO: 366 | 142293_9329697 | TGTTGGGGTTACAACTTCTCTAACATTTGTAGCGA |
| | | CTTTAATTTTGTTGGGCCCAAAATGTAGCCAAGTT |
| | | GTGTAATGATCTCTTAGCCTATTTTGTTGGG |
| SEQ ID NO: 367 | 142293_9343100 | AAAAAGTACTCAACCATGACACAAGTTTCCACTAA |
| | | ACTTCATGGTATTGCCAAATACTTTTCGATTTGCT |
| | | TTTCATTTTTAATTTGCCAACTTGAATTTTG |
| SEQ ID NO: 368 | 141076_384537 | AATAGGGTTCTATGGGGTCTTGGATTTGCGAGCTT |
| | | CTTGAGCCATGATTTCAAAATCCAAGTCACCTAGT |
| | | GAAAAAGTAACAGTGATTGTAGGTAAAAACA |
| SEQ ID NO: 369 | un5002_64_65 | ATTTCAGGGAAGTGGAATCTCCACCAACTGCTTGT |
| | | AAGTCATTTATCTAGTTCCTTAGCAAAGTTTTCTT |
| | | AGCTATCCTTGCAAAATATTTTGGACTTGAG |
| SEQ ID NO: 370 | 170_1217515 | AGAGTGAGACAGAAACGAGTGGCTGCAGTTATGAG |
| | | TTCCATAGGCCAAAGCATATTGATGGCATTCACTG |
| | | TCACCATGAACAAATACGCTTCTTCAAATAT |
| SEQ ID NO: 371 | 170_3873246 | GGAAAAAAAAATTGTTTTCTAAATTAGATTGCAAG |
| | | AACAAAAATTATACAATCAAAATCATTCAAACTAC |
| | | CAATCAAGTCATACGAATAATCATTCAAATT |

TABLE 28
Comparison of physical and genetic positions for linkage map
SNPs inside the low recombinogenic region on chromosome 7.

| SNP | Position (bp) | Position (cM) |
|---|---|---|
| 142465_122071 | 48,484,097 | 40.242 |
| 142465_44455 | 48,587,800 | 40.242 |
| Cannabis.v1_scf8762-3895_100 | 48,685,524 | 40.242 |
| 141293_760753 | 48,806,170 | 40.242 |
| 141293_620463 | 48,949,721 | 40.242 |
| 141293_474861 | 49,114,154 | 40.242 |
| Cannabis.v1_scf2913-43590_99 | 49,294,749 | 40.242 |
| 141293_165411 | 49,478,177 | 40.242 |
| 141293_11669 | 49,628,859 | 40.242 |
| 137284_13456 | 49,653,821 | 40.242 |
| 137284_19732 | 49,661,572 | 40.242 |
| 140888_36259 | 49,870,778 | 40.242 |
| 140888_106681 | 49,952,765 | 40.242 |
| 140888_166494 | 50,016,225 | 40.242 |
| 140888_242320 | 50,119,685 | 40.242 |
| 140888_262002 | 50,139,367 | 40.242 |
| 140888_309099 | 50,190,890 | 40.242 |
| 140888_471184 | 50,388,971 | 40.242 |
| 140888_575969 | 50,528,030 | 40.413 |
| 199432_1874 | 50,582,962 | 40.413 |
| 140888_696769 | 50,657,361 | 40.413 |
| 140888_729340 | 50,689,281 | 40.413 |
| 142269_104610 | 50,822,516 | 40.413 |
| 142269_135398 | 50,854,826 | 40.413 |
| 142269_141638 | 50,861,059 | 40.413 |
| 142269_264262 | 51,022,401 | 40.413 |
| 142269_279954 | 51,054,719 | 40.413 |
| 142269_389457 | 51,173,524 | 40.413 |
| 142269_462851 | 51,262,584 | 40.413 |
| 140250_15736 | 52,285,032 | 40.413 |
| 140250_4497 | 52,296,271 | 40.413 |
| 140726_298459 | 52,322,834 | 40.413 |
| 140726_288665 | 52,332,628 | 40.413 |
| 107140_596 | 52,412,182 | 40.413 |
| 126812_12506 | 52,435,669 | 40.413 |
| 140726_102031 | 52,552,192 | 40.413 |
| 140726_92974 | 52,561,249 | 40.413 |
| 141735_240214 | 53,202,016 | 40.413 |
| 141735_320834 | 53,283,191 | 40.583 |
| 132241_2253 | 53,396,185 | 40.583 |
| 141735_502635 | 53,519,257 | 40.583 |
| 141735_686668 | 53,733,437 | 40.583 |
| 140997_31928 | 53,951,467 | 40.583 |
| 140997_269499 | 54,202,331 | 40.753 |
| 140997_308933 | 54,252,600 | 40.753 |
| 170870_14856 | 54,375,898 | 40.753 |
| 170875_4272 | 54,382,604 | 40.753 |
| 140997_357365 | 54,394,199 | 40.753 |
| 140997_375794 | 54,412,627 | 40.753 |
| 140997_455597 | 54,500,708 | 40.753 |
| 140997_462559 | 54,507,757 | 40.753 |
| 140997_467859 | 54,513,057 | 40.753 |
| Cannabis.v1_scf6303-13839_101 | 54,533,789 | 40.753 |
| 132368_5391 | 54,608,549 | 40.753 |
| 140997_628227 | 54,693,540 | 40.753 |
| 140997_718310 | 54,781,485 | 40.753 |
| 140997_736720 | 54,799,921 | 40.753 |
| 120592_7080 | 54,881,146 | 40.753 |
| 137156_6696 | 55,029,197 | 40.753 |
| 142086_207043 | 55,277,957 | 40.753 |
| 142086_314166 | 55,431,865 | 40.753 |
| 142086_381103 | 55,506,824 | 40.753 |
| 142086_444384 | 55,578,032 | 40.753 |
| Cannabis.v1_scf3975-33501_101 | 55,608,584 | 40.924 |
| 142086_486862 | 55,628,130 | 40.924 |

First column: SNP name; Second column: location on Abacus (version CsaAba2) reference genome (bp); Third column: location on the linkage map (cM).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the invention as defined in the appended claims.

1. A method for selecting one or more Cannabis plants having one or more modified cannabinoids, the method comprising:
(i) obtaining nucleic acids from a sample plant or its germplasm;
(ii) detecting one or more markers that indicate the modified cannabinoids; and
(iii) selecting the one or more plants comprising the one or more markers indicating the modified cannabinoids.

2. The method of claim 1, further comprising crossing the one or more plants comprising the one or more markers indicating the modified cannabinoids to produce one or more F1 or additional progeny plants.

3-17. (canceled)

18. The method of claim 1 wherein the modified cannabinoids correlate to the ratio of total CBC to the total THC, total CBD, total CBG, total THCV, total CBDV, total CBC, and total CBGV or their acidic cannabinoid forms thereof.

19. The method of claim 18, wherein the one or more markers comprises a polymorphism relative to a reference genome at nucleotide position:
(a) 52923743 on chromosome 7; or
(b) 55970630 on chromosome 7; or
(c) 55984088 on chromosome 7; or
(d) 56018989 on chromosome 7; or
(e) 56032988 on chromosome 7; or
(f) 56076209 on chromosome 7; or
(g) 56171548 on chromosome 7; or
(h) 56301604 on chromosome 7; or
(i) 56426824 on chromosome 7; or
(j) 56430375 on chromosome 7; or
(k) 56440283 on chromosome 7; or
(l) 56488125 on chromosome 7; or
(m) 56492081 on chromosome 7; or
(n) 56538007 on chromosome 7; or
(o) 56700085 on chromosome 7; or
(p) 56782341 on chromosome 7; or
(q) 56872960 on chromosome 7; or
(r) 56910768 on chromosome 7; or
(s) 56967275 on chromosome 7; or
(t) 57069404 on chromosome 7; or
(u) 57080583 on chromosome 7; or
(v) 57089709 on chromosome 7; or
(w) 57104188 on chromosome 7; or
(x) 57120122 on chromosome 7; or
(y) 57152672 on chromosome 7; or
(z) 57228643 on chromosome 7; or
(aa) 57233796 on chromosome 7; or
(ab) 58275951 on chromosome 7; or
(ac) 58538433 on chromosome 7;
wherein the reference genome is the Abacus Cannabis reference genome (version CsaAba2).

20. The method of claim 19, wherein the nucleotide position comprises:
(a) a C/C or C/A genotype at position 52923743 on chromosome 7;
(b) a A/A or A/T genotype at position 55970630 on chromosome 7;
(c) a A/A or A/T genotype at position 55984088 on chromosome 7;
(d) a G/G or G/A genotype at position 56018989 on chromosome 7;
(e) a G/G or G/A genotype at position 56032988 on chromosome 7;
(f) a C/C or C/T genotype at position 56076209 on chromosome 7;
(g) a A/A or A/G genotype at position 56171548 on chromosome 7;
(h) a G/G or G/A genotype at position 56301604 on chromosome 7;
(i) a T/T or T/G genotype at position 56426824 on chromosome 7;
(j) a G/G or A/G genotype at position 56430375 on chromosome 7;
(k) a C/C or C/T genotype at position 56440283 on chromosome 7;
(l) a G/G or A/G genotype at position 56488125 on chromosome 7;
(m) a G/G or T/G genotype at position 56492081 on chromosome 7;
(n) a C/C or C/A genotype at position 56538007 on chromosome 7;
(o) a C/C or T/C genotype at position 56700085 on chromosome 7;
(p) a G/G or G/C genotype at position 56782341 on chromosome 7;
(q) a C/C or C/A genotype at position 56872960 on chromosome 7;
(r) a T/T or T/C genotype at position 56910768 on chromosome 7;
(s) a A/A or A/T genotype at position 56967275 on chromosome 7;
(t) a C/C or C/T genotype at position 57069404 on chromosome 7;
(u) a C/C or C/T genotype at position 57080583 on chromosome 7;
(v) a A/A or T/A genotype at position 57089709 on chromosome 7;
(w) a C/C or C/T genotype at position 57104188 on chromosome 7;
(x) a T/T or C/T genotype at position 57120122 on chromosome 7;
(y) a A/A or G/A genotype at position 57152672 on chromosome 7;
(z) a G/G or A/G genotype at position 57228643 on chromosome 7;
(aa) a A/A or A/G genotype at position 57233796 on chromosome 7;
(ab) a A/A or T/A genotype at position 58275951 on chromosome 7; or
(ac) a C/C or C/T genotype at position 58538433 on chromosome 7;
wherein the reference genome is the Abacus Cannabis reference genome (version CsaAba2).

21. (canceled)

22. The method of claim 18, wherein the one or more markers comprises a polymorphism at position 26 of any one or more of: SEQ ID NO: 321; SEQ ID NO: 322; SEQ ID NO: 323; SEQ ID NO: 324; SEQ ID NO: 326; SEQ ID NO: 327; SEQ ID NO: 328; SEQ ID NO: 329; SEQ ID NO: 330; SEQ ID NO: 331; SEQ ID NO: 332; SEQ ID NO: 333; SEQ ID NO: 334; SEQ ID NO: 335; SEQ ID NO: 336; SEQ ID NO: 343; and SEQ ID NO: 348.

23. The method of claim 22, wherein the nucleotide position comprises:
(a) a C/C or C/A genotype at position 51 of SEQ ID NO: 288;
(b) a A/A or A/T genotype at position 51 of SEQ ID NO: 307;
(c) a A/A or A/T genotype at position 51 of SEQ ID NO: 308;
(d) a G/G or G/A genotype at position 51 of SEQ ID NO: 309;
(e) a G/G or G/A genotype at position 51 of SEQ ID NO: 310;
(f) a C/C or C/T genotype at position 51 of SEQ ID NO: 311;
(g) a A/A or A/G genotype at position 51 of SEQ ID NO: 313;
(h) a G/G or G/A genotype at position 51 of SEQ ID NO: 314;
(i) a T/T or T/G genotype at position 51 of SEQ ID NO: 316;
(j) a G/G or A/G genotype at position 51 of SEQ ID NO: 317;
(k) a C/C or C/T genotype at position 51 of SEQ ID NO: 318;
(l) a G/G or A/G genotype at position 51 of SEQ ID NO: 320;
(m) a G/G or T/G genotype at position 51 of SEQ ID NO: 321;
(n) a C/C or C/A genotype at position 51 of SEQ ID NO: 322;
(o) a C/C or T/C genotype at position 51 of SEQ ID NO: 323;
(p) a G/G or G/C genotype at position 51 of SEQ ID NO: 324;
(q) a C/C or C/A genotype at position 51 of SEQ ID NO: 326;
(r) a T/T or T/C genotype at position 51 of SEQ ID NO: 327;
(s) a A/A or A/T genotype at position 51 of SEQ ID NO: 328;
(t) a C/C or C/T genotype at position 51 of SEQ ID NO: 329;
(u) a C/C or C/T genotype at position 51 of SEQ ID NO: 330;
(v) a A/A or T/A genotype at position 51 of SEQ ID NO: 331;
(w) a C/C or C/T genotype at position 51 of SEQ ID NO: 332;
(x) a T/T or C/T genotype at position 51 of SEQ ID NO: 333;
(y) a A/A or G/A genotype at position 51 of SEQ ID NO: 334;
(z) a G/G or A/G genotype at position 51 of SEQ ID NO: 335;
(aa) a A/A or A/G genotype at position 51 of SEQ ID NO: 336;
(ab) a A/A or T/A genotype at position 51 of SEQ ID NO: 343; or
(ac) a C/C or C/T genotype at position 51 of SEQ ID NO: 348;
wherein the reference genome is the Abacus Cannabis reference genome (version CsaAba2).

24. The method of claim 18, wherein the one or more markers comprises a polymorphism relative to a reference genome within any one or more haplotypes and wherein the haplotypes comprise the region:
(a) between positions 52544592 and 53396185 on chromosome 7;
(b) between positions 55939712 and 56081903 on chromosome 7;
(c) between positions 55939712 and 56081903 on chromosome 7;
(d) between positions 55939712 and 56081903 on chromosome 7;
(e) between positions 55939712 and 56081903 on chromosome 7;
(f) between positions 55939712 and 56081903 on chromosome 7;
(g) between positions 56166983 and 56434732 on chromosome 7;
(h) between positions 56166983 and 56434732 on chromosome 7;
(i) between positions 56166983 and 56434732 on chromosome 7;
(j) between positions 56166983 and 56434732 on chromosome 7;
(k) between positions 56434732 and 56593122 on chromosome 7;
(l) between positions 56434732 and 56593122 on chromosome 7;
(m) between positions 56434732 and 56593122 on chromosome 7;
(n) between positions 56434732 and 56593122 on chromosome 7;
(o) between positions 56620519 and 56809638 on chromosome 7;
(p) between positions 56620519 and 56809638 on chromosome 7;
(q) between positions 56809638 and 57276534 on chromosome 7;
(r) between positions 56809638 and 57276534 on chromosome 7;
(s) between positions 56809638 and 57276534 on chromosome 7;
(t) between positions 56809638 and 57276534 on chromosome 7;
(u) between positions 56809638 and 57276534 on chromosome 7;
(v) between positions 56809638 and 57276534 on chromosome 7;
(w) between positions 56809638 and 57276534 on chromosome 7;
(x) between positions 56809638 and 57276534 on chromosome 7;
(y) between positions 56809638 and 57276534 on chromosome 7;
(z) between positions 56809638 and 57276534 on chromosome 7;
(aa) between positions 56809638 and 57276534 on chromosome 7;
(ab) between positions 58244327 and 58353998 on chromosome 7; or
(ac) between positions 58428139 and 58607780 on chromosome 7;
wherein the reference genome is the Abacus Cannabis reference genome (version CsaAba2).

25-31. (canceled)

32. The method of claim 1, wherein:
(i) the modified cannabinoids correlate to an increased ratio of the combination of total THC, total CBD, total CBG, total THCV, total CBDV, total CBC, and total CBGV to the combination of total CBG and CBGV;
(ii) the modified cannabinoids correlate to an increased ratio of the combination of total THC and total THCV to the combination of total CBG and total CBGV; or
(iii) the modified cannabinoids correlate to an increased ratio of total CBC to the combination of total CBG and total CBGV.

33. The method of claim 32, wherein:
(i) the modified cannabinoids correlate to an increased ratio of the combination of total THC, total CBD, total CBG, total THCV, total CBDV, total CBC, and total CBGV to the combination of total CBG and CBGV, and the one or more markers comprises a polymorphism relative to a reference genome at nucleotide position:
(a) 32398208 on chromosome 1; or
(b) 1276491 on chromosome 3; or
(c) 4023145 on chromosome 6; or
(d) 6147814 on chromosome 7; or
(e) 7857512 on chromosome 7; or
(f) 9595805 on chromosome 7; or
(g) 9668980 on chromosome 7; or
(h) 18616969 on chromosome 7; or
(i) 20028244 on chromosome 7; or
(j) 29686440 on chromosome 7; or
(k) 34956997 on chromosome 7; or
(l) 35637532 on chromosome 7; or
(m) 35653590 on chromosome 7; or
(n) 36003907 on chromosome 7; or
(o) 37994590 on chromosome 7; or
(p) 38736896 on chromosome 7; or
(q) 41296609 on chromosome 7; or
(r) 41986329 on chromosome 7; or
(s) 42051322 on chromosome 7; or
(t) 42152092 on chromosome 7; or
(u) 42289736 on chromosome 7; or
(v) 42415983 on chromosome 7; or
(w) 42793823 on chromosome 7; or
(x) 44942488 on chromosome 7; or
(y) 46093742 on chromosome 7; or
(z) 48399881 on chromosome 7; or
(aa) 48949721 on chromosome 7; or
(ab) 49294749 on chromosome 7; or
(ac) 50582962 on chromosome 7; or
(ad) 50745259 on chromosome 7; or
(ae) 50820763 on chromosome 7; or
(af) 50854826 on chromosome 7; or
(ag) 51054719 on chromosome 7; or
(ah) 51173524 on chromosome 7; or
(ai) 52285032 on chromosome 7; or
(aj) 52296271 on chromosome 7; or
(ak) 52322834 on chromosome 7; or
(al) 52332628 on chromosome 7; or
(am) 52439705 on chromosome 7; or
(an) 52554676 on chromosome 7; or
(ao) 52561249 on chromosome 7; or
(ap) 54400345 on chromosome 7; or
(aq) 58233054 on chromosome 7; or
(ar) 59740097 on chromosome 7; or
(ii) the modified cannabinoids correlate to an increased ratio of the combination of total THC and total THCV to the combination of total CBG and total CBGV, and the one or more markers comprises a polymorphism relative to a reference genome at nucleotide position:
(a) 32398208 on chromosome 1; or
(b) 1276491 on chromosome 3; or
(c) 1845149 on chromosome 5; or
(d) 2038965 on chromosome 5; or
(e) 2364964 on chromosome 5; or
(f) 4023145 on chromosome 6; or
(g) 6147814 on chromosome 7; or
(h) 7085058 on chromosome 7; or
(i) 7857512 on chromosome 7; or
(j) 8656466 on chromosome 7; or
(k) 9595805 on chromosome 7; or
(l) 9625781 on chromosome 7; or
(m) 9668980 on chromosome 7; or
(n) 9716257 on chromosome 7; or
(o) 18616969 on chromosome 7; or
(p) 20028244 on chromosome 7; or
(q) 24653415 on chromosome 7; or
(r) 25703806 on chromosome 7; or
(s) 29420823 on chromosome 7; or
(t) 29686440 on chromosome 7; or
(u) 30033193 on chromosome 7; or
(v) 30196905 on chromosome 7; or
(w) 30306362 on chromosome 7; or
(x) 30318939 on chromosome 7; or
(y) 30411594 on chromosome 7; or
(z) 30696835 on chromosome 7; or
(aa) 33474698 on chromosome 7; or
(ab) 33580732 on chromosome 7; or
(ac) 33990610 on chromosome 7; or
(ad) 34059134 on chromosome 7; or
(ae) 34956997 on chromosome 7; or
(af) 35637532 on chromosome 7; or
(ag) 35653590 on chromosome 7; or
(ah) 36003907 on chromosome 7; or
(ai) 38736896 on chromosome 7; or
(aj) 41296609 on chromosome 7; or
(ak) 41852842 on chromosome 7; or
(al) 41986329 on chromosome 7; or
(am) 42051322 on chromosome 7; or
(an) 42152092 on chromosome 7; or
(ao) 42289736 on chromosome 7; or
(ap) 42415983 on chromosome 7; or
(aq) 42793823 on chromosome 7; or
(ar) 44700019 on chromosome 7; or
(as) 44942488 on chromosome 7; or
(at) 46093742 on chromosome 7; or
(au) 48399881 on chromosome 7; or
(av) 48949721 on chromosome 7; or
(aw) 49294749 on chromosome 7; or
(ax) 50745259 on chromosome 7; or
(ay) 50820763 on chromosome 7; or
(az) 50854826 on chromosome 7; or
(ba) 51054719 on chromosome 7; or
(bb) 51173524 on chromosome 7; or
(bc) 52285032 on chromosome 7; or
(bd) 52296271 on chromosome 7; or
(be) 52322834 on chromosome 7; or
(bf) 52332628 on chromosome 7; or
(bg) 52412182 on chromosome 7; or
(bh) 52439705 on chromosome 7; or
(bi) 52554676 on chromosome 7; or
(bj) 52561249 on chromosome 7; or
(bk) 52932343 on chromosome 7; or
(bl) 54400345 on chromosome 7; or
(bm) 56018989 on chromosome 7; or
(bn) 56076209 on chromosome 7; or
(bo) 56081903 on chromosome 7; or
(bp) 56448133 on chromosome 7; or
(bq) 57770425 on chromosome 7; or
(br) 58178218 on chromosome 7; or
(bs) 58190719 on chromosome 7; or
(bt) 58233054 on chromosome 7; or
(bu) 58244327 on chromosome 7; or
(bv) 58271366 on chromosome 7; or
(bw) 58317539 on chromosome 7; or
(bx) 58450928 on chromosome 7; or
(by) 58481147 on chromosome 7; or
(bz) 59740097 on chromosome 7; or
(ca) 60366254 on chromosome 7; or
(cb) 32398208 on chromosome 1; or
(cc) 4023145 on chromosome 6; or
(cd) 5908268 on chromosome 7; or
(ce) 7639988 on chromosome 7; or
(cf) 13731414 on chromosome 7; or
(cg) 18616969 on chromosome 7; or
(ch) 30411594 on chromosome 7; or
(ci) 37505904 on chromosome 7; or
(cj) 41659351 on chromosome 7; or
(ck) 44916971 on chromosome 7; or
(cl) 49114154 on chromosome 7; or
(cm) 50745259 on chromosome 7; or
(cn) 50767962 on chromosome 7; or
(co) 50820763 on chromosome 7; or
(cp) 50854826 on chromosome 7; or
(cq) 51054719 on chromosome 7; or
(cr) 51173524 on chromosome 7; or
(cs) 52285032 on chromosome 7; or
(ct) 52296271 on chromosome 7; or
(cu) 52322834 on chromosome 7; or
(cv) 52332628 on chromosome 7; or
(cw) 52554676 on chromosome 7; or
(cx) 52561249 on chromosome 7; or
(cy) 58317539 on chromosome 7; or
(cz) 58528791 on chromosome 7; or
(da) 2366529 on chromosome 5; or
(db) 2899343 on chromosome 5; or
(dc) 3074649 on chromosome 5; or
(dd) 3086874 on chromosome 5; or
(de) 3432405 on chromosome 5; or
(df) 3485895 on chromosome 5; or
(dg) 3535297 on chromosome 5; or
(dh) 3599637 on chromosome 5; or
(di) 3673686 on chromosome 5; or
(dj) 3961139 on chromosome 5; or
(dk) 4118245 on chromosome 5; or
(dl) 4384123 on chromosome 5; or
(dm) 6491377 on chromosome 5; or
(dn) 6666604 on chromosome 5; or
(do) 7129622 on chromosome 5; or
(dp) 7245930 on chromosome 5; or
(dq) 7904873 on chromosome 5; or
(dr) 8078921 on chromosome 5; or
(ds) 9111167 on chromosome 5; or
(dt) 9113994 on chromosome 5; or
(du) 9472805 on chromosome 5; or
(dv) 9565471 on chromosome 5; or
(dw) 9734723 on chromosome 5; or
(dx) 9752109 on chromosome 5; or
(dy) 10155920 on chromosome 5; or
(dz) 10439554 on chromosome 5; or
(ea) 10451559 on chromosome 5; or
(eb) 10469451 on chromosome 5; or
(ec) 10548740 on chromosome 5; or
(ed) 11161216 on chromosome 5; or
(ee) 11245818 on chromosome 5; or
(ef) 11257676 on chromosome 5; or
(eg) 11310169 on chromosome 5; or
(ch) 12446524 on chromosome 5; or
(ei) 12932181 on chromosome 5; or
(ej) 13378138 on chromosome 5; or
(ek) 13758916 on chromosome 5; or
(el) 14847788 on chromosome 5; or
(em) 15194741 on chromosome 5; or
(en) 16033894 on chromosome 5; or
(eo) 18706160 on chromosome 5; or
(ep) 19988534 on chromosome 5; or
(eq) 20017410 on chromosome 5; or
(er) 20148519 on chromosome 5; or
(es) 20537946 on chromosome 5; or
(et) 20790022 on chromosome 5; or
(eu) 21524826 on chromosome 5; or
(ev) 21563573 on chromosome 5; or
(ew) 23962791 on chromosome 5; or
(ex) 23968827 on chromosome 5; or
(ey) 23975759 on chromosome 5; or
(ez) 24897787 on chromosome 5; or
(fa) 25356228 on chromosome 5; or
(fb) 25364836 on chromosome 5; or
(fc) 25387741 on chromosome 5; or
(fd) 25536887 on chromosome 5; or
(fe) 28880563 on chromosome 5; or
(ff) 32311818 on chromosome 5; or
(ga) 32387532 on chromosome 5; or
(gb) 36148442 on chromosome 5; or
(gc) 40532113 on chromosome 5; or
(gd) 42410724 on chromosome 5; or
(ge) 42557566 on chromosome 5; or
(gf) 43499675 on chromosome 5; or
(gg) 44062719 on chromosome 5; or
(gh) 45687721 on chromosome 5; or
(gi) 48977178 on chromosome 5; or
(gj) 52819178 on chromosome 5; or
(gk) 53489757 on chromosome 5; or
(gl) 53719271 on chromosome 5; or
(gm) 54339213 on chromosome 5; or
(gn) 56258287 on chromosome 5; or
(go) 57537902 on chromosome 5; or
(gp) 57827101 on chromosome 5; or
(gq) 58026390 on chromosome 5; or
(gr) 58596336 on chromosome 5; or
(gs) 59820696 on chromosome 5; or
(gt) 60280385 on chromosome 5; or
(gu) 60295701 on chromosome 5; or
(gv) 61249054 on chromosome 5; or
(gw) 64186528 on chromosome 5; or
(gx) 64884077 on chromosome 5; or
(gy) 23406044 on chromosome 7; or
(gz) 38978759 on chromosome 1; or
(ha) 48365472 on chromosome 7; or
(hb) 48366628 on chromosome 7; or
(hc) 48587800 on chromosome 7; or
(hd) 48688297 on chromosome 7; or
(he) 48694159 on chromosome 7; or
(hf) 49044262 on chromosome 7; or
(hg) 49065141 on chromosome 7; or
(hh) 49134977 on chromosome 7; or
(hi) 49139036 on chromosome 7; or
(hj) 49272668 on chromosome 7; or
(hk) 49297082 on chromosome 7; or
(hl) 49304484 on chromosome 7; or
(hm) 49331724 on chromosome 7; or
(hn) 49344499 on chromosome 7; or
(ho) 49394880 on chromosome 7; or
(hp) 49628859 on chromosome 7; or
(hq) 49634834 on chromosome 7; or
(hr) 49664112 on chromosome 7; or
(hs) 49666319 on chromosome 7; or
(ht) 49843878 on chromosome 7; or
(hu) 49979803 on chromosome 7; or
(hv) 50084463 on chromosome 7; or
(hw) 50302149 on chromosome 7; or
(hx) 50515764 on chromosome 7; or
(hy) 50582962 on chromosome 7; or
(hz) 50630217 on chromosome 7; or
(ia) 50662578 on chromosome 7; or
(ib) 50715700 on chromosome 7; or
(ic) 50767962 on chromosome 7; or
(id) 50818092 on chromosome 7; or
(ie) 52439705 on chromosome 7; or
(if) 52441872 on chromosome 7; or
(ig) 52507913 on chromosome 7; or
(ih) 52510830 on chromosome 7; or
(ii) 52517301 on chromosome 7; or
(ij) 52923743 on chromosome 7; or
(ik) 53449873 on chromosome 7; or
(il) 53505022 on chromosome 7; or
(im) 54400345 on chromosome 7; or
(in) 54464358 on chromosome 7; or
(io) 54509399 on chromosome 7; or
(ip) 54513057 on chromosome 7; or
(ig) 54555051 on chromosome 7; or
(ir) 54693540 on chromosome 7; or
(is) 54722302 on chromosome 7; or
(it) 54915189 on chromosome 7; or
(iu) 55277957 on chromosome 7; or
(iv) 55452244 on chromosome 7; or
(iw) 55716705 on chromosome 7; or
(ix) 55735556 on chromosome 7; or
(iy) 55836464 on chromosome 7; or
(iz) 55970630 on chromosome 7; or
(ja) 55984088 on chromosome 7; or
(jb) 56018989 on chromosome 7; or
(jc) 56032988 on chromosome 7; or
(jd) 56076209 on chromosome 7; or
(je) 56081903 on chromosome 7; or
(jf) 56171548 on chromosome 7; or
(ig) 56301604 on chromosome 7; or
(jh) 56426824 on chromosome 7; or
(ji) 56430375 on chromosome 7; or
(jj) 56440283 on chromosome 7; or
(jk) 56488125 on chromosome 7; or
(jl) 56492081 on chromosome 7; or
(jm) 56538007 on chromosome 7; or
(jn) 56700085 on chromosome 7; or
(jo) 56782341 on chromosome 7; or
(ip) 56872960 on chromosome 7; or
(jq) 56910768 on chromosome 7; or
(jr) 56967275 on chromosome 7; or
(js) 57069404 on chromosome 7; or
(it) 57080583 on chromosome 7; or
(ju) 57089709 on chromosome 7; or
(jv) 57104188 on chromosome 7; or
(jw) 57120122 on chromosome 7; or
(jx) 57152672 on chromosome 7; or
(jy) 57228643 on chromosome 7; or
(jz) 57233796 on chromosome 7; or
(ka) 58538433 on chromosome 7; or
(iii) the modified cannabinoids correlate to increased ratio of total CBC to the combination of total CBG and total CBGV, and the one or more markers comprises a polymorphism relative to a reference genome at nucleotide position:
(a) 4023145 on chromosome 6; or
(b) 7639988 on chromosome 7; or
(c) 41659351 on chromosome 7; or
(d) 50854826 on chromosome 7; or
(e) 51054719 on chromosome 7; or
(f) 51173524 on chromosome 7; or
(g) 52296271 on chromosome 7; or
(h) 52554676 on chromosome 7; or
(i) 52561249 on chromosome 7; or
(j) 58528791 on chromosome 7; or
(k) 20017410 on chromosome 5; or
(l) 36148442 on chromosome 5; or
(m) 38978759 on chromosome 1; or
(n) 67769631 on chromosome 1; or
(o) 28081703 on chromosome 7; or
(p) 28685688 on chromosome 7; or
(q) 50822516 on chromosome 7; or
(r) 52923743 on chromosome 7; or
(s) 53505022 on chromosome 7; or
(t) 54400345 on chromosome 7; or
(u) 54464358 on chromosome 7; or
(v) 54509399 on chromosome 7; or
(w) 54513057 on chromosome 7; or
(x) 54555051 on chromosome 7; or
(y) 54693540 on chromosome 7; or
(z) 54722302 on chromosome 7; or
(aa) 54915189 on chromosome 7; or
(ab) 55277957 on chromosome 7; or
(ac) 55452244 on chromosome 7; or
(ad) 56032988 on chromosome 7; or
(ae) 56301604 on chromosome 7; or
(af) 56967275 on chromosome 7; or
(ag) 58538433 on chromosome 7;
wherein the reference genome is the Abacus Cannabis reference genome (version CsaAba2).

34-37. (canceled)

38. The method of claim 32, wherein:
(i) the modified cannabinoids correlate to an increased ratio of the combination of total THC, total CBD, total CBG, total THCV, total CBDV, total CBC, and total CBGV to the combination of total CBG and CBGV, and the one or more markers comprises a polymorphism relative to a reference genome within any one or more haplotypes, wherein the haplotypes comprise the region:
(a) between positions 32382605 and 32414393 on chromosome 1;
(b) between positions 1276073 and 1284641 on chromosome 3;
(c) between positions 4002406 and 4037935 on chromosome 6;
(d) between positions 6125186 and 6165666 on chromosome 7;
(e) between positions 7822322 and 7864334 on chromosome 7;
(f) between positions 9585902 and 9598562 on chromosome 7;
(g) between positions 9662890 and 9677657 on chromosome 7;
(h) between positions 18581032 and 18640383 on chromosome 7;
(i) between positions 20022570 and 20050272 on chromosome 7;
(j) between positions 29661512 and 29686508 on chromosome 7;
(k) between positions 34941497 and 34997619 on chromosome 7;
(l) between positions 35592132 and 35695775 on chromosome 7;
(m) between positions 35592132 and 35695775 on chromosome 7;
(n) between positions 35799742 and 36153842 on chromosome 7;
(o) between positions 37898780 and 38009095 on chromosome 7;
(p) between positions 38729407 and 38794976 on chromosome 7;
(q) between positions 41248971 and 41343647 on chromosome 7;
(r) between positions 41982953 and 41988552 on chromosome 7;
(s) between positions 42043493 and 42096029 on chromosome 7;
(t) between positions 42096029 and 42156112 on chromosome 7;
(u) between positions 42175605 and 42379636 on chromosome 7;
(v) between positions 42379636 and 42418406 on chromosome 7;
(w) between positions 42758132 and 42819971 on chromosome 7;
(x) between positions 44924842 and 44959410 on chromosome 7;
(y) between positions 46090347 and 46097781 on chromosome 7;
(z) between positions 48393114 and 48443534 on chromosome 7;
(aa) between positions 48862965 and 49016897 on chromosome 7;
(ab) between positions 49288761 and 49297082 on chromosome 7;
(ac) between positions 50541967 and 50605020 on chromosome 7;
(ad) between positions 50737827 and 50747700 on chromosome 7;
(ae) between positions 50818092 and 50822516 on chromosome 7;
(af) between positions 50832568 and 50861059 on chromosome 7;
(ag) between positions 51022401 and 51062076 on chromosome 7;
(ah) between positions 51141514 and 51226826 on chromosome 7;
(ai) between positions 51909282 and 52291349 on chromosome 7;
(aj) between positions 52291349 and 52412182 on chromosome 7;
(ak) between positions 52291349 and 52412182 on chromosome 7;
(al) between positions 52291349 and 52412182 on chromosome 7;
(am) between positions 52435669 and 52441872 on chromosome 7;
(an) between positions 52552192 and 52760459 on chromosome 7;
(ao) between positions 52552192 and 52760459 on chromosome 7;
(ap) between positions 54394199 and 54410973 on chromosome 7;
(aq) between positions 58220261 and 58237329 on chromosome 7; or
(ar) between positions 59728563 and 59762135 on chromosome 7; or
(ii) the modified cannabinoids correlate to an increased ratio of the combination of total THC and total THCV to the combination of total CBG and total CBGV, and the one or more markers comprises a polymorphism relative to a reference genome within any one or more haplotypes, wherein the haplotypes comprise the region:
(a) between positions 32379705 and 32422316 on chromosome 1;
(b) between positions 1271584 and 1284641 on chromosome 3;
(c) between positions 1840325 and 1848408 on chromosome 5;
(d) between positions 2011766 and 2065182 on chromosome 5;
(c) between positions 2360380 and 2366529 on chromosome 5;
(f) between positions 4002406 and 4037935 on chromosome 6;
(g) between positions 6125186 and 6165666 on chromosome 7;
(h) between positions 7083154 and 7103045 on chromosome 7;
(i) between positions 7822322 and 7864334 on chromosome 7;
(j) between positions 8652992 and 8669729 on chromosome 7;
(k) between positions 9585902 and 9598562 on chromosome 7;
(l) between positions 9619898 and 9646812 on chromosome 7;
(m) between positions 9662890 and 9677657 on chromosome 7;
(n) between positions 9701317 and 9716591 on chromosome 7;
(o) between positions 18581032 and 18640383 on chromosome 7;
(p) between positions 20022570 and 20050272 on chromosome 7;
(q) between positions 24593497 and 24673734 on chromosome 7;
(r) between positions 25478783 and 25784004 on chromosome 7;
(s) between positions 29416077 and 29482333 on chromosome 7;
(t) between positions 29661512 and 29686508 on chromosome 7;
(u) between positions 29985750 and 30047823 on chromosome 7;
(v) between positions 30115726 and 30346994 on chromosome 7;
(w) between positions 30115726 and 30346994 on chromosome 7;
(x) between positions 30115726 and 30346994 on chromosome 7;
(y) between positions 30346994 and 30489245 on chromosome 7;
(z) between positions 30670182 and 30816363 on chromosome 7;
(aa) between positions 33455822 and 33524411 on chromosome 7;
(ab) between positions 33577092 and 33700189 on chromosome 7;
(ac) between positions 33969097 and 34028788 on chromosome 7;
(ad) between positions 34028788 and 34136591 on chromosome 7;
(ac) between positions 34941497 and 34997619 on chromosome 7;
(af) between positions 35592132 and 35695775 on chromosome 7;
(ag) between positions 35592132 and 35695775 on chromosome 7;
(ah) between positions 35799742 and 36153842 on chromosome 7;
(ai) between positions 38729407 and 38794976 on chromosome 7;
(aj) between positions 41248971 and 41343647 on chromosome 7;
(ak) between positions 41820799 and 41931751 on chromosome 7;
(al) between positions 41982953 and 41988552 on chromosome 7;
(am) between positions 42043493 and 42096029 on chromosome 7;
(an) between positions 42096029 and 42156112 on chromosome 7;
(ao) between positions 42175605 and 42379636 on chromosome 7;
(ap) between positions 42379636 and 42418406 on chromosome 7;
(aq) between positions 42758132 and 42819971 on chromosome 7;
(ar) between positions 44692277 and 44723890 on chromosome 7;
(as) between positions 44924842 and 44959410 on chromosome 7;
(at) between positions 46090347 and 46097781 on chromosome 7;
(au) between positions 48393114 and 48443534 on chromosome 7;
(av) between positions 48870733 and 49016897 on chromosome 7;
(aw) between positions 49288761 and 49297082 on chromosome 7;
(ax) between positions 50724154 and 50747700 on chromosome 7;
(ay) between positions 50818092 and 50822516 on chromosome 7;
(az) between positions 50822516 and 50861059 on chromosome 7;
(ba) between positions 51022401 and 51062076 on chromosome 7;
(bb) between positions 51141514 and 51226826 on chromosome 7;
(bc) between positions 51909282 and 52435669 on chromosome 7;
(bd) between positions 51909282 and 52435669 on chromosome 7;
(be) between positions 51909282 and 52435669 on chromosome 7;
(bf) between positions 51909282 and 52435669 on chromosome 7;
(bg) between positions 51909282 and 52435669 on chromosome 7;
(bh) between positions 52435669 and 52441872 on chromosome 7;
(bi) between positions 52552192 and 52760459 on chromosome 7;
(bj) between positions 52552192 and 52760459 on chromosome 7;
(bk) between positions 52926238 and 53035545 on chromosome 7;
(bl) between positions 54394199 and 54410973 on chromosome 7;
(bm) between positions 56001500 and 56032988 on chromosome 7;
(bn) between positions 56067360 and 56102656 on chromosome 7;
(bo) between positions 56067360 and 56102656 on chromosome 7;
(bp) between positions 56440283 and 56450990 on chromosome 7;
(bq) between positions 57760733 and 57805989 on chromosome 7;
(br) between positions 58146715 and 58181665 on chromosome 7;
(bs) between positions 58186525 and 58197878 on chromosome 7;
(bt) between positions 58220261 and 58237329 on chromosome 7;
(bu) between positions 58237682 and 58252232 on chromosome 7;
(bv) between positions 58266320 and 58275951 on chromosome 7;
(bw) between positions 58305695 and 58324561 on chromosome 7;
(bx) between positions 58450413 and 58456572 on chromosome 7;
(by) between positions 58467957 and 58522091 on chromosome 7;
(bz) between positions 59728563 and 59762135 on chromosome 7;
(ca) between positions 60363767 and 60366472 on chromosome 7;
(cb) between positions 32363260 and 32422316 on chromosome 1;
(cc) between positions 4002406 and 4087346 on chromosome 6;
(cd) between positions 5900788 and 5921575 on chromosome 7;
(ce) between positions 7630020 and 7643910 on chromosome 7;
(cf) between positions 13715325 and 13740309 on chromosome 7;
(cg) between positions 18581032 and 18640383 on chromosome 7;
(ch) between positions 30318939 and 30489245 on chromosome 7;
(ci) between positions 37495060 and 37639835 on chromosome 7;
(cj) between positions 41651577 and 41675164 on chromosome 7;
(ck) between positions 44911280 and 44924842 on chromosome 7;
(cl) between positions 49085607 and 49134977 on chromosome 7;
(cm) between positions 50724154 and 50765250 on chromosome 7;
(cn) between positions 50765250 and 50777808 on chromosome 7;
(co) between positions 50818092 and 50822516 on chromosome 7;
(cp) between positions 50822516 and 50959554 on chromosome 7;
(cq) between positions 51022401 and 51062076 on chromosome 7;
(cr) between positions 51141514 and 51226826 on chromosome 7;
(cs) between positions 51262584 and 52291349 on chromosome 7;
(ct) between positions 52291349 and 52412182 on chromosome 7;
(cu) between positions 52291349 and 52412182 on chromosome 7;
(cv) between positions 52291349 and 52412182 on chromosome 7;
(cw) between positions 52552192 and 52760459 on chromosome 7;
(cx) between positions 52552192 and 52760459 on chromosome 7;
(cy) between positions 58305695 and 58328372 on chromosome 7;
(cz) between positions 58524187 and 58538433 on chromosome 7;
(da) between positions 2364964 and 2534579 on chromosome 5;
(db) between positions 2844078 and 2908474 on chromosome 5;
(dc) between positions 3061958 and 3081773 on chromosome 5;
(dd) between positions 3081773 and 3089662 on chromosome 5;
(de) between positions 3395357 and 3454995 on chromosome 5;
(df) between positions 3454995 and 3493107 on chromosome 5;
(dg) between positions 3526980 and 3541316 on chromosome 5;
(dh) between positions 3585965 and 3604863 on chromosome 5;
(di) between positions 3656543 and 3680073 on chromosome 5;
(dj) between positions 3945751 and 3965771 on chromosome 5;
(dk) between positions 4109676 and 4130254 on chromosome 5;
(dl) between positions 4376633 and 4391586 on chromosome 5;
(dm) between positions 6489377 and 6516042 on chromosome 5;
(dn) between positions 6636230 and 6668100 on chromosome 5;
(do) between positions 7067915 and 7147487 on chromosome 5;
(dp) between positions 7226971 and 7257339 on chromosome 5;
(dq) between positions 7897923 and 7933626 on chromosome 5;
(dr) between positions 8074202 and 8089196 on chromosome 5;
(ds) between positions 9091191 and 9164971 on chromosome 5;
(dt) between positions 9091191 and 9164971 on chromosome 5;
(du) between positions 9453509 and 9478633 on chromosome 5;
(dv) between positions 9542524 and 9570961 on chromosome 5;
(dw) between positions 9718369 and 9771730 on chromosome 5;
(dx) between positions 9718369 and 9771730 on chromosome 5;
(dy) between positions 10143433 and 10200275 on chromosome 5;
(dz) between positions 10402308 and 10486467 on chromosome 5;
(ca) between positions 10402308 and 10486467 on chromosome 5;
(cb) between positions 10402308 and 10486467 on chromosome 5;
(cc) between positions 10531323 and 10586049 on chromosome 5;
(ed) between positions 11131783 and 11193898 on chromosome 5;
(cc) between positions 11217176 and 11252809 on chromosome 5;
(ef) between positions 11252809 and 11322876 on chromosome 5;
(eg) between positions 11252809 and 11322876 on chromosome 5;
(ch) between positions 12444383 and 12472642 on chromosome 5;
(ci) between positions 12829839 and 12944302 on chromosome 5;
(ej) between positions 13375598 and 13383733 on chromosome 5;
(ck) between positions 13754147 and 13766692 on chromosome 5;
(cl) between positions 14759093 and 14860844 on chromosome 5;
(cm) between positions 15085497 and 15201574 on chromosome 5;
(en) between positions 15975544 and 16037097 on chromosome 5;
(eo) between positions 18699991 and 18716207 on chromosome 5;
(ep) between positions 19976402 and 20026964 on chromosome 5;
(cq) between positions 19976402 and 20026964 on chromosome 5;
(cr) between positions 20145940 and 20173122 on chromosome 5;
(cs) between positions 20483019 and 20546290 on chromosome 5;
(ct) between positions 20721287 and 20835904 on chromosome 5;
(cu) between positions 21516323 and 21554970 on chromosome 5;
(cv) between positions 21554970 and 21626778 on chromosome 5;
(cw) between positions 23872960 and 24016565 on chromosome 5;
(ex) between positions 23872960 and 24016565 on chromosome 5;
(cy) between positions 23872960 and 24016565 on chromosome 5;
(cz) between positions 24799598 and 24906306 on chromosome 5;
(fa) between positions 25342928 and 25387614 on chromosome 5;
(fb) between positions 25342928 and 25387614 on chromosome 5;
(fc) between positions 25387614 and 25391623 on chromosome 5;
(fd) between positions 25391623 and 25548921 on chromosome 5;
(fc) between positions 28847049 and 28956941 on chromosome 5;
(ff) between positions 32296352 and 32322620 on chromosome 5;
(ga) between positions 32322620 and 32390660 on chromosome 5;
(gb) between positions 36089359 and 36288826 on chromosome 5;
(gc) between positions 40385082 and 40557005 on chromosome 5;
(gd) between positions 42375856 and 42528615 on chromosome 5;
(gc) between positions 42550360 and 42563140 on chromosome 5;
(gf) between positions 42604248 and 43518393 on chromosome 5;
(gg) between positions 44026725 and 44095222 on chromosome 5;
(gh) between positions 45678680 and 45734373 on chromosome 5;
(gi) between positions 48970067 and 48984121 on chromosome 5;
(gj) between positions 52798015 and 52858896 on chromosome 5;
(gk) between positions 53299267 and 53496045 on chromosome 5;
(gl) between positions 53705956 and 53815660 on chromosome 5;
(gm) between positions 54304731 and 54406395 on chromosome 5;
(gn) between positions 56185902 and 56294577 on chromosome 5;
(go) between positions 57493670 and 57540049 on chromosome 5;
(gp) between positions 57816878 and 57845773 on chromosome 5;
(gg) between positions 57996384 and 58043891 on chromosome 5;
(gr) between positions 58582581 and 58598359 on chromosome 5;
(gs) between positions 59813998 and 59876892 on chromosome 5;
(gt) between positions 60254151 and 60285659 on chromosome 5;
(gu) between positions 60285659 and 60313853 on chromosome 5;
(gv) between positions 61225099 and 61253927 on chromosome 5;
(gw) between positions 64168576 and 64227025 on chromosome 5;
(gx) between positions 64881518 and 64888996 on chromosome 5;
(gy) between positions 23372911 and 23432690 on chromosome 7;
(gz) between positions 38944628 and 39073783 on chromosome 1;
(ha) between positions 48198578 and 49711589 on chromosome 7;
(hb) between positions 48198578 and 49711589 on chromosome 7;
(hc) between positions 48198578 and 49711589 on chromosome 7;
(hd) between positions 48198578 and 49711589 on chromosome 7;
(he) between positions 48198578 and 49711589 on chromosome 7;
(hf) between positions 48198578 and 49711589 on chromosome 7;
(hg) between positions 48198578 and 49711589 on chromosome 7;
(hh) between positions 48198578 and 49711589 on chromosome 7;
(hi) between positions 48198578 and 49711589 on chromosome 7;
(hj) between positions 48198578 and 49711589 on chromosome 7;
(hk) between positions 48198578 and 49711589 on chromosome 7;
(hl) between positions 48198578 and 49711589 on chromosome 7;
(hm) between positions 48198578 and 49711589 on chromosome 7;
(hn) between positions 48198578 and 49711589 on chromosome 7;
(ho) between positions 48198578 and 49711589 on chromosome 7;
(hp) between positions 48198578 and 49711589 on chromosome 7;
(hq) between positions 48198578 and 49711589 on chromosome 7;
(hr) between positions 48198578 and 49711589 on chromosome 7;
(hs) between positions 48198578 and 49711589 on chromosome 7;
(ht) between positions 49711589 and 50350437 on chromosome 7;
(hu) between positions 49711589 and 50350437 on chromosome 7;
(hv) between positions 49711589 and 50350437 on chromosome 7;
(hw) between positions 49711589 and 50350437 on chromosome 7;
(hx) between positions 50350437 and 50528030 on chromosome 7;
(hy) between positions 50528030 and 50822516 on chromosome 7;
(hz) between positions 50528030 and 50822516 on chromosome 7;
(ia) between positions 50528030 and 50822516 on chromosome 7;
(ib) between positions 50528030 and 50822516 on chromosome 7;
(ic) between positions 50528030 and 50822516 on chromosome 7;
(id) between positions 50528030 and 50822516 on chromosome 7;
(ic) between positions 50822516 and 52523987 on chromosome 7;
(if) between positions 50822516 and 52523987 on chromosome 7;
(ig) between positions 50822516 and 52523987 on chromosome 7;
(ih) between positions 50822516 and 52523987 on chromosome 7;
(ii) between positions 50822516 and 52523987 on chromosome 7;
(ij) between positions 52544592 and 53396185 on chromosome 7;
(ik) between positions 53396185 and 54375898 on chromosome 7;
(il) between positions 53396185 and 54375898 on chromosome 7;
(im) between positions 54375898 and 54452078 on chromosome 7;
(in) between positions 54452078 and 54646726 on chromosome 7;
(io) between positions 54452078 and 54646726 on chromosome 7;
(ip) between positions 54452078 and 54646726 on chromosome 7;
(iq) between positions 54452078 and 54646726 on chromosome 7;
(ir) between positions 54683300 and 54714058 on chromosome 7;
(is) between positions 54714058 and 54860264 on chromosome 7;
(it) between positions 54860264 and 55029197 on chromosome 7;
(iu) between positions 55255666 and 55316629 on chromosome 7;
(iv) between positions 55316629 and 55939712 on chromosome 7;
(iw) between positions 55316629 and 55939712 on chromosome 7;
(ix) between positions 55316629 and 55939712 on chromosome 7;
(iy) between positions 55316629 and 55939712 on chromosome 7;
(iz) between positions 55939712 and 56102656 on chromosome 7;
(ja) between positions 55939712 and 56102656 on chromosome 7;
(jb) between positions 55939712 and 56102656 on chromosome 7;
(jc) between positions 55939712 and 56102656 on chromosome 7;
(jd) between positions 55939712 and 56102656 on chromosome 7;
(je) between positions 55939712 and 56102656 on chromosome 7;
(jf) between positions 56166983 and 56434732 on chromosome 7;
(jg) between positions 56166983 and 56434732 on chromosome 7;
(jh) between positions 56166983 and 56434732 on chromosome 7;
(ji) between positions 56166983 and 56434732 on chromosome 7;
(jj) between positions 56434732 and 56593122 on chromosome 7;
(jk) between positions 56434732 and 56593122 on chromosome 7;
(jl) between positions 56434732 and 56593122 on chromosome 7;
(jm) between positions 56434732 and 56593122 on chromosome 7;
(jn) between positions 56620519 and 56809638 on chromosome 7;
(jo) between positions 56620519 and 56809638 on chromosome 7;
(jp) between positions 56809638 and 57276534 on chromosome 7;
(jg) between positions 56809638 and 57276534 on chromosome 7;
(jr) between positions 56809638 and 57276534 on chromosome 7;
(js) between positions 56809638 and 57276534 on chromosome 7;
(jt) between positions 56809638 and 57276534 on chromosome 7;
(ju) between positions 56809638 and 57276534 on chromosome 7;
(jv) between positions 56809638 and 57276534 on chromosome 7;
(jw) between positions 56809638 and 57276534 on chromosome 7;
(jx) between positions 56809638 and 57276534 on chromosome 7;
(jy) between positions 56809638 and 57276534 on chromosome 7;
(jz) between positions 56809638 and 57276534 on chromosome 7; or
(ka) between positions 58428139 and 58607780 on chromosome 7; or
(iii) the modified cannabinoids correlate to increased ratio of total CBC to the combination of total CBG and total CBGV, and the one or more markers comprises a polymorphism relative to a reference genome within any one or more haplotypes, wherein the haplotypes comprise the region:
(a) between positions 4002406 and 4087346 on chromosome 6;
(b) between positions 7630020 and 7643910 on chromosome 7;
(c) between positions 41651577 and 41675164 on chromosome 7;
(d) between positions 50822516 and 50959554 on chromosome 7;
(c) between positions 51022401 and 51062076 on chromosome 7;
(f) between positions 51141514 and 51226826 on chromosome 7;
(g) between positions 52291349 and 52322834 on chromosome 7;
(h) between positions 52552192 and 52760459 on chromosome 7;
(i) between positions 52552192 and 52760459 on chromosome 7;
(j) between positions 58524187 and 58538433 on chromosome 7;
(k) between positions 19988534 and 20026964 on chromosome 5;
(l) between positions 36089359 and 36288826 on chromosome 5;
(m) between positions 38944628 and 39073783 on chromosome 1;
(n) between positions 67761686 and 67892254 on chromosome 1;
(o) between positions 27759260 and 28263307 on chromosome 7;
(p) between positions 28594408 and 29061134 on chromosome 7;
(q) between positions 50818092 and 52439705 on chromosome 7;
(r) between positions 52544592 and 53396185 on chromosome 7;
(s) between positions 53449873 and 54375898 on chromosome 7;
(t) between positions 54375898 and 54452078 on chromosome 7;
(u) between positions 54452078 and 54646726 on chromosome 7;
(v) between positions 54452078 and 54646726 on chromosome 7;
(w) between positions 54452078 and 54646726 on chromosome 7;
(x) between positions 54452078 and 54646726 on chromosome 7;
(y) between positions 54683300 and 54714058 on chromosome 7;
(z) between positions 54714058 and 54860264 on chromosome 7;
(aa) between positions 54860264 and 55029197 on chromosome 7;
(ab) between positions 55255666 and 55316629 on chromosome 7;
(ac) between positions 55316629 and 55716705 on chromosome 7;
(ad) between positions 56018989 and 56076209 on chromosome 7;
(ac) between positions 56171548 and 56426824 on chromosome 7;
(af) between positions 56910768 and 57069404 on chromosome 7; or
(ag) between positions 58428139 and 58607780 on chromosome 7;
wherein the reference genome is the Abacus Cannabis reference genome (version CsaAba2).

39-53. (canceled)

54. The method of claim 1, wherein the detecting comprises use of an oligonucleotide probe.

55. The method of claim 2, wherein the progeny plants comprises the indicated modified cannabinoids.

56. The method of claim 2, wherein the crossing comprises selfing, sibling crossing, or backcrossing.

57-60. (canceled)

61. A method for producing selecting one or more Cannabis plants having modified cannabinoids, the method comprising replacing a nucleic acid sequence of a parent plant with a nucleic acid sequence conferring modified cannabinoids.

62. A processed Cannabis product comprising the progeny plant, or a plant part thereof, of claim 2.

63. (canceled)

64. The product of claim 62, wherein the product is a kief, hashish, bubble hash, an edible product, solvent reduced oil, sludge, e-juice, or tincture.

65. The method of claim 1, wherein the modified cannabinoids correlate to:
(i) elevated levels of the combination of total THC and total THCV; or
(ii) elevated levels of the combination of total CBG and total CBGV.

66. The method of claim 65, wherein:
(i) the modified cannabinoids correlate to elevated levels of the combination of total THC and total THCV, and the one or more markers comprises a polymorphism relative to a reference genome at nucleotide position:
(a) 1605949 on chromosome 1; or
(b) 2187135 on chromosome 1; or
(c) 68168149 on chromosome 3; or
(d) 1981515 on chromosome 7; or
(e) 8445770 on chromosome 6; or
(f) 15287401 on chromosome 6; or
(g) 26717252 on chromosome 6; or
(h) 29145396 on chromosome 6; or
(i) 43665911 on chromosome 6; or
(j) 57867261 on chromosome 6; or
(k) 67382064 on chromosome 6; or
(l) 60624 on chromosome 7; or
(m) 3441223 on chromosome 7; or
(n) 5752776 on chromosome 7; or
(o) 6225622 on chromosome 7; or
(p) 6340996 on chromosome 7; or
(q) 7643910 on chromosome 7; or
(r) 23406044 on chromosome 7; or
(s) 7624628 on chromosome 9; or
(t) 38978759 on chromosome 1; or
(u) 67769631 on chromosome 1; or
(v) 16010588 on chromosome 3; or
(w) 39837146 on chromosome 3; or
(x) 2127802 on chromosome 4; or
(y) 6705244 on chromosome 7; or
(z) 11063067 on chromosome 7; or
(aa) 11067412 on chromosome 7; or
(ab) 17000256 on chromosome 7; or
(ac) 17008461 on chromosome 7; or
(ad) 28081703 on chromosome 7; or
(ae) 28685688 on chromosome 7; or
(af) 30520237 on chromosome 7; or
(ag) 32259550 on chromosome 7; or
(ah) 36589991 on chromosome 7; or
(ai) 37130207 on chromosome 7; or
(aj) 47324655 on chromosome 7; or
(ak) 52923743 on chromosome 7; or
(al) 54375898 on chromosome 7; or
(am) 56032988 on chromosome 7; or
(an) 56301604 on chromosome 7; or
(ao) 56967275 on chromosome 7; or
(ap) 58538433 on chromosome 7; or
(aq) 46475498 on chromosome 9; or
(ii) the modified cannabinoids correlate to elevated levels of the combination of total CBG and total CBGV, and the one or more markers comprises a polymorphism relative to a reference genome at nucleotide position:
(a) 32398208 on chromosome 1; or
(b) 1961209 on chromosome 2; or
(c) 166141 on chromosome 3; or
(d) 174381 on chromosome 3; or
(e) 9387181 on chromosome 4; or
(f) 1173474 on chromosome 5; or
(g) 1181979 on chromosome 5; or
(h) 2038965 on chromosome 5; or
(i) 2208629 on chromosome 5; or
(j) 4023145 on chromosome 6; or
(k) 858349 on chromosome 7; or
(l) 948328 on chromosome 7; or
(m) 954115 on chromosome 7; or
(n) 1155154 on chromosome 7; or
(o) 1928237 on chromosome 7; or
(p) 2243260 on chromosome 7; or
(q) 2612238 on chromosome 7; or
(r) 5348707 on chromosome 7; or
(s) 5851251 on chromosome 7; or
(t) 5900788 on chromosome 7; or
(u) 9079344 on chromosome 7; or
(v) 9272731 on chromosome 7; or
(w) 9421290 on chromosome 7; or
(x) 9595805 on chromosome 7; or
(y) 9598562 on chromosome 7; or
(z) 9619898 on chromosome 7; or
(aa) 9668980 on chromosome 7; or
(ab) 9716257 on chromosome 7; or
(ac) 9868420 on chromosome 7; or
(ad) 9922946 on chromosome 7; or
(ae) 12851206 on chromosome 7; or
(af) 13928304 on chromosome 7; or
(ag) 13933289 on chromosome 7; or
(ah) 14271073 on chromosome 7; or
(ai) 14579677 on chromosome 7; or
(aj) 14940971 on chromosome 7; or
(ak) 15036406 on chromosome 7; or
(al) 15091266 on chromosome 7; or
(am) 15105660 on chromosome 7; or
(an) 15187940 on chromosome 7; or
(ao) 16603812 on chromosome 7; or
(ap) 17355416 on chromosome 7; or
(aq) 17450215 on chromosome 7; or
(ar) 18095653 on chromosome 7; or
(as) 21035025 on chromosome 7; or
(at) 22964819 on chromosome 7; or
(au) 24653415 on chromosome 7; or
(av) 36579046 on chromosome 7; or
(aw) 42289736 on chromosome 7; or
(ax) 44227026 on chromosome 7; or
(ay) 48089881 on chromosome 7; or
(az) 49618132 on chromosome 7; or
(ba) 50213053 on chromosome 7; or
(bb) 50221231 on chromosome 7; or
(bc) 50854826 on chromosome 7; or
(bd) 51054719 on chromosome 7; or
(be) 51173524 on chromosome 7; or
(bf) 52285032 on chromosome 7; or
(bg) 52296271 on chromosome 7; or
(bh) 52554676 on chromosome 7; or
(bi) 52561249 on chromosome 7; or
(bj) 53231544 on chromosome 7; or
(bk) 55716705 on chromosome 7; or
(bl) 56018989 on chromosome 7; or
(bm) 56076209 on chromosome 7; or
(bn) 56309021 on chromosome 7; or
(bo) 56802628 on chromosome 7; or
(bp) 58933090 on chromosome 7; or
(bq) 8502077 on chromosome 8; or
(br) 8679838 on chromosome 8; or
(bs) 9091526 on chromosome 8; or
(bt) 9196738 on chromosome 8; or
(bu) 9309715 on chromosome 8; or
(bv) 21389309 on chromosome 8; or
(bw) 4684198 on chromosome 9; or
(bx) 55358400 on chromosome X; or
(by) 55956503 on chromosome X; or
(bz) 56164045 on chromosome X; or
(ca) 56268500 on chromosome X; or
(cb) 56281906 on chromosome X; or
(cc) 56610941 on chromosome X; or
(cd) 78814483 on chromosome X; or
(ce) 79583866 on chromosome X; or
(cf) 32398208 on chromosome 1; or
(cg) 166081 on chromosome 5; or
(ch) 796653 on chromosome 5; or
(ci) 1181979 on chromosome 5; or
(cj) 2038965 on chromosome 5; or
(ck) 2208629 on chromosome 5; or
(cl) 4023145 on chromosome 6; or
(cm) 1755387 on chromosome 7; or
(cn) 9421290 on chromosome 7; or
(co) 9598562 on chromosome 7; or
(cp) 9619898 on chromosome 7; or
(cq) 9668980 on chromosome 7; or
(cr) 15105660 on chromosome 7; or
(cs) 44227026 on chromosome 7; or
(ct) 50745259 on chromosome 7; or
(cu) 50854826 on chromosome 7; or
(cv) 51054719 on chromosome 7; or
(cw) 51173524 on chromosome 7; or
(cx) 52285032 on chromosome 7; or
(cy) 52296271 on chromosome 7; or
(cz) 52322834 on chromosome 7; or
(da) 52332628 on chromosome 7; or
(db) 52439705 on chromosome 7; or
(dc) 52554676 on chromosome 7; or
(dd) 52561249 on chromosome 7; or
(de) 53231544 on chromosome 7; or
(df) 56018989 on chromosome 7; or
(dg) 56076209 on chromosome 7; or
(dh) 8679838 on chromosome 8; or
(di) 9309715 on chromosome 8; or
(dj) 2690026 on chromosome 9; or
(dk) 78814483 on chromosome X; or
(dl) 122735 on chromosome 2; or
(dm) 188542 on chromosome 2; or
(dn) 1785269 on chromosome 2; or
(do) 48670446 on chromosome 1; or
(dp) 64341256 on chromosome 1; or
(dq) 67666507 on chromosome 1; or
(dr) 2132683 on chromosome 5; or
(ds) 2177531 on chromosome 5; or
(dt) 2366529 on chromosome 5; or
(du) 2899343 on chromosome 5; or
(dv) 3074649 on chromosome 5; or
(dw) 3086874 on chromosome 5; or
(dx) 3485895 on chromosome 5; or
(dy) 3535297 on chromosome 5; or
(dz) 3599637 on chromosome 5; or
(ea) 3961139 on chromosome 5; or
(eb) 4384123 on chromosome 5; or
(ec) 19988534 on chromosome 5; or
(ed) 20017410 on chromosome 5; or
(ee) 20148519 on chromosome 5; or
(ef) 36148442 on chromosome 5; or
(eg) 53489757 on chromosome 5; or
(eh) 81104593 on chromosome X; or
(ei) 38978759 on chromosome 1; or
(ej) 67769631 on chromosome 1; or
(ek) 16010588 on chromosome 3; or
(el) 39837146 on chromosome 3; or
(em) 2127802 on chromosome 4; or
(en) 12788565 on chromosome 6; or
(eo) 6705244 on chromosome 7; or
(ep) 11063067 on chromosome 7; or
(eq) 11067412 on chromosome 7; or
(er) 12636172 on chromosome 7; or
(es) 17000256 on chromosome 7; or
(et) 17008461 on chromosome 7; or
(eu) 28081703 on chromosome 7; or
(ev) 28685688 on chromosome 7; or
(ew) 30520237 on chromosome 7; or
(ex) 32259550 on chromosome 7; or
(ey) 36589991 on chromosome 7; or
(ez) 37130207 on chromosome 7; or
(fa) 52923743 on chromosome 7; or
(fb) 56032988 on chromosome 7; or
(fc) 56301604 on chromosome 7; or
(fd) 56967275 on chromosome 7; or
(fe) 58538433 on chromosome 7; or
(ff) 46475498 on chromosome 9;
wherein the reference genome is the Abacus Cannabis reference genome (version CsaAba2).

67. The method of claim 65, wherein:
(i) the modified cannabinoids correlate to elevated levels of the combination of total THC and total THCV, and the one or more markers comprises a polymorphism relative to a reference genome within any one or more haplotypes, wherein the haplotypes comprise the region:
(a) between positions 1593859 and 1611839 on chromosome 1;
(b) between positions 2183741 and 2194270 on chromosome 1;
(c) between positions 68162527 and 68173892 on chromosome 3;
(d) between positions 1959582 and 2009523 on chromosome 7;
(e) between positions 8437332 and 8460311 on chromosome 6;
(f) between positions 15261229 and 15310117 on chromosome 6;
(g) between positions 26684684 and 26723996 on chromosome 6;
(h) between positions 29097877 and 29158571 on chromosome 6;
(i) between positions 43662000 and 44089274 on chromosome 6;
(j) between positions 57860326 and 57877559 on chromosome 6;
(k) between positions 67379156 and 67421842 on chromosome 6;
(l) between positions 15258 and 115293 on chromosome 7;
(m) between positions 3412885 and 3477646 on chromosome 7;
(n) between positions 5746452 and 5845317 on chromosome 7;
(o) between positions 6221113 and 6256430 on chromosome 7;
(p) between positions 6337560 and 6410830 on chromosome 7;
(q) between positions 7639988 and 7658574 on chromosome 7;
(r) between positions 23372911 and 23432690 on chromosome 7;
(s) between positions 7596844 and 7632863 on chromosome 9;
(t) between positions 38944628 and 39073783 on chromosome 1;
(u) between positions 67761686 and 67892254 on chromosome 1;
(v) between positions 15652280 and 16182525 on chromosome 3;
(w) between positions 39699406 and 40350847 on chromosome 3;
(x) between positions 2082401 and 2155110 on chromosome 4;
(y) between positions 6622471 and 6808016 on chromosome 7;
(z) between positions 10965365 and 11193689 on chromosome 7;
(aa) between positions 10965365 and 11193689 on chromosome 7;
(ab) between positions 16992324 and 17419297 on chromosome 7;
(ac) between positions 16992324 and 17419297 on chromosome 7;
(ad) between positions 27759260 and 28263307 on chromosome 7;
(ae) between positions 28594408 and 29061134 on chromosome 7;
(af) between positions 29891019 and 30608774 on chromosome 7;
(ag) between positions 30608774 and 32279982 on chromosome 7;
(ah) between positions 36579046 and 36880336 on chromosome 7;
(ai) between positions 37049888 and 37211605 on chromosome 7;
(aj) between positions 46967630 and 48198578 on chromosome 7;
(ak) between positions 52544592 and 53396185 on chromosome 7;
(al) between positions 53505022 and 54400345 on chromosome 7;
(am) between positions 56018989 and 56076209 on chromosome 7;
(an) between positions 56171548 and 56426824 on chromosome 7;
(ao) between positions 56910768 and 57069404 on chromosome 7;
(ap) between positions 58428139 and 58607780 on chromosome 7; or
(aq) between positions 46392138 and 46584908 on chromosome 9; or
(ii) the modified cannabinoids correlate to elevated levels of the combination of total CBG and total CBGV, and the one or more markers comprises a polymorphism relative to a reference genome within any one or more haplotypes, wherein the haplotypes comprise the region:
(a) between positions 32414393 and 32414393 on chromosome 1;
(b) between positions 1966129 and 1966129 on chromosome 2;
(c) between positions 168861 and 168861 on chromosome 3;
(d) between positions 175515 and 175515 on chromosome 3;
(e) between positions 9395794 and 9395794 on chromosome 4;
(f) between positions 1194306 and 1194306 on chromosome 5;
(g) between positions 1194306 and 1194306 on chromosome 5;
(h) between positions 2065182 and 2065182 on chromosome 5;
(i) between positions 2288919 and 2288919 on chromosome 5;
(j) between positions 4037935 and 4037935 on chromosome 6;
(k) between positions 866330 and 866330 on chromosome 7;
(l) between positions 975143 and 975143 on chromosome 7;
(m) between positions 975143 and 975143 on chromosome 7;
(n) between positions 1170814 and 1170814 on chromosome 7;
(o) between positions 1932407 and 1932407 on chromosome 7;
(p) between positions 2262669 and 2262669 on chromosome 7;
(q) between positions 2670280 and 2670280 on chromosome 7;
(r) between positions 5389490 and 5389490 on chromosome 7;
(s) between positions 5871508 and 5871508 on chromosome 7;
(t) between positions 5908268 and 5908268 on chromosome 7;
(u) between positions 9082114 and 9082114 on chromosome 7;
(v) between positions 9295164 and 9295164 on chromosome 7;
(w) between positions 9432681 and 9432681 on chromosome 7;
(x) between positions 9609554 and 9609554 on chromosome 7;
(y) between positions 9609554 and 9609554 on chromosome 7;
(z) between positions 9625781 and 9625781 on chromosome 7;
(aa) between positions 9677657 and 9677657 on chromosome 7;
(ab) between positions 9716591 and 9716591 on chromosome 7;
(ac) between positions 9891604 and 9891604 on chromosome 7;
(ad) between positions 9951332 and 9951332 on chromosome 7;
(ae) between positions 12855919 and 12855919 on chromosome 7;
(af) between positions 14002688 and 14002688 on chromosome 7;
(ag) between positions 14002688 and 14002688 on chromosome 7;
(ah) between positions 14288814 and 14288814 on chromosome 7;
(ai) between positions 14592996 and 14592996 on chromosome 7;
(aj) between positions 14945873 and 14945873 on chromosome 7;
(ak) between positions 15048022 and 15048022 on chromosome 7;
(al) between positions 15110059 and 15110059 on chromosome 7;
(am) between positions 15110059 and 15110059 on chromosome 7;
(an) between positions 15223104 and 15223104 on chromosome 7;
(ao) between positions 16609331 and 16609331 on chromosome 7;
(ap) between positions 17363692 and 17363692 on chromosome 7;
(aq) between positions 17459892 and 17459892 on chromosome 7;
(ar) between positions 18109800 and 18109800 on chromosome 7;
(as) between positions 21047228 and 21047228 on chromosome 7;
(at) between positions 22984143 and 22984143 on chromosome 7;
(au) between positions 24673734 and 24673734 on chromosome 7;
(av) between positions 36584469 and 36584469 on chromosome 7;
(aw) between positions 42379636 and 42379636 on chromosome 7;
(ax) between positions 44231810 and 44231810 on chromosome 7;
(ay) between positions 48095767 and 48095767 on chromosome 7;
(az) between positions 49628859 and 49628859 on chromosome 7;
(ba) between positions 50302149 and 50302149 on chromosome 7;
(bb) between positions 50302149 and 50302149 on chromosome 7;
(bc) between positions 50861059 and 50861059 on chromosome 7;
(bd) between positions 51062076 and 51062076 on chromosome 7;
(be) between positions 51226826 and 51226826 on chromosome 7;
(bf) between positions 52291349 and 52291349 on chromosome 7;
(bg) between positions 52322834 and 52322834 on chromosome 7;
(bh) between positions 52760459 and 52760459 on chromosome 7;
(bi) between positions 52760459 and 52760459 on chromosome 7;
(bj) between positions 53283191 and 53283191 on chromosome 7;
(bk) between positions 55731905 and 55731905 on chromosome 7;
(bl) between positions 56032988 and 56032988 on chromosome 7;
(bm) between positions 56081903 and 56081903 on chromosome 7;
(bn) between positions 56329245 and 56329245 on chromosome 7;
(bo) between positions 56806750 and 56806750 on chromosome 7;
(bp) between positions 58943140 and 58943140 on chromosome 7;
(bq) between positions 8507767 and 8507767 on chromosome 8;
(br) between positions 8717782 and 8717782 on chromosome 8;
(bs) between positions 9094186 and 9094186 on chromosome 8;
(bt) between positions 9199573 and 9199573 on chromosome 8;
(bu) between positions 9316329 and 9316329 on chromosome 8;
(bv) between positions 21404217 and 21404217 on chromosome 8;
(bw) between positions 4692766 and 4692766 on chromosome 9;
(bx) between positions 55391085 and 55391085 on chromosome X;
(by) between positions 55962857 and 55962857 on chromosome X;
(bz) between positions 56169204 and 56169204 on chromosome X;
(ca) between positions 56305767 and 56305767 on chromosome X;
(cb) between positions 56305767 and 56305767 on chromosome X;
(cc) between positions 56652118 and 56652118 on chromosome X;
(cd) between positions 78823688 and 78823688 on chromosome X;
(ce) between positions 79599466 and 79599466 on chromosome X;
(cf) between positions 32379705 and 32422316 on chromosome 1;
(cg) between positions 158841 and 176716 on chromosome 5;
(ch) between positions 790537 and 801459 on chromosome 5;
(ci) between positions 1173474 and 1210033 on chromosome 5;
(cj) between positions 2011766 and 2065182 on chromosome 5;
(ck) between positions 2177531 and 2288919 on chromosome 5;
(cl) between positions 4002406 and 4037935 on chromosome 6;
(cm) between positions 1739821 and 1760293 on chromosome 7;
(cn) between positions 9404565 and 9432681 on chromosome 7;
(co) between positions 9595805 and 9609554 on chromosome 7;
(cp) between positions 9609554 and 9625781 on chromosome 7;
(cq) between positions 9662890 and 9677657 on chromosome 7;
(cr) between positions 15091266 and 15110059 on chromosome 7;
(cs) between positions 44203159 and 44231810 on chromosome 7;
(ct) between positions 50724154 and 50747700 on chromosome 7;
(cu) between positions 50822516 and 50861059 on chromosome 7;
(cv) between positions 51022401 and 52322834 on chromosome 7;
(cw) between positions 51022401 and 52322834 on chromosome 7;
(cx) between positions 51022401 and 52322834 on chromosome 7;
(cy) between positions 51022401 and 52322834 on chromosome 7;
(cz) between positions 52296271 and 52332628 on chromosome 7;
(da) between positions 52322834 and 52441872 on chromosome 7;
(db) between positions 52322834 and 52441872 on chromosome 7;
(dc) between positions 52552192 and 52561249 on chromosome 7;
(dd) between positions 52554676 and 52760459 on chromosome 7;
(de) between positions 53210452 and 53283191 on chromosome 7;
(df) between positions 56001500 and 56032988 on chromosome 7;
(dg) between positions 56067360 and 56081903 on chromosome 7;
(dh) between positions 8670961 and 8717782 on chromosome 8;
(di) between positions 9305133 and 9316329 on chromosome 8;
(dj) between positions 2680294 and 2697330 on chromosome 9;
(dk) between positions 78812503 and 78823688 on chromosome X;
(dl) between positions 119953 and 136319 on chromosome 2;
(dm) between positions 181346 and 196868 on chromosome 2;
(dn) between positions 1767164 and 1791640 on chromosome 2;
(do) between positions 48665164 and 48727602 on chromosome 1;
(dp) between positions 64338238 and 64349232 on chromosome 1;
(dq) between positions 67660656 and 67691271 on chromosome 1;
(dr) between positions 2120881 and 2167503 on chromosome 5;
(ds) between positions 2167503 and 2208629 on chromosome 5;
(dt) between positions 2364964 and 2534579 on chromosome 5;
(du) between positions 2844078 and 2908474 on chromosome 5;
(dv) between positions 3061958 and 3081773 on chromosome 5;
(dw) between positions 3081773 and 3089662 on chromosome 5;
(dx) between positions 3454995 and 3493107 on chromosome 5;
(dy) between positions 3526980 and 3541316 on chromosome 5;
(dz) between positions 3585965 and 3604863 on chromosome 5;
(ea) between positions 3945751 and 3965771 on chromosome 5;
(eb) between positions 4376633 and 4391586 on chromosome 5;
(ec) between positions 19976402 and 20026964 on chromosome 5;
(ed) between positions 19976402 and 20026964 on chromosome 5;
(ee) between positions 20145940 and 20173122 on chromosome 5;
(ef) between positions 36089359 and 36288826 on chromosome 5;
(eg) between positions 53299267 and 53496045 on chromosome 5;
(ch) between positions 81098479 and 81109058 on chromosome X;
(ei) between positions 38944628 and 39073783 on chromosome 1;
(ej) between positions 67761686 and 67892254 on chromosome 1;
(ek) between positions 15652280 and 16182525 on chromosome 3;
(el) between positions 39699406 and 40350847 on chromosome 3;
(em) between positions 2082401 and 2155110 on chromosome 4;
(en) between positions 12779880 and 12825612 on chromosome 6;
(eo) between positions 6622471 and 6808016 on chromosome 7;
(ep) between positions 10965365 and 11193689 on chromosome 7;
(eq) between positions 10965365 and 11193689 on chromosome 7;
(er) between positions 12588065 and 12851206 on chromosome 7;
(es) between positions 16992324 and 17419297 on chromosome 7;
(et) between positions 16992324 and 17419297 on chromosome 7;
(eu) between positions 27759260 and 28263307 on chromosome 7;
(ev) between positions 28594408 and 29061134 on chromosome 7;
(ew) between positions 29891019 and 30608774 on chromosome 7;
(ex) between positions 30608774 and 32279982 on chromosome 7;
(ey) between positions 36579046 and 36880336 on chromosome 7;
(ez) between positions 37049888 and 37211605 on chromosome 7;
(fa) between positions 52544592 and 53396185 on chromosome 7;
(fb) between positions 56018989 and 56076209 on chromosome 7;
(fc) between positions 56171548 and 56426824 on chromosome 7;
(fd) between positions 56910768 and 57069404 on chromosome 7;
(fe) between positions 58428139 and 58607780 on chromosome 7; or
(ff) between positions 46392138 and 46584908 on chromosome 9;
wherein the reference genome is the Abacus Cannabis reference genome (version CsaAba2).
